(19) United States
(12) Patent Application Publication
(10) Pub. No.: US 2020/0354777 A1
(43) Pub. Date: Nov. 12, 2020

(54) SINGLE MOLECULE DETECTION OR QUANTIFICATION BY DNA NANOTECHNOLOGY IN MICROWELLS

(71) Applicant: LUDWIG-MAXIMILIANS-UNIVERSITÅT MÜNCHEN, München (DE)

(72) Inventors: Heinrich Grabmayr, München (DE); Johannes Woehrstein, München (DE)

(21) Appl. No.: 16/771,612

(22) PCT Filed: Dec. 14, 2018
(86) PCT No.: PCT/EP2018/085046

§ 371 Date: Jun. 10, 2020

(30) Foreign Application Priority Data
Dec. 14, 2017 (EP) 17207476.7

(51) Int. Cl. C12Q 1/6816; G01N 33/58; B01L 3/00

(52) U.S. Cl. CPC C12Q 1/6816; G01N 33/582; C12Q 2563/159; C12Q 2563/157; B01L 3/50853

(57) ABSTRACT

The invention relates to a method and a DNA nanostructure for detecting a target structure. The invention relates in particular to a DNA nanostructure, which, by skillful selection of the shape of the DNA nanostructure and the placement of marker molecules fastened thereon, ensures a preferably linear dependence on the number of marker molecules and measurement signal, independent of the local arrangement of a plurality of such DNA nanostructures. The invention further relates to using said DNA nanostructures and other nano reporters, preferably in combination with adapters specifically binding to target molecules, in a method for the preferably simultaneous quantification of a plurality of target molecules, by means of a multiplex method. The method relates in particular to a single cell analysis.

The present invention relates to a method and a DNA nanostructure for detecting a target structure. In particular, the present invention relates to a DNA nanostructure, which ensures a, preferably linear, dependence on the number of marker molecules and the measurement signal regardless of the physical arrangement of a plurality of such DNA nanostructures by virtue of the skilled selection of the shape of the DNA nanostructure and the placement of the marker molecules attached to it. The invention additionally relates to the use of said DNA nanostructures and other nanoreporters, preferably in combination with adapters which bind specifically to target molecules, in a method for quantifying a plurality of target molecules using a multiplex method, preferably in a simultaneous manner.

A frequently occurring question in research as well as in applied medicine and biotechnology is the analysis, i.e. the detection, of particular target structures. For example, in the case of sepsis of a patient, the detection of the relevant toxins may be necessary or, in the case of a disease entailing altered cellular constitution, e.g. with cancer, a quantitative analysis of the conversion of genetic information may be necessary.

The quantitative analysis of the conversion of genetic information, the so-called gene expression profiling (GEP) requires the detection and identification of a number of different target mRNA molecules (Stadler, Z. K. et al. Critical Reviews in Oncology Hematology 69, 1-11 (2009)). mRNA is the established abbreviation for the English term messenger RNA (in German “Boten-RNA”), which is also used in German. Since the mRNA sequences are specifically produced in the expression process of the corresponding genes, the gene expression of the analyzed material can be quantified in that manner.

GEP is able to provide information on the state or type of individual cells or also a tissue sample (plurality of cells) and thereby enable, e.g., the molecular diagnosis of tumor tissue. The GEP comprises the analysis of the expression of at least one gene, preferably the analysis of the expression of a plurality of genes. In order to achieve this ultimate objective, a technology is required, which at the same time allows for quantitative analysis having the highest possible specificity and sensitivity, preferably for a number of genes that is as high as possible. For comprehensive applicability in clinical practice, this technology must additionally be characterized by expeditious analysis times, low costs and simple use.

There is a plurality of GEP techniques, which have different advantages and disadvantages, depending on the question at issue.

Microarrays.

GEP analysis using microarrays is based on the surface hybridization of fluorescent DNA molecules to specific, physically separate target sequences on a chip (Trevino, V. et al. Mol Med 13, 527-541 (2007)). These molecules are generated in a preceding step by reverse transcription and polymerase chain reaction (PCR) amplification of RNA molecules. Thereby, microarrays allow for the parallel detection of up to several thousand sequences. However, an exact quantification is impossible, since enzymatic reactions such as reverse transcription and PCR amplification entail systematic errors. Moreover, microarray-based methods require a relatively long time of process of about one day and are therefore not suited for a large number of clinical applications.

qPCR/dPCR.

Using PCR, specific DNA molecules may be exponentially duplicated in an enzymatic process (VanGuilder, H. D. et al. Biotechniques 44, 619-626 (2008)). For PCR reactions, mRNA targets must be generally converted enzymatically using reverse transcription of RNA into DNA. The amplification can be measured by adding fluorescent, DNA-binding molecules and the original concentration can be roughly determined (qPCR). Since the amplification rate significantly depends on various parameters (inter alia target sequence, target length, primer, instruments, reagents, reaction volume), said quantification must be calibrated precisely. For compensation of these disadvantages, digital PCR (dPCR) was developed. It allows for a more precise quantification by compartmentalization of individual target molecules (Baker, M. Nature Methods 9, 541-544 (2012)). However, said high preciseness of dPCR is offset by limitation to typically two targets. dPCR is therefore not suited for GEP analyses.

RNA-Seq/NGS.

With the so-called next generation sequencing (NGS), on which also RNA sequencing (RNA seq) is based, sequences are read out and identified in several steps per base (Wang, Z. et al. Nat Rev Genet 10, 57-63 (2009)). With RNA seq, too, RNA must initially be enzymatically converted into DNA. The advantage and the unique feature of NGS in transcription analysis is the possibility to recognize novel or altered sequences. Due to the great number of complex process steps, the analysis devices are complex and expensive and the process duration is lengthy. This excludes its use in the field of point-of-care. Just as in the above-described methods, enzymatic processes prevent exact quantifiability.

nCounter.

The nCounter system of NanoString Technologies is based on the detection of RNA molecules by hybridization using a fluorescent reporter (barcode), on the one hand, as well as a second DNA molecule (capture strand) for attachment to a microscopy chip, on the other hand (Geiss, G. K. et al. Nat Biotechnol 26, 317-325 (2008)). The fluorescent reporters are linear geometric barcodes that are micrometers in length and that consist of the arrangement of different areas labeled with fluorescence dyes. One of the advantages of the nCounter system is the enzyme-free and quantitative detection of up to 800 different RNA target molecules. One disadvantage of the method is the necessity to stretch the geometric barcode molecule after target hybridization and surface immobilization, in order to be able to read the respective barcode. This has two important implications: (1) The electrophoretic stretching of the reporters is a complex process step, (2) Conditioned by the low efficiency of this step, approximately 80% of the target molecules are excluded due to non-identifiable barcodes. Furthermore, complex and time-consuming purification steps are necessary, e.g., using magnetic particles. In sum, these process steps lead to expensive analysis devices (>200,000 euros) and long measuring times (24-48 hours).

Thus, these traditional methods do not meet all of the above-mentioned preferred requirements for GEP and sacrifice e.g. partial quantifiability for necessary sensitivity.

A further promising analysis approach for GEPs is based on direct labeling of individual target mRNA sequences by so-called hybridization using fluorescent reporters. Fluorescent methods allow for the sensitive detection of single molecules (Joo, C. et al. Annu Rev Biochem 77, 51-76 (2008)). However, established methods are not able to determine a great number of different reporters.

Quantitative GEP on single-cell basis employing the available methods is possible only in a limited manner but would be highly advantageous for both scientific research and industrial research and development as well as for clinical applications.

Important desirable features of a GEP would be the exact quantification and the efficient implementation of the analysis. This results in the following preferred criteria for an improved GEP:
direct detection of the mRNA, in particular no translation into DNA, e.g. by reverse transcription
no amplification of the target molecules, since an amplification that is not precise impairs quantification
thereby: detection of individual target molecules
fast-as-possible analysis (order of minutes)
simple-as-possible analysis, in particular: few working steps
possibility of correlation of analysis results with the original components of the sample

Apart from nucleic acids and proteins, there is a myriad of further problems with which units in the order of a few nanometers to several tens of nanometers must be detected and preferably counted and/or the concentration be determined; such as inorganic complexes and nanoparticles.

All these problems have in common that molecules or other structures should be detected in a fast, simple and exact manner and be preferably counted. Exact counting in particular entails that the probability of the false positive and/or false negative counting events must be kept as low as possible. The present invention aims at an improvement of one or more of said properties, in particular at a reduction of the probability of the false positive and/or false negative counting events.

This problem is solved by the present invention according to the independent claims.

The present invention allows the solution of the aforementioned problems due to a plurality of aspects that are advantageous in comparison to the prior art.

The present invention relates to a method which allows the analysis of individual cells or other structures to be analyzed on this scale or similar scales. One aspect of the method having a particularly advantageous effect in this respect is the isolation of the structures to be analyzed in microwells. This allows the reduction of distortions of the results due to diffusion losses, external input and/or mixing of the structures that actually are to be analyzed individually. The use of the microwells further has the advantage that incorrectly loaded microwells may be quickly and simply discarded, thus resulting in the enhancement of the exactness of the analysis results.

According to a further aspect of the invention, it is possible to analyze a plurality of structures to be analyzed in parallel notwithstanding the individual analysis of the structures to be analyzed.

Compared with the already known methods mentioned at the beginning, the method involves a small number of steps, thus reducing the risk of artifact formation by the analysis process itself.

The steps of the method for isolating the structures to be analyzed are kept advantageously simple which significantly simplifies the execution of the method in comparison with other isolation techniques such as for example the separation into single droplets.

A particularly advantageous aspect of the present invention can be seen in the fact that the individual structures to be analyzed cannot only be analyzed molecularly, but that it is also possible to analyze their phenotype for example with respect to their morphology. These two analysis results may be assigned to each other so that the molecular properties may be assigned to other phenotypical features of the structures to be analyzed allowing further evaluations, such as e.g. correlation analyses.

Thus, the present invention is directed to a method for the detection (preferably the quantification) of a target structure, comprising:
p1) introduction of a group of host bodies including a host body with the target structure into a microwell array such that exactly one host body is present in at least one microwell;
p2) introduction of at least two 3D DNA nanostructures into the at least one microwell wherein each of the 3D DNA nanostructures comprises one or more inwardly disposed fluorescence dye molecules;
a) forming an identification structure in the at least one microwell, comprising:
   (i) the target structure, and
   (ii) the at least two 3D DNA nanostructures, wherein each of the 3D DNA nanostructures is specifically bound to the target structure and wherein the 3D DNA nanostructures are bound to pairwise different regions of the target structure;
b) detection of the target structure by measuring at least one fluorescence signal, wherein the 3D DNA nanostructures and the parameters of the fluorescence measurement are selected such that the at least one measured fluorescence signal of the identification structures formed in a) is distinguishable from the fluorescence signal of each of the at least two isolated 3D DNA nanostructures, when these are not bound in the respective identification structure, wherein the identification structure is bound to a first surface, preferably the bottom of the at least one microwell.

Preferably, the 3D DNA nanostructures, considered individually, have the same fluorescence signal regardless of whether they are present isolated (i.e. not bound in an identification structure) or in an identification structure. Thus, the at least one fluorescence signal measured in step b) differs from the individual fluorescence signals of the unbound (i.e. isolated) 3D DNA nanostructures by virtue of the combination of the at least two 3D DNA nanostructures. Thus, it is not necessary, even though it is also possible, that a 3D DNA nanostructure changes its fluorescence signal by binding in the identification structure, for example by a shift of the spectrum and/or a modification of the intensity.

Herein, host body refers to the structure to be analyzed, which may comprise the target structure to be analyzed. For example, the host body may be a cell, for example a prokaryotic, eukaryotic, bacterial, plant and/or animal cell, a virus, an exosome, a vesicle and/or a droplet. The target structure to be analyzed can be localized on the surface of the host body and/or inside the host body.

Thus, the invention is also directed to a variant of the method in which the target structure is present inside a host body, wherein the method further comprises:
e) disruption of the host bodies, preferably by liquid exchange with a disruption buffer in order to release the target structure from the host body.

In case that the group of host bodies contains cells, vesicles and/or exosomes, the disruption step e) preferably comprises a lysis, particularly preferably a hypotonic or a detergent-based lysis (see e.g. “Thermo Scientific Pierce Cell Lysis Technical Handbook” (2^{nd} ed.), Thermo Scientific; D. Liu and L. Huang, “Tripsin-induced lysis of lipid vesicles: effect of surface charge and lipid composition”, Anal Biochem 1992, 202(1), 1-5; L. Turnbull et al., “Explosive cell lysis as a mechanism for the biogenesis of bacterial membrane vesicles and biofilms”, Nat Commun 2016 Apr. 14; 7:11220; C. Lässer et al., “Isolation and Characterization of RNA-Containing Exosomes”, J Vis Exp 2012, 59 3037 and/or Pin Li et al., “Progress in Exosome Isolation Techniques”, Theranostics 2017, 7(3): 789-804), preferably using a lysis buffer, or a physical lysis, for example a laser-induced lysis (see e.g. MD. Dhawan et al., “Development of a laser-induced cell lysis system”, Anal Bioanal Chem 2002, 373(3): 421-6; K. Rau et al., “Pulsed Laser Microbeam-induced Cell Lysis: Time-Resolved Imaging and Analysis of Hydrodynamic Effects”, Biophys. J. 2006, 91(1): 317-329) or acoustic lysis (see e.g. “Thermo Scientific Pierce Cell Lysis Technical Handbook” (2^{nd} ed.), Thermo Scientific). In case that the group of host bodies comprises droplets of a water-in-oil-in-water emulsion, a method similar to the lysis of cells and/or an equivalent method known to the skilled person can be used (see e.g. M. Chavez-Paez et al., “Coalescence in double emulsions”, Langmuir 2012, 28, 5934-5939). For host bodies in form of droplets of a water-in-oil emulsion, the disruption occurs automatically since such a droplet directly fuses to the hydrophilic microwell boundary, i.e. wall and/or bottom, so that this step of the method only comprises waiting until an incubation period ends, preferably an incubation period of 5-120 minutes, particularly preferably of 15 minutes. In case that the group of host bodies comprises viruses, a method for virus lysis may be used comprising for example a lysis buffer containing for example sodium dodecyl sulfate (SDS) and/or formamide (see e.g. G. F. Steward and A. I. Culley, “Extraction and purification of nucleic acids from viruses”, Manual of aquatic viral ecology, Chapter 16, American Society of Limnology and Oceanography, 2010, pp. 154-165).

The lysis can be carried out e.g. mechanically, by lysis buffer, enzymatically and/or chemically or by light. A method for the lysis of cells that is known in the technical field is mentioned, for example, in “Thermo Scientifi Pierce Cell Lysis Technical Handbook” (2^{nd} ed.), Thermo Scientific) and comprises the use of the surfactant Triton X as lysis buffer. For this reason, details of the method will not be further discussed in the present document. Similar methods are also known for the disruption of droplets. M. Chavez-Paez et al. “Coalescence in double emulsions”, Langmuir 2012, 28, 5934-5939, for example, describes the disruption of droplets using surfactants, for example SDS or Span 80.

Another aspect of the present invention relates to a microwell array. The microwell array isolates the host bodies from one another and localizes the majority (depending of the geometry e.g. 70%, preferably 85%, particularly preferably 99%) of the target structures of the individual host bodies into the associated microwell. The microwell array may be a preferred embodiment of a carrier. In particular the first layer of the microwell array may function as carrier. Carriers will be described in greater detail in the course of the present description and all features which are mentioned in association with a carrier also apply in particular to the carrier embodiment as microwell array.

The microwell array is preferably suited for fluorescence microscopy. The microwell array is an array of wells, i.e. recesses, having preferably dimensions of 10 to 500 micrometers, wherein each well comprises a bottom and a circumferential wall. The bottoms of the microwells are preferably suited for fluorescence microscopy. Each of the microwells preferably comprises an open top side.

The microwell array preferably comprises a first layer which preferably forms the bottoms of the microwells, wherein the first layer is preferably suited for fluorescence microscopy and is preferably a cover slip. All materials known in the technical field, for example glass or special plastics can be considered as materials for the first layer. The microwell array further preferably comprises a second layer which is applied onto the first layer, wherein the second layer forms the walls of the microwells. In a preferred embodiment, the bottoms of the microwells are formed by the first layer and are free of material of the second layer. However, in another embodiment the bottoms of the microwells may be covered, at least in segments, preferably with a thin layer of material of the second layer of 0-5 micrometers, more preferably 0-1 micrometers, even more preferably 0-100 nanometers. In a preferred embodiment, the first layer comprises a material other than that of the second layer.

The material of the second layer is preferably selected in such a way that it has an as low as possible autofluorescence at the wave lengths that are relevant for analysis so that the analysis is not disturbed by the autofluorescence of the microwell array. In a fluorescence measurement according to the analysis method, the fluorescence signal due to the autofluorescence of the second layer is preferably not higher than the fluorescence signal due to a 3D DNA nanostructure with 5 fluorescence molecules, preferably 4 fluorescence molecules, more preferably 3 or less fluorescence molecules.

In order to measure a phenomenological autofluorescence δ of the second layer of a microwell array, the experimental steps of the method of a single-cell gene expression analysis are carried out according to Example 6 described below (i.e. with the exclusion of the evaluation steps described in Example 6) with the difference that the second layer of the microwell array consists of the material to be analyzed. Imaging is carried out using a Nikon Eclipse Ti epifluorescence microscope. The light source is a Sola SE by Lumencor, Inc. Beaverton, Oreg., USA. The camera is a pco.edge 4.2 by PCO GmbH, Kehlheim, Germany. Article F73-832 by AHF Analysentechnik AG, Tubingen, Germany is used as beam splitter. For the blue channel, the excitation and emission filters are F39-480 and F37-527; for the green channel, the filters are F39-563 and F39-637; for the red channel, F39-640 and F37-698 are used. The FI Apochromat TIRF 60× oil 1.49 by Nikon GmbH, Dusseldorf, Germany is used as objective. For measurement, focusing is to the upper surface of the first layer so that the 3D DNA nanostructures are in the focus. For each channel, two images are taken with an exposure time of 1 second with maximum intensity of the light source: one image of an area of a microwell bottom with 3D DNA nanostructures and one image of an area that is filled with the second layer. The phenomenological autofluorescence δ of the second layer is calculated for each channel separately and is calculated based on the ratio of the mean pixel intensity of the image of the second layer and the mean value of maximum pixel values of all 3D DNA nanostructures visible in the image of the relevant channel.

The phenomenological autofluorescence δ is preferably 0-0.1, more preferably 0-10⁻², even more preferably 0-10⁻⁴ and particularly preferably 0-10⁻⁶.

In order to analyze a material, which is to be considered for the second layer of the microwell array, with respect to the autofluorescence that is relevant for the method of the invention, the following considerations regarding absorption and fluorescence are useful:

In general, absorption in homogenous materials, which is caused by absorbing particles, as can be found for example in Demtröder, “Experimentalphysik 2-Elektrizität und Optik”, Springer 1995, ISBN 3-540-57095-0, pp. 212 et seqq. and P. Atkins: Physikalische Chemie, 5^{th} edition, 2013, Wiley-VHC, Weinheim, can be described through the reduction of radiation intensity I

I=I₀*exp(−A)

compared to the initial value I₀, wherein the absorbance A can be represented as

A=b*d=ε_{abs}*c*d N*σ_{abs}

wherein d is the thickness of the irradiated material, b the absorption coefficient, ε_{abs} the concentration related absorption coefficient (usually referred to as absorption coefficient, with the difference between b and ε_{abs} is being clear from the context for the skilled person) and c the concentration of the absorbing type of particles.

In keeping with the scattering cross-section (see e.g. Demtröder, “Experimentalphysik 2-Elektrizität und Optik”, Springer 1995, ISBN 3-540-57095-0, pp. 218 et seqq.), it is possible to define an absorption cross-section σ_{abs} for individual absorbing particles, i.e. a notional surface around the particle within which any incident photon is absorbed. Thus, the absorption is

A=N*σ_{abs}/F=N*σ_{abs}*d/V

wherein N refers to the number of irradiated particles, F to the surface of the irradiated sample, d to the thickness of the irradiated material and V to the volume of the irradiated sample.

These relations allow the correlation of concentration-related absorption coefficient and absorption cross-section:

σ_{abs}=ε_{abs}/N_{A}

wherein N_{A} is the Avogadro constant of 6.022*10²³ mol⁻¹.

If a single fluorescent particle is irradiated with an excitation intensity Iₑₓ, wherein

Iₑₓ=(h*νₑₓ)*Nₑₓₚₕₒₜ/(t*F),

i.e. a flow of Nₑₓₚₕₒₜ photons, the energy of which is h*νₑₓ, over time t across a surface F,

according to the above definition of the absorption cross-section σ_{abs}, in the present case relating to the fluorescent particle designated σ_{abs,fluo},

then N_{absphot}=Nₑₓₚₕₒₜ*σ_{abs,fluo}/F

photons are absorbed over said time. The number of emitted photons Nₑₘₚₕₒₜ is the product of the number of absorbed photons and the fluorescence quantum efficiency η_{fluo} of the fluorescent particle (see e.g. P. Atkins: Physikalische Chemie, 5^{th} edition, 2013, Wiley-VCH, Weinheim):

Nₑₘₚₕₒₜ=η_{fluo}*N_{absphot}

Accordingly, for the emitted photon flow Nₑₘₚₕₒₜ/t of a fluorescent particle, which is proportional to the fluorescence signal that was captured with the microscope and represented by the microscopy image, it holds:

Nₑₘₚₕₒₜ/t=η_{fluo}*σ_{abs,fluo}*Iₑₓ/(h*νₑₓ)=η_{fluo}*(ε_{abs,fluo}/N_{A})*Iₑₓ/(h*νₑₓ)

ε_{abs, fluo} (ε_{abs} for the fluorescent particle) and η_{fluo} are parameters for fluorescence markers and both are indicated by the manufacturer. They may also be determined independently by fluorescence and absorption spectroscopy using a method the skilled person is familiar with. To this end, devices that are commercially available and known to the skilled person, for example the following devices, may be used: FluoroMax-4 and Quanta-Phi by Horiba Scientific (distribution by Horiba Jobin Yvon GmbH, Bensheim, Germany). FluorMax-4 is necessary for the measurement of ε_{abs,fluo} and both devices in combination are necessary for the measurement of η_{fluo}.

For one unit of material with the absorption coefficient bₘₐₜ (in the following also b_mat), it holds, as described above, that the incident intensity I decreases, depending on the material thickness d, from the value of incident intensity Iₑₓ as follows:

I=Iₑₓ*e^{-b}^{_{_}}^{mat*d}

Due to energy conservation, the following intensity is absorbed:

I_{abs}^{mat}=Iₑₓ*(1−e^{-b}^{_{_}}^{mat*d})

Then, the number of photons N_{absphot}^{mat} that are absorbed in an area F_{b} over time t is

N_{absphot}^{mat}/t=I_{abs}/(h*νₑₓ)*F_{b}=Iₑₓ*(1−e^{-b}^{_{_}}^{mat*d})/(h*νₑₓ)*F_{b}

The number of emitted photons Nₑₘₚₕₒₜ^{mat} is calculated on the basis of the number of absorbed photons reduced by the fluorescence quantum yield of the material ηₘₐₜ

Nₑₘₚₕₒₜ^{mat}/t=ηₘₐₜ*N_{absphot}/t=ηₘₐₜ*Iₑₓ*(1−e^{-b}^{_{_}}^{mat*d})/(h*νₑₓ)*F_{b}

The emitted photon flow Nₑₘₚₕₒₜ^{mat}/t of a unit of material is proportional to the fluorescence signal obtained with the microscope and represented by the microscopy image.

In order to test a material for autofluorescence that might impede measurement, it is necessary to determine its absorption coefficient bₘₐₜ as well as its fluorescence quantum yield mat. To this end, as common practice for the skilled person, a sample unit of the material is prepared in a form that is advantageous for fluorescence spectroscopy, in many cases for example in form of a cuboid of 1 cm×1 cm×3 cm. This sample unit is measured with devices that are commercially available and known to the skilled person, e.g. FluoroMax-4 and Quanta-Phi by Horiba Scientific (distribution by Horiba Jobin Yvon GmbH, Bensheim, Germany) and bₘₐₜ as well as ηₘₐₜ are determined for different wavelength ranges. The wavelength ranges which are relevant for the method of the invention are excitation and emission wavelength ranges used with the fluorescence dye molecules attached to the 3D DNA nanostructures. Excitation wave lengths may for example be in the ranges 510-550 nanometers, 570-630 nanometers and/or 660-720 nanometers.

The measurement of b an η for a material may be carried out with the quantum efficiency measurement system by Horiba consisting of FluoroMax-4 with CCD and Quanta-Phi. To this end, the material cuboid is placed into the cuvette holder of the liquid sample holder for the Quanta-Phi. Subsequently, the sample holder is placed into the photometer sphere of the Quanta-Phi and the latter is closed. Subsequently the optical fibre between Quanta-Phi and FluoroMax is installed according to the operation manual. The measurement of the material is carried out using the FluorEssence™ software by Horiba Scientific according to the Quanta-Phi operation manual (“Quanta-Phi F-3029 Integrating Sphere Operation Manual—Part number J81089 rev. C”, Horiba Scientific 2010). A void sample chamber is measured as background reference sample. For this reference sample, the Rayleigh excitation peak is set to 10⁶ counts/s in the software. Excitation wavelengths are set to 488 nanometers, 561 nanometers and 647 nanometers for measurements in the channels Blue, Green and Red. For the background reference measurement, the emission range is set to 290 nanometers to 1100 nanometers. Integration time and the number of accumulations is set, as described in the manual, in such a way that the measurement value is 10⁶ counts/s for the excitation wavelength. Subsequently, the measurement is carried out on the material sample cuboid with the same parameters. The evaluation is carried out using the software as indicated in the user manual and the excitation wavelengths are set at 480-495, 555-570 and 640-655 nanometers for blue, green and red and the emission wavelengths at 500-540, 575-620, and 660-700 nanometers. Subsequently, the software calculates a value for the quantum efficiency η.

In order to measure the absorption constant b, the normal cuvette sample holder is again mounted into the FluoroMax-4 instead of the optical fibre and an absorption measurement is carried out according to the operation manual (“FluoroMax-4 & FluoroMax-4P with USB Operation Manual—Part number J810005 rev. D”, Horiba Scientific 2012) using the excitation wavelengths indicated above for the different channels. The measurement value for background measurement, which again takes place without sample, is again set to 10⁶ counts/s.

The degree of disturbance of the autofluorescence of the material of the second layer on the measurement can be determined by the relation γ between the fluorescence signal of a diffraction limited area F_{b} of material of the second layer having thickness d, corresponding to the height of the microwell, and the fluorescence signal of a fluorescence dye:

γ=ηₘₐₜ*N_{Avogadro}*F_{b}*(1−e^{-b}^{_{_}}^{mat*d})/(ε_{abs,fluo}*ηfluo)

An airy disk ((Y. Soskind, “Field Guide to Diffractive Optics”, SPIE Press 2011, Bellingham, Wash. USA, ISBN 978-0-8194-8690-5, p. 14) with a radius of r_{airy} is used for the diffraction limited area:

F_{b}=πr_{airy}²=π*(0,61*λ/NA)²

wherein π is the mathematical constant pi (3,1415 . . . ), λ the emission wavelength and/or the central wavelength of the emission wavelength range and NA the numeric aperture of the microscope used.

In the following, γ is referred to as fluorophor-related autofluorescence and is preferably lower than 5, more preferably lower than 2, particularly preferably lower than 0.5, most particularly preferably lower than 0.01 for at least one of the fluorescence channels.

The second layer comprises preferably SU-8, preferably PEG-DA, particularly preferably PDMS, more preferably Cytop and particularly preferably liquid glass. It is particularly preferred that the second layer consists of one of these materials. Liquid glass refers to a nanocomposite of solvent, plastics and silica nanopowder which is liquid at room temperature and is baked to nearly pure, preferably pure silicon dioxide/glass by sintering in the oven (see e.g. F. Kotz et al., “Three-dimensional printing of transparent fused silica glass”, Nature 2017, 544 (7650), 337-339 as well as F. Kotz et al., “Liquid Glass: A Facile Soft Replication Method for Structuring Glass”, Advanced Materials 2016, 28, 4646-4650).

In a preferred embodiment, the second layer consists of an elastic material, preferably PDMS and closes the microwell at the top side, while the first and second layers are reversibly connected with each other, preferably by pressure.

Alternatively or additionally, a microwell may also be etched into a layer, in particular if the layer consists of glass. As a result, there is a first continuous bottom layer and a second top layer with an etched recess as microwell, wherein the second layer is firmly bonded with the first layer and which forms the circumferential wall of the microwell.

The forms of the individual wells are preferably uniform, however, they may differ from one another. The form of a well in plan view can for example be square, circular, elliptic, hexagonal, round, triangular, polygonal and/or of irregular shape. The bottom of a microwell preferably has a diameter of an inscribed circle of 10 μm-1,000 μm, preferably 20 μm-500 μm, more preferably 50 μm-200 μm and/or an area of 1,000,000 μm² or less, preferably 250,000 μm² or less, particularly preferably 40,000 μm² or less. The depth of a microwell is preferably equal to or greater than the diameter of the inscribed circle of the relevant microwell.

In one aspect, the present invention is also directed to a microwell array, wherein one or more carrier adapter(s) is/are bound to the bottom of at least one of the microwells. Preferably, the target structure is bound and/or is being bound to the carrier and/or the first surface of the carrier by mediation of a carrier adapter, which binds specifically to the target structure. Thus, preferably the target structure is and/or is being bound to the bottom of the at least one microwell, wherein this binding is preferably mediated by a carrier adapter specifically binding to the target structure.

In a particularly preferred embodiment, the microwell array may be formed as part of a microwell chamber.

In the microwell chamber, the microwell array comprises a third layer which is attached to the second layer. The arrangement of the first, second and third layer is preferably such that the second layer is located between the first and the third layer. The third layer comprises an additional cavity which is directly adjacent to the microwells of the microwell array, i.e. the additional cavity of the third layer and the cavities formed by the microwells form a continuous working cavity which is closed towards the surrounding environment by the first layer, the second layer and the third layer.

Preferably, the cavity of the third layer is further connected with the surrounding environment via an inlet and an outlet extending through the third layer. The inlet and the outlet are preferably intended to efficiently fill the working cavity with fluid and/or discharge excess fluid, in particular in the steps of the method of the present invention. To this end, the inlet and/or the outlet can be provided with a standard connector, for example a Luer connector.

Said embodiment is in particular advantageous for the reason that on the one hand, it maintains the simplicity of the process steps already described due to the microwells that are open at the top and, on the other hand, it is possible to better control the flow behavior and the volume of the fluid used so that, ultimately, the use of reagents and sample material can be minimized.

The method step of introducing a group of host bodies including a host body with the target structure into a microwell array such that exactly one host body is present in at least one microwell represents a step of host body isolation. In this context, it is preferred that a volume of a suitable fluid with the group of host bodies in suitable concentration is used. Preferably, the host body fluid is added to the microwells and subsequently, the host bodies are allowed time to sink to the bottom of the microwells due to the effect of gravity. For HeLa Cells in PBS (recipe see e.g. Example 6), the time of the sinking may be for example 15-30 minutes. The introduction of the host body fluid into the microwells is preferably carried out by applying the host body fluid to the open side of the microwell array. In case that the host bodies are bacteria, viruses or other host bodies which do not sink due to gravity, the surface of the first and/or second layer of the microwell array may be provided with a cationic polymer such as for example poly-L-lysine (PLL) so that, based on statistic movement and/or diffusion of such host bodies, a sufficient number of the host bodies comes sufficiently close to the cationic polymer so that the host bodies adhere thereto (see e.g. B. Fang et al., “Surfaces for Competitive Selective Bacterial Capture form Protein Solutions”, ACS Appl. Mater. Interfaces, 2015, 7(19), pp. 10275-10282). The provision of the microwell array with a cationic polymer is preferably carried out at the same time with, before and/or after an optionally given step of providing the microwell array with carrier adapters, preferably before method step p1). For example, it is possible to add a solution with 0.01-5 mg/ml, preferably 0.1-1 mg/ml poly-L-lysine having a molecular mass of preferably 20000 g/mol (as e.g. commercially available from Sigma-Aldrich) in a buffer, preferably RX07, and to incubate for 5-120 minutes, preferably for 15-45 minutes in an additional method step cc) and prior to method step p1).

In order to prevent a possible undesired binding of the 3D DNA nanostructures to the cationic polymer, a further method step dd) may optionally be introduced after method step p1) and prior to method step p2) in which the positive charges of the cationic polymer present on the microwell array are saturated by an anionic polymer. For example, in this step, a solution of 0.01-5 mg/ml, preferably of 0.1-1 mg/ml PSS (polystyrene sulfonate) having a molecular mass of preferably 10,000-200,000 g/mol, more preferably 20,000 g/mol in a buffer, preferably RX07, may be added and incubated for 5-120 minutes, preferably for 15-45 minutes.

The introduction of the host body fluid into the microwells may be carried out by pipetting with standard pipettes and pipette tips, in particular with a sufficient size of the microwell array of preferably at least 1 mm×1 mm, more preferably at least 2 mm×2 mm, more preferably at least 5 mm×5 mm, more preferably 1 cm×1 cm, particularly preferably 1.5 cm×1.5 cm.

Alternatively or additionally, the introduction of the host body fluid into the microwells can be carried out with a tube and/or a different fluid channel.

The suitability of a fluid for the host body fluid depends on the type of the host body as well as on the other substances involved in the method and it is appropriately selected by the skilled person. Preferably, the fluid is an aqueous and/or hydrophilic fluid. It can for example be a cell suspension in which cells (e.g. HeLa cells) are present in PBS. Suitable host body concentrations depend on the geometry of the microwell array, i.e. on the form, size and arrangement (in particular the distances) of the microwells and on the form and size of the host bodies. The concentration is to be high enough so that a sufficient number of microwells contains an individual host body. Yet, the concentration is to be reduced to such a degree that the number of microwells containing more than one host body is as small as possible. A good starting point for the concentration to be selected is to have one cell per microwell in the filling solution. For example, a microwell array with a surface of 1 cm² and one million microwells can be filled with a cell suspension which covers the bottom with a depth of 1 millimeter and contains 10 million cells per milliliter. Another filling option is to fill a microwell array having ten thousand microwells per square centimeter with a host body suspension that has a depth of 100 micrometers and contains 1 million host bodies per milliliter.

In a preferred embodiment, the filling of the microwell can be carried out using a stencil. To this end, a preformed layer, preferably of PDMS, with recesses may be prepared, wherein the recesses have 0.2-5× the mean volume, preferably 0.5-2×, more preferably 0.8-1.2× of the mean volume of the host bodies (see e.g. Y. Wang et al., “Trapping cells on a stretchable microwell array for single cell analysis”, Anal Bioanal Chem 2012, 402(3): 1065-72; C. Probst et al., “Polydimethylsiloxane (PDMS) Sub-Micron Traps for Single-Cell Analysis of Bacteria). The distances between the recesses can have the same size as the distances between the microwells on the microwell array. This stencil can be filled by pouring a dense host body suspension (for example 1 million-100 billion host bodies per milliliter, preferably 10 million-1 billion host bodies per milliliter), wherein the majority of recesses (80%, preferably, 90%, more preferably 95%, even more preferably 99%) are occupied by exactly one host body since the wells do not offer space for more than one host body. Subsequently, the stencil can be placed with the open side down onto the microwell array and the microwells can optionally be closed with the stencil by pressure. Due to the exactly corresponding distances of the recesses and the microwells, exactly one recess is directly above a microwell and the host bodies descent from the stencil into the microwells due to gravity. This embodiment has the advantage of a more efficient single occupancy of the microwells by host bodies.

In a preferred embodiment of the method of the invention, it is possible to carry out a method step h) after step e) in which the circumferential walls of the microwells, preferably of the second layer, are removed. To this end, it is for example possible to use an embodiment of the microwell array of the invention in which the second layer consist of an elastic material, preferably PDMS, and closes the microwells at the top. As described above, the microwells of this second layer may be filled upside down with host bodies in step p1) and subsequently, be reversely closed with a first layer that is provided with carrier adapters, for example, by pressure. Thereupon, the host bodies can be lysed in step e) preferably physically, particularly preferably by laser induction or acoustically. In particular, the microwell array can be illuminated for example with a laser of 400 nanometers to 2,000 nanometers, preferably of 1.064 nanometers, with 50-500 milliwatts output for 1 minute-120 minutes, preferably 15 minutes, thereby lysing the cells. The then free cell structures can then bind to the carrier adapters of the first layer within an incubation period of 5 minutes to 24 hours, preferably of 10 to 120 minutes, more preferably of 15 to 45 minutes. Subsequently, in method step h), the second layer can be removed from the first layer. In a preferred variant, it can be completely removed so that the further methods steps take place without it. Alternatively, in a particularly preferred variant, it can be removed by 10 micrometers to 5 millimeters, preferably 100 to 200 micrometers so that a fluid chamber is formed through which the subsequent methods steps involving fluid exchange can be carried out.

This embodiment has the advantage that the fluid and component exchange in the following method steps can take place more freely and are not hindered by the barriers of the second layer. Thus, the respective method steps can take place in a quicker and more economic way.

The step of introducing of at least two 3D DNA nanostructures into the at least one microwell can take place in the same way as the introduction of the host body fluid, i.e. for example by pipetting and/or applying with a tube or the like as described above and in the following. The introduction of the 3D DNA nanostructures can take place before, at the same time and/or after the introduction of the host bodies. The order depends inter alia on whether and optionally which of the structures involved in the method are to be bound to the carrier.

In a preferred embodiment of the invention, the method further comprises:
f) closing the top side of the microwells by applying a preferably fluorinated oil layer onto the second layer.

The step of applying a preferably fluorinated oil layer onto the second layer takes place after step p1) and preferably by pouring preferably fluorinated oil over the microwell array that is preferably filled with aqueous host body fluid. Thereby, excess aqueous fluid is washed away and only the aqueous fluid which was already present in the microwells before remains there. The aqueous fluid already present in the microwells before remains there since, due to its hydrophobicity, the preferably fluorinated oil is prevented from entering into the microwells through the aqueous fluid within the microwells. Thus, the preferably fluorinated oil has no target for rinsing the aqueous fluid within the microwells out of the microwells. In addition, the density of the preferably fluorinated oil is lower than the density of the aqueous fluid and thus, counteracts a filling of the microwells with preferably fluorescent oil. Once the pouring of the preferably fluorinated oil over the microwells is terminated, a layer of preferably fluorinated oil remains which seals the microwells against the surrounding environment. Mixing with the aqueous fluid within the microwells does not occur due to the already mentioned hydrophobicity of the preferably fluorinated oil.

In a preferred embodiment, the oil comprises lipids. As is generally known, lipids have a hydrophobic and a hydrophilic segment. For this reason, a lipid layer is formed at the interface between the oil and the microwells. When the oil layer is replaced with aqueous solution, a second lipid layer covers the single lipid layer so that a lipid bilayer is formed.

Hence, a preferred embodiment of the method further comprises:
g) removing the oil by pouring aqueous solution over the microwell array

Thereby the oil that is no longer required is removed from the microwell array with a lipid bilayer remaining. The microwells remain closed by lipid bilayer.

In a preferred embodiment, the aqueous solution contains membrane channels, for example ion channels, in method step g). These incorporate autonomously into the lipid bilayer which closes the microwell (see e.g. R. Wanatabe et al., “Arrayed lipid bilayer chambers allow single molecule analysis of membrane transporter activity”, Nat. Commun. 5, ncomms5519 (2014)). Thereby, it is possible to render the lipid bilayer permeable for ions but impermeable for larger molecules. Thus, it is possible to subsequently reduce the salt concentration in the microwells by exchanging the aqueous solution outside the microwells by a hypotonic solution (with lower salt concentration) and thus to lyse the host bodies hypotonically (i.e. to disrupt them by osmosis, see e.g. C. J. Schröter et al., “A rapid method to separate endosomes from lysosomal contents using differential centrifugation and hypotonic lysis of liposomes”, Journal of immunological methods 1999, 227 (1-2), pages 161-168) while ensuring that the target structures remain in the microwell and do not diffuse out of it.

Thus, in a preferred embodiment of the method, step g) comprises:
g) Removing the oil by pouring aqueous solution comprising membrane channels over the microwell array

In order to add membrane channels to the lipid bilayer, it is possible to use for example 0.01-5 mg/ml α-hemolysin (αHL, commercially available, for example, from by Sigma-Aldrich), preferably 0.5-1 mg/ml αHL in a buffer, preferably PBS or RX07, in method step g) and to incubate for 5-120 minutes, preferably 15-45 minutes (see e.g. X. Zhang et al., “Natural channel protein inserts and functions in a completely artificial, solid-supported bilayer membrane”, Scientific Reports 2013, 3: 2196, DOI: 10.1038/srep02196). In an alternative embodiment of the invention, it is possible to incorporate membrane channel TRPM8 into the lipid bilayer in step g). To this end, micellar RGPM8 solution (described in E. Zakharian, “Recording of Ion Channel Activity in Planar Lipid Bilayer Experiments”, Methods Mol Biol. 2013, 998, pp. 109-118) is used as aqueous solution that contains membrane channels in method step g). At least a portion of the aqueous solution that contains membrane channels remains on the microwell array and is incubated for 5 minutes to 24 hours, preferably 2 hours so that membrane channels are incorporated from the membrane channel containing aqueous solution into the lipid bilayer.

The closing of the microwells by means of an oil layer and/or a lipid bilayer (method steps f) and g)) preferably takes place before the DNA nanostructures are introduced and/or after the disruption of the host bodies in order to prevent the still unbound target structures from escaping and to increase their efficiency in binding the carrier adapters. After a sufficient incubation period of 15 minutes, preferably 30 minutes, particularly preferably 1 hour, very particularly preferably 24 hours between method steps f) and g), almost all, preferably all target structures are bound to the carrier adapter and are no longer at risk to diffuse from the microwell. Thus, the 3D DNA nanostructures can be added after method step g). Here, it has to be taken into account that in case of an oil without lipids, this can be carried out after step g) since the sealing oil layer is already rinsed off in step g). In case that the method comprises the formation of a lipid layer (i.e. the use of a lipid-containing oil), it is necessary to first remove the lipid (bi)layer before the 3D DNA nanostructures are added. The removal of the lipid bilayer can be carried out by rinsing with a lysis buffer for a surfactant-based lysis method (see e.g. “Thermo Scientific Pierce Cell Lysis Technical Handbook” (2^{nd} ed.), Thermo Scientific) and incubating for 5-120 minutes, preferably 15-45 minutes. Subsequently, the 3D DNA nanostructures can be introduced into the microwells which are now free of oil layer and/or lipid bilayer.

The above-mentioned embodiments of closing the microwell by means of an oil layer and/or a lipid bilayer (method steps f) and g)) take preferably place using a microwell chamber. To this end, all steps are carried out as already described with the introduction of all fluids into the microwells taking place via the inlet of the microwell chamber and excess fluid and/or rinsed off fluid escaping via the outlet of the microwell chamber.

As already described, the microwell chamber has the advantage that the flow direction of the fluid(s) washed in and rinsed off is particularly controlled and is particularly advantageous. This applies in particular to the alternating rinsing with aqueous solutions and oil. The rinsing of the excess aqueous solution off the surface of the microwell array with oil is particularly facilitated by the shearing effect of a flow moving parallel to the surface of the second layer as promoted by the arrangement in the microwell chamber. The same applies to rinsing off the oil by means of an aqueous solution with retention of the lipid bilayer.

Furthermore, the invention is inter alia directed to a 3D DNA nanostructure, on which at least one fluorescence dye molecule is attached, wherein the shape of the 3D DNA nanostructure prevents that when approaching a second 3D DNA nanostructure, on which at least one fluorescence dye molecule is attached, the fluorescence dye molecules of the two 3D DNA nanostructures interact significantly.

Preferably, the 3D DNA nanostructure of the invention is a 3D DNA nanostructure having a cavity and at least one inwardly disposed fluorescence dye molecule, wherein the distance of the at least one inwardly disposed fluorescence dye molecule to the rim of the 3D DNA nanostructure is at least 2 nm, preferably at least 3 nm and particularly preferably at least 5 nm.

The skilled choice of the shape of the 3D nanostructures and the arrangement of the fluorescence dye molecules in the inside of/in the cavity of the 3D nanostructure ensures that the dye molecules do not sterically interact with the surroundings. As shown in the experimental Examples as attached, the 3D nanostructures of the invention thereby interact, e.g., to a significantly lesser extent with surfaces, such as a glass surface, than 2D nanostructures comprising the same dyes. This is highly advantageous for the use of such a 3D DNA nanostructure in the method according to the invention. For example, the rate of false positive detection of a target structure can be reduced in that manner, in particular if a carrier structure, e.g. the microwell array, is used. Moreover, an advantage of shielding the fluorescence dye molecules is that the fluorescence dye molecules of the two 3D DNA nanostructures do not significantly interact (optically and/or sterically). This permits the fluorescence signal of a 3D DNA nanostructure of the invention not being significantly affected by a very close adjacent 3D DNA nanostructure. Moreover, the unspecific interaction of 3D DNA nanostructures which is mediated by fluorescence dye molecules is prevented.

Taken as a whole, this is highly advantageous in the use of said 3D nanostructures for the detection of target structures. In particular, like in the method of the invention, this allows for the binding of at least two 3D DNA nanostructures to a target structure, without the fluorescence signal of the individual 3D DNA nanostructures being influenced due to their proximity (e.g. by quenching, fluorescence quenching or FRET).

Depending on the measurement method, the use of at least two DNA nanostructures may even be necessary in order to distinguish the identification structure made of target structure and bound DNA nanostructures from the free DNA nanostructures. This is in particular the case with measurement methods which are implemented in solution. When the identification structure is bound to a carrier and/or a carrier surface in the method for detection (as in a preferred embodiment of the method of the invention, in particular using the microwell array as a carrier), this allows for the removal of free DNA nanostructures using one or more washing steps. The use of at least two DNA nanostructures, which bind to a target structure, in a method that uses a carrier has the advantage that the rate of false positive detections may be lowered. First, an identification structure having at least two DNA nanostructures can be clearly distinguished from DNA nanostructures which interact with the surface in an unspecific manner. This is not possible in an analogous method, in which only one DNA nanostructure recognizes the target structure. Second, the use of at least two DNA nanostructures, both of which recognize different regions of the target structure, may significantly reduce and in many cases exclude the probability that other similar target structures are erroneously detected as an alleged target structure.

Moreover, with independent coupling of at least two 3D DNA nanostructures (e.g. different orthogonal measurable nanoreporters) to target molecules, the sensitivity of measuring may be significantly increased. It increases with the potency of the number of independent coupling reactions. This is highly advantageous in particular for high dynamics, i.e., for example, for simultaneously measuring of genes with a very low expression and a very high expression.

In a preferred embodiment, the present invention is also directed to a method for the detection and/or the quantification of at least two different target structures (e.g. two mRNAs, which are derived from different genes, i.e. which comprise a different nucleic acid sequence), preferably of a plurality of different target structures. The different target structures are pairwise distinguishable.

Preferably, said method comprises the following steps:
a) formation of an identification structure for each of the at least two different target structures, comprising:
   (i) the respective target structure, and
   (ii) at least two 3D DNA nanostructures, wherein each of the at least two 3D DNA nanostructures comprises one or more inwardly disposed fluorescence dye molecule and wherein each of the at least two 3D DNA nanostructures is specifically bound to the respective target structure, and wherein the at least two 3D DNA nanostructures are bound to regions of the respective target structure which are pairwise different;
b) detection of the at least two target structures by measuring at least one fluorescence signal,

wherein all 3D DNA nanostructures and the parameters of fluorescence measurement are selected such that the at least one measured fluorescence signal of the identification structures formed in a) is distinguishable from the fluorescence signal of all isolated 3D DNA nanostructures, when these are not bound in one of the respective identification structures, and that the measured fluorescence signals of the identification structures that are formed for the individual different target structures are pairwise distinguishable from each other.

Preferably, each of the different target structures is detected several times, i.e. each of the identification structures that is assigned to a specific target structure is formed several times.

In particular, a combination of method steps p1) and p2) is preferred.

All of the above-mentioned advantages of the method according to the invention apply mutatis mutandis in this case. Having the relevant form, DNA nanostructures offer the possibility to attach a plurality of identical and/or different fluorescence dye molecules. As is known from WO2016/1407727 A2 and WO2016/1407726 A2, a plurality of DNA nanostructures having a distinguishable fluorescence signal can thus be generated. However, the amount of the distinguishable combinations critically depends on the size of the DNA nanostructure, which limits the number of fluorescence dye molecules which may be attached thereon, without interacting with each other. However, large DNA nanostructures require more effort and are more expensive as regards their production and furthermore poorer than smaller structures as regards kinetics in a method for the detection of a target structure. Due to the use of at least two DNA nanostructures, which become part of the identification structure, the method of the invention has the advantage that also with significantly smaller DNA nanostructures, a similar plurality of fluorescence molecule combinations, which are bound to a target structure, may be generated. Furthermore, the use of at least two DNA nanostructures in an identification structure allows to further increase the number of the different distinguishable combinations of fluorescence dye molecules on a target structure and/or in an identification structure having the same maximum size as described in WO2016/1407727 A2 and WO2016/1407726 A2. Thereby, the number of different target structures that can be detected in a method can in principle be considerably increased. Apart from the use of at least two DNA nanostructures, in particular the form of the 3D nanostructure and the orientation of the fluorescence dye molecules into the inside of the structure are decisive. As mentioned above, it is only then that an interaction of the dyes of two DNA nanostructures is prevented and the signal of a combination of two or more 3D DNA nanostructures is reliably predictable and measurable.

According to the invention, nucleic acid nanostructures, also referred to as DNA nanostructures or DNA origami, are used. Nucleic acid nanostructures, also referred to as DNA nanostructures or DNA origami, are two- or three-dimensional structures, which inter alia consist of nucleic acids. The term “origami” describes that one or more strands or components of nucleic acids, preferably DNA, may be folded into almost any pre-defined structure or shape. Such a DNA strand is also referred to as scaffold strand. The one or the more scaffold strands is/are kept in shape by shorter nucleic acid strands (shorter relative to the at least one scaffold strand), which are also referred to as staple strands. Here, it is of major importance that the shorter strands (staple strands) are placed very precisely on well-defined positions on the DNA origami. DNA origami are described in more detail for example in Rothemund, “Folding DNA to create nano-scale shapes and patterns”, Nature, March 2006, pp. 297-302, vol. 440; Douglas et al., Nature, 459, pp. 414-418 (2009); and Seeman, “Nanomaterials based on DNA”, An. Rev. Biochem. 79, pp. 65-87 (2010). Reference to all of these documents is made herein in their entirety as part of the application.

Said DNA origami nanostructures can be functionalized with photoactive molecules, in particular with one or more fluorescent dye molecules. Thereby, the DNA origami as a whole becomes a fluorescent particle. Such DNA nanostructures, which are based on DNA origami, are described in WO 2016/140727 A2 and WO2016/1407726 A2. Reference to all of these documents is made herein in their entirety as part of the application.

Preferably, a DNA nanostructure of the invention is a 3D DNA nanostructure on which at least one fluorescence dye molecule is attached, wherein the shape of the 3D DNA nanostructure prevents that with approaching a second similar or identical 3D DNA nanostructure, on which at least one fluorescence dye molecule is attached, the fluorescence dye molecules of the two 3D DNA nanostructures significantly interact. Thus, by means of steric effects, the geometry of the DNA nanostructures prevents fluorescence dye molecules of different DNA nanostructures from coming too close to each other.

As can be inferred from its name, a DNA nanostructure preferably consists of DNA. In principle, a DNA nanostructure of the invention may, however, also contain other nucleic acids such as e.g. RNA, RNA analogues such as LNA, or DNA analogues or consist of these. Therefore, the present invention also comprises all embodiments described herein, in which the DNA nanostructure(s) and/or the 3D DNA nanostructure(s) are nucleic acid nanostructures and/or 3D nucleic acid nanostructures. A nanostructure formed of DNA is preferred, e.g. due to significantly lower production costs for such nanostructures.

A fluorescence dye molecule may be, for example, RFP, GFP, YFP or any of their derivatives (see for example P J Cranfill et al., “Quantitative assessment of fluorescent proteins”, Nature Methods, 13, 557-562 (2016)), an Atto dye (e.g. Atto647N, Atto565 or Atto488), an Alexa dye, DAPI, rhodamine, rhodamine derivative, cyanine, cyanine derivative, coumarin, coumarin derivative (for different organic fluorescence dyes see for example Q. Zheng and L Lavis, “Development of photostable fluorophores for molecular imaging”, Curr. Op. Chem. Biol. 39 p32-38 (2017)) or quantum dot (quantum dot, see E. Petryayeva et al, “Quantum dots in Bioanalysis: a review of applications across various platforms for fluorescence spectroscopy and imaging”, Appl. spectroscopy 67 (2013).

A 3D DNA nanostructure preferably refers to a structure, which substantially extends into three dimensions and is therefore different from an essentially two-dimensional structure such as, e.g., a slightly bent, rough or rippled surface. In other words, the minimal dimensions in three spatial directions that are perpendicular to each other are at least 2 nm, preferably at least 5 nm, more preferably at least 10 nm and particularly preferably at least 20 nm.

The interaction of two fluorescence dye molecules may in particular comprise fluorescence quenching and/or FRET.

In particular, at least two fluorescence dye molecules may be attached on the 3D DNA nanostructure, wherein the distance between the at least two fluorescence dye molecules is pairwise greater than that at which the fluorescence dye molecules significantly interact pairwise. For the exemplary case of a 3D DNA nanostructure having two fluorescence dye molecules, this means that the two fluorescence dye molecules have a distance, which is greater than the distance of interaction. For the exemplary case of a 3D DNA nanostructure having three fluorescence dye molecules, this means that each fluorescence dye molecule has a distance to the other two fluorescence dye molecules, which is greater than the respective interaction distance. For more fluorescence dye molecules, the same applies accordingly. That means, irrespective of which fluorescence dye molecule pair of the 3D DNA nanostructure is observed, the distance of the fluorescence dye molecules of the observed pair is always greater than the interaction radius of the observed pair. “Interaction radius” refers to the distance, which fluorescence dye molecules must have at the minimum in order to show negligible interactions (such as, e.g., fluorescence quenching or FRET). Which kind of dye pair has which kind of interaction radius is known to the skilled person. For example, corresponding interaction radii are described in Novotny, Lukas: Principles of nano-optics 2.ed, Cambridge Univ. Press, 2013, Kapitel “Optical Interactions”, pp 224 et seqq.; Peter Atkins: Physikalische Chemie, 5th edition, 2013, Wiley-VCH, Weinheim, Chapter 17 “Wechselwirkungen zwischen Molekülen”, pp 657 et seqq.; Förster T: Zwischenmolekulare Energiewanderung and Fluoreszenz. In: Ann. Physik. 437, 1948, S. 55. doi:10.1002/andp.19484370105. With regard to FRET, an interaction radius may be e.g. the distance at which a FRET rate of 50% is given.

The DNA nanostructures preferably serve the purpose of precise arrangement of a well-defined number of marker molecules (preferably fluorescence dye molecules) of one or more kinds. The geometric arrangement is of crucial importance, in order to prevent interactions between marker molecules (preferably fluorescence dye molecules). The defined number of marker molecules (preferably fluorescence dye molecules) allows for programming the intensity values and therefore the subsequent definite identification. The number of different combinations is N=k\hat{ }m, wherein k is the number of intensity levels and m is the number of different (in this case orthogonally measurable) marker molecules (preferably fluorescence dye molecules). By this multiplexing, a great number of different DNA nanostructures can be distinguished, without multiplexing, the number would only be m. In an Example, there are 5 intensity levels and three types of marker molecules and therefore 124 instead of 3 simultaneously distinguishable species. Thus, the DNA nanostructures are nanoreporters.

Thus, the term “nanoreporter” is used herein in order to define structures with attached marker molecules, which have measurable and identifying signatures, with dimensions in the nanometer range. In particular, the DNA nanostructures of the invention may be nanoreporters. Other DNA origami, e.g. according to Rothemund, can also be nanoreporters, as well as cascades of fluorescent proteins.

The term “orthogonally measurable” describes the property that it can be clearly extrapolated from a linear combination of measurement values of marker molecules (e.g. fluorescence dye molecules) to a linear combination of the underlying marker molecules and/or a combination of nanostructures. With the example of fluorescence detection, this may be realized by spectrally far-apart dyes and hence far-separated excitement and detection wave lengths (e.g. blue and red) or by closer-adjacent dyes in combination with multispectral detection, so that the orthogonal components may be calculated by “linear unmixing”.

A DNA nanostructure of the invention is preferably characterized in that one or multiple marker molecules (e.g. fluorescence dye molecules) of one or more kinds of marker molecules are attached such that the distance between two marker molecules is greater than that at which they significantly interact, and its form prevents that with approaching a similar DNA nanostructure, its marker molecules significantly interact with those of the other DNA nanostructure. A preferred DNA nanostructure of the invention is a DNA nanostructure, on which one or more marker molecules of one or more kinds of marker molecules is/are attached such that the distance between identical marker molecules is greater than that at which fluorescence quenching occurs and the distance between different marker molecules is greater than the Förster resonant energy transfer (FRET) radius, and its shape prevents that with approaching a similar DNA nanostructure, its marker molecule(s) interact with one or more marker molecules of the other DNA nanostructure by fluorescence quenching or FRET.

“Marker molecules” refer to particles which emit measurable signals. In particular, fluorescence dye molecules are marker molecules. The signal of fluorescence dye molecules may be the intensity, but also lifetime, polarization, blink kinetics or similar quantities. Gold particles are also marker molecules within this meaning.

Two marker molecules, in particular two fluorescence dye molecules, significantly interact, when the signal emitted by at least one of them significantly depends on the presence or absence of the other. For our purposes, for example a decline in intensity of the signal by 1% due to the presence of another molecule can be neglected, whereas a decline by 20% cannot be neglected. In the first case, the marker molecules (in particular fluorescence dye molecules) do not interact significantly, whereas they do so in the second case. In the case of similar fluorescence dye molecules, the interaction mechanism prevalent for us is fluorescence quenching, which is different depending on the fluorescence dye molecule, but which becomes negligible starting from a distance between the fluorescence dye molecules of about 2-3 nanometers. For fluorescence dye molecules of different types, the prevalent interaction mechanism is the Förster resonant energy transfer (FRET) and the distance from which the fluorescence dye molecule pairs no longer interact significantly (also referred to as FRET distance) varies in the range of 2-20 nanometers, depending on the individual pair. FRET distances are known for a plurality of fluorescence dye pairs, for example many dye producers publish lists for FRET distances of their dyes (e.g.: //www.atto-tec.com/fileadmin/user_upload/Katalog_Flyer_Support/R_0_-Tabelle_2016_web.pdf). Moreover, FRET distances may be calculated using the spectral properties of the fluorescence dye molecules (see Atkins, Physikalische Chemie).

Preferably, a significant interaction between two fluorescence dye molecules is an interaction, in which the measured fluorescence signal (with common excitation) of the one molecule is reduced by the presence of the other molecule in comparison to a measured fluorescence signal of the one molecule in the absence of the other molecule to 80% or less, preferably 90% or less, particularly preferably 95% or less, highly particularly preferably 99% or less.

A 3D DNA nanostructure of the invention preferably comprises a cavity, wherein the cavity has a volume of at least 0.1 zeptoliters (1e-22 liters), preferably at least 10 zeptoliters and particularly preferably at least 100 zeptoliters.

A 3D DNA nanostructure of the invention may essentially be formed as a hollow cylindrical DNA nanostructure, i.e. as hollow cylinder, wherein at least one fluorescence dye molecule is attached on the inside of the hollow-cylindrical DNA nanostructure. The cavity of the hollow cylinder preferably has a volume as indicated above.

The part of the 3D DNA nanostructure, which corresponds to the shell of the hollow cylinder, may comprise gaps. Those parts of the 3D DNA nanostructure, which correspond to the top and bottom surface of the hollow cylinder, are preferably open but may also each be at least partially closed and/or entirely closed. The barrel and optionally the top and bottom surface of the hollow cylinder are formed by the 3D DNA nanostructure, which means that it is not a mathematically precise cylinder, since e.g. the individual helices of the nanostructure form uneven surfaces with projections and recesses. However, the envelope of the 3D nanostructure is essentially the form of a cylinder.

Preferably, in a 3D DNA nanostructure of the invention, at least 2, more preferably at least 3, more preferably at least 4, more preferably at least 5, more preferably at least 6, more preferably at least 15, particularly preferably at least 30, particularly preferably at least 60 fluorescence dye molecules are attached. Preferably, the fluorescence dye molecules on a 3D DNA nanostructure are uniform, i.e. of the same type.

Preferably, a 3D DNA nanostructure of the invention comprises a cavity and at least one inwardly disposed fluorescence dye molecule, wherein the distance of the at least one inwardly disposed fluorescence dye molecule to the rim and/or the outer surface of the envelope of the 3D DNA nanostructure is at least 2 nm, preferably at least 3 nm and particularly preferably at least 5 nm. This has the advantage that due to the arrangement in the inside of the 3D DNA nanostructure and the minimum distance to the rim of the 3D DNA nanostructure, the dyes of the 3D DNA nanostructure also comprise said minimum distance to other structures with which the 3D DNA nanostructures may interact in the course of a method. For example, to a surface of a carrier, for example a substrate, the dyes have a distance that is greater than or equal to the minimum distance, when the 3D DNA nanostructure contacts said surface.

Preferably, a 3D DNA nanostructure of the invention comprises at least two inwardly disposed fluorescence dye molecules, wherein the pairwise distance of the at least two inwardly disposed fluorescence dye molecules is at least 2 nm, preferably at least 5 nm and particularly preferably at least 9 nm. For the exemplary case of a 3D DNA nanostructure with two dye molecules, this means that the two dye molecules comprise a distance of at least 2 nm, preferably at least 5 nm and particularly preferably at least 9 nm. For the exemplary case of a 3D DNA nanostructure with three dye molecules, this means that each dye comprises a distance to the two other dyes of at least 2 nm, preferably at least 5 nm and particularly preferably at least 9 nm. For more dyes the same applies mutatis mutandis. That means, irrespective of which dye pair of the 3D DNA nanostructure is observed, the distance of the dyes of the observed pair is always at least 2 nm, preferably at least 5 nm and particularly preferably at least 9 nm.

The distance of the dye molecules may, for example, be determined by identifying the structure of the 3D DNA nanostructure, from which can be inferred at which positions the dye molecules are positioned. To this end, sequence analysis may, for example, be required. For this purpose, a solution with DNA nanostructures is transferred to a solution with individual nucleic acid stands for example by reduction of the salt concentration. With a reduction of the salt concentration, the negative charges of the DNA backbone become more prevalent due to reduced shielding as compared to the permanently constant binding energies of the base pair-forming hydrogen bonds. Thus, with the reduction of the salt concentration, the DNA nanostructures are initially destabilized and with further reduction, the individual DNA double helices break and are sequenced, for example, with MySeq or another method provided by Illumina, Inc. and with the protocol as indicated by the manufacturer. Thus, the sequences of all nucleic acid strands present in the sample get known. The form of the DNA nanostructure may be reconstructed with this sequence information. To this end, any sequence editor (e.g. ApE 2.0 (http://biologylabs.utah.edu/jorgensen/wayned/ape/) can be used. The longest DNA strand of the analysis is the scaffold strand. It is loaded into the editor and the sequence regions complementary to the scaffold strand are calculated and noted for each single staple sequence. There are two different regions on the scaffold strand for each staple strand. As described at a different point in this specification, with this information, the topology can in turn be defined in CaDNAno and thus the DNA nanostructure design can be reconstructed. In the next step, it must be determined which of the staple strands are provided with dyes. This is simple with the use of dye adapters, since a particular number of staple strands have further identical sequences, which remain single-stranded after the above reconstruction of the DNA nanostructure. If the staple strands are directly modified, the individual staple strands must be isolated and be analyzed for their fluorescence properties. Each individual staple strand may be loaded with additional molecular weight by hybridization to a complementary strand and be isolated in an agarose gel electrophoresis from the above solution with individual nucleic acid strands. Finally, a respective fluorescence measurement shows which of the staple strands is labeled with a fluorophore. These can be identified in caDNAno and the distances between fluorescence molecules can be calculated.

As an alternative to this method, using mass spectrometry (Sauer S, Lechner D, Berlin K et al. (2000) Full flexibility genotyping of single nucleotide polymorphisms by the GOOD assay. Nucleic Acids Res 28:E100; Haff L A, Smirnov IP (1997) Single-nucleotide polymorphism identification assays using a thermostable DNA polymerase and delayed extraction MALDI-TOF mass spectrometry. Genome Res 7:378-388; Wenzel T, Elssner T, Fahr K et al. (2003) Genosnip: SNP genotyping by MALDI-TOF MS using photocleavable oligonucleotides. Nucleosides Nucleotides Nucleic Acids 22:1579-1581; Braun A, Little D P, Koster H (1997) Detecting CFTR gene mutations by using primer oligo base extension and mass spectrometry. Clin Chem 43:1151-1158; Sun X, Ding H, Hung K et al. (2000) A new MALDI-TOF based mini-sequencing assay for genotyping of SNPS. Nucleic Acids Res 28:E68), the solution with DNA nanostructures can be directly sequenced and it can be simultaneously established which staple strands have fluorescent molecules attached. In analogy to the above, the topology of the DNA structure can subsequently be reconstructed in CaDNAno using complementary sequence analysis of staple strands and scaffold strand (which is again the longest strand). The co-measured information, on which strands the fluorescence molecules are attached, can be directly used to calculate the distances of the fluorescence molecules using CaDNAno.

In a preferred embodiment, the 3D DNA nanostructure is essentially formed as elliptic cylinder, preferably essentially as circular cylinder.

As already set forth above, the structures according to the invention are not a mathematically precise cylinder, since the surface of the structure at the single-atom level is extremely uneven and, e.g. at the level of the individual helices of the nanostructure, may comprise unevenness of the order of a helix radius or helix diameter. However, these structures are cylindrical to a skilled person nonetheless, since, e.g., the envelope of the 3D nanostructure may essentially have the form of a cylinder.

In a preferred embodiment, the hollow cylinder of the 3D DNA nanostructure is a circular cylinder and comprises an outer radius of at least 5 nm, at least 10 nm, preferably of at least 20 nm, preferably of at least 20 nm, preferably an outer radius of 30 nm to 80 nm, preferably of 50 nm to 70 nm and particularly preferably an outer radius of 60 nm. The outer radius refers to the structure of the outer radius of the envelope.

In a preferred embodiment, the hollow cylinder of the 3D DNA nanostructure, which is preferably a circular cylinder, has a height of at most 200 nm, preferably a height of 60 nm-30 nm, particularly preferably a height of 30 nm. The height of the structure refers to the height of the envelope.

The preferred embodiments ensure a sufficient size of the 3D DNA nanostructure in order to attach a sufficient number of dye molecules at sufficient distance of the dye molecules from each other and preferably in the inside of the 3D DNA nano structure.

In a preferred embodiment, the hollow cylinder of the 3D DNA nanostructure comprises a wall thickness of at least 2 nm, preferably 2 nm to 7 nm, particularly preferably 5 nm. The wall thickness of the structure refers to the difference between outer radius and inner radius. In other words, the wall thickness corresponds to the distance between the outer envelope of the cylinder and the inner envelop of the cylinder cavity.

Preferably, a 3D DNA nanostructure of the invention comprises a wall thickness that at least corresponds to the diameter of a DNA double helix. Preferably, the wall thickness has space for 2-4, particularly preferably 3 DNA double strands, wherein the DNA strands are preferably arranged perpendicularly to the wall thickness in their length and are additionally arranged on adjacent lattice sites of a honeycomb lattice.

The inner diameter of the hollow cylinder is defined as the difference of outer diameter of the hollow cylinder and the wall thickness. Thus, preferred inner diameters result from the details on the outer diameter and on wall thickness as indicated herein.

The described minimum wall thicknesses in combination with the shape of the 3D DNA nanostructure, which shields the dyes from the surroundings, also contribute to the fact that an interaction of the dyes of the 3D DNA nanostructure with a surface of a carrier and/or another dye, which may be attached in another 3D DNA nanostructure, is prevented by steric hindrance. Thus, in particular, quenching and/or FRET with another dye and/or unspecific binding to a carrier, in particular a substrate surface, is prevented.

As is common for DNA origami, the 3D nanostructures of the invention preferably comprise a DNA single strand as “scaffold strand”. The scaffold strand preferably comprises at least 3000 bases, preferably 5000-50000 bases, particularly preferably 10000-11000 bases. However, a different number of bases is possible. Preferably, the scaffold strand is circular. Here, circular means that the DNA strand comprises no open 5′-end and no open 3′-end. A non-limiting example for a scaffold strand is mentioned in the attached Examples and is shown in SEQ ID NO:1258.

Moreover, as is common for DNA origami, the 3D nanostructures preferably comprise a plurality of further shorter single-stranded DNA molecules. These are preferably referred to as “staple strands”. According to the invention, the number of the staple strands of the 3D DNA nanostructure is preferably selected that it is adjusted to the length of the at least one scaffold strand of the 3D DNA nanostructure. Preferably, the 3D DNA nanostructure comprises 100-500 staple strands. A staple strand preferably comprises 30-100 bases. Non-limiting examples for staple strands are mentioned in the attached Examples and/or shown in SEQ ID NOs.:1 to 504.

The 3D DNA nanostructures may be of cylindrical shape, in which the marker molecules are inwardly disposed with a distance to the rim of at least half of the interaction radius. This ensures that the marker molecules of different DNA structures are not closer to each other than the interaction radius, irrespective of the position of the DNA structures relative to each other. To this end, a cylinder with side view that is approximately square provides a good ratio of steric hindrance (and thus shielding), small hydrodynamic radius (and thus diffusion velocity) and inwardly disposed marker molecule positions with sufficient distance between marker molecules. In particular, a DNA nanostructure hollow cylinder with a diameter of about 30-90 nanometers and a height of about 30-90 nanometers is envisaged.

Alternatively, the DNA structures may be basket-shaped, with dyes positioned on the “bottom” of the basket and a basket rim that is at least as large as the largest interaction radius of all combinations of marker molecule types. The basket rim may have any angle to the basket bottom, preferably the angle is in a range of −45° to +45°, however. Preferably, the basket rim has a height of at most 200 nm, preferably a height of 60 nm to 30 nm, particularly preferably a height of 30 nm. The height of the structure again refers to the height of the envelope.

The open side of the basket preferably comprises an outer diameter of at least 5 nm, preferably an inner radius of 30 nm to 60 nm, particularly preferably an inner radius of 60 nm. A hollow cylinder with an open and a closed top side is a specific case of a basket.

The 3D DNA nanostructure of the invention may comprise a substantially square projection, i.e. a ratio of height to width and/or diameter is in the range of 0.8 to 1.2. In particular, a 3D DNA nanostructure designed as a hollow cylinder and/or basket may comprise a substantially square projection.

A 3D DNA nanostructure of the invention may comprise the form of a hollow ball and preferably be provided with inwardly disposed marker molecules.

3D DNA nanostructures of the invention are also wireframe geometries such as tetrahedron, octahedron, cube, hexahedron, pyramids etc.

The general principle of the production of DNA origami and thus DNA nanostructures in different shapes is well-established, e.g. from Rothemund, “Folding DNA to create nano-scale shapes and patterns”, Nature, March 2006, pp. 297-302, vol. 440; Douglas et al., Nature, 459, pp. 414-418 (2009); and Seeman, “Nanomaterials based on DNA”, An. Rev. Biochem. 79, pp. 65-87 (2010). As already mentioned, the production principle for DNA origami is based on the joint incubation of at least one scaffold strand, which is preferably a single strand, and a plurality of staple strands. The staple strands comprise at least two binding segments that have the purpose of binding each to complementary segments of the scaffold strand. During incubation, which is preferably initiated at a temperature of 50-70° C. that is subsequently reduced, the staple strands and the scaffold strand bind via their respective complementary binding segments. Thereby, the generated DNA nanostructure folds into a conformation. By directed design of the scaffold strand and the staple strands as well as their complementary binding segments, a DNA origami may be designed and produced in accordance with the need of the user. For designing the DNA origami, freely-accessible software is available. For example, the program CaDNAno 2.5 (source code available at: https://github.com/cadnano; user manuals available at: http://cadnano.org/docs.html and/or http://cando-dna-origami.org/tutorial/ (see also S. M. Douglas et al, “Rapid prototyping of 3D DNA-origami shapes with caDNAno, Nucleic Acids Res., 37(15), 2009) may be used.

In this program, any scaffold strand sequence, which is preferably available for production of the DNA nanostructures, is predefined; particularly preferred are scaffold strands p7308 (SEQ ID NO: 1258) or p7249 (SEQ ID NO: 1257). In addition, the desired topology of the DNA nanostructure is specified.

Specifically, CaDNAno calculates the number and sequences of the required scaffold strands and staple strands with the specification of (1) length and (2) sequence of the scaffold strand, (3) spatial shape of the scaffold strand and (4) starting position of the scaffold strand. The spatial shape of the scaffold strand defines the shape of the envisaged structure and contains the number and arrangement of the helices and the course of the scaffold strand through these helices. On the push of a button, the program is able to connect the determined scaffold strand with the staple strands in an autonomous manner. Subsequently, the staple strands can be output in tabular form. The table contains in particular starting and end position of the staple strand (defined by helix and number of bases from the rim of the helix, e.g. 7[128] refers to a position on the 7th helix, 128 bases from the arbitrarily but uniformly defined left rim) for unambiguous identification, the number of bases (i.e. the length of the staple strand) and the sequence of the staple strand. It is worth noting that with identical structure design (point (1)-(3)) but different starting position of the circular scaffold strand, other staple strand sequences are generated. These are equally usable without limitation as those having a different starting position, yet not compatible with these.

For bends of a DNA helix that are occasionally useful in 3D structures, positions along the scaffold strand may be defined at which the corresponding staple strand comprises one base more or less. Additional staple strand-staple strand pairings may also be defined, which add further DNA-helix contour length to the structure beyond the prescribed length of the scaffold strand. It is of note that CaDNAno merely shows representations on one plane, and 3D representation must be made by the spatial imagination of the user or a plugin of CaDNAno for the design program Autodesk Maya (D Selnihin and E S Andersen, “Computer-aided design of DNA origami structures”, Comp. Meth. Synth. Biol. 1244, pp 23-44 (2014)). CaDNAno eventually provides a list of staple strands, by means of which the desired structure can be produced. Each of these staple strands may in principle be provided with a fluorophore/fluorescence dye molecule. However, the program does not envisage a function for the definition of fluorescence dye molecule positions for direct labeling in the exported staple strand list. However, the program assists in the visualization of this process, because it shows the starting and end position of the strands as well as the shape of the scaffold strand and the arrangement of the helices. Using this tool, the user can readily calculate relative distances of possible positions on the staple strands within the designed 3D DNA nanostructure. Concretely and preferably, the user selects positions on staple strands that are to be modified. These are, for example, the 5′-ends or the 3′-ends. Subsequently, the user selects the staple strands which meet the positioning requirements for the fluorescence dye molecules, for example, that they are positioned inside the cavity structure and have the desired, sufficient distance to the rim of the structure. As described above, this can be inferred from CaDNAno. Finally, the user calculates the minimum distancees of the positions to be modified on the staple strands that can still be selected, which is limited to 2-3 calculations and can be readily accomplished due to the lattice structure of the design and the visualization in CaDNAno. Finally, depending on the number of fluorescence dye molecules to be realized, the user selects an arbitrary subset of the selectable staple strands, notes the staple strand IDs and orders the corresponding sequences in the exported staple strand list with fluorescence dye molecule modification at the selected position. For an attachment of fluorescence dye molecules on the DNA nanostructures based on fluorescence dye molecule adapters, the strategy is analogous, with the only difference that the exported staple strand sequences are not used with fluorescence dye molecule modification but with a sequence that is extended by the adapter sequence complementary to the fluorescence dye molecule adapter(s). Moreover, the selected position along the staple strands is preferably an end of the staple strands which particularly preferably protrudes from the structure into the inside (and/or into the cavity). With this adapter-based fluorescence dye molecule attachment on the DNA structures, many adapter strands of similar kind provided with fluorescence dye molecule(s) (and SEQ ID NO, for example 1259-1261 for the different fluorescence dye molecule colors) bind to many different staple strands, which are each extended with the same complementary sequence for the adapters, on the DNA nanostructure. When using fluorescence dye molecule adapters, the fluorescence dye molecule is preferably bound on the side of the structure of the fluorescence dye molecule adapter that is directed to the rim and/or the wall. This allows a reduced mobility. For example, this is the 3′ end of the fluorescence dye molecule adapter, when the 5′ end of the staple strands as defined by CaDNAno is extended by the complementary adapter sequence. However, the opposite case is also conceivable.

According to the same model as for the staple strands for fluorophore adapters, one staple strand is designed for target adapters for each 3D DNA nanostructure, wherein the selection criteria for the staple strand position may be different, for example exposedness for good binding efficiency with target structures.

The production of 3D nanostructures in different 3D shapes, e.g., in a structure of a hollow cylinder, is also known from Knudsen J. et al. (Nature Nanotechnology 10, 892-898 (2015) doi:10.1038/nnano.2015.190). In order to produce 3D DNA nanostructures of the invention that are based on the design therein, preferably all or a subset of the oligomers, which exclusively bind helices that point to the inside of the hollow cylinder and that are not located at one of the edges (rims) of the hollow cylinder, are provided with a dye at one end and/or extended by adapter sequences (to which a fluorescence adapter, which comprises a fluorescence dye molecule, can be bound). For the target adapter or carrier adapter, an oligomer can be selected which is incorporated in a helix that is located at the rim of the hollow cylinder.

Moreover, in the attached Examples (in particular in Example 1), an exemplary example is explained for a production method of a 3D DNA nanostructure of the invention. Based on the sequences used for this method and based on the method provided for the production, a skilled person may also produce similar 3D DNA nanostructures (e.g. with entirely different sequences (which, however, e.g., have complementary DNA sequences at similar or at the same positions)).

In another aspect, the present invention relates to a set of multiple 3D DNA nanostructures (wherein these are preferably 3D DNA nanostructures as described in this application), wherein the set comprises N pairwise different 3D DNA nanostructures and wherein the N different 3D DNA nanostructures of the set are pairwise different from each other in the fluorescence dye molecules. Preferably, the N pairwise different 3D DNA nanostructures contain a different number of fluorescence dye molecules and/or different fluorescence dye molecules, so that with the N pairwise different 3D DNA nanostructures of the set, k intensity levels that are distinguishable from each other and/or m color levels that are distinguishable from each other can be generated. Preferably, at least a part of the N pairwise different 3D DNA nanostructures are contained in the set multiple times, so that each of the k intensity levels is formed by intensity distribution, and wherein the k intensity distributions are distinguishable from each other, preferably statistically. Preferably, at least a part of the N pairwise different 3D DNA nanostructures are contained in the set multiple times, so that each of the m color levels is formed by color distribution, and wherein the m color distributions are distinguishable from each other, preferably statistically. The overlap of adjacent distributions is lower than 30%, preferably lower than 20%, preferably lower than 10%, more preferably lower than 5%, even more preferably lower than 2% and particularly preferably lower than 1%.

In this context it applies preferably that: k>2, preferably k>3, more preferably k>4, even more preferably k>5, particularly preferably k>6; and/or m>2, preferably m>3, more preferably m>4, even more preferably m>5, particularly preferably m>6.

In another aspect, the present invention is also directed to a method for the detection of a target structure. This method comprises:
a) formation of an identification structure, comprising:
   (i) the target structure, and
   (ii) at least two 3D DNA nanostructures, wherein each of the 3D DNA nanostructures comprises one or more inwardly disposed fluorescence dye molecules and wherein each of the 3D DNA nanostructures is (specifically) bound to the target structure,
b) detection of the target structure by measuring at least one fluorescence signal, wherein the 3D DNA nanostructures and the parameters of fluorescence measurement are selected such that the at least one measured fluorescence signal of the identification structure formed in a) is distinguishable from the fluorescence signal of each of the at least two isolated 3D DNA nanostructures, when these are not bound in the identification structure.

Preferably, the at least two 3D DNA nanostructures bind to regions/segments of the target molecule that are pairwise different. Thus, a higher specificity for the detection of a target structure is achieved.

In particular, a combination of said method with method steps p1) and p2) is preferred.

A target structure that is detected with a method of the invention may be a molecule, a complex of several molecules or a particle. In particular, a target structure may be a DNA (preferably an at least partially single-stranded DNA), an RNA (preferably an at least partially single-stranded RNA, e.g. mRNA), an LNA (preferably an at least partially single-stranded LNA) or a protein. However, a target structure that is a complex, which contains and/or consists of one or more DNAs (preferably at least an at least partially single-stranded DNA), one or more RNAs (preferably at least an at least partially single-stranded RNA), one or more LNAs (preferably at least an at least partially single-stranded LNA) and/or one or more proteins, is also applicable. In principle, the target structure may also be an inorganic particle. Preferably, the target structure comprises or is a polynucleotide (i.e., e.g., a DNA, an RNA or LNA), particularly preferably an at least partially single-stranded polynucleotide and particularly preferably a single-stranded polynucleotide. Particularly preferably, the target structure comprises or is a single-stranded DNA, a single-stranded RNA or a single-stranded LNA. In a preferred aspect, the method of the invention is used for gene expression analysis. In this case, the target structure preferably is an mRNA or a protein, particularly preferably mRNA. Consequently, the target structure may be a protein or an mRNA. “Partially” single-stranded means that the poly nucleic acid (i.e., e.g., a DNA, an RNA or LNA) comprises a single-stranded area of at least 10 bases, preferably at least 15 bases and particularly preferably at least 21 bases.

Consequently, in a preferred embodiment of the method, the target structure can have or be a polynucleotide, preferably a partially single-stranded polynucleotide, preferably a single-stranded polynucleotide and particularly preferably an mRNA.

Consequently, in a preferred embodiment, the method of the invention may be a method for gene expression analysis, wherein an mRNA is detected (thus, the mRNA is the target structure) and the method comprises:
a) Formation of an identification structure, comprising:
   (i) an mRNA, and
   (ii) at least two 3D DNA nanostructures, wherein each of the 3D DNA nanostructures comprises one or more inwardly disposed fluorescence dye molecules and wherein each of the 3D DNA nanostructures is (specifically) bound to sequence segments of the mRNA that are pairwise different,
b) detection of the target structure by measuring at least one fluorescence signal, wherein the 3D DNA nanostructures and the parameters of fluorescence measurement are selected such that the at least one measured fluorescence signal of the identification structure formed in a) is distinguishable from the fluorescence signal of each of the at least two isolated 3D DNA nanostructures, when these are not bound in the identification structure.

In particular, a combination with method steps p1) and p2) is preferred so that a preferred embodiment is a method for gene expression analysis, wherein an mRNA is detected (thus, the mRNA is the target structure) and the method comprises:

p1) introduction of a group of host bodies including a host body with the mRNA into a microwell array such that exactly one host body is present in at least one microwell;

p2) introduction of at least two 3D DNA nanostructures into the at least one microwell wherein each of the 3D DNA nanostructures comprises one or more inwardly disposed fluorescence dye molecules;
a) forming an identification structure, comprising:
   (i) an mRNA, and
   (ii) at least two 3D DNA nanostructures, wherein each of the 3D DNA nanostructures comprises one or more inwardly disposed fluorescence dye molecules and wherein each of the 3D DNA nanostructures is (specifically) bound to sequence segments of the mRNA that are pairwise different;
b) detection of the mRNA by measuring at least one fluorescence signal,
   wherein the 3D DNA nanostructures and the parameters of fluorescence measurement are selected such that the at least one measured fluorescence signal of the identification structure formed in a) is distinguishable from the fluorescence signal of each of the at least two isolated 3D DNA nanostructures, when these are not bound in the identification structure.

With respect to the method of the invention, a “3D DNA nanostructure” refers to the same as defined above in connection with the 3D DNA nanostructures of the invention. In other words, a 3D DNA nanostructure may be referred to as DNA origami.

Preferably, at least one, preferably all, of the at least two 3D DNA nanostructures, which are part of the identification structure, are 3D DNA nanostructures of the invention as described herein.

The skilled person knows how to select the 3D DNA nanostructures and the measurement parameters in dependence of each other and what fluorescence signals can be differentiated with what kinds of measurement methods and what kinds of measurement parameters. The 3D DNA nanostructures that are different from each other may be different from each other in their fluorescence response, for example by color and/or intensity. It is clear to the skilled person that when using different colors, accordingly suitable excitation wave lengths and filters adjusted to the colors must be used. The maximum number of obtainable color levels m depends inter alia on how precisely two adjacent color distributions can be technically resolved. The same applies to the maximum number of achievable intensity levels, since the method is based on the fact that two adjacent intensity distributions (the intensity levels k1 and k1+1) can still be separated (at least statistically). For example, individual intensity distributions can be modeled and based on the mixture distribution of multiple partially overlapping intensity distributions relative population sizes of the individual distributions can be calculated by deconvolution or statistical interference, preferably Bayesian inference.

However, instead of color and/or intensity also other variables can be employed, which are suited to distinguish the fluorescence signals of different identification structures such as e.g. the bleaching rate of the color molecule or fluorescence lifetime.

The detection of a target structure may comprise the detection that a target structure is present. However, in principle, the detection may also comprise the exclusion that a target structure is not present. In particular, the method of the invention is also suited for the quantification of the target structure. In other words, the detection of the target structure preferably comprises the quantification of a target structure (e.g. in a sample solution). The quantification may be relative, i.e. in relation to another component (e.g. a second structure/target structure in a sample solution) or absolute (i.e. in form of a concentration or absolute number). For example, it may be quantified in absolute terms when the detection of the identification structure(s) is in solution, such as in flow cytometry, FCS or light sheet microscopy-based measurement geometries. Then, all identification structures in a given sample volume can be measured and an absolute number and/or concentration can be indicated. For more precise statements, the sample can be measured multiple times in different dilution steps and/or the number of the target structures not considered can be estimated by statistical methods, preferably sorted out measurement events (for example due to the presence of less than the expected number of DNA nanostructures in a measurement event) may be estimated by Bayesian inference. For the measurement of identification structures that are bound to a carrier and/or a carrier surface, an analogous approach is conceivable. To this end, the parameters must be selected such that the probability that identification structures do not bind to the carrier and/or the (first) carrier surface is low. This may be achieved, for example, by a high ratio of carrier surface and/or the first carrier surface to the sample volume (for example 0.01/μm, preferably 0.1/μm, more preferably 0.5/μm, highly preferably 1/μm) and/or by a long incubation period (for example 30 min, preferably 2 h, more preferably 10 h, highly preferably 24 h). Then, all identification structures can again be measured and their number can optionally be divided by the sample volume. As in the above case, further steps in the analysis can refine the estimation. Preferably, the measurement can be performed in a sample chamber where only one of the surfaces comprises carrier adapters (preferably the surface easiest to measure) and all other surfaces are passivated (e.g. as described elsewhere herein) so that the measurement of all identification structures is more simple.

The quantification can also be carried out on the basis of an internal standard or on empirical data. Preferably, the internal standard defines a comparative value with a known concentration.

The method of the invention for the detection of a target structure may further comprise that the identification structure formed in a) is bound or is being bound to a carrier. Consequently, the formed identification structure can be bound to a carrier, preferably to a first surface of the carrier. Thus, the method may comprise the step of binding of the formed identification structure to a carrier, preferably to a first surface of the carrier.

If the identification structure is bound to carrier, it preferably means that the identification structure is formed on the carrier. The binding of the identification structure to the carrier may be mediated in that the target structure and/or one of the at least two 3D DNA nanostructures (preferably the target structure) is/are pre-bound or is/are bound to the carrier already prior to formation of the identification structure. In other words, the target structure and/or one of the at least two 3D DNA nanostructures (preferably the target structure) may be bound to the carrier (prior to formation of the identification structure). Thus, the method may further comprise the step of binding of the target structure and/or at least one of the at least two 3D DNA nanostructures (preferably the target structure) to the carrier. For example, this step may comprise the incubation of the carrier and/or the first carrier surface with a sample solution that contains the target structure (and optionally also further components such as e.g. further polynucleotides, preferably mRNAs). Furthermore, this step can comprise one or more washing steps (with a buffer solution). The pre-binding of the target structure to the carrier and/or the first carrier surface in combination with the at least one washing step has the advantage that the target structure can already be removed from the context of the sample solution prior to formation of the identification structure. This is particularly advantageous with complex samples having many and optionally similar components.

The buffer solution can contain 1× to 8×SSC, preferably 3× to 5×SSC, and particularly preferably 4×SSC. Therein, SSC refers to the so-called saline sodium citrate buffer such as SSC, which consists of an aqueous solution of 150 mM sodium chloride and 15 mM trisodiumcitrate, which is adjusted to pH 7.0 with HCl. As a buffer basis, also Tris or PBS can be used as an alternative to a citrate buffer like SSC. The buffer can also comprise NaCl and/or MgCl₂ (preferably as an alternative to SSC). The concentration of NaCl is preferably 50 mM to 1200 mM, particularly preferably 200 mM to 800 mM. For example, the NaCl concentration can be 600 mM, preferably 500 mM and particularly preferably 300 mM. The concentration of MgCl₂ can be 2 mM to 20 mM, preferably 5 mM to 15 mM, preferably 8 mM to 12 mM and particularly preferably 10 mM. Moreover, the buffer solution can comprise 4% to 6%, 2% to 10%, 15% or 20% dextran sulfates. Preferably, the buffer comprises, for example, 5% dextran sulfates. The buffer solution can also comprise polyethylene glycol (PEG), e.g. PEG8000, PEG2000, PEG4000, PEG1000. The buffer can also comprise 0.01 to 5% Tween-20. Optionally, the buffer can comprise EDTA, preferably at a concentration of 0.1 mM to 5 mM, particularly preferably 1 mM. A further optional component of the buffer is “sheared salmon sperm” (commercially available), preferably at a concentration of 0.1 mg/ml. “Sheared salmon sperm” can potentially increase specificity. The buffer can also comprise Denhardts medium (consisting of an aqueous solution of 0.02% (w/v) BSA (Fraction V), 0.02% Ficoll 400 (commercially available) as well as 0.02% polyvinylpyrrolidone (PVP), see also Cold Spring Harb Protoc 2008, doi:10.1101/pdb.rec11538), preferably in 1-fold, 2-fold, 3-fold, 4-fold or 5-fold concentration. A particularly preferred buffer comprises or has the composition 4×SSC, 5% dextran sulfate and 0.1% Tween 20. This buffer is used particularly preferably, when at least one of, preferably all of, the target structures to be detected are a polynucleic acid, e.g. an mRNA.

If the identification structure is being bound to a carrier, this preferably means that the identification structure is not only formed but also gets bound on the carrier, preferably on the first surface of the carrier. In other words, none of the components of the identification structure is pre-bound. Therefore, the method can comprise the step of binding of the identification structure to the carrier, preferably the first surface of the carrier. The binding can occur in one step with the formation of the identification structure in a) (i.e. the carrier and all components required for the formation of the identification structure (and optionally components that are necessary for binding to the carrier, e.g. the carrier adapter described below) are mixed in one step and incubated). Alternatively, the binding to the carrier can occur subsequently (preferably prior to measurement in b)). In both cases, after the binding of the identification structure to the carrier and/or the first carrier surface, the method can further comprise at least one washing step (e.g. with one of the above-mentioned buffer solutions) to remove non-bound components (e.g. 3D DNA nanostructures and/or other components contained in a sample solution) from the carrier. When the identification structure is being bound, the binding can be mediated by the target structure and/or one of the at least two 3D DNA nanostructures (preferably the target structure) in analogy to “is bound”.

The bond (if the identification structure is bound to carriers) or the binding (if the identification structure is being bound to carriers) of the identification structure can be mediated by the target structure and/or one of the at least two 3D DNA nanostructures (preferably target structure). It is particularly preferred that the bond and/or the binding is mediated by the target structure.

The bond and/or the binding of the identification structure to the carrier and/or the first surface of the carrier can be mediated directly or by a carrier adapter. Consequently, the bond and/or the binding of the target structure and/or at least one of the 3D DNA nanostructures bound in the identification structure (preferably the target structure) can be directly to the carrier and/or the first carrier surface (e.g. by a covalent or non-covalent bond) or be mediated by a carrier adapter (i.e. be indirect). To this end, the carrier adapter binds the target structure (specifically) and is bound and/or is being bound to the carrier and/or the first carrier surface. The binding of the carrier adapter to the carrier and/or the first carrier surface can again be direct (e.g. by a covalent or non-covalent bond) or mediated by an intermediate carrier adapter which specifically binds the carrier adapter and which specifically binds or is directly or indirectly bound to the carrier and/or the first surface thereof itself. In principle, further intermediate carrier adapters according to the same concept are conceivable. It is preferred that the carrier adapter itself is directly bound/is being bound to the carrier and/or the first carrier surface. The carrier and/or the first carrier surface can, for example, be coated with the carrier adapter.

“Carrier adapter” preferably refers to a particle that can bind specifically to subregions of target structures (e.g. target molecules) or a nanoreporter (i.e. one of the at least two 3D DNA nanostructures) and that can be coupled to a carrier (directly or indirectly). The specific binding to the target structure can be achieved, for example, by Watson-Crick base pairing, by Van Der Waals interactions or hydrogen bridges and be realized, inter alia, with nucleic acids, antibodies, aptamers, adhirons or nanobodies (depending on the target structure).

A carrier on which the identification structure is/is being bound can have different shapes and be formed of different materials. Preferably, the carrier has a first carrier surface and the identification structure is bound on the first carrier surface. Consequently, in the method for the detection of a target structure, the identification structure preferably is bound and/or is being bound to a carrier surface. The first carrier surface of a carrier can comprise a complete lateral surface of a carrier structure. Alternatively, only a part of a lateral surface of a carrier structure can be the first surface. The first carrier surface can be a surface of at least 0.01 mm², preferably at least 1 mm² or particularly preferably at least 10 mm². The first carrier surface can comprise an area of at most 1000 mm², preferably at most 400 mm² or particularly preferably at most 100 mm². A carrier surface and/or the first surface of the carrier preferably comprises a glass surface (e.g. a borosilicate glass surface or a quartz glass surface) or a polymer surface (such as e.g. μ-Slide VI^{0.1} of ibidi GmbH, or surfaces that are based on other polymers suited for fluorescence microscopy). Further, a carrier surface and/or the first surface of the carrier can be a glass surface (e.g. a borosilicate glass surface or a quartz glass surface) or a polymer surface (such as e.g. μ-Slide VI^{0.1} of ibidi GmbH, or surfaces that are based on other polymers suited for fluorescence microscopy). In other words, the first surface of the carrier preferably is a glass surface (e.g. a borosilicate glass surface or a quartz glass surface) or a polymer surface (such as e.g. μ-Slide VI^{0.1} of ibidi GmbH, or surfaces that are based on other polymers suited for fluorescence microscopy). Consequently, a carrier can comprise a first surface which comprises or consists of a glass surface (e.g. a borosilicate glass surface or a quartz glass surface) or a polymer surface (such as e.g. μ-Slide VI^{0.1} of ibidi GmbH, or surfaces that are based on other polymers suited for fluorescence microscopy). The carrier can also comprise a polymer network which preferably comprises one of or a combination of the following materials: biopolymer, agarose, collagen. A preferred carrier in the context of the invention is a microscopy chip, a well or a plate (preferably suited for high-resolution fluorescence microscopy; e.g. a μ plate of Ibidi) or a cover slip, most preferred a microwell array and/or a microwell chamber.

The carrier and/or the first carrier surface is preferably passivated. “Passivated” means that the carrier and/or the first carrier surface are coated or treated such that unspecific binding of a target structure and/or a 3D DNA nanostructure and/or optionally further components, which get in contact with the carrier and/or the first carrier surface (e.g. further components of the sample which contains the target structure), is minimized. Preferably, passivate can mean that the surface is contacted with and/or washed with a BSA solution having a concentration of 0.1 mg/ml to 10 mg/ml, preferably 0.5 mg/ml to 1 mg/ml. Passivating can also be carried out by PEGylation (for example described in the following publications: S Chandradoss et al, Jove 2014, “Surface Passivation for Single-molecule Protein Studies”, J Pichler et al, Biosensors & Bioelectronics 2000, “A high-density poly(ethylene glycol) polymer brush for immobilization on glass-type surfaces”, R Schlapak et al, Langmuir 2006, “Glass Surfaces Grafted with High-Density Poly(ethylene glycol) as Substrates for DNA Oligonucleotide Microarrays”) or silanization (for example described in the following publications: B Hua et al. and Tj Ha, Nature Methods 2014, “An improved surface passivation method for single-molecule studies”, A. Kumar et al, Nuc Ac Research 2000, “Silanized nucleic acids: a general platform for DNA immobilization”, H Labit et al, BioTechniques 2008, “A simple and optimized method of producing silanized surfaces for FISH and replication mapping on combed DNA fibers”). Moreover, passivating can also comprise washing the carrier or the carrier surface with a solution that comprises structures that are of the same type as the target structure (i.e. single-stranded polynucleotides), but not identical to the target structure. The carrier and/or the first surface of the carrier is particularly preferably passivated, when the first carrier surface is or comprises a glass surface. With glass surfaces, it is preferably passivated with a BSA solution, PEGylation or silanization.

A carrier can also comprise a matrix, such as, e.g., a polymer matrix. This is particularly advantageous for analyses at a single-cell level. In this case, it is preferred that the identification structure is/or is being embedded in the polymer matrix. Alternatively or additionally, the identification structure can be bound/being bound to a surface of the polymer matrix. As starting substance for the matrix and/or polymer matrix, in particular agarose, collagen, DNA and/or a combination of the same are suited. Agarose is particularly preferred. Methods for the production and embedding of an identification structure and/or a component of an identification structure (in the case of pre-binding of the target structure and/or one of the at least two 3D DNA nanostructures) are well-known to the skilled person. To this end, the agarose matrix can be produced of a mixture of agarose and biotinylated agarose (commercially available) or streptavidin agarose (commercially available), and target adapters or carrier adapters can be bound to these functionalizations (in the case of biotinylated agarose with previous streptavidin rinsing). Alternatively and for the other optional matrix materials, the adapter can be coupled (directly or via biotin-streptavidin) to an antibody that specifically binds to the matrix material (i.e. anti-agarose, anti-collagen, etc.). It is further possible that the adapter is bought in maleimid or NHS ester modified form and is directly covalently bound to the corresponding chemical groups of the matrix material. In the case of a DNA polymer matrix as a carrier, it can per se contain single-stranded segments having carrier sequences.

The use of such a matrix can be in combination with the microwell array. The above-mentioned polymer matrix can limit the diffusion of target structures which were released from a cell or another host body by lysis. The use of such a matrix is therefore advantageous in particular as an alternative but also in addition to method steps f) and g), in order to prevent that the content of a microwell to be analyzed changes by entry from outside and/or outflow from a microwell, e.g. by diffusion, and thereby distorting the analysis result. When using such a carrier matrix, it must be ensured that the polymer matrix is to be produced in a manner so fast that the host body cannot change its target structure composition prior to host body disruption or that the polymer matrix is to be produced such that the target structure composition of the host body is not influenced by this. The latter can, for example, be ensured by using low melting point agarose.

In a preferred embodiment, in a method step aa), preferably after method step p1), a carrier matrix is produced in the microwell array and carrier adapters are preferably fixed to the carrier matrix in a method step bb).

In particular, a combination of method steps aa) and bb) with already described method steps is preferred so that a preferred embodiment is a method for gene expression analysis, wherein an mRNA is detected (thus, the mRNA is the target structure) and the method comprises:

p1) introduction of a group of host bodies including a host body with the mRNA into a microwell array such that exactly one host body is present in at least one microwell;

aa) Producing a carrier matrix in the at least one microwell.

bb) Attaching carrier adapters to the carrier matrix.

p2) introduction of at least two 3D DNA nanostructures into the at least one microwell, wherein each of the 3D DNA nanostructures comprises one or more inwardly disposed fluorescence dye molecules;
a) forming an identification structure, comprising:
   (i) an mRNA, and
   (ii) the at least two 3D DNA nanostructures, wherein each of the 3D DNA nanostructures comprises one or more inwardly disposed fluorescence dye molecules and wherein each of the 3D DNA nanostructures is (specifically) bound to sequence segments of the mRNA that are pairwise different;
b) detection of the mRNA by measuring at least one fluorescence signal,
   wherein the 3D DNA nanostructures and the parameters of fluorescence measurement are selected such that the at least one measured fluorescence signal of the identification structure formed in a) is distinguishable from the fluorescence signal of each of the at least two isolated 3D DNA nanostructures, when these are not bound in the identification structure.

It is also possible to use said method mutatis mutandis for other target structures than mRNA.

The polymer network preferably comprises a medium mesh size of 1 μm to 50 μm, preferably 2 μm to 10 μm. This is to limit diffusion and to provide sufficient binding sites for carrier adapters and/or carrier adapter binding sites. The pairwise minimum distance of the carrier adapters and/or carrier adapter binding sites may essentially be 200 nm to 10 μm, preferably 500 nm to 5 μm, particularly preferably 2 μm to 3 μm. In this context, essentially means that at least 80%, preferably at least 90% and particularly preferably at least 99% of the carrier adapter and/or carrier adapter binding sites have at least the above-mentioned distance of adjacent carrier adapters and/or carrier adapter binding sites.

The bond and/or the binding of the identification structure to the carrier and/or the carrier surface (e.g. via target structure or at least one of the at least two DNA nanostructures of an identification structure) can be carried out via different covalent and non-covalent bonds known to the skilled person irrespective of whether it is direct or via a carrier adapter or an intermediate carrier adapter bound to the carrier adapter. For example, the binding can be achieved via biotin-streptavidin coupling. Consequently, the component that directly interacts with the carrier (e.g. carrier adapter, target structure, one of the DNA nanostructures or an intermediate carrier adapter) can either comprise biotin or streptavidin. Accordingly, the carrier and/or the carrier surface can comprise streptavidin and/or biotin as a counterpart so that a streptavidin-biotin interaction is possible. Apart from streptavidin-biotin interactions, also other interactions such as, e.g., antibody bindings can be used. It is also conceivable that the component which directly interacts with the carrier (e.g. carrier adapter, target structure, one of the DNA nanostructures or an intermediate carrier adapter) is (being) attached via a carrier adapter by NHS reaction on an amine-modified carrier and/or an amine-modified carrier surface. The application of click-chemistry methods (for example described in H. C. Kolb; M. G. Finn; K. B. Sharpless (2001). “Click Chemistry: Diverse Chemical Function from a Few Good Reactions”. Angewandte Chemie International Edition. 40 (11): 2004-2021) is also conceivable.

By means of the method of the invention, several copies of the target structure can be detected. If several copies of the target structure are detected (i.e. if the identification structure forms multiple times), preferably the majority of, preferably all, identification structures formed for said target structure is/are bound to the carrier, preferably to the first surface of the carrier. Preferably the majority or essentially all of the identification structures is/are attached to the carrier such that these are still resolvable by the measurement method used for the measurement of the at least one fluorescence signal in b). This may mean that all or essentially all identification structures and/or their measurement events do not overlap spatially (e.g. the diffraction-limited images of the identification structures when using fluorescence microscopy). Preferably all or essentially all identification structures have a distance of at least 250 nm, preferably at least 500 nm and particularly preferably at least 1 μm. This may mean that several carrier adapters or intermediate carrier adapters are attached with the mentioned minimum distance on the carrier and/or the first carrier surface. Essentially all means that at least 80%, preferably at least 90% and particularly preferably at least 95% of the formed identification structures fulfill these requirements.

Preferably, several identification structures are/are being bound to the carrier and/or the carrier surface such that the fluorescence signal of the individual identification structures can be measured separately. Via the density and/or the distance of the binding sites on the surface, i.e., for example, via the density of the streptavidin and/or biotin and/or the carrier adapters or intermediated carrier adapters, which is attached to/in the carrier and/or its surface, it can be regulated in which spatial distance identification structures bind to the carrier and/or carrier surface.

Preferably, the binding sites, for example, the streptavidin, the biotin, the carrier adapter or the intermediate carrier adapter are arranged on the carrier and/or the carrier surface in the following distance/pattern/density.

Preferably, all or essentially all binding sites have a distance of at least 250 nm, preferably of at least 500 nm and particularly preferably of at least 1 μm. Preferably, the binding sites are arranged in a hexagonal lattice having the above-mentioned edge length. The binding sites can preferably also be arranged on the carrier and/or its surface in an arbitrary manner, with a surface density of 1e-5/μm²-1e3/μm², preferably 1e-3/μm²-4/μm² and particularly preferably 0.1/μm²-1/μm². Said surface density ensures sufficient distance of the binding sites.

Additionally or alternatively, the regulation of the spatial distance of the identification structures on the carrier and/or its surface can be regulated via the concentration of the identification structures. If the concentration is selected such that no saturation of the binding sites on the carrier occurs, every further reduction of the concentration (i.e. dilution) causes a reduction in the attachment density on the carrier. Optionally, in order to determine the necessary dilution, the method is carried out once or several times in a dilution series until the suitable dilution/concentration has been established. Accordingly, after the formation of the identification structure(s), the method of the invention can also comprise the step of diluting the solution in which the identification structure formed, the step being prior to binding of the identification structure to the carrier.

As mentioned above, the target structure in the present method can be or comprise a partially single-stranded polynucleotide or single-stranded polynucleotide. In this case, it is particularly preferred that the carrier adapter comprises or is an oligonucleotide, the nucleic acid sequence of which is designed such that it specifically binds to a first single-stranded segment of the nucleic acid sequence of the target structure of claim 2. It is particularly preferred that the target structure is an mRNA having a poly-(A) tail, wherein the carrier adapter comprises or is an oligonucleotide, the nucleic acid sequence of which is designed such that it specifically binds to the poly-(A) tail of the mRNA target structure. Being designed in order to bind to a poly-(A) tail can include comprising a poly-(T) sequence segment.

As mentioned above, the target structure in the present method can also be a protein or comprise a protein. In this case, it is particularly preferred that the carrier adapter comprises or is an antibody or an antigen-binding domain of an antibody, which (specifically) binds to the target structure.

The method for the detection of a target structure after a) and prior to b) can further comprise washing of the carrier and/or the first carrier surface with a buffer solution. Such a washing step can have the purpose of removing components that are not bound to the surface. In particular, such a washing step can ensure that free 3D DNA nanostructure that are not bound on the identification structure are washed off the carrier and/or the first carrier surface. This has, inter alia, the advantage that a possibly disruptive background of fluorescence signal by free 3D nanostructures is reduced or prevented.

The washing step is preferably carried out with a buffer solution. The composition of the buffer is preferred as defined already above in connection with other washing steps.

In an identification structure, a 3D DNA nanostructure can be bound either directly or via a target adapter to the target structure.

Therefore, at least one, preferably all of the at least two 3D DNA nanostructures may be designed for direct binding to the target structure and be directly bound to the target molecule. Preferably, at least one, particularly preferably all of the at least two 3D DNA nanostructures are designed such that it/they is/are directly designed to the respective region(s) of the target structure and directly bound to the target molecule. Direct binding can for example occur via base pairing (e.g., with a partially single-stranded or single-stranded polynucleotide as target structure). Consequently, at least one, preferably all of the at least two 3D DNA nanostructures can contain a single-stranded sequence segment, which can specifically bind to the target structure (e.g. a partially single-stranded or single-stranded polynucleic acid). Preferably, said sequence segment is disposed outwardly and/or arranged at an outer surface of the 3D DNA nanostructure. It is thereby ensured that the sequence segment is accessible in the best possible way.

Alternatively, the specific binding of at least one, preferably all 3D DNA nanostructures can be mediated by a respectively assigned target adapter. The target adapter or each of the target adapters is designed to bind to the respective DNA nanostructure and to the respective region(s) of the target structure. Thus, a target adapter can comprise a first segment that is designed to (specifically) bind to the respective 3D DNA nanostructure. Moreover, a target adapter can comprise a second segment (also referred to as target binding segment) which is designed to (specifically) bind to the target structure. Preferably, a 3D DNA nanostructure, the binding of which to the target structure is mediated by a target adapter, comprises at least one single-stranded DNA segment that is designed such that it specifically binds the target adapter. This single-stranded DNA segment is preferably disposed outwardly, i.e. is accessible from the outside. The above-mentioned first segment of the target adapter preferably is a polynucleic acid sequence (e.g. a DNA, LNA or RNA sequence) which is designed such that it specifically binds to the single-stranded DNA segment of the 3D DNA nanostructure by hybridization (i.e. is complementary to at least a part of the single-stranded DNA segment of the 3D DNA nanostructure). The binding occurs via base pairing. Preferably, at least 15, preferably at least 18 and particularly preferably 21 bases of the two single-stranded segments hybridize.

A target binding adapter preferably comprises one (and/or at least one) target binding segment. The target binding segment is preferably designed such that it mediates the specific binding to the target structure and/or the respective region of the target structure, to which the respective identification structure binds.

If the target structure is or comprises a partially single-stranded polynucleotide or preferably a single-stranded polynucleotide (e.g. mRNA), the target binding segment preferably comprises a nucleic acid sequence that is designed such that it specifically binds to a single-stranded segment of the target structure. Preferably, the target binding segment is complementary to the single-stranded segment of the target structure. The binding to the target structure preferably occurs via base pairing. Preferably, at least 14, preferably at least 18 and particularly preferably 21 bases of the two single-stranded segments hybridize.

If the target structure is or comprises a protein, the target binding segment of the target adapter can comprise or be a peptide/protein or an antibody (and/or an antigen-binding fragment of an antibody) which specifically binds to the protein.

The method for the detection of a target structure can further comprise the provision of a preferably aqueous sample solution, which contains the target structure, and of the at least two 3D DNA nanostructures. The sample solution can, for example, comprise cell lysate, nucleic acids extracted from cell suspension and/or tissue and/or mRNA. Optionally, the method can further comprise the provision of the carrier, the carrier adapter(s) and/or the target adapter(s). One or both of said provision steps can be comprised by the step of the formation of the identification structure.

The method can further comprise mixing of a preferably aqueous sample solution, which contains the target structure, with the at least two 3D DNA nanostructures and optionally the carrier adapter and/or the target adapters. The sample solution can for example comprise cell lysate, nucleic acids extracted from cell suspension and/or tissue and/or mRNA. Furthermore, the method can comprise contacting said mixture with the carrier and/or the first carrier surface. The mixing of the components can also be stepwise.

A sample solution that contains the target structure can be a cell lysate, preferably the cell lysate of an individual cell. The sample solution can also be a mixture of nucleic acids, e.g. purified nucleic acids. In particular, the sample solution can also be a purified whole-RNA of cells. Said whole-RNA can be obtained by commercially available kits from cells (e.g. TRIzol® Reagent, TRIzol® LS, PureLink™ Total RNA Blood Kit, RecoverAll™ Total Nucleic Acid Isolation Kit for FFPE by Thermo Scientific, or RNeasy Kits, PAXgene Blood RNA Kit, or RNAlater reagent by Qiagen GmbH).

Preferably, the method of the invention for the detection of a target structure is also configured for the detection of further target structures that are different from the first target structure (wherein the different target structures are pairwise different). Consequently, the method can also be referred to as a method for the detection of at least two different target structures, wherein the at least two different target structures are pairwise distinguishable from each other.

In a further aspect, the invention is in particular directed to a method, which is further additionally suited
for the detection of one or more further target structures which are different from each other, wherein the different target structures are pairwise different,
wherein for each of the target structures, the group of host bodies in step p1) comprises at least one host body having the respective target structure;
and wherein the method further comprises:
c) for each of the one or more further target structures that are different from each other: forming of a respectively assigned identification structure, wherein each of the further identification structures comprises:
   (i) the respective assigned further target structure, and
   (ii) at least two 3D DNA nanostructures, wherein each of the at least two 3D DNA nanostructures comprises one or more inwardly disposed fluorescence dye molecules and wherein each of the at least two 3D DNA nanostructures is specifically bound to the respective further target structure and wherein the at least two 3D DNA nanostructures are bound to pairwise different regions of the respective target structure;
and wherein step a) further comprises:
d) detection of the one or more further target structures by measuring the at least one fluorescence signal,
wherein all 3D DNA nanostructures and the parameters of fluorescence measurement are selected such that the at least one measured fluorescence signal of the identification structures formed in a) and c) is distinguishable from the fluorescence signal of all isolated 3D DNA nanostructures, when these are not bound in one of the identification structures, and that the measured fluorescence signals of the identification structures formed are pairwise distinguishable from each other, wherein each of the different target structures may be present multiple times and the method may comprise the multiple detection of one or more of the different target structures.

Thus, this is a generalization of the previous detection and/or the quantification of one or more similar target structures of a first type to the detection and/or the quantification of one or more similar target structures of one or more further types which are pairwise different from each other and from the first type. In this case, the method is carried out for each of the target structures as described above for one target structure. However, the methods for the multiple target structures to be detected and/or quantified are carried out in an essentially parallel manner and identical method steps are each preferably summarized.

Preferably, each of the different target structures is detected multiple times, i.e. each of the identification structures is preferably formed multiple times. In other words, for example N different target structures can be detected, which are each pairwise distinguishable from each other, wherein each of the one or more of the N different target structures is detected multiple times. This implies that those identification structures that are assigned to the target structures that are detected multiple times are formed multiple times accordingly. As a matter of course, the fluorescence signals of the same identification structures that were formed for the respective same target structures do not have to be different from each other. Rather, the repeated measurement of the same fluorescence signal serves for quantification of the corresponding target structure.

For example, the method of the invention can have the purpose of detecting the target structures A, B and C that are different from each other in an aqueous solution. To this end, the 3D DNA nanostructures a1, a2, b1, b2, c1 and c2 assigned to these are provided, which together with the target structures A, B and C form the identification structures Aa1a2, Bb1b2 and Cc1c2. Here, the fluorescence signals of identification structures Aa1a2, Bb1b2 and Cc1c2 are pairwise different from each other and different from the isolated 3D DNA nanostructures a1, a2, b1, b2, c1 and c2. If the target structures A, B and C are each provided multiple times, the fluorescence signals of, for example, the same identification structures Aa1a2 do not have to be different from each other. Each measurement of a fluorescence signal of the identification structure Aa1a2 then corresponds to the detection of a target structure A so that the concentration can be derived from the number of measurement points.

What was mentioned above for the detection of a target structure applies mutatis mutandis to the detection of the further and/or each further target structure of the different target structures. In particular, everything above with regard to the target structure, identification structure, the bond or the binding of the identification structure to a carrier, the carrier adapter, the 3D DNA nanostructure(s), the target adapter and the further optional method steps is applicable mutatis mutandis to at least one, preferably all the further identification structures.

With respect to the preferred detection of multiple different target structures, the present invention is in one aspect also directed to a method for the detection of at least two different target structures, wherein the at least two different target structures are all pairwise distinguishable from each other and the method comprises:
a) Formation of an identification structure for each of the at least two target structures, comprising:
   (i) the respective target structure, and
   (ii) at least two 3D DNA nanostructures, wherein each of the 3D DNA nanostructures comprises one or more inwardly disposed fluorescence dye molecules and wherein each of the 3D DNA nanostructures is (specifically) bound to the target structure,
b) detection of the at least two target structures by measuring at least one fluorescence signal,

wherein all 3D DNA nanostructures and the parameters of fluorescence measurement are selected such that the at least one measured fluorescence signal of the identification structures formed in a) is distinguishable from the fluorescence signal of all isolated 3D DNA nanostructures, when these are not bound in one of the respective identification structures, and that the measured fluorescence signals of the identification structures formed for the respective different target structures are pairwise distinguishable from each other.

Also in this case, the preferred features specified above for the method for the detection of a target structure can be applied mutatis mutandis. In particular, everything mentioned above with regard to the target structure, identification structure, the bond or the binding of the identification structure to a carrier, the carrier adapter, the 3D DNA nanostructure(s), the target adapter and the further optional method steps is applicable mutatis mutandis to at least one, preferably each of the at least two identification structures. In particular, a combination with the method steps p1) and p2) is preferred.

If the method for the detection of a target structure is designed for the detection of further target structures which differ from the first target structure, or if the method is for the detection of at least two different target structures, the target structures can be target structures of the same type (e.g. several mRNAs) with pairwise different structure/sequence, or the target structures comprise several different target structures of the same type. Alternatively, also target structures of different types (e.g., at least one partially single-stranded DNA or single-stranded DNA and at least one partially single-stranded or single-stranded RNA; or at least one partially single-stranded or single-stranded RNA) are conceivable. Preferably, however, the target structures are of the same type and are pairwise distinguishable from each other in structure or in sequence. In particular, it is intended that the method of the invention is a method for gene expression analysis. For gene expression analysis, preferably all the target structures are RNAs, particularly preferably mRNAs, or proteins. In the case of gene expression analysis, particularly preferably all the target structures are RNAs, particularly preferably mRNAs.

If the method is for the detection of more than one target structure, particularly preferably at least one further identification structure comprising one of the further target structures, preferably all the further identification structures, are/are being bound to the carrier. Particularly preferably, all the identification structures are bound to the same carrier. The above “is being bound” and “is bound” mentioned in connection with an identification structure applies mutatis mutandis for the “are bound” and “are being bound” in the case of more/further identification structures.

In the embodiments where one, more or all the identification structures are bound/being bound to a carrier and/or a first carrier surface, preferably all or essentially all the identification structures are being bound/bound to the carrier in such a way that they can still be resolved by means of the measurement method used in b) for measuring the at least one fluorescence signal. This may mean that all or essentially all the identification structures and/or their measurement events (e.g. (diffraction-limited) images in the case of fluorescence microscopy) do not overlap spatially. Preferably, all the identification structures (of the same target structure or several target structures) comprise a distance of at least 250 nm, preferably at least 500 nm and particularly preferably at least 1 μm. Essentially all, in this context, means that at least 80%, preferably at least 90% and particularly preferably 95% of the identification structures formed meet this requirement.

The methods explained above, based on the example of the method for the detection of a target structure, as to how such density of the identification structures can be achieved, apply accordingly. In particular, a carrier and/or the first carrier surface may comprise (pre-)bound carrier adapters binding one or more target structures. These may be bound to the carrier and/or the first carrier surface with the distances mentioned above.

How “specifically bind” or “specifically bound” is to be understood in a method of the invention for the detection of one or more target structures is known to the person skilled in the art. In particular, in the context of hybridisation of polynucleotide single-strands, “specifically bind” can mean that at least 14, preferably at least 18 and particularly preferably at least 21 complementary bases pair. In particular, “specifically bind” can also mean that at least 90%, preferably 95%, particularly preferably 99% of the bases of the two complementary polynucleotide single-strands pair (preferably with the minimal number of base pairs mentioned in the previous sentence). In the context of protein-protein interactions (e.g. antibody antigen binding), “specifically bind” may preferably mean a binding affinity (−Δ ΔG) of at least 5 kcal/mol, preferably at least 8 kcal/mol and particularly preferably at least 11 kcal/mol. Binding affinities can be determined, e.g., by means of thermophoretic measurements (M. Jerabek-Willemsen et al., “Molecular Interaction Studies Using Microscale Thermophoresis”, Assay Drug Dev Technol, 9(4): 342-353 (2011)). Another possibility are electrodynamic measurements as offered by e.g. Dynamic Biosensors GmbH (A. Langer et al, “Protein analysis by time-resolved measurements with an electro-switchable DNA chip”, Nat. Comm. 4:2099 (2013)).

In the method of the invention, the one or more different target structures can be present in cells of a cell suspension, a droplet emulsion, an aqueous solution with bacteria, viruses or exosomes, a whole blood sample, saliva sample or any liquid of animal or human origin. Preferably, multiple or all target structures are present in host bodies in the same liquid. Preferably, the concentration of the host bodies in the liquid is high to such a degree that the probability of single occupancy of microwells is high. Preferably, when filling the microwell array, 0.5-1.5 host body per microwell, more preferably 0.8-1.2, more preferably 0.9-1.1 and particularly preferably 1 host body per microwell is present in the liquid. If a higher concentration of one or more host bodies is to be expected in a solution, the method can further comprise the dilution of the sample solution prior to formation of the identification structure(s). Preferably, each target structure is present in at least one host body in an amount of at least 1, preferably at least 10 and particularly preferably 100.

In the method of the invention, the formation of one or more identification structures preferably takes place in a buffer solution which is preferably designated hybridization buffer. The buffer solution can contain 1× to 8×SSC, preferably 3× to 5×SSC and particularly preferably 4×SSC. “SSC” refers to the so-called saline sodium citrate buffer consisting of an aqueous solution of 150 mM sodium chloride and 15 mM trisodium citrate, which is adjusted to pH 7.0 with HCl. As an alternative to a citrate buffer such as SCC, Tris or PBS can be also used as buffer bases. The buffer can (preferably as an alternative to SSC) also comprise NaCl and/or MgCl₂. The concentration of NaCl preferably is 50 mM to 1200 mM, particularly preferably 200 mM to 800 mM. For example, the NaCl concentration can be 600 mM, preferably 500 mM and particularly preferably 300 mM. The concentration of MgCl₂ can be 2 mM to 20 mM, preferably 5 mM to 15 mM, preferably 8 mM to 12 mM and particularly preferably 10 mM. Furthermore, the buffer solution can comprise 4% to 6%, 2% to 10%, 15% or 20% dextran sulphate. Preferably, the buffer comprises, for example, 5% dextran sulphate. The buffer solution can also comprise polyethylenglycol (PEG), e.g. PEG8000, PEG2000, PEG4000, PEG1000. The buffer can also comprise 0.01 to 5% Tween-20. Optionally, the buffer can also comprise EDTA, preferably in a concentration of 0.1 to 5 mM, particularly preferred 1 mM. A further optional component of the buffer is “sheared salmon sperm” (commercially available), preferably in a concentration of 0.1 mg/ml. Sheared salmon sperm can optionally increase specificity. The buffer can also comprise Denhardt's medium (consisting of an aqueous solution of 0.02% (w/v) BSA (Fraction V), 0.02% Ficoll 400 (commercially available) and 0.02% polyvinylpyrrolidone (PVP), see also Cold Spring Harb Protoc 2008, doi:10.1101/pdb.rec11538), preferably in one-fold, two-fold, three-fold, four-fold or five-fold concentration. A particularly preferred buffer comprises or has the composition 4×SSC, 5% dextran sulphate and 0.1% Tween 20. This buffer is used particularly preferably if at least one, preferably all, the target structures to be detected is/are (a) polynucleic acid(s), e.g. an mRNA.

In the method of the invention, the formation of the identification structure(s) in the method of the invention preferably takes place at a temperature of 4° C. to 50° C., preferably 18° C. to 40° C. For example, the temperature can be 40° C. 30° C. is used particularly preferably. The temperatures stated are particularly preferred if one or more partially single-stranded or single-stranded polynucleotides are to be detected.

The formation of the identification structure can comprise an incubation period of a mixture of the components bound therein. Preferably, the incubation period can be 5 min to 20 h, preferably 1 h to 20 h (e.g. 1 h, 2 h, 4 h, 8 h) and particularly preferably 10 h to 20 h.

In the methods of the invention, the one or several identification structure(s) can also be formed in one step instead of sequentially (preferably in the buffer solution mentioned above) and be bound to the carrier.

For the formation of the identification structure in one step, the DNA nanostructures (at least 2 per target structure; preferably in the concentration mentioned above) are added to the liquid containing host bodies. Optionally, also one or more carrier adapters can be added to the solution. Carrier adapters can, however, already be pre-bound to the carrier. After disruption of the host bodies in the microwells, all components of the identification structures are then already present in the microwells and can form them. Thus, the time period from begin of the preparation until the result of the analysis can be reduced drastically and the effort be reduced. This can be important, in particular, with single-cell gene expression analysis of time-sensitive clinical samples such as, for example, for the determination of sepsis pathogens. Also in general, however, it is advantageous economically.

For example, the carrier adapter can be added at a concentration of 1 nM per target structure to be bound to the carrier adapter. Preferably, carrier adapters are added in the total concentration of the 3D DNA nanostructures per target structure (e.g. 3 nM, with 3D DNA nanostructures for a target with 1 nM each). A total volume for the formation of the identification structure(s) can, for example, be 1 μl to 500 μl. For the binding of the formed identification structure, preferably, the mixture described above for the formation of the identification structure is added to the carrier and/or the first carrier surface. The carrier adapter can either be pre-bound (if it is not part of the solution for the formation of the identification structure) or be bound to the carrier surface during the incubation. The binding/the bond is either covalently or non-covalently (e.g. biotin streptavidin interaction). The binding to the carrier can comprise an incubation period of 1 min to 2 h, 5 min to 1 h, 8 min to 25 min and particularly preferably 10 min. Further examples for incubation periods are 2 min, 5 min, 20 min. Incubation can take place at 4° C. to 30° C., preferably room temperature (e.g. 20° C. to 25° C.). Prior to application to the carrier surface, the solution can be diluted again (e.g. with PBS or the above-mentioned buffer used for the formation of the identification structure) to, e.g., reduce the amount of dextran, cover a larger area or adjust the concentration. Prior to measurement, the surface can be washed with a buffer. Preferably, a buffer as defined above for the washing steps can be used.

Preferably, measurement of at least one fluorescence signal in the method of the invention comprises: q) Creating a data set containing data concerning fluorescence signals emitted from a sample portion/section, by means of fluorescence microscopy, preferably in epifluorescence, TIRF, light sheet and/or confocal microscopy. In other words, the measurement of at least one fluorescence signal can comprise the measurement of several fluorescence signals or a plurality of fluorescence signals in that, for example, the intensity emitted by a two- or three-dimensional sample portion is measured and stored in pixels or in voxels. In this context, measurement can also comprise a simultaneous or sequential measurement with several excitation wavelengths and/or several detection wavelengths (bands).

Preferably, the detection of the target structure and/or the one or more further target structures comprises: w) Identification of the one or more datum contained in the data set, representing the fluorescence signal of one of the identification structures.

This identification can comprise, for example, the comparison of the measured intensity of a pixel or a voxel with a specific excitation wavelength and/or detection wavelength with a set of such predetermined combinations so that the comparison is indicative of the identification structure to which the combination is assigned.

Accordingly, step q) can comprise: recording of at least one image file containing fluorescence data of the sample portion/section. Furthermore, the identification in step w) can comprise the following steps:
w1) readout of a color and/or an intensity information (and preferably) of a datum and/or image element; and
w2) comparing the color and/or intensity information (and preferably) the datum and/or image element with a color and/or intensity information, being representative for the identification structure.

Preferably, the samples are visualized by means of fluorescence microscopy technique. For further processing of the information, the provided image can be analyzed directly. Preferably, however, it is saved. Images are preferably saved digitally; however, images can also be recorded analogously. Image information can comprise the illustration of one or more image elements. An image element is part of an image. An image element can be, for example, an extended point or another structure of a first intensity on a background of a second intensity.

Depending on the selection of the fluorescence dyes on the DNA nanostructures, the target structures are distinguishable by their color and/or by their intensity. Preferably, the sample portion is recorded in every color individually and saved in a separate image. However, the sample portion can also be recorded in several colors simultaneously, wherein the recordings of different colors are saved in a common image or in separate images.

These steps can take place manually, manually but supported by software, or automatically by analysis software.

In a manual analysis, a user knowing the color and/or intensity information of the target structure to be detected can search and identify the corresponding color and/or intensity information in the image. Optionally, the user can analyze the number of such identified target structures and/or the corresponding location information. If the color and/or intensity information in multicolored identification structures are present in different images, the image elements can be analyzed for each color, i.e. each image, separately, including the location information. Subsequently the colored elements can be recognized by identification of image elements of different colors with sufficiently similar location information. Alternatively, first, a colored image can be created by superimposing the individual single-colored images. A colored identification structure can then be identified as image element with the corresponding mixed color. Care has to be taken in that the images do not have to be stored in the real color. Color can of course be added to the images, be it the real color or a false color, at a later point in time.

The manual analysis can take place by software support by means of image analysis software, for example Fiji or the like. There is a smooth transition towards fully automated analysis. In general, it is possible to carry out any individual step manually or with software.

One aspect of the invention relates to the analysis with software, wherein the analysis is based on the density based spatial clustering of applications with noise method (in short DBSCAN). The description is based on the application for the case that the image to be analyzed has several fluorescence elements. The analysis, of course, works in the same way if only one or no fluorescence element is present in the image.

Preferably, the analysis software programmed for this purpose is python-based. The python-based analysis software loads image data. Preferably, it determines local maxima in image boxes. Preferably, the size of the image boxes is adjusted dependent on the magnification and the numeric aperture of the objective used and is for example 9 to 15 pixels. Preferably, the software calculates the cumulative absolute value of the gradient as background-independent measurement value of the signal. In order to differentiate between image boxes with and without DNA nanostructures, preferably DBSCAN (density based spatial clustering of applications with noise) (published in: Ester, Martin; Kriegel, Hans-Peter; Sander, Jörg; Xu, Xiaowei “A density-based algorithm for discovering clusters in large spatial databases with noise”, Proceedings of the Second International Conference on Knowledge Discovery and Data Mining (KDD-96). AAAI Press. pp. 226-231 (1996)) is used. Alternatively, also other clustering algorithms such as HDBSCAN (Ricardo J. G. B. Campello, Davoud Moulavi, Joerg Sander; “Density-Based Clustering Based on Hierarchical Density Estimates”, Advances in Knowledge Discovery and Data Mining 2013, pp. 160-172), k-means or hierarchical clustering (Rokach, Lior, and Oded Maimon “Clustering methods.” Data mining and knowledge discovery handbook. Springer US, 2005. 321-352) can be used. Optionally, the image boxes are divided into groups of different values of the cumulative absolute value of the gradient, which corresponds to a division according to different numbers of fluorophores. This can take place in particular if different target structures are identified by means of identification structures of different intensities. Due to their transversal (x, y) positions, the image boxes with DNA nanostructures of a color channel can be compared with all the image boxes with DNA nanostructures of other color channels in order to recognize multicolored fluorescence elements, for example spots, in the image. This, of course, does not have to take place at all if only identification structures of a single color are present. The software can store the determined values for the number of single-color spots as well as optionally their locations in the image and the values for the number of the multicolored spots as well as optionally their locations in the image for further use. The software can also store the intensity values of the spots. For example, the software can be used to determine the number of the single-color fluorescence elements in comparison with the multicolored fluorescence elements. For example, the software can be used to compare the number of the fluorescence elements detected in experiments with samples with or without target structure. This is explained in detail in the appended Examples 1 to 3.

Due to the variation in the number of color dye molecules per 3 D nanostructure that can occasionally occur during their production as well as due to measurement errors, one is typically confronted with, for example, intensity distributions. If two adjacent intensity distributions have a non-neglectable overlap, one might not be able to conclude, simply due to an identity measurement, without doubt that there is exactly one identification structure. In order to address this problem, statistical methods are preferred by means of which a statistical assignment can be achieved. These distributions can extend one- or multi-dimensionally along intensity or color gradients and can each be described by means of one or more measured values, such as the mentioned cumulative gradient, the average value over the box, the maximum pixel value within the box or other measured values. The measured, optionally multi-dimensional distribution is a mixture distribution of the individual distributions of the identification structures with the same target structures. The aim of the analysis is to calculate the contribution of these individual distributions to the mixture distribution and, thus, to determine the relative number of the different target structures. These individual distributions can be determined either by separate measurements or approximated by means of models (for example Gaussian distributions and/or Poisson distributions or mixtures thereof, possibly different in different dimensions). Thus, the different contributions can be calculated by deconvolution or by statistical methods, preferably based on Bayesian inference (cf. D. S. Sivia “Data Analysis, a bayesian tutorial”, Oxford science publications, second edition, ISBN: 0198568320 and/or A. B. Downey, “Think Bayes”, O'Reilly, Sebastopol C A, fourth edition 2013 ISBN: 9781449370787). In this context, there is the possibility to initially assume a uniform distribution of the number of target structures and, s starting from there, to calculate—for each measurement event—the probability to originate from each of the distributions, thereby updating the assumption as to the distribution of the numbers of target structures. With a sufficiently high number of identification structure measurement events (over 50, preferably more than 100, particularly preferably more than 1000), this method is independent from the initial assumption of the distribution of the numbers of target structures.

We allow for the case that the intensity distributions of different nanoreporters overlap and are only clearly distinguishable from each other by statistical means by taking the distribution histograms into account as described in method step (h/ii). This adds complexity to the analysis because for each measurement event the probability to be derived from the various nanoreporters is calculated, and with greater overlap and the same amount of measurement events, the result becomes more uncertain. However, the number of different nanoreporter combinations and thus the number of target molecules can be increased since the intensity levels are reduced. Thus, there is a weighing between the size of overlap of the intensity distributions and the number of target molecules quantifiable simultaneously. Thus, the optimum configuration can be selected for each application.

Preferably, the fluorescence signals of the identification structures formed for the individual different target structures are different from the fluorescence signal of all the isolated 3D DNA nanostructures when those are not bound in one of the identification structures, and the fluorescence signals of the identification structures formed for the individual different target structures are pairwise different in that the corresponding fluorescence signals comprise a different distinguishable combination of color and/or intensity information. As explained above, a “different distinguishable combination” can be a combination that can be distinguished statistically, i.e. the distinguishability is guaranteed by means of statistical methods.

Preferably, in the 3D DNA nanostructures, k pairwise (statistically) distinguishable intensity levels and/or m pairwise (statistically) distinguishable color levels are used, wherein preferably, each of the k intensity levels is formed by an intensity distribution and wherein the k intensity distributions are distinguishable from each other, preferably statistically, and/or wherein preferably, each of the m color levels is formed by a color distribution and wherein the m color distributions, are distinguishable from each other, preferably statistically. For this purpose, preferably, the overlap of adjacent distributions is less than 30%, preferably less than 10%, particularly preferably less than 5%, particularly preferably less than 2% and highly particularly preferably less than 1%.

In this context, the following preferably applies: k>2, preferably k>3, more preferably k>4, even more preferably k>5, particularly preferably k>6. Furthermore, the following preferably applies: m>2, preferably m>3, more preferably m>4, even more preferably m>5, particularly preferably m>6.

It is further preferred that step w1) comprises one or a combination of the following steps and/or techniques:

Determining the average value of the image element, determining a maximum of the image element, calculating a cumulative gradient, calculating one or more statistical moments such as, e.g., variance of the pixels in the image element, variation coefficient, fano factor. It is further preferred that step w2) is based on a clustering method, preferably a DBSCAN (density based spatial clustering of applications with noise) method. It is further preferred that step w1) and/or w2) are based on probabilistic consideration, preferably taking in to account the Bayesian theorem.

In particular, the intensity gradations of the nanoreporters and/or the 3D DNA nanostructures of each orthogonal measurable type can be selected differently, for example, by variation of number, distance or orientation. The gradations can be so great that the intensity distributions which are extended due to factors such as Poisson statistics of the photons or incomplete marker molecule attachment do not overlap. Thus, the analysis is simple and the measurement values can be identified directly by means of nanoreporters. On the other hand, the gradiations can also be smaller so that the intensity distributions of adjacent intensity gradations overlap up to 0.1 to 10% and even 5 to 80%. Then, after measurement, the vectors from distributions of the orthogonal measurement results can be compared with those to be expected, and the relative frequency of the target molecules can be determined by means of Least Squares- or Maximum Likelihood-based methods. The same can be achieved with Cluster-based methods of machine learning with or without including the expected distributions.

It is also preferred that one of the orthogonal measurable nanoreporters is designed in a fixed amplitude value, the measurement value of which is clearly distinguishable from the one of the double amplitude value, and is designed onto each target molecule. Thus, the number of target molecules in a measurement event can be measured directly, which facilitates the detection and exclusion of measurement events, which are caused by target structures which are bound to the carrier in close proximity to each other.

For the method discussed above, preferably, a set of several 3D DNA nanostructures is used (wherein the 3D DNA nanostructures preferably are the ones as described in this application), wherein the set comprises N 3D DNA nanostructures which are different from each other and wherein the N 3D DNA nanostructures of the set which are different from each other are pairwise different due to the fluorescence dye molecules. Preferably, the N 3D DNA nanostructures which are different from each other comprise a different number of fluorescence dye molecules and/or different fluorescence dye molecules so that k distinguishable intensity levels and/or m distinguishable color levels can be generated by means of the N distinguishable 3D DNA nanostructures of the set. Preferably, at least a part of the N distinguishable 3D DNA nanostructures is contained several times in the set so that each of the k intensity levels is formed by an intensity distribution and wherein the k intensity distributions are distinguishable from each other, preferably statistically. Preferably, at least a part of the N different 3D DNA nanostructures is contained several times in the set so that each of the m color levels is formed by a color distribution and wherein the m color distributions are distinguishable from each other, preferably statistically. The overlap of the adjacent distributions is less than 30%, preferably less than 10%, more preferably less than 5%, even more preferably less than 2% and particularly preferably less than 1%.

Also in this context, the following applies preferably: k>2, preferably k>3, more preferably k>4, even more preferably k>5, particularly preferably k>6; and/or m>2, preferably m>3, more preferably m>4, even more preferably m>5, particularly preferably m>6.

In one aspect, the present invention is directed to a kit, comprising:
a microwell array, preferably of any one of the embodiments already described;
at least two 3D DNA nanostructures, wherein each of the 3D DNA nanostructures comprises at least one inwardly disposed fluorescence dye molecule.

The 3D DNA nanostructures are preferably 3D DNA nanostructures which are described above or hereinafter.

In particular, the present invention relates to a kit, wherein at least one of the 3D DNA nanostructures comprises at least 2 inwardly disposed fluorescence dye molecules and wherein the pairwise distance of the at least two inwardly disposed fluorescence dye molecules is at least 2 nm, preferably at least 5 nm and particularly preferably at least 9 nm.

One aspect of the invention relates to a kit which is provided for the detection and/or quantification of multiple different target structures. Preferably, a kit of the invention therefore comprises a set of multiple 3D DNA nanostructures, wherein the set comprises N different 3D DNA nanostructures and wherein the N different 3D DNA nanostructures of the set are pairwise different from each other in the fluorescence dye molecules.

In the method of the invention, the measuring of the at least one fluorescence signal can be carried out by fluorescence microscopy, preferably by one or a combination of the following techniques: point scanning, wide field microscopy, (spinning disc) confocal microscopy; two photons spectroscopy, epifluorescence, light sheet microscopy; TIRF; wherein each time preferably an alternating laser excitation (ALEX) or an excitation by means of pulsed-interleaved light sources takes place.

An exemplary method to achieve the desired objective with said DNA nanostructures or other nanoreporters has the following steps or consists in that
a. to each type of target structure, an unambiguous combination of one or more orthogonally measurable nanoreporters (e.g. 3D DNA nanostructures of the invention) is assigned in such a way that each type of target structure is distinguishable and identifiable in an unambiguous manner,
b. for each type of target structure, at least as many binding regions, as nanoreporters, are identified and adapters are selected which are able to bind specifically and unambiguously to the binding regions,
c. nanoreporters and adapters which are assigned to one type of target structure are unambiguously paired and coupled,
d. optionally, the adapters which are not assigned to nanoreporters are coupled to substrate, carrier or matrix,
e. the complexes consisting of nanoreporters and adapters are incubated with the target structures in solution,
f. the resulting complexes consisting of target molecules, adapters and nanoreporters are measured with measuring apparatuses for the markers present in such a way that, only with an acceptable low probability, more than one complex is responsible for a measuring event,
g. measuring events which do not have the minimum number of orthogonal signals are filtered out,
h. the other measuring events are either
   i. directly assigned a combination of nanoreporters by the individual signals, or
   ii. statistically assigned a probability of being derived from a combination of nanoreporters by comparison of the measured distribution of measuring signals and the distribution expected for each nanoreporter, or
   iii. assigned a probability of being derived from a combination of nanoreporters by cluster-based algorithms;
i. the relative number of target structures is determined by the total number of calculated combinations of nanoreporters or the probabilities thereof; and
j. optionally, with known absolute concentration of at least one of the target structures or with known measuring volumes, the absolute number of target structures is determined; and
k. optionally, a nanoreporter which can be measured orthogonally with respect to all other nanoreporters, can be designed for all target structures so that its measuring signal provides information on the number of target structures on which one measuring event is based.

In particular, it is envisaged that the methods of the invention and/or the 3D DNA nanostructures are used for the gene expression analysis, preferably the gene expression analysis at a single cell level.

For the analysis of gene expression at single level, a polymer matrix is preferably used as carrier which preferably envelops the host body partially or completely. In this case, the formation of the identification structure preferably comprises:

1) Providing a host body in a microwell;

2) Enveloping the host body with a polymer matrix (i.e. the carrier); it must be ensured that the polymer matrix is to be produced in a manner so fast that the host body cannot change its target structure composition prior to host body disruption or that the polymer matrix is to be produced such that the target structure composition of the host body is not influenced by this. The latter can, for example, be ensured by using low melting point agarose.

3) Disrupting the host body, e.g. in the case the host body is a cell, by lysing (e.g. mechanically by ultrasound, osmosis or freezing; or chemically, for example by modification of the pH value, introduction of EDTA, enzymes such as for example lysozyme, toluol, Triton-X or Trizol, which all destabilize the cell walls or membranes or induces autolysis), so that the mRNAs are released from the host body, e.g. the cell.

With corresponding selection of host body (e.g. cells) concentration and density of the polymer matrix, the polymer matrix limits the diffusion of target structures released from the host body in such a way that they do not diffuse out of the corresponding microwells. Preferably, the polymer network has a medium mesh size of 1 μm to 50 μm, preferably 2 μm to 10 μm. This is to limit diffusion and to provide sufficient binding sites for carrier adapters and/or carrier adapter binding sites. The pairwise minimum distance of the carrier adapters and/or carrier adapter binding sites can essentially be 200 nm to 10 μm, preferably 500 nm to 5 μm, particularly preferably 2 μm to 3 μm. In this context, essentially means that at least 80%, preferably at least 90% and particularly preferably at least 99% of the carrier adapter and/or carrier adapter binding sites have at least the above-mentioned distance of adjacent carrier adapters and/or carrier adapter binding sites.

For the analysis of the target structure composition of individual host bodies, it can be preferred
(a) to envelop a cell partially or completely with a polymer matrix in a microwell;
(b) to decorate the polymer matrix with elements which are able to bind to the target molecules, such as complementary oligonucleotides;
(c) to disrupt the host body so that the target structures diffuse and bind to the decoration elements;
(d) to add 3D DNA nanostructures or other elements and to wash these out after an appropriate incubation period;
(e) to image the sample for example on an epifluorescence, light sheet or confocal microscope and to analyze it as described above.

In this context, it is important to bear in mind that the decoration density of the elements binding target structures on the polymer matrix should be sufficiently low so that the majority of complexes do not overlap in the image. A suitable starting material for the polymer matrix is in particular agarose, however also collagen, DNA or other materials.

Herein, the term ‘cells’ includes all biologic organisms, in particular human cells, cells from tissue samples, animal cells, bacteria, fungi, algae. In particular, all features described for cells apply mutatis mutandis for other host bodies.

The term ‘matrix’ includes a gel from agarose or other substances the purpose of which is:
to limit the diffusion of target molecules in order to minimize target structure loss from the micro wells due to diffusion;
and/or to provide more anchor points for the target structures so that more target structures per microwell can be analyzed.

A gene expression analysis having single-cell accuracy may comprise:
1. Deposit of the host bodies to be analyzed in a microwell array.
2. Enveloping the host bodies with a polymer matrix.
3. Disruption of the host bodies so that the target structures may be released.
4. Formation of identification structures on the carrier (polymer matrix and/or surface of the 1^{st} and 2^{nd} layer of the microwell).
5. Detection of the identification structures with corresponding instruments
6. Analysis and assignment of the detected identification structures and the corresponding target structures to their original host body.

The present invention is inter alia directed to the following aspects:
1. A method for the detection (preferably quantification) of a target structure, comprising:
   p1) introduction of a group of host bodies including a host body with a target structure into a microwell array such that exactly one host body is present in at least one microwell;
   p2) introduction of at least two 3D DNA nanostructures into the at least one microwell wherein each of the 3D DNA nanostructures comprises one or more inwardly disposed fluorescence dye molecules;
   a) forming an identification structure in the at least one microwell, comprising:
      (i) the target structure, and
      (ii) the at least two 3D DNA nanostructures, wherein each of the 3D DNA nanostructures is specifically bound to the target structure and wherein the 3D DNA nanostructures are bound to pairwise different regions of the target structure;
   b) detection of the target structure by measuring at least one fluorescence signal,
   wherein the 3D DNA nanostructures and the parameters of the fluorescence measurement are selected such that the at least one measured fluorescence signal of the identification structure formed in a) is distinguishable from the fluorescence signal of each of the at least two isolated 3D DNA nanostructures, when these are not bound in the identification structure, wherein the identification structure is bound to a first surface, preferably the bottom of the at least one microwell.
2. The method of aspect 1, wherein the target structure comprises or is a partially single-stranded polynucleotide, preferably a single-stranded polynucleotide and particularly preferably an mRNA.
3. The method of any one of the preceding aspects, wherein the identification structure is bound to a carrier, preferably to a first surface of the carrier, and/or wherein the method further comprises the step of binding the formed identification structure to a carrier, preferably to a first surface of the carrier.
4. The method of aspect 3, wherein the carrier and/or the first surface of the carrier comprises and/or consists of a glass surface and/or a polymer surface, wherein the carrier and/or the first surface of the carrier is/are optionally passivated and/or wherein the carrier comprises a polymer network, which preferably comprises one of or a combination of the following materials: biopolymer, agarose, collagen.
5. The method of aspect 3 or 4, wherein the carrier is a microscopy chip or a cover slip.
6. The method of aspects 3 to 5, wherein the bond and/or the binding of the identification structure to the carrier and/or the first surface of the carrier is mediated and/or is being mediated via the target structure.
7. The method of aspect 6, wherein the target structure is bound and/or is being bound to the carrier and/or the first surface of the carrier, wherein the bond and/or binding is mediated by a carrier adapter that specifically binds and/or is bound to the target structure.
8. The method of aspect 7, wherein the carrier adapter is bound and/or is being bound directly to the carrier or the first surface of the carrier.
9. The method of aspect 7 or 8, wherein the carrier adapter is bound and/or is being bound to the carrier or the first surface of the carrier via a covalent or via a non-covalent bond.
10. The method of any one of aspects 7 to 9, wherein the carrier adapter is bound and/or is being bound to the carrier or the first surface of the carrier via a biotin streptavidin bond, wherein either the carrier adapter comprises biotin and the carrier and/or the first surface of the carrier comprises streptavidin, or wherein the carrier adapter comprises streptavidin and the carrier and/or the first surface of the carrier comprises biotin.
11. The method of any one of aspects 7 to 10, wherein the target structure is a target structure of aspect 2, and wherein the carrier adapter is an oligonucleotide or a polynucleotide, the nucleic acid sequence of which is designed such that it specifically binds, preferably by hybridization, to a first single-stranded segment of the nucleic acid sequence of the target structure of aspect 2.
12. The method of any one of aspects 7 to 10, wherein the target structure is an mRNA having a poly-(A) tail, and wherein the carrier adapter is an oligonucleotide, the nucleic acid sequence of which is designed such that it specifically binds to the poly-(A) tail of the mRNA.
13. The method of any one of aspects 3 to 12, wherein the method after a) and prior to b) further comprises washing the carrier and/or the first carrier surface with a buffer solution.
14. The method of any one of the preceding aspects, wherein one, preferably more, and particularly preferably all of the 3D DNA nanostructures is/are designed for direct binding to the respective region(s) of the target structure and is/are directly bound to the target molecule.
15. The method of any of the preceding aspects, wherein the specific binding of at least one, preferably of all 3D DNA nanostructures is mediated by a target adapter assigned to a corresponding 3D DNA nano structure, wherein the target adapter and/or each of the target adapters is designed to bind to the respective DNA nanostructure and to the respective region(s) of the target structure, and/or wherein the target adapter and/or each of the target adapters comprises or is preferably an oligonucleotide or polynucleotide, particularly preferably DNA.
16. The method of aspect 15, wherein the 3D DNA nanostructure(s) comprise(s) at least one single-stranded DNA segment which is arranged at or on the exterior of the nanostructure and which is designed such that it specifically binds to the respective target adapter.
17. The method of any one of aspects 15 or 16, wherein the one or more target adapters each comprise a single-stranded DNA segment, which is each designed such that it mediates the specific binding to the respective 3D DNA nanostructure.
18. The method of any one of aspects 15 to 17, wherein the one or more target adapters each comprise a target binding segment, which is each designed such that it mediates the specific binding to the respective region(s) of the target structure.
19. The method of aspect 18, wherein the target structure is the target structure as defined in aspect 2, and wherein the respective target binding segment comprises a nucleotide sequence that specifically binds to the one single-stranded segment of the target structure.
20. The method of aspect 18, wherein the target structure comprises or is a protein, and wherein the respective target binding segment comprises a peptide, preferably an antibody, which specifically binds to the protein of the target structure.
21. The method of any one of the preceding aspects, wherein the method further comprises providing a preferably aqueous sample solution which contains the target structure and providing the at least two 3D DNA nanostructures, and optionally further comprises providing the carrier, the carrier adapter and/or the target adapter, wherein the sample solution preferably comprises cell lysate, nucleic acids extracted from cell suspension and/or tissue and/or mRNA.
22. The method of any one of the preceding aspects, wherein the method further comprises mixing of a preferably aqueous sample solution, which contains the target structure, with the at least two 3D DNA nanostructures and optionally the carrier adapter and/or the target adapters, wherein the sample solution preferably comprises cell lysate, nucleic acids extracted from cell suspension and/or tissue and/or mRNA.
23. The method of any one of the preceding aspects, wherein the method is further additionally suited for the detection of one or more further target structures that are different from each other, wherein the different target structures are pairwise different, and wherein the method further comprises:
   c) for each of the one or more further target structures that are different from each other: Formation of a respectively assigned identification structure, wherein each of the further identification structures comprises:
      (i) the further target structure that was assigned, and
      (ii) at least two 3D DNA nanostructures, wherein each of the at least two 3D DNA nanostructures comprises one or more inwardly disposed fluorescence dye molecules and wherein each of the at least two 3D DNA nanostructures is specifically bound to the respective further target structure, and wherein the at least two 3D DNA nanostructures are bound to regions of the respective target structure that are pairwise different;
   and wherein step b) further comprises:
   d) detection of the one or more further target structures by measuring the at least one fluorescence signal,
   wherein all 3D DNA nanostructures and the parameters of the fluorescence measurement are selected such that the at least one measured fluorescence signal of the identification structures formed in a) and c) is distinguishable from the fluorescence signal of all isolated 3D DNA nanostructures, when these are not bound in one of the identification structures, and that the measured fluorescence signals of all formed identification structures are pairwise distinguishable from each other, wherein each of the different target structures may be present multiple times and the method may comprise the multiple detection of one or more of the different target structures.
24. The method of aspect 23, wherein the further target structures each comprise or are a partially single-stranded polynucleotide, preferably a single-stranded polynucleotide and particularly preferably an mRNA.
25. The method of any of aspect 23 or 24, wherein at least one, preferably all, identification structures are bound to a carrier, preferably to a first surface of the carrier, and/or wherein the method further comprises the step of binding at least one, preferably all formed identification structure(s) to a carrier, preferably to a first surface of the carrier.
26. The method of aspect 25, wherein the surface of the carrier comprises or is a glass surface and/or a polymer surface and is optionally passivated and/or wherein the carrier comprises a polymer network, which preferably comprises one of or a combination of the following materials: biopolymer, agarose, collagen.
27. The method of aspect 25 or 26, wherein the carrier is a microscopy chip or a cover slip.
28. The method of aspects 25 to 27, wherein the bond and/or the binding of at least one, preferably all of the identification structure(s), which are bound/are being bound to the carrier and/or the first surface of the carrier is mediated and/or is being mediated via the respective target structure.
29. The method of aspect 28, wherein the bond and/or binding of the respective target structure to the carrier and/or the first surface of the carrier is mediated by a respectively assigned carrier adapter that specifically binds to the respective target structure.
30. The method of aspect 29, wherein the respective carrier adapter is bound and/or is being bound, directly to the carrier or the first surface of the carrier.
31. The method of aspect 29 or 30, wherein the respective carrier adapter is bound and/or is being bound via a covalent or via a non-covalent bond to the carrier or the first surface of the carrier.
32. The method of any one of aspects 29 to 31, wherein the respective carrier adapter is bound and/or is being bound to the carrier or the first surface of the carrier via a biotin-streptavidin bond, wherein either the respective carrier adapter comprises biotin and the carrier and/or the first surface of the carrier comprises streptavidin, or wherein the respective carrier adapter comprises streptavidin and the carrier and/or the first surface of the carrier comprises biotin.
33. The method of any one of aspects 29 to 32, wherein the respective target structure is a target structure of aspect 2 and wherein the respective carrier adapter is a polynucleotide or an oligonucleotide, the nucleic acid sequence of which is designed such that it specifically binds to a first single-stranded segment of the nucleic acid sequence of the respective target structure of aspect 2.
34. The method of any one of aspects 29 to 33, wherein the target structure(s) bound to the carrier and/or the first surface of the carrier is/are an mRNA having a poly-(A) tail, and wherein the respective carrier adapter for said bound target structure(s) is an oligonucleotide, the nucleic acid sequence of which is designed such that it specifically binds to the poly-(A) tail of the mRNA.
35. The method of any one of aspects 23 to 34, wherein a) and c) are carried out in one step, preferably concomitantly.
36. The method of any one of aspects 25 to 35, wherein the method after a) and c) and prior to b) further comprises washing of the carrier and/or the first carrier surface with a buffer solution.
37. The method of any one of aspects 23 to 36, wherein one, preferably more and particularly preferably all of the 3D DNA nanostructures is/are designed for direct binding to the respective region(s) of the respective target structure and is/are directly bound to the target molecule.
38. The method of any one of aspects 23 to 37, wherein the specific binding of at least one, preferably all of the 3D DNA nanostructure(s) is mediated by a respectively assigned target adapter, wherein the target adapter and/or each of the target adapters is designed to bind to the respective DNA nanostructure and to the respective region(s) of the respective target structure.
39. The method of aspect 38, wherein the at least one, preferably all of the 3D DNA nanostructure(s) comprise(s) at least one single-stranded DNA segment, which is arranged at or on the exterior of the nanostructure and which is designed such that it specifically binds to the respective target adapter.
40. The method of any one of aspects 38 or 39, wherein the one or more target adapters each comprise a single-stranded DNA segment, which is each designed such that it mediates the specific binding to the respective 3D DNA nanostructure.
41. The method of any one of aspects 38 to 40, wherein the one or more target adapters each comprise a target binding segment, which is each designed such that it mediates the specific binding to the respective region(s) of the target structure.
42. The method of any one of aspects 23 to 41, wherein all target structures are present in the same sample and are preferably provided in a sample solution.
43. The method of any one of the preceding aspects, wherein the measuring of the at least one fluorescence signal using fluorescence microscopy is preferably carried out by one or a combination of the following techniques: point scanning, broad field microscopy, (spinning-disc) confocal microscopy; wherein preferably an alternating excitation (ALEX) or an excitation using pulsed light sources is carried out pulse-interleaved.
44. The method of any one of aspects 1, 2, 14 to 24, 35 or 37 to 42, wherein the measuring of the at least one fluorescence signal is carried out by flow cytometry and/or fluorescence correlation spectrometry (FCS), preferably by one or a combination of the following techniques: confocal microscopy, two-photon spectroscopy, epifluorescence, light sheet microscopy, TIRF; wherein preferably an alternating excitation (ALEX) or an excitation using pulsed light sources is carried out pulse-interleaved.
45. The method of any one of the preceding aspects, wherein the identification structure or at least one and/or all of the identification structures is/are formed multiple times.
46. A method for the detection of at least two different target structures, wherein the at least two different target structures are pairwise distinguishable from each other and the method comprises:
   p1) introduction of a group of host bodies into a microwell array such that exactly one host body is present in at least one microwell; wherein for each of the target structures the group of host bodies comprises at least one host body having the relevant target structure;
   p2) introduction of at least two 3D DNA nanostructures into the at least one microwell, wherein each of the 3D DNA nanostructures comprises one or more inwardly disposed fluorescence dye molecules;
   a) forming a respective identification structure for each of the at least two different target structures, comprising:
      (i) the respective target structure, and
      (ii) at least two 3D DNA nanostructures, wherein each of the at least two 3D DNA nanostructures comprises one or more inwardly disposed fluorescence dye molecules and wherein each of the at least two 3D DNA nanostructures is specifically bound to the respective target structure and wherein the at least two 3D DNA nanostructures are bound to pairwise different regions of the respective target structure;
   b) detection of the one or more different target structures by measuring at least one fluorescence signal,
   wherein all 3D DNA nanostructures and the parameters of the fluorescence measurement are selected such that the at least one measured fluorescence signal of the identification structures formed in a) is distinguishable from the fluorescence signal of all isolated 3D DNA nanostructures, when these are not bound in one of the identification structures, and that the measured fluorescence signals of the different identifications structures formed for the individual target structures are pairwise distinguishable from one another, wherein each of the different target structures may be present multiple times and the method may comprise the multiple detection of one or more of the different target structures
47. The method of aspect 46, wherein the at least two target structures are present in the same sample.
48. The method of aspect 1, wherein at least one, preferably each of the target structures comprises or is a partially single-stranded polynucleotide, preferably a single-stranded polynucleotide and particularly preferably an mRNA.
49. The method of any one of aspects 46 to 48, wherein at least one, preferably all identification structure(s) is/are bound to a carrier, preferably to a first surface of the carrier, and/or wherein the method further comprises the step of binding at least one, preferably all formed identification structure(s) to a carrier, preferably to a first surface of the carrier.
50. The method of aspect 49, wherein the carrier and/or the first surface of the carrier comprises and/or consists of a glass surface and/or polymer surface and is optionally passivated.
51. The method of aspect 49 or 50, wherein the carrier is a microscopy chip or a cover slip.
52. The method of aspects 49 to 51, wherein the bond and/or the binding of at least one, preferably all of the identification structure(s) that are bound to/are being bound to the carrier and/or the first surface of the carrier is mediated or is being mediated via the respective target structure.
53. The method of aspect 52, wherein the respective target structure is bound and/or is being bound to the carrier and/or the first surface of the carrier, wherein the bond and/or the binding is mediated by a carrier adapter assigned to the respective target structure and which specifically binds to the respective target structure.
54. The method of aspect 53, wherein the respective carrier adapter is bound and/or is being bound directly to the carrier or the first surface of the carrier.
55. The method of aspect 53 or 54, wherein the respective carrier adapter is bound and/or is being bound via a covalent or via a non-covalent bond to the carrier or the first surface of the carrier.
56. The method of any one of aspects 53 to 55, wherein the respective carrier adapter is bound and/or is being bound via a biotin-streptavidin bond to the carrier or the first surface of the carrier, wherein either the respective carrier adapter comprises biotin and the carrier and/or the first surface of the carrier comprises streptavidin, or wherein the respective carrier adapter comprises streptavidin and the carrier and/or the first surface of the carrier comprises biotin.
57. The method of any one of aspects 53 to 56, wherein the respective target structure is a target structure of aspect 2, and wherein the respective carrier adapter is an oligonucleotide, the nucleic acid sequence of which is designed such that it specifically binds to a first single-stranded segment of the nucleic acid sequence of the respective target structure of aspect 2.
58. The method of any one of aspects 53 to 57, wherein the target structure(s) bound to the carrier and/or the first surface of the carrier is/are an mRNA having a poly-(A) tail, and wherein the respective carrier adapter for said bound target structure(s) is an oligonucleotide, the nucleic acid sequence of which is designed such that it specifically binds to the poly-(A) tail of the mRNA.
59. The method of any one of aspects 49 to 58, wherein the method after a) and prior to b) further comprises washing the carrier and/or the first carrier surface with a buffer solution.
60. The method of any one of aspects 46 to 59, wherein one, preferably more and particularly preferably all of the 3D DNA nanostructures is/are designed for direct binding to the respective region(s) of the respective target structure and is/are directly bound to the target molecule.
61. The method of any one of aspects 46 to 60, wherein the specific binding of at least one, preferably all of the 3D DNA nanostructure(s) is mediated by a respectively assigned target adapter, wherein the target adapter and/or each of the target adapters is designed to bind to the respective DNA nanostructure and to the respective region(s) of the respective target structure.
62. The method of aspect 61, wherein the at least one, preferably all of the 3D DNA nanostructure(s) comprise(s) at least one single-stranded DNA segment, which is disposed outwardly and which is designed such that it specifically binds to the respective target adapter.
63. The method of any one of aspects 61 or 62, wherein the one or more target adapters each comprise a single-stranded DNA segment, which is each designed such that it mediates the specific binding to the respective 3D DNA nanostructure.
64. The method of any one of aspects 61 to 63, wherein the one or more target adapters each comprise a target binding segment, which is each designed such that it mediates the specific binding to the respective region(s) of the respective target structure.
65. The method of aspect 64, wherein a first of the at least two target structures is the target structure as defined in aspect 48, and wherein the target binding segment of the target binding adapter which binds to the first target structure comprises a nucleotide sequence, which specifically binds to the one single-stranded segment of the first target structure.
66. The method of aspect 65, wherein the feature of aspect 63 also applies to all further target structures and the respective target binding segments of the respective target binding adapters.
67. The method of aspect 64, wherein a first of the at least two target structures comprises or is a protein, and wherein the target binding segment of the target binding adapter which binds to the first target structure comprises a peptide, preferably an antibody, which specifically binds to the first target structure.
68. The method of aspect 67, wherein the feature of aspect 63 also applies to all further target structures and the respective target binding segments of the respective target binding adapters.
69. The method of any one of aspects 46 to 68, wherein the method further comprises providing a sample solution, which contains the at least two target structures, and which comprises providing at least two 3D DNA nanostructures, and optionally further comprises providing the carrier, the carrier adapter(s) and/or the target adapter(s).
70. The method of any one of aspects 46 to 69, wherein the method further comprises mixing of a sample solution, which contains the at least two target structures, with the at least two 3D DNA nanostructures and optionally the carrier adapter(s) and/or the target adapter(s).
71. The method of any one of the preceding aspects, wherein measuring at least one fluorescence signal comprises:
   q) creating a data set, which contains data of fluorescence signals emitted by a section of a sample by using a fluorescence microscope, preferably in epifluorescence, TIRF, light sheet and/or confocal microscopy;
   and wherein the detection of the target structure and/or the one or more further target structures comprises:
   w) identifying one or more of the datums contained in the data set, which represent(s) the fluorescence signal of one of the identification structures.
72. The method of aspect 71, wherein step q) comprises:
   recording of at least one image file containing fluorescence data of the sample section.
73. The method of aspect 71 or 72, wherein the identifying in step w) comprises the following steps:
   w1) readout of a color and/or an intensity information of a datum and/or image element; and
   w2) comparing the color and/or intensity information of the datum and/or image element with a color and/or intensity information being representative for the identification structure.
74. The method of aspect 73, wherein the identifying in step w) comprises the following steps:
   w1) readout of a color and an intensity information of a datum and/or image element; and
   w2) comparing the color and intensity information of the datum and/or image element with a color and intensity information being representative for the identification structure.
75. The method of aspect 73 or 74, wherein the fluorescence signals of the identification structures formed for the individual different target structures differ from the fluorescence signal of all isolated 3D DNA nanostructures, when these are not bound in one of the identification structures, and the fluorescence signals of the identification structures which were formed for the individual different target structures are pairwise different, in that the corresponding fluorescence signals comprise a distinguishably different combination of color and/or intensity information.
76. The method of aspect 75, wherein in the 3D DNA nanostructures k intensity levels distinguishable from each other and/or m color levels distinguishable from each other are used, wherein preferably each of the k intensity levels is formed by intensity distribution and wherein the k intensity distributions are distinguishable from each other, preferably statistically, and/or wherein preferably each of the m color levels is formed by color distribution and wherein the m color distributions are distinguishable from each other, preferably statistically, wherein the respective overlap of adjacent distributions is preferably lower than 30%, more preferably lower than 10%, even more preferably lower than 5%, particularly preferably lower than 2% and highly particularly preferably lower than 1%.
77. The method of aspect 76, wherein k>2, preferably k>3, more preferably k>4, even more preferably k>5, particularly preferably k>6.
78. The method of aspect 76 or 77, wherein m>2, preferably m>3, more preferably m>4, even more preferably m>5, particularly preferably m>6.
79. The method of any one of aspects 73 to 78, wherein step w1) comprises one or a combination of the following steps and techniques: determining a mean value of the image element, determining a maximum of the image element, calculating a cumulative gradient, calculating one or more statistical moments such as e.g. variance of pixels in the image element, variance coefficient, Fano factor; and/or wherein step w2) is carried out based on a clustering method, preferably a DBSCAN (density based spatial clustering of applications with noise); and/or wherein step f1) and/or f2) are based on probabilistic observation, preferably by taking into consideration the Bayes' theorem.
80. A 3D DNA nanostructure, on which at least one fluorescence dye molecule is attached, wherein the shape of the 3D DNA nanostructure prevents that when approaching a second 3D DNA nanostructure, on which at least one fluorescence dye molecule is attached, the fluorescence dye molecules of the two 3D DNA nanostructures interact significantly.
81. The 3D DNA nanostructure of aspect 80, wherein the interaction comprises quenching and/or FRET.
82. The 3D DNA nanostructure of aspect 80 or 81, wherein at least two fluorescence dye molecules are attached on the 3D DNA nanostructure, and wherein the distance between the at least two fluorescence dye molecules is pairwise greater than that at which they interact significantly.
83. The 3D DNA nanostructure of any one of aspects 80 to 82, wherein the 3D DNA nanostructure is essentially formed as a hollow cylinder, and wherein the at least one fluorescence dye molecule or the at least two fluorescence dye molecules are attached on the inside of the cylindrical DNA nanostructure.
84. A 3D DNA nanostructure having a cavity and at least one inwardly disposed fluorescence dye molecule, wherein the distance of the at least one inwardly disposed fluorescence dye molecule to the rim of the 3D DNA nanostructure is at least 2 nm, preferably at least 3 nm and particularly preferably at least 5 nm.
85. The 3D DNA nanostructure of aspect 84, wherein the 3D DNA nanostructure comprises at least two inwardly disposed fluorescence dye molecules, and wherein the distance of the at least two inwardly disposed fluorescence dye molecules is at least 2 nm, preferably at least 5 nm and particularly preferably at least 9 nm.
86. The 3D DNA nanostructure of any one of aspects 80 to 85, wherein the 3D DNA nanostructure is essentially formed as elliptic hollow cylinder, preferably as circular hollow cylinder.
87. The 3D DNA nanostructure of aspect 86, wherein the hollow cylinder is a circular cylinder and comprises an inner radius of at least 5 nm, preferably an inner radius of 30 nm to 60 nm, particularly preferably an inner radius of 60 nm and/or a height of at most 200 nm, preferably a height of 30 nm to 60 nm, particularly preferably a height of 30 nm.
88. The 3D DNA nanostructure of any one of aspects 86 to 87, wherein the hollow cylinder comprises a wall thickness of at least 2 nm, preferably 2 nm to 7 nm, particularly preferably 5 nm and/or wherein the hollow cylinder comprises DNA helices having a helix diameter and wherein the wall thickness measures at least one, preferably at least two helix diameters and/or wherein the wall thickness measures at most four, preferably at most three helix diameters.
89. The 3D DNA nanostructure of any one of aspects 80 to 88, wherein the 3D DNA nanostructure comprises a single-stranded DNA scaffold strand of at least 3000 bases, preferably 5000-50000 bases, particularly preferably 10000-11000 bases, wherein the DNA scaffold strand is preferably circular.
90. The 3D DNA nanostructure of aspect 89, wherein the 3D DNA nanostructure further comprises a plurality of staple strands, preferably comprises 100 to 500 staple strands having preferably 30 to 100 bases each.
91. The 3D DNA nanostructure of aspect 90, wherein one of the inwardly disposed fluorescence dye molecules is bound to an inwardly disposed segment of a staple strand, preferably wherein each of the inwardly disposed fluorescence dye molecules is bound to a respective inwardly disposed segment of a staple strand.
92. The 3D DNA nanostructure of any one of aspects 89 to 91, further comprising at least one inwardly disposed single-stranded dye adapter DNA oligomer, which is designed such that it is specifically bound to an inwardly disposed single-stranded sequence segment of the scaffold strand or one of the staple strands, and wherein the at least one dye adapter DNA oligomer comprises one of the inwardly disposed dye molecules.
93. The 3D DNA nanostructure of aspect 92, wherein the inwardly disposed single-stranded sequence segment of the staple strand is an overhang, which is disposed inwardly and which is not required for the assembly of the 3D DNA nano structure.
94. The 3D DNA nanostructure of any one of aspects 80 to 93, wherein the 3D DNA nanostructure comprises a single-stranded DNA segment that may specifically bind to a target structure, preferably to a polynucleotide target structure and particularly preferably to a single-stranded polynucleotide target structure.
95. The 3D DNA nanostructure of any one of aspects 80 to 94, wherein the 3D DNA nanostructure further comprises at least one target adapter, wherein the target adapter may mediate the specific binding to a target structure.
96. The 3D DNA nanostructure of aspect 95, wherein the target adapter comprises a first single-stranded polynucleotide sequence, and wherein the first single-stranded polynucleotide sequence is bound to a single-stranded sequence segment of the 3D DNA nanostructure, preferably to a single-stranded sequence segment of the scaffold strand or one of the staple strands and particularly preferably to a single-stranded sequence segment of one of the staple strands.
97. The 3D DNA nanostructure of aspect 95 or 96, wherein the target adapter further comprises a target binding segment which may specifically bind a target structure.
98. The 3D DNA nanostructure of aspect 97, wherein the target binding segment comprises or is a second single-stranded polynucleotide sequence, and wherein the target structure comprises a single-stranded polynucleotide, preferably an mRNA.
99. The 3D DNA nanostructure of aspect 97, wherein the target binding segment comprises a protein, which specifically binds to the target structure, preferably an antibody or an antibody-binding fragment of an antibody.
100. The 3D DNA nanostructure of any one of aspects 95 to 99, wherein the target adapter, preferably at least the target binding segment, is pointing outward.
101. The 3D DNA nanostructure of any one of aspects 80 to 100, wherein the 3D nanostructure comprises no fluorescence dye molecules which are positioned outwardly.
102. Use of the 3D DNA nanostructure of any one of aspects 80 to 101 for a method of any one of aspects 1 to 79.
103. A set comprising multiple 3D DNA nanostructures of any one of aspects 80 to 101, wherein the set comprises N 3D DNA nanostructures that are different from each other and wherein the N different 3D DNA nanostructures of the set are pairwise different from each other due to the fluorescence dye molecules.
104. The set of aspect 103, wherein the N 3D DNA nanostructures that are different from each other contain a different number of fluorescence dye molecules and/or different fluorescence dye molecules, so that with the N 3D DNA nanostructures of the set that are different from each other, k intensity levels that are distinguishable from each other and/or m color levels that are distinguishable from each other can be generated.
105. The set of aspect 104, wherein at least one part of the N 3D DNA nanostructures that are different from each other are contained in the set multiple times, so that each of the k intensity levels is formed by an intensity distribution, and wherein the k intensity distributions are distinguishable from each other, preferably statistically.
106. The set of aspect 105 or 106, wherein at least a part of the N 3D DNA nanostructures that are different from each other are contained in the set multiple times, so that each of the m color levels is formed by a color distribution, and wherein the m color distributions are distinguishable from each other, preferably statistically.
107. The set of aspect 105 or 106, wherein the overlap of adjacent distributions is lower than 30%, preferably lower than 10%, more preferably lower than 5%, even more preferably lower than 2% and particularly preferably lower than 1%.
108. The set of any one of aspects 104 to 107, wherein k>2, preferably k>3, more preferably k>4, even more preferably k>5, particularly preferably k>6.
109. The set of any one of aspects 104 to 108, wherein m>2, preferably m>3, more preferably m>4, even more preferably m>5, particularly preferably m>6.
110. The use of the set of any one of aspects 103 to 109 for a method of any one of aspects 1 to 79.

In the following, preferred embodiments of the invention are explained in more detail with reference to the figures. The figures show:

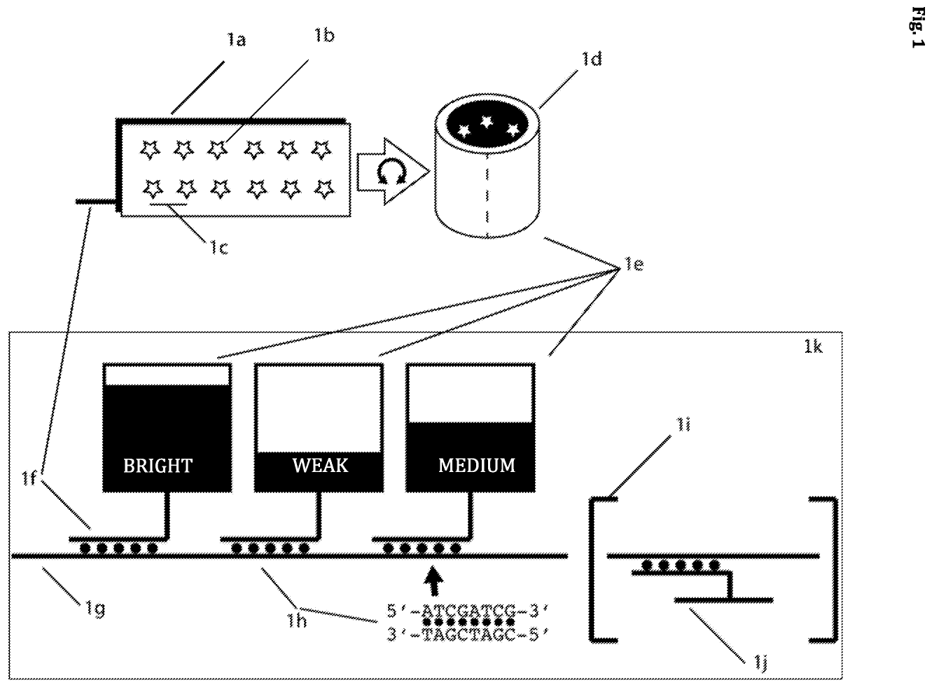

FIG. 1 schematically, the basic principle underlying the invention for the exemplary case of gene expression analysis;

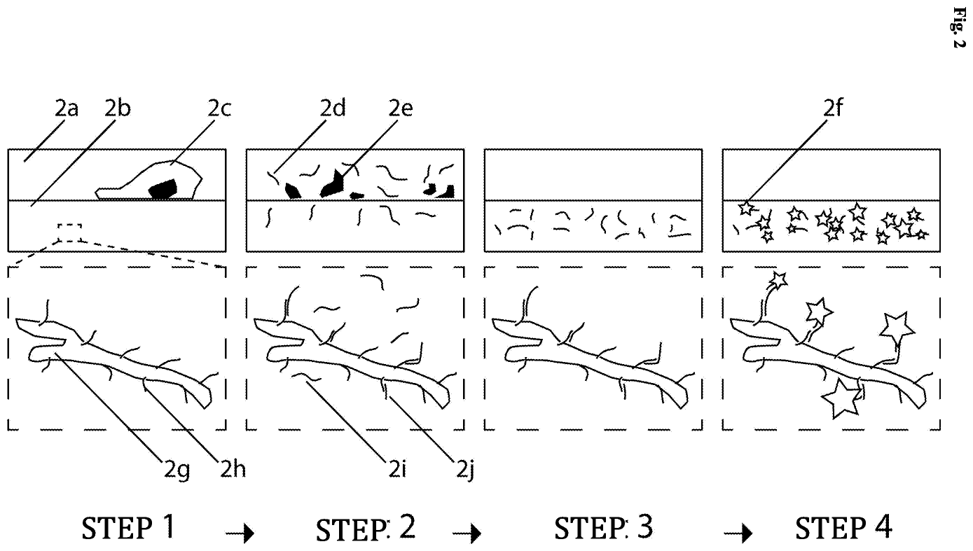

FIG. 2 schematically, the course of a single-cell gene expression analysis according to a preferred embodiment of the method of the invention;

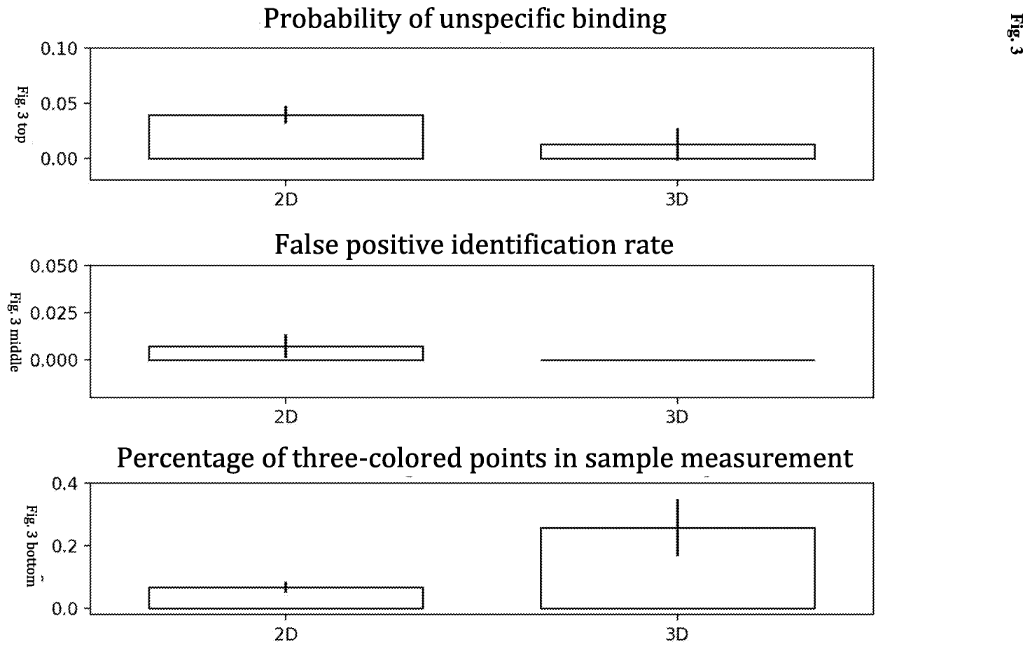

FIG. 3 the results of an experiment; and

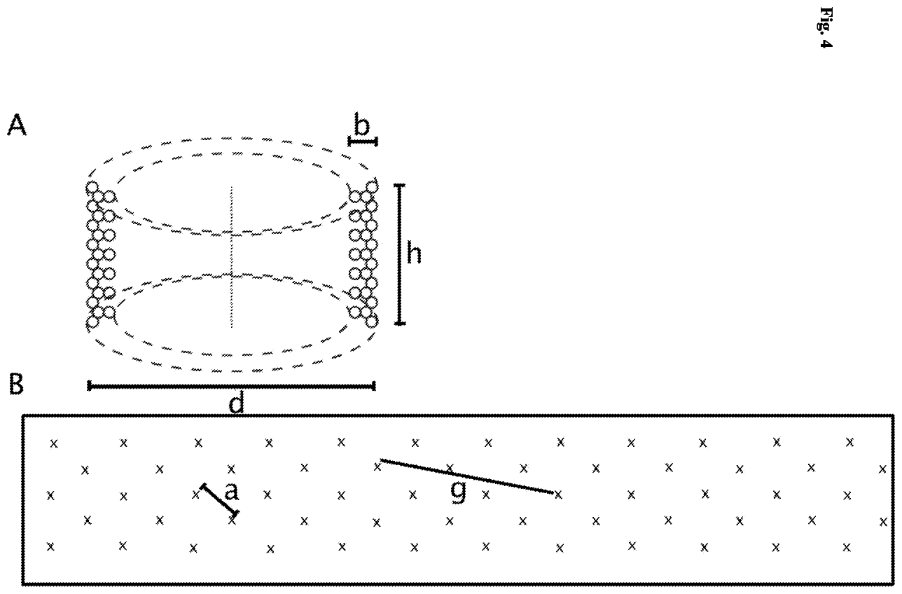

FIG. 4 a 3D DNA nanostructure according to a preferred embodiment,

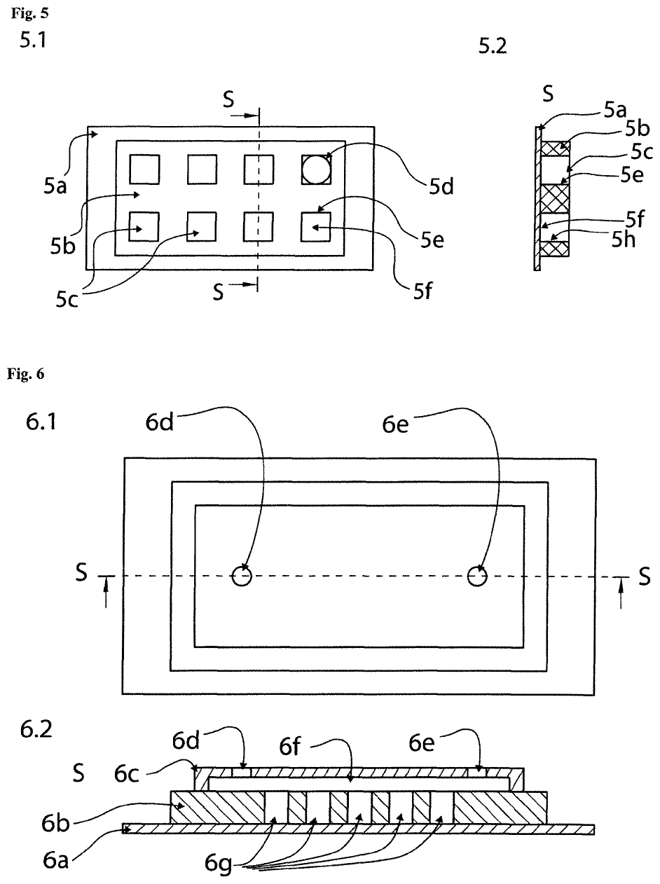

FIG. 5 schematically, a microwell array according to a preferred embodiment;

FIG. 6 schematically, a microwell chamber according to a preferred embodiment; and

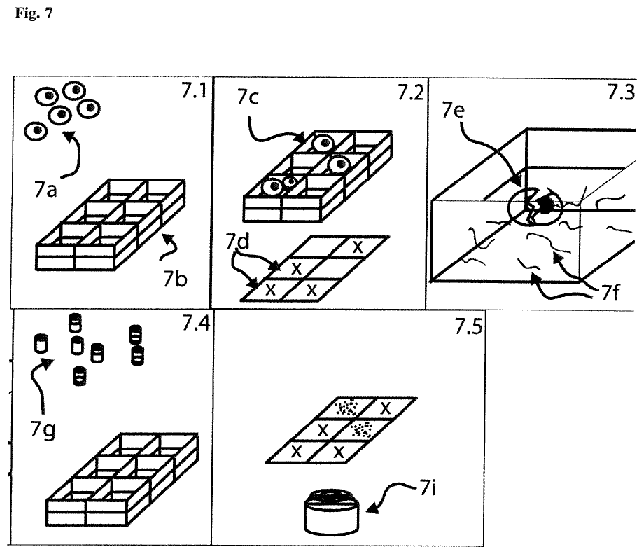

FIG. 7 schematically, method steps for single-cell gene expression analysis.

FIG. 1 shows, schematically, the basic principle underlying the invention for the exemplary case of gene expression analysis. The cylindrical 3D DNA nanostructure 1d of the case shown (see top of FIG. 1) with inwardly disposed fluorescence dye molecules 1b (with a distance 1c) is formed, as schematically indicated, by bending and/or rolling a 2D DNA nanostructure 1a forming the cylinder wall and the cylinder is stabilized by means of corresponding staple strands in its form. The cylinder comprises an adapter if coupled to it by means of which the 3D DNA nanostructure 1d can bind to a target structure 1g (see bottom of. FIG. 1), in this case to mRNA by hybridisation. The reference number 1h is to indicate, by way of example, a Watson Crick base pairing for the specific binding of the 3D DNA nanostructure and the target structure. The sequence indicated is to illustrate the mechanism only in an exemplary manner and does not reflect the actual sequence. The optional situation that the target structure is coupled via an adapter to a substrate 1j is represented in square brackets 1i.

At the bottom of FIG. 1 three 3D DNA nanostructures 1e are bound to the target structure 1g in an exemplary manner, wherein the intensities of the three 3D DNA nanostructures 1e measured under fluorescence conditions are different from each other, which is indicated by “BRIGHT”, “WEAK” and “MEDIUM”, and are caused by a different number of fluorescence molecules present in the 3D DNA nanostructures.

FIG. 2 shows, schematically, the course of a single-cell gene expression analysis according to a preferred embodiment of the method of the invention. A single cell 2c (here, the host body is a cell) in a microwell 2a limited by a 2^{nd} layer 2j is located under and/or in a polymer matrix 2b. A zoom into the polymer matrix (see Figure at the bottom) shows a polymer filament 2g of this matrix on which carrier adapters 2h are located. Between step 1 and step 2 the cells are lysed so that target structures, e.g. mRNAs (2d) as well as other cell components (2e) may move freely. Target structures bind to the carrier adapters and form target structure-carrier adapter complexes 2i. In step 3 some of the other cell components are washed away. Step 4 shows the added 3D DNA nanostructures 2f with target adapters which bind to the target structures. Due to the high local density of the carrier adapters, the target structures of individual cells can be particularly well captured. In particular, the polymer matrix impedes diffusion of the target structures out of their microwells and/or into other microwells. However, due to the three-dimensionality of the polymer matrix, it is possible to image densely arranged identification structures, for example, by using confocal microscopy, separately from each other in a satisfying manner.

FIG. 4A shows, schematically, a 3D DNA nanostructure according to a preferred embodiment in form of a hollow cylinder with a diameter d of 60 nm and a height h of 30 nm. In the example shown, the cylinder is formed by 22 DNA double-helices with a diameter of approximately 2 nm, with each circle representing the cross-section of a helix. As can easily be seen, the external face of the wall of the hollow cylinder is not smooth (or even mathematically perfect). Rather, the external face of the wall comprises projections and recesses in circumferential direction. The same applies to the inner wall. The helices are arranged on a grid-like or honeycomb-form structure in such a way that the wall thickness b corresponds to about 2.5 helix diameters (or approximately 5 nm).

FIG. 5.1 shows a schematic illustration of a microwell array according to the present invention in plan view. FIG. 5.2 shows a section through the microwell array of FIG. 5.1 along markers S. The microwell array comprises a 1^{st} (first) layer 5a at the bottom. The 1^{st} layer is preferably a microscopy cover slip, preferably a cover slip suited for fluorescence microscopy. The material of the 1st layer preferably comprises glass but may also comprise plastic.

On the 1^{st} layer, a 2^{nd} (second) layer 5b is applied, which forms the circumferential walls 6e of the microwells 6c. Hence, the microwells 5c are formed as recesses in the 2^{nd} layer 5b. The microwells 5c are illustrated here with a rectangular outline, i.e. rectangular bottoms 5f and circumferential walls 5e perpendicular to it. The bottoms 5f of the microwells 5c are formed by the 1^{st} layer 5a, herein a microscopy cover slip. At the top, i.e. above the 2^{nd} layer 5b, the microwells 5c are open. The depth 5h is also depicted in FIG. 5.2. However, as already described, the microwells 5c can also adopt entirely different forms. In particular for microwells with irregularly formed outline, the size of the microwells can be indicated by means of the diameter of an inscribed circle 5d or also by indicating the volume of the microwell 5c. The circumferential wall 5b of a microwell does not have to be perpendicular to its bottom 5f. The circumferential wall 5e can also be in an angle 90° to the bottom 5f. It is important that the gradient of the circumferential wall 5b relative to the bottom 5f is sufficient to transfer a host body, which sinks towards the bottom 5f of the microwell 5c influenced by gravity, towards the bottom 5f of the microwell 5c. Preferably, the angle deviates ±15° or less, more preferably ±10° or less, more preferably ±5° or less, particularly preferably ±2° or less from the perpendicular to the bottom 5f.

FIG. 6.1 shows a schematic illustration of a microwell chamber according to the present invention in plan view. FIG. 6.2 shows a schematic illustration of a section through the microwell chamber of FIG. 6.1 according to markers S. In the illustrated embodiment, the microwell chamber comprises a 1^{st} (first) layer 6a of a cover slip, preferably of a cover slip which is suited for microscopy, in particular for fluorescence microscopy. The 1^{st} layer preferably consists of glass, but may also consist of plastic. A 2^{nd} (second) layer 6b is applied on the 1^{st} layer 6a. The 2^{nd} layer 6b comprises recesses 6g. The recesses 6g are limited by the layer at their bottom. Thereby, microwells are formed, which each comprise a bottom, which is formed by the 1^{st} layer 6a, and a circumferential wall, which is formed by the 2^{nd} layer 6b. The microwells are open at their top. The 1^{st} and 2^{nd} layer together form a microwell array. On the top side of the 2^{nd} layer 6b and/or the microwell array, a 3^{rd} (third) layer 6c is attached in the embodiment illustrated in FIG. 6. The 3^{rd} layer 6c comprises a cavity 6f. The cavity 6f extends adjacent to and across the microwells. Thus, a direct connection between the cavity 6f of the 3^{rd} layer 6c and the microwells, which are open at their top, of the microwell array is given. This results in a working cavity, which contains the cavity 6f of the 3^{rd} layer and the microwells. The working cavity is sealed from the surroundings by the 1^{st}, 2^{nd} and 3^{rd} layer. An intended connection between the working cavity and the surroundings is provided in form of an inlet 6d and an outlet 6e. Via the inlet 6d, liquids required for the method can be added to the working volume and, thus, be applied to the microwell array. Excess liquid volume, and in particular also air, can flow out through the outlet 6e. Thereby, sequential adding and rinsing of liquids is ensured.

FIG. 7 shows a schematic illustration of a series of method steps according to the present invention. In FIG. 7.1, a group of host bodies 7a is introduced into a microwell array 7b. Preferably, this is done in that the host bodies 7a are present in a suitable liquid and the host body liquid is applied to the top side of the microwell array. The host bodies 7a are preferably used in this step in a form in which the individual host bodies 7a are not attached to each other. The host bodies 7a are illustrated herein as cells in an exemplary manner. In FIG. 7.2, the host bodies 7a which were applied to the microwell array fall into the individual microwells 7c influenced by gravity and sink to the bottom. An incubation period required for this depends on the depth of the microwells and on the host bodies 7a used and on the viscosity of the liquid used. The skilled person will readily find a suitable incubation period. To this end, each microwell can be occupied by no, exactly one or more than one host body 7a. For later evaluation, the microwells are each preferably occupied by exactly one host body 7a. In order to ensure this condition, microwells are analyzed for their content. This can be carried out, for example, by means of bright-field microscopy and/or any other suitable imaging technique. Those microwells 7d which comprise several or no cells are identified in the Figure with an X and are excluded from the subsequent analysis. Those microwells 7c which comprise exactly one host body 7a are taken into consideration in the subsequent analysis (no additional identification in the Figure).

In FIG. 7.3., the host bodies 7a (herein cells) are disrupted by adding disruption buffer 7e (herein lysis buffer). In this manner, target structures 7f present in the host bodies 7a are released. The released target structures 7f bind to the bottom of the microwells 7c. Said bond can be mediated by carrier adapters, which each bind both the bottom and a target structure.

Subsequently, 3D DNA nanostructures 7g are added in FIG. 7.4. The 3D DNA nanostructures are adjusted to the one or more target structures to be analyzed. The 3D DNA nanostructures are selected such that for each of the one or more target structures that are different from each other a respectively assigned identification structure can be formed, wherein each of the identification structures comprises the respective assigned target structure and at least two 3D DNA nanostructures, wherein each of the at least two 3D DNA nanostructures comprises one or more inwardly disposed fluorescence dye molecules and wherein each of the at least two 3D DNA nanostructures binds specifically to the respective target structure, and wherein the at least two 3D DNA nanostructures are bound to pairwise different regions of the respective target structure.

FIG. 7.5 schematically shows the analysis with a microscope, wherein only the objective 7i is shown. Here, only the microwells 7c previously approved for analysis in FIG. 7.2 are analyzed. The analysis is done by means of fluorescence microscopy. The 3D DNA nanostructures 7g and the parameters of fluorescence measurement are selected such that the at least one measured fluorescence signal of the identification structures is distinguishable from the fluorescence signal of all isolated 3D DNA nanostructures when these are not bound in one of the identification structures and such that the measured fluorescence signals of all pairwise different identification structures are pairwise distinguishable from each other. As a matter of course, each of the different target structures may be present multiple times so that the signal of each of the different target structures may be present in the fluorescence measurement multiple times.

The following non-limiting examples are to illustrate the present invention.

### Example 1: Preparation of 3D Nanostructures of the Invention

1. Description of the 3D DNA Nanostructures Prepared

3D DNA nanostructures having the form of a hollow cylinder with 60 inwardly disposed dye molecules each, which show pairwise distances of at least 9 nm to the adjacent dye molecules within the same 3D DNA nanostructure, were prepared in three different dye variants, i.e. variants 3D_1, 3D_2 and 3D_3:
3D DNA nanostructure variant 3D_1: red fluorescent:
The 3D_1 DNA nanostructure comprises 60 inwardly disposed DNA staple strands with additional single-stranded sequences complementary to fluorophore adapters for red dyes (designated as sequence S1), and 60 related fluorophore adapters which are each provided with an Atto647N dye molecule (oligomers SEQ_ID_NO 1259). The staple strands to be extended for fluorophore adapters binding were selected in the design program CaDNAno based on the criteria that they are located within the hollow cylinder, that they are located at a distance of more than 2 nm from the rim of the hollow cylinder, have a free end on the internal surface of the hollow cylinder and have a distance of more than 5 nm from each other (due to the design and the sequence lengths, the latter condition applies to all staple strands which meet the above criteria). The dye adapters are dye-modified at the 3′ end. This end was selected to ensure that the dyes are located as close as possible to the internal surface of the hollow cylinder and have little leeway and thus, can approach each other as little as possible. Several modifications per dye adapter would be conceivable, however, they would imply higher costs and a certain loss of control of their positioning. Moreover, the 3D_1 nanostructure was provided with a T1 target adapter (oligomer SEQ_ID_NO 1263). The SEQ ID NOs of all staple strands used for the 3D_1 nanostructure are summarized in the definition oligopool3D_S1 below. Strand p7308 (SEQ ID NO 1258) was used as scaffold strand.
3D DNA nanostructure variant 3D_2: green fluorescent:
The 3D_2 DNA nanostructure comprises 60 inwardly disposed DNA staple strands with additional single-stranded sequences complementary to fluorophore adapters for red dyes (designated as sequence S2), and 60 related fluorophore adapters which are each provided with an Atto565 dye molecule (oligomers SEQ_ID_NO 1260). The staples strands to be extended for fluorophore adapters binding were selected in the design program CaDNAno based on the criteria that they are located within the hollow cylinder, that they are located at a distance of not more than 2 nm from the rim of the hollow cylinder, have a free end on the internal surface of the hollow cylinder and have a distance of more than 5 nm from each other (due to the design and the sequence lengths, the latter condition applies to all staple strands which meet the above criteria). The dye adapters are dye-modified at the 3′ end. This end was selected to ensure that the dyes are located as close as possible to the internal surface of the hollow cylinder and have little leeway and thus, can approach each other as little as possible. Several modifications per dye adapter would be also conceivable, however, they would imply higher costs and a certain loss of control of their positioning. The 3D_2 nanostructure was provided with a T2 target adapter (oligomer SEQ_ID_NO 1264). The SEQ ID NOs of all staple strands used for the 3D_2 nanostructure are summarized in the definition oligopool3D_S2 below. Strand p7308 (SEQ ID NO 1258) was used as scaffold strand.
3D DNA nanostructure variant 3D_3: blue fluorescent:
A 3D_3 DNA nanostructure comprises 60 inwardly disposed DNA staple strands with additional single-stranded sequences complementary to fluorophore adapters for red dyes (designated as sequence S3), and 60 related fluorophore adapters which are each provided with an Atto488 dye (oligomers SEQ_ID_NO 1261). The staple strands to be extended for fluorophore adapter binding were selected in the design program CaDNAno based on the criteria that they are located within the hollow cylinder, that they are located at a distance of not more than 2 nm from the rim of the hollow cylinder, have a free end on the internal surface of the hollow cylinder and have a distance of more than 5 nm from each other (due to the design and the sequence lengths, the latter condition applies to all staple strands which meet the above criteria). The dye adapters are dye-modified at the 3′ end. This end was selected to ensure that the dyes are located as close as possible to the internal surface of the hollow cylinder and have little leeway and thus, can approach each other as little as possible. Several modifications per dye adapter would also be conceivable, however, they would imply higher costs and a certain loss of control of their positioning. The 3D_3 nanostructure was provided with a T3 target adapter (oligomer SEQ_ID_NO 1265). The SEQ ID NOs of all staple strands used for the 3D_3 nanostructure are summarized in the definition oligopool3D_S3 below. Strand p7308 (SEQ ID NO 1258) was used as scaffold strand.

All DNA oligomers (also the DNA oligomers provided with fluorescence dye) were acquired from Eurofins Genomics GmbH.

An illustration of a 3D DNA nanostructure of Example 1 is shown in FIG. 5 already discussed above. FIG. 5A shows a schematic representation of 3D DNA nanostructures 3D_1, 3D_2 and 3D_3 which differ only with respect to the type of the attached fluorescence dye molecules. FIG. 5B shows the hollow cylinder of FIG. 5A cut and unrolled (for illustration purposes), with the internal surface of the hollow cylinder of FIG. 5A being shown in plan view. Each x represents a fluorescence dye molecule. Between the nearest neighbors the pairwise dye molecules distances a are 9 nm. Between the other dye molecules, the pairwise distances, for example the distance g, are greater. The dye molecules are each located at the innermost helix in FIG. 5A so that the distance of the dye molecules from the outer rim of the hollow cylinder corresponds approximately to the wall thickness of the cylinder wall, i.e. approximately 2.5 helix diameters (corresponding approximately to 5 nm).

2. Preparation Protocol:

In the present example, the 3D DNA nanostructures 3D_1-3 were prepared according to the preparation protocol described in the following.

For the preparation of the 3D DNA nanostructures 3D_1, 3D_2 and 3D_3, first the following components were mixed to obtain a final volume of 200 μl:
10 nM p7308 scaffold strand (SEQ ID NO 1258)
100 nM of each of the staple strands (SEQ ID NOs from oligopool3D_S1 for DNA nanostructure 3D_1, SEQ ID NOs from oligopool3D_S2 for DNA nanostructure 3D_2 and SEQ ID NOs from oligopool3D_S3 for DNA nanostructure 3D_3)
120 nM fluorescence dye modified DNA oligos (SEQ ID NO 1259 for DNA nanostructure 3D_1, SEQ ID NO 1260 for DNA nanostructure 3D_2 and SEQ ID NO 1261 for DNA nanostructure 3D_3)
400 nM target-binding DNA oligos (SEQ ID NO 1263 for DNA nanostructure 3D_1, SEQ ID NO 1264 for DNA nanostructure 3D_2 and SEQ ID NO 1265 for DNA nanostructure 3D_3)
1× buffer FB02 (buffer composition see below)

In order to have the DNA nanostructures fold into their shape, the above-mentioned mixtures were first heated to melt secondary structures possibly present, then slowly cooled so that the base pairings were able to find the thermal equilibrium and accordingly the planned conformation. To this end, the following thermocycler program was used (using Mastercycler Nexus X2 by Eppendorf GmbH, Hamburg, Germany):
Maintain 15 minutes at 65° C. and cool within one minute to 50° C.
Decrease from 50° C. to 40° C. in 66 hours with constant rate
At the end of 66 h (optionally, it is possible to incubate for a few additional hours at 40° C.) cool within one minute to 4° C. and store at 2-8° C.

Subsequently, the correctly folded 3D DNA nanostructures were separated from single DNA oligomers or smaller DNA oligomer complexes. To this end, the following PEG purification (purification with polyethylene glycol) was used:
Mix the liquid derived from the thermocycler with the same volume of PEGX01 (for buffer composition see below)
Centrifugation for 35 min at 13000-16000 rfc at room temperature (20-25° C.)
Remove the supernatant with a pipette
Resuspend in 200 μl FB02 (for buffer composition see below) and 200 μl PEGX01 (for buffer composition see below)
Centrifugation for 35 min at 13000-16000 rfc at room temperature (20-25° C.)
Resuspend in 100 μl FB01 (for buffer composition see below)
Incubation on the shaker for 8 to 16, preferably 10 hours (in this case over night) in the dark at room temperature and with 600 rpm in order to dissolve the pellet completely
Store at 4° C. for further use (up to 12 months possible, however, in the present case, the storage was only a few days)

While in the present case, PEG purification of the DNA nanostructures was used, in the alternative, it would in principle be possible to carry out purification with agarose gel electrophoresis with subsequent extraction of the DNA nanostructures from the gel. An agarose gel electrophoresis-based purification could for example be carried out as follows:
Prepare 1.5% w/v agarose gel
Add 5× Orange G loading buffer to the sample
Electrophoresis at 4.5 V/cm voltage for 1.5 h with ice cooling
Excise pieces of gel with DNA origami band. The latter has a breadth of approximately 2 mm, and it is optionally possible to identify it more easily by loading an adjacent lane with scaffold strand, place it in Freeze 'N Squeeze™ DNA Gel Extraction Spin Columns (BioRad Laboratories GmbH) and centrifuge for 4.5 min at room temperature and at 1050 rcf.

Buffer Solutions Used

PEGX01:
15% PEG 8000
1×TAE (Tris acetate EDTA buffer: 40 mM Tris, 20 mM acetic acid, 1 mM EDTA)
12.5 mM MgCl2
500 mM NaCl

FB01:
10 mM Tris pH8.0
1 mM EDTA
12.5 mM MgCl2

FB02:
10 mM Tris pH8.0
1 mM EDTA
10 mM MgCl2

DNA Oligo Pools:

For the preparation of the respective 3D DNA nanostructures, oligomers were combined based on their SEQ_ID_NOs (numbers in square brackets indicate the respective SEQ ID NOs) in the following pools:

oligopool_3D-S1: [1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 277, 286, 287, 288, 278, 279, 280, 281, 282, 283, 284, 285, 286, 287, 288, 469, 478, 479, 480, 470, 471, 472, 473, 474, 475, 476, 477, 478, 479, 480, 265, 274, 275, 276, 266, 267, 268, 269, 270, 271, 272, 273, 274, 275, 276, 301, 310, 311, 312, 302, 303, 304, 305, 306, 307, 308, 309, 310, 311, 312, 253, 262, 263, 264, 254, 255, 256, 257, 258, 259, 260, 261, 262, 263, 264, 289, 298, 299, 300, 290, 291, 292, 293, 294, 295, 296, 297, 298, 299, 300, 241, 250, 251, 252, 242, 243, 244, 245, 246, 247, 248, 249, 250, 251, 252]

oligopool_3D-S2: [1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 277, 286, 287, 288, 278, 279, 280, 281, 282, 283, 284, 285, 286, 287, 288, 481, 490, 491, 492, 482, 483, 484, 485, 486, 487, 488, 489, 490, 491, 492, 337, 346, 347, 348, 338, 339, 340, 341, 342, 343, 344, 345, 346, 347, 348, 361, 370, 371, 372, 362, 363, 364, 365, 366, 367, 368, 369, 370, 371, 372, 325, 334, 335, 336, 326, 327, 328, 329, 330, 331, 332, 333, 334, 335, 336, 349, 358, 359, 360, 350, 351, 352, 353, 354, 355, 356, 357, 358, 359, 360, 241, 250, 251, 252, 242, 243, 244, 245, 246, 247, 248, 249, 250, 251, 252]

oligopool_3D-S3: [1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 277, 286, 287, 288, 278, 279, 280, 281, 282, 283, 284, 285, 286, 287, 288, 493, 502, 503, 504, 494, 495, 496, 497, 498, 499, 500, 501, 502, 503, 504, 385, 394, 395, 396, 386, 387, 388, 389, 390, 391, 392, 393, 394, 395, 396, 409, 418, 419, 420, 410, 411, 412, 413, 414, 415, 416, 417, 418, 419, 420, 373, 382, 383, 384, 374, 375, 376, 377, 378, 379, 380, 381, 382, 383, 384, 397, 406, 407, 408, 398, 399, 400, 401, 402, 403, 404, 405, 406, 407, 408, 241, 250, 251, 252, 242, 243, 244, 245, 246, 247, 248, 249, 250, 251, 252]

### Example 2: Preparation of 2D DNA Nanostructures for Comparison

1. Description of the 2D DNA Nanostructures Prepared

Rectangular 2D DNA nanostructures, each with 48 dye molecules with a pairwise distance of 5 nm to the respective nearest dye molecules within the same 2D DNA nanostructure (such a 2D DNA nanostructure is for example shown in FIG. 1A of WO 002016140727) were prepared in three variants, i.e. in variants 2D1, 2D_2 and 2D_3:
2D DNA nanostructure variant 2D1: red fluorescent:
A 2D_1 DNA nanostructure comprises 48 DNA staple strands with additional single-stranded sequences complementary to fluorophore adapters for red dyes (designated sequence S1), as well as 48 related fluorophore adapters which are each provided with an Atto647N dye, (oligomers SEQ_ID_NO 1259). The staple strands to be extended for fluorophore adapter binding were selected in the design program CaDNAno according to the criteria that they all are located at the same side of the rectangle and have a distance from each other that is sufficient that they do not significantly interact with each other. The dye adapters are dye-modified at the 3′ end. This end is selected to ensure that the dyes are located as close as possible to the surface of the rectangle and have little leeway and thus, can approach each other as little as possible. Several modifications per dye adapter would also be conceivable, however, they would imply higher costs and a certain loss of control of their positioning. The 2D_1 DNA nanostructure was provided with a T1 target adapter (oligomer SEQ_ID_NO 1266) which, according to the design, is located along the rim of the structure. The SEQ ID NOs of all staple strands used for the 2D_1 DNA nanostructure are summarized in the definition oligopool2D_S1 further below. Strand p7249 (SEQ ID NO 1257) was used as scaffold strand.
2D DNA nanostructure variant 2D_2: green fluorescent:
A 2D_2 DNA nanostructure comprises 48 DNA staple strands with additional single-stranded sequences complementary to fluorophore adapters for red dyes (designated sequence S2) as well as 48 related fluorophore adapters which are each provided with an Atto565 dye (oligomers SEQ_ID_NO 1260). The staple strands to be extended for fluorophore adapter binding were selected in the design program CaDNAno according to the criteria that they all are located at the same side of the rectangle and have a distance from each other that is sufficient that they do not significantly interact with each other. The dye adapters are dye-modified at the 3′ end. This end is selected to ensure that the dyes are located as close as possible to the inner surface of the rectangle and have little leeway and thus, can approach each other as little as possible. Several modifications per dye adapter would also be conceivable, however, they would imply higher costs and a certain loss of control of their positioning. The 2D_2 DNA nanostructure was provided with a T2 target adapter (oligomer SEQ_ID_NO 1267) which, according to the design, is located along the rim of the structure. SEQ ID NOs of all staple strands used are summarized in the definition oligopool2D_S2 further below. Strand p7249 (SEQ ID NO 1257) was used as scaffold strand.
2D DNA nanostructure variant 2D_3: blue fluorescent:
A 2D_3 DNA nanostructure comprises 48 DNA staple strands with additional single-stranded sequences complementary to fluorophore adapters for red dyes (designated sequence S3) as well as 48 related fluorophore adapters which are each provided with an Atto488 dye (oligomers SEQ_ID_NO 1261). The staple strands to be extended for fluorophore adapter binding were selected in the design program CaDNAno according to the criteria that they all are located at the same side of the rectangle and have a distance from each that is sufficient that they do not significantly interact with each other. The dye adapters are dye-modified at the 3′ end. This end is selected to ensure that the dyes are located as close as possible to the internal surface of the rectangle and have little leeway and thus, can approach each other as little as possible. Several modifications per dye adapter would also be conceivable, however, they would imply higher costs and a certain loss of control of their positioning. The 2D_3 DNA nanostructure was provided with a T3 target adapter (oligomer SEQ_ID_NO 1268) which, according to the design, is located along the rim of the structure. SEQ ID NOs of all staple strands used are summarized in the definition oligopool2D_S3 further below. Strand p7249 (SEQ ID NO 1257) was used as scaffold strand.

2. Preparation Protocol:

In the present example, the 2D DNA nanostructures 2D_1-3 were prepared according to the preparation protocol described in the following.

For the preparation of the 2D DNA nanostructures 2D_1-3, first the following components were mixed to obtain a final volume of 200 μl:
10 nM p7249 scaffold strand (SEQ ID NO 1257)
100 nM of each of the staple strands (SEQ ID NOs from oligopool2D_S1 for DNA nanostructure 2D_1, SEQ ID NOs from oligopool2D_S2 for DNA nanostructure 2D_2 and SEQ ID NOs from oligopool2D_S3 for DNA nanostructure 2D_3)
120 nM fluorescence dye modified DNA oligos (SEQ ID NO 1259 for DNA nanostructure 2D_1, SEQ ID NO 1260 for DNA nanostructure 2D_2 and SEQ ID NO 1261 for DNA nanostructure 2D_3)
400 nM target-binding DNA oligos (SEQ ID NO 1266 for DNA nanostructure 2D_1, SEQ ID NO 1267 for DNA nanostructure 2D_2 and SEQ ID NO 1268 for DNA nanostructure 2D_3)
1× buffer FB02 (buffer composition see Example 1)

In order to have the DNA nanostructures fold into their shape, the above-mentioned mixtures were first heated to melt secondary structures possibly present, then slowly cooled so that the base pairings were able to find the thermal equilibrium and accordingly the planned conformation. To this end, the following thermocycler program was used (using Mastercycler Nexus X2 by Eppendorf GmbH, Hamburg, Germany):
Maintain 15 minutes at 65° C. and cool within one minute to 60° C.
Decrease from 60° C. to 20° C. in 1 hour at constant rate
cool within one minute to 4° C. and keep at 4° C.

Subsequently, correctly folded 2D DNA nanostructures were separated from single DNA oligomers or smaller DNA oligomer complexes. To this aim, the following PEG purification (purification with polyethylene glycol) was used:
Mix the liquid derived from the thermocycler with the same volume of PEGX01 (for buffer composition see below)
Centrifugation for 35 min at 13000-16000 rfc at room temperature (20-25° C.)
Resuspend in 200 μl FB01 and 200 μl FB02 (for buffer composition see Example 1).
Centrifugation for 35 min at 13000-16000 rfc at room temperature (20-25° C.)
Resuspend in 100 μl FB01
Incubate on a shaker over night in the dark, at room temperature (20-25° C.) and at 600 rpm.
Store at −20° C. (e.g. for up to 3 years) or at 4° C. (e.g. for up to 12 months) for further use (in the present case, the storage was for a few days)

Also in this case, it would in principle be possible to use the agarose gel electrophoresis-based purification mentioned in Example 1 as an alternative to the PEG-based purification.

DNA Oligo Pools Used:

For the preparation of the respective 2D DNA nanostructures, oligomers were combined based on their SEQ_ID_NOs (numbers in square brackets indicate the respective SEQ ID NOs) in the following pools:

oligopool_2D-S1: [529, 538, 539, 540, 530, 531, 532, 533, 534, 535, 536, 537, 538, 539, 540, 563, 572, 573, 574, 564, 565, 566, 567, 568, 569, 570, 571, 572, 573, 574, 623, 632, 633, 634, 624, 625, 626, 627, 628, 629, 630, 631, 632, 633, 634, 657, 666, 667, 668, 658, 659, 660, 661, 662, 663, 664, 665, 666, 667, 668, 693, 702, 703, 704, 694, 695, 696, 697, 698, 699, 700, 701, 702, 703, 704, 705, 714, 715, 716, 706, 707, 708, 709, 710, 711, 712, 713, 714, 715, 716, 787, 796, 797, 798, 788, 789, 790, 791, 792, 793, 794, 795, 796, 797, 798, 799, 808, 809, 810, 800, 801, 802, 803, 804, 805, 806, 807, 808, 809, 810, 575, 584, 585, 586, 576, 577, 578, 579, 580, 581, 582, 583, 584, 585, 586, 587, 596, 597, 598, 588, 589, 590, 591, 592, 593, 594, 595, 596, 597, 598, 669, 678, 679, 680, 670, 671, 672, 673, 674, 675, 676, 677, 678, 679, 680, 681, 690, 691, 692, 682, 683, 684, 685, 686, 687, 688, 689, 690, 691, 541, 548, 549, 550, 542, 543, 544, 545, 546, 547, 548, 549, 550, 551, 560, 561, 562, 552, 553, 554, 555, 556, 557, 558, 559, 560, 561, 562, 635, 642, 643, 644, 636, 637, 638, 639, 640, 641, 642, 643, 644, 645, 654, 655, 656, 646, 647, 648, 649, 650, 651, 652, 653, 654, 655, 656]

oligopool_2D-S2: [529, 538, 539, 540, 530, 531, 532, 533, 534, 535, 536, 537, 538, 539, 540, 563, 572, 573, 574, 564, 565, 566, 567, 568, 569, 570, 571, 572, 573, 574, 623, 632, 633, 634, 624, 625, 626, 627, 628, 629, 630, 631, 632, 633, 634, 657, 666, 667, 668, 658, 659, 660, 661, 662, 663, 664, 665, 666, 667, 668, 505, 514, 515, 516, 506, 507, 508, 510, 512, 514, 515, 516, 517, 526, 527, 528, 518, 519, 520, 522, 524, 526, 527, 528, 599, 608, 609, 610, 600, 601, 602, 604, 606, 608, 609, 610, 611, 620, 621, 622, 612, 613, 614, 616, 618, 620, 621, 622, 697, 699, 701, 709, 711, 713, 791, 793, 795, 803, 805, 807, 951, 960, 961, 962, 952, 953, 954, 955, 956, 957, 958, 959, 960, 961, 962, 963, 972, 973, 974, 964, 965, 966, 967, 968, 969, 970, 971, 972, 973, 974, 1045, 1054, 1055, 1056, 1046, 1047, 1048, 1049, 1050, 1051, 1052, 1053, 1054, 1055, 1056, 1057, 1066, 1067, 1068, 1058, 1059, 1060, 1061, 1062, 1063, 1064, 1065, 1066, 1067, 1068, 541, 548, 549, 550, 542, 543, 544, 545, 546, 547, 548, 549, 550, 551, 560, 561, 562, 552, 553, 554, 555, 556, 557, 558, 559, 560, 561, 562, 635, 642, 643, 644, 636, 637, 638, 639, 640, 641, 642, 643, 644, 645, 654, 655, 656, 646, 647, 648, 649, 650, 651, 652, 653, 654, 655, 656]

oligopool_2D-S3: [529, 538, 539, 540, 530, 531, 532, 533, 534, 535, 536, 537, 538, 539, 540, 563, 572, 573, 574, 564, 565, 566, 567, 568, 569, 570, 571, 572, 573, 574, 623, 632, 633, 634, 624, 625, 626, 627, 628, 629, 630, 631, 632, 633, 634, 657, 666, 667, 668, 658, 659, 660, 661, 662, 663, 664, 665, 666, 667, 668, 505, 514, 515, 516, 506, 507, 508, 510, 512, 514, 515, 516, 517, 526, 527, 528, 518, 519, 520, 522, 524, 526, 527, 528, 599, 608, 609, 610, 600, 601, 602, 604, 606, 608, 609, 610, 611, 620, 621, 622, 612, 613, 614, 616, 618, 620, 621, 622, 697, 699, 701, 709, 711, 713, 791, 793, 795, 803, 805, 807, 575, 584, 585, 586, 576, 577, 578, 579, 580, 581, 582, 583, 584, 585, 586, 587, 596, 597, 598, 588, 589, 590, 591, 592, 593, 594, 595, 596, 597, 598, 669, 678, 679, 680, 670, 671, 672, 673, 674, 675, 676, 677, 678, 679, 680, 681, 690, 691, 692, 682, 683, 684, 685, 686, 687, 688, 689, 690, 691, 1105, 1112, 1113, 1114, 1106, 1107, 1108, 1109, 1110, 1111, 1112, 1113, 1114, 1115, 1124, 1125, 1126, 1116, 1117, 1118, 1119, 1120, 1121, 1122, 1123, 1124, 1125, 1126, 1199, 1206, 1207, 1208, 1200, 1201, 1202, 1203, 1204, 1205, 1206, 1207, 1208, 1209, 1218, 1219, 1220, 1210, 1211, 1212, 1213, 1214, 1215, 1216, 1217, 1218, 1219, 1220]

### Example 3: Comparison of the Detection of a Target Structure with the 3D Nanostructures of Example 1 and the 2D DNA Nanostructures of Example 2

1. Outline

In order to compare how well target structures can be detected with the 2D DNA nanostructures or the 3D DNA nanostructures of the invention by means of the method of the invention in its embodiment in which the identification structure with target structures and the respective bound DNA nanostructures is bound and/or is being bound to the surface of a carrier (in the present case, the surface of a cover slip), in the example described herein, the same target DNA oligomers were used as target structures and were detected on the one hand, based on the binding of red, green and blue 2D DNA nanostructures of the variants 2D_1, 2D_2 and 2D_3 as described in Example 2 (2D sample), and on the other hand, based on the binding of red, green and blue 3D DNA nanostructures of the variants 3D_1, 3D_2 and 3D_3 as described in Example 1 (3D sample). In addition, a control experiment was carried out for both experiments wherein it was checked whether and to which degree a false positive detection occurs on the surface of the cover slip if a target structure is absent. The comparison shows that, due to the optimized and well-conceived positioning of the dye molecules, the 3D DNA nanostructures adhere to a much lesser extent to the surface of the microscopy cover slip.

2. Material and Methods:

For the detection of target structures (in the present case, target DNA oligomers, in short: target oligomers), these were first incubated in a hybridization reaction with the respective DNA nanostructures (i.e. 2D_1, 2D_2 and 2D_3 or 3D_1, 3D_2 and 3D_3) and a capture strand in a suited buffer so that all these components were able to bind to each other and to form the identification structure (see FIG. 1, 1k). This was carried out separately for the 2D and 3D DNA nanostructures.

For the sample with 3D DNA nanostructures, hybridisation was carried out in a solution with the following components:
4 μl mixture of DNA nanoparticles 3D_1-3 with 200 pM per DNA nanoparticle set according to Example 1
10 μl 2× buffer RX07 (composition see below)
1 μ20 nM carrier adapter (herein also referred to as capture strand) (SEQ ID NO 1262) with biotin adapter, wherein the capture strand with biotin adapter (modified with biotin at the 5′ end) was provided by the company Eurofins
1 μl 750 pM single-stranded target DNA oligomer (SEQ ID NO 1269). This oligomer has complementary regions for T1, T2 and T3 so that the 3D DNA nanostructures 3D_1, 3D_2 and 3D_3 can bind with their respective T1, 2 and 3 regions to the corresponding complementary regions of the target DNA oligomer, as well as a complementary region for the capture strand. The target structure and the respective binding regions were designed in such a way that all three 3D DNA oligomers as well as the capture strand could concomitantly bind to a target DNA oligomer. For the control experiment, this volume was replaced with H₂O.
4 μl H₂O

This was incubated for 16 hours at 30° C. in order to allow capture strands, target oligomers and 3D DNA nanostructures to bind to each other.

For the case of the sample with the 2D DNA nanostructures, hybridisation was carried out in a solution with the same components, however, 4 μl mixture of DNA nanoparticles 2D_1-3, with 200 pM per DNA nanoparticle set according to Example 2, instead of 4 μl mixture of DNA nanoparticles 3D_1-3 with 200 pM per DNA nanoparticle set according to Example 1 were used. In this case also, it was ensured that all three 2D DNA nanostructures as well as the capture strand could bind to the target oligomer. The target DNA oligomer used also had the sequence with SEQ ID NO 1269. For the 2D DNA nanostructures too, a control reaction, which did not contain a target DNA oligomer, was carried out. Thus, in total, four experiments were carried out.

For the measurement, the capture strands of each experiment were placed on a microscopy surface of a respective microscopy sample carrier. A μ-slide of type VI^{0.1} from ibidi GmbH (Martinsried, Germany) was used as microscopy sample carrier. For this purpose, it was treated according to the following protocol for the preparation of microscopy slides as sample:
μ-slides VI^{0.1} (ibidi GmbH, Martinsried) were rinsed with 100 μl buffer A (composition, see below)
pipetting 40 μl 1 mg/ml biotinylated BSA (Sigma-Aldrich GmbH) into a first channel end of the corresponding channel of the μ-slide, incubating for 2 min, removing the fluid via the other, second channel end of the corresponding channel of the μ-slide by pipetting, pipetting 100 μl buffer A into the above first channel end, removing the fluid via the above second channel end by pipetting.
pipetting 40 μl 0.5 mg/ml streptavidin (Thermo Scientific) in the above first channel end, incubating for 2 min, removing via the above second channel end by pipetting, pipetting 100 μl buffer A in the above first channel end and removing the fluid via the above second channel end by pipetting.
pipetting 100 μl buffer B (composition see below) into the above first channel end and removing the fluid via the above second channel end by pipetting
pipetting 40 μl of the hybridized solution from the above step (solution with 2D nanostructures for the 2D sample Probe or solution with 3D DNA nanostructures for the 3D sample or corresponding control experiments without target DNA oligomer) in the above first channel end. Incubation for 15 min. During this time, the biotin of the capture strands is bound to the surface-bound streptavidin. In addition, some of the DNA nanostructures adhere unspecifically to the surface. This is unintended and occurs with 2D DNA nanostructures significantly more frequently than with 3D DNA nanostructures. Removing via the above second channel end by pipetting.
Washing out the unbound DNA nanostructures by pipetting 200 μl buffer RX07 (composition see below) into the above first channel end and removing via the above second channel end by pipetting
Pipetting 50 μl buffer RX07 (composition see below) into the above first channel end

With completion of the above-described steps, there were two samples, one 2D sample and one 3D sample. Moreover, there were respective controls (without target DNA) for both the 2D and 3D nanostructures. The combination of the selected concentrations of biotin-BSA, streptavidin and target structures in the above described method was selected such that the individual identification structures could be easily resolved in the subsequent analysis.

Subsequently, the identification structures in the two samples were imaged with an epifluorescence microscope. To this end, an “Elite” microscope (DeltaVision) was employed. The use of a microscope of the type “Ti Eclipse” (Nikon) would also be conceivable. The camera used was a sCMOS camera (edge 4.2 by PCO) with a pixel size of 6.5 μm. With an objective magnification of 100× and a numeric aperture of 1.4, a pixel resolution of 60 nm was given, i.e. one pixel in the image corresponds to 60 nm in reality. Thus, diffraction limited images of the DNA nanostructures were taken. The filter sets were Chroma 49914 for red, Chroma 49008 for green, Chroma 49020 for blue excitation.

10 images were taken per color for both the 2D sample and the 3D sample as well as for the two controls without target DNA. The colors were imaged sequentially. Each of these images was taken at a specific section of the sample which did not overlap with the sections of the other images and which was located in the mean half along the channel width.

The related 10 images of each sample were analysed. The objective of the analysis presented herein is to compare the number of detected spots in the respective sample with target DNA and the respective sample without target DNA and/or to specify the number of the single-colored in comparison to multi-colored spots. The analysis was carried out manually. To this end, the images were opened with the image processing program FIJI (www.fiji.sc) and the number of single-colored, three-colored as well as the total number of fluorescent spots were counted. For 2D DNA nanostructures and 3D DNA nanostructures, mean value and standard deviation of different parameters of the respective 10 images were determined and plotted in FIG. 3.

Even though it was not applied in the present example, it is possible to use specifically programmed analysis software instead of manual analysis. In the present example, the programmed analysis software is python-based. The python-based analysis software loads the measurement data, determines local maximum values in image boxes of 9-15 pixel size (depending on magnification and numeric aperture of the objective) and calculates the cumulative absolute value of the gradient as noise-independent measurement value of the signal. In order to distinguish between image boxes with and without DNA nanostructures DBSCAN (density based spatial clustering of applications with noise) (published in Ester, Martin; Kriegel, Hans-Peter; Sander, Jorg; Xu, Xiaowei “A density-based algorithm for discovering clusters in large spatial databases with noise”, Proceedings of the Second International Conference on Knowledge Discovery and Data Mining (KDD-96) AAAI Press. pp. 226-231 (1996)) is used. Optionally, the image boxes are partitioned into groups of different values of the cumulative absolute value of the gradient, which corresponds to a classification according to different numbers of fluorophores. Based on their transversal (x, y) positions, the image boxes with DNA nanostructures of each color channel are compared with all image boxes with DNA nanostructures of other color channels in order to recognize multi-colored spots in the image. The aim of the analysis using python-based software as presented herein is also to compare the number of the detected spots in experiments with and without target DNA oligomers and/or to specify the number of single-colored spots in comparison to multi-colored spots. The software stores the determined values for the number of single-colored spots as well as their locations in the image and the values for the number of multi-colored spots as well as their locations in the image for further use.

3. Results and Discussion:

To evaluate the suitability of the DNA nanostructures for the method described, it was measured how many DNA nanostructures were found in the respective control (without target DNA oligomer) in comparison to the corresponding sample with target DNA oligomer (top of FIG. 3). This allowed conclusions which proportion of the DNA nanostructures measured in the sample in the presence of the target structure was due to unspecific nanostructures bound to the surface. It was found that the 3D DNA nanostructures show a significantly lower degree (1.2% for 3D compared to 4.0% for 2D) of unspecific adherence to the microscopy surface of the microscopy sample carrier. The error bar (standard deviation) for the 3D DNA nanostructures crosses the zero line and thus indicates that with 3D DNA nanostructures, the rate of unspecific binding is below the limit of resolution of the measurement series.

It was further measured how many three-colored spots, i.e. data points which could be interpreted as target molecules, can be found in the control in comparison to the sample with target DNA oligomer, again for the 2D sample in comparison to the 2D control and for the 3D sample in comparison to the 3D control. This provides information regarding the false-positive recognized target structures in the case of the 2D DNA nanostructures and in the case of the 3D DNA nanostructures. The presence of three-colored measuring points in the control could, for example, be caused by the interaction of outwardly exposed dyes of different DNA nanostructures. This was avoided by the design of the 3D DNA nanostructures with inwardly disposed dyes. FIG. 3 (middle) shows the result of 0.7% for the 2D DNA nanostructures and 0.0% for the 3D DNA nanostructures. In the 3D case, not a single false-positive measurement was made. Thus, the result shows that due to the use of a plurality of DNA nanostructures binding to a target structure a very low rate of false-positive signals is measured. The result further shows that the 3D DNA nanostructures with inwardly disposed fluorescence dye molecules of the invention interact less with the carrier surface used in the example. This is due to their position inside the 3D structure.

Another parameter is the percentage of three-colored measuring points in the total number of measuring points with target DNA oligomers. It was calculated for both the 2D sample and the 3D sample based on the results of the image analysis and is represented in FIG. 3 (bottom). Due to the reaction kinetics, it is to be expected that identification structures are only partially hybridized under certain circumstances. This may for example be the case if the concentration of the DNA nanostructures is not sufficiently high for saturation of the reaction within the duration of the hybridization reaction. If the reaction is not saturated at the time of measurement, absolute quantification of an internal standard or based on empirical data is possible. The internal standard preferably indicates a reference value with known concentration, whereas empirical data preferably allow a comparison between unsaturated and saturated measurement. However, for relative measurements (as carried out herein), this is not relevant, because the ratio of partially hybridized identification structures to target structures does not depend on the target structure and the embodiments of the 3D DNA nanostructures and, thus, the relative frequencies of the completely hybridized identification structures among these are the same as the relative frequencies of the target structures among the latter. Yet, in this respect 2D and 3D nanostructures are comparable due the similar size. However, DNA nanostructures that are bound to each other as well as DNA nanostructures that are unspecifically bound to the surface may alter the result drastically. This is clear in FIG. 3 (bottom) where only 6.8% of the measuring points on the surface may be considered recognizable structures for 2D DNA nanostructures whereas 25.7% can be considered such for 3D DNA nanostructures. Thus, the microscopy surface can be used more efficiently with 3D DNA nanostructures than with 2D DNA nanostructures.

In summary, it can be noted that the 3D DNA nanostructures of the invention have a more than three-times lower tendency to unspecifically adhere to the microscopy surface of the microscopy sample carrier and a drastic and unquantifiable lower tendency to adhere to each other compared to the 2D DNA nanostructures. The area use efficiency of 3D DNA nanostructures is almost four times higher.

The method of the invention uses multiple binding of DNA nanostructures to increase the specificity, i.e. it reduces the false-positive identification rate. With the measurement method with only one DNA nanostructure binding, this rate would be as high as shown in FIG. 3 (top), i.e. 4.0% (2D) and 1.2% (3D), while it is 0.7% (2D) and 0% (3D) in the method of the invention.

Buffers Used

RX07buffer
4×SSC (saline sodium citrate buffer consisting of an aqueous solution of 150 mM sodium chloride and 15 mM trisodium citrate, which is adjusted to pH 7.0 with HCl)
5% dextrane sulfate
0.1% Tween20
5×Denhardts

Buffer A
10 mM Tris-HCl at pH 7.5
100 mM NaCl
0.05% Tween 20

Buffer B
10 mM Tris-HCl at pH 8.0
10 mM MgCl₂
1 mM EDTA
0.05% Tween 20

### Example 4: Gene Expression Analysis Based on Tissue Samples

Here, a tissue sample, e.g. of a breast tumor, is to be analyzed with respect to the expression of a number of marker genes, e.g. 100 genes such as Her2/neu, estrogen receptor, progesterone receptor, TFRC, GAPDH, etc.

The tissue sample can first be dissolved into a suspension of single cells, e.g. enzymatically and/or by shearing forces.

Subsequently, the cells may be disrupted, e.g. mechanically, by lysis buffer, enzymatically and/or chemically or by light.

The lysate can be further processed, e.g. RNA can be extracted, components can be filtered, nucleic acids can be isolated for example by ethanol precipitation. Alternatively, the lysate can be directly subject to further use.

Lysate, the at least two nanoreporters (designed for the respective mRNA sequences which correspond to the genes to be analyzed), in some applications also substrate-binding adapters (target adapters) and reaction buffers are mixed and incubated for a sufficient time period, e.g. 12 h, wherein complexes (identification structures) are formed.

Subsequently, the complexes are detected, in some applications on a surface or in solution.

It is possible to determine relative gene expression by counting the individual mRNA sequences detected and identified, e.g. if mRNA 1 is detected 200 times and mRNA 2 is detected 300 times, gene expression of gene 2 is 3/2 higher than that of gene 1.

In addition, it is possible to add nucleic acids which bind to defined nanoreporter (exactly comparable with an mRNA sequence) in known concentrations as reference in the incubation step. This allows the normalization and absolute qualification of the other detected mRNA sequences. If this reference is e.g. identified 100 times with an initial concentration of 100 pM, the concentration of mRNA 1 can be determined to be 200 pM.

### Example 5: Protein Detection

Detection and identification of protein target molecules can be carried out in a way similar to the detection and identification of nucleic acid target molecules.

Precondition is that the protein target molecule possesses at least two distinguishable epitopes which can be bound by corresponding binders (antibodies, aptameres, nanobodies, adhirons).

These adapters can be specifically bound to nanoreporters which identify them. To this end, protein-based binders (antibodies, adhirons, nanobodies) can be modified with a specific, short (15-35 nt) DNA sequence (via SNAP tags, HALO tags, click chemistry, SMCC linker, NHS/amino reactions or the like), the reverse complement of which is attached to the corresponding DNA nanostructures (nanoreporters) and, thus, becomes an adapter. Nucleic acid-based binders (RNA or DNA aptameres) can be elongated with a corresponding sequence into an adapter, wherein their reverse complement is attached to the corresponding DNA nanostructures.

By admixing protein target molecules and adapters at DNA nanostructures and with sufficient reaction time, detectable complexes consisting of target protein and at least two adapters which are each coupled at a DNA nanostructure form.

The complexes can be identified by simultaneous detection of the at least two DNA nanostructures.

Example of the detection of two protein targets:

Protein target 1 with epitope A and epitope B which bind to antibody A and antibody B, respectively.

Protein target 2 with epitope C and epitope D which bind to antibody C and antibody D, respectively.

Antibody A is coupled to a DNA nanostructure which is labeled with red fluorescence dyes.

Antibody B is coupled to a DNA nanostructure which is labeled with green fluorescence dyes.

Antibody C is coupled to a DNA nanostructure which is labeled with yellow fluorescence dyes.

Antibody D is coupled to a DNA nanostructure which is labeled with blue fluorescence dyes.

Admixing the protein targets and the coupled antibody/DNA nanostructures and reaction for a sufficient time period, e.g. 12 hours.

Measurement of the complexes in flow detection. The reaction solution is diluted such that only in extremely rare cases, more than one complex is detected. Simultaneous detection of red/green identifies one single protein target 1, simultaneous detection of yellow/blue identifies one single protein target 2. After previous calibration, it is possible to quantify protein targets 1 and 2 by counting the different detections.

With the same method, it is possible to identify clusters of proteins, wherein, in this case, different antibodies identify different proteins (instead of different epitopes) in the target cluster.

### Example 6: Gene Expression Analysis of Three Genes

In order to demonstrate a particularly preferred embodiment of the method, an analysis of the gene expression of genes GAPDH, TFRC and ACTB of HeLa cells is described in more detail. Thus, the mRNAs of the genes GAPDH, TFRC and ACTB are the target structures and the HeLa cells are the host bodies.

To this end, a suitable set of 3D DNA nanostructures is prepared. It consists of the 3D DNA nanostructures 3D_1_1, 3D_1_2, 3D_2_1, 3D_2_2, 3D_3_1 and 3D_3_2. These are similar to the 3D DNA nanostructures 3D_1, 3D_2 and 3D_3 as described in Example 1, but with the following difference: in the case of 3D_1_1, the composition is as for 3D_1 with the difference that the target-binding DNA oligos used are partially complementary to a region of an mRNA of GAPDH (SEQ_ID_NO 1270 instead of SEQ_ID_NO 1263), in the case of 3D_1_2, the composition is as for 3D_2 with the difference that the target-binding DNA oligos used are partially complementary to another region of an mRNA of GAPDH (SEQ_ID_NO 1276 instead of SEQ_ID_NO 1264), in the case of 3D_2_1, the composition is as for 3D_1 with the difference that the target-binding DNA oligos used are partially complementary to a region of an mRNA of TFRC (SEQ_ID_NO 1271 instead of SEQ_ID_NO 1263), in the case of 3D_2_2, the composition is as for 3D_3 with the difference that the target-binding DNA oligos used are partially complementary to a portion of another region of an mRNA of TFRL (SEQ_ID_NO 1277 instead of SEQ_ID_NO 1265), in the case of 3D_3_1, the composition is as for 3D_2 with the difference that the target-binding DNA oligos used are partially complementary to a region of an mRNA of ACTB (SEQ_ID_NO 1272 instead of SEQ_ID_NO 1264) and in the case of 3D_3_2, the composition is as for 3D_3 with the difference that the target-binding DNA oligos used are partially complementary to another region of an mRNA of ACTB (SEQ_ID_NO 1278 instead of SEQ_ID_NO 1265).

A microwell chamber is used for this Example. Said chamber comprises a microwell array with a 1^{st} layer of a Quarz Wafer of 170 μm thickness and a 2^{nd} layer of Liquid Glass. A composition of the Liquid Glass that is generally possible for all embodiments of the invention is as follows: 49 vol % HEMA (monomer, hydroxyethylmethacrylate); 5 vol % TEGDA (polymer, tetra(ethyleneglycol)diacrylate); 18 vol % POE (lysis medium, phenoxyethanol), 28 vol % aerosol OX50 (silica nanopowder); 0.5 wt % DMPAP (photoinitiator, 2,2dimethoxy-2-phenylacetophenon (see, e.g., F. Kotz et al, “Three-dimensional printing of transparent fused silica glass”, Nature 2017, 544 (7650), 337-339 and F. Kotz et al. “Liquid Glass: A Facile Soft Replication Method for Structuring Glass”, Advanced Materials 2016, 28, 4646-4650). Here, the 3^{rd} layer of the microwell chamber is formed by an ibidi sticky-slide VI 0.4 (ibidi GmbH), which is adhered to the 2^{nd} layer. Alternatively, other adhesives or other techniques of adhering can be used. The 3^{rd} layer comprises a recess to form a cavity. The 1^{st}, 2^{nd} and 3^{rd} layer are arranged such that they form a working cavity with microwells according to the manner already described. The volume of the working cavity including the microwells in this case is, for example, 40 microliters.

As subsequent step, a solution of 1 mg/ml biotinylated BSA in buffer A (biotin-BSA: product A8549, Sigma-Aldrich GmbH) is flushed into the working cavity and incubated for 10 minutes. Subsequently, a rinsing step with 0.5 ml buffer A (formulation see below) follows. Then, a solution of streptavidin in buffer A with a concentration of 0.5 mg/ml is flushed in and incubated for 10 minutes. During the incubation period, at least a part of the streptavidin binds to the surfaces of the microwells.

Subsequently, a rinsing step with 0.5 ml buffer A and a further rinsing step with 0.5 ml buffer RX07 (formulation see below) follows. Afterwards, a solution of 1 nM biotin-adapter oligo (SEQ_ID_NO 1262) in RX07 is flushed in and incubated for 10 minutes. During the incubation period, at least a part of the biotin-adapter oligos binds via biotin-streptavidin bonds to the streptavidins that are bound to the surfaces and, thus, itself binds to the surfaces.

Subsequently, a rinsing step with 0.5 ml buffer RX07 follows. Afterwards, a solution with SEQ_ID_NO 1273, 1274 and 1275 (see below), each with a concentration of 1 nM, in buffer RX07 is flushed in and incubated for 10 minutes so that SEQ_ID_NO 1273, 1274 and 1275 bind to biotin-adapter oligos SEQ_ID_NO 1262.

Subsequently, after a rinsing step with 0.5 ml buffer RX07, a cell suspension having a density of 1 million cells per milliliter is flushed in. The buffer of the cell suspension is PBS (Phosphate Buffered Saline, composition see below). The cells are given 10 minutes to sink into the microwells.

Subsequently, the microwell chamber and the selection of the microwells occupied by exactly one cell are placed on a microscope for further analysis. Arranging the microwell chamber on the microscope can also be done at an earlier point, e.g. at the very beginning of the described steps. Preferably, the microscope is suitable for all subsequent microscopy analysis steps. Alternatively different microscopes can be used for different microscope techniques. Then, the individual microwells are categorized by whether they comprise multiple cells, exactly one cell or no cell. Only those microwells which comprise exactly one cell are considered in the subsequent analysis. Said categorization can be done by any suitable microscopy technique, preferably by bright-field, phase contrast, dark-field or DIC (differential interference contrast) microscope or fluorescence microscopy, inter alia preferably epifluorescence microscopy, TIRF microscopy, dot scanning confocal microscopy, spinning-disc confocal microscopy, light sheet microscopy.

Subsequently, a disruption buffer, more precisely a lysis buffer (formulation see below), is added to the microwell chamber and incubated for 30 minutes. Thereby the cells are lysed and target structures which are potentially contained therein are released. The target structures now bind to the carrier adapters at the bottoms of individual microwells, in which the target structures were released.

After a rinsing step with 0.5 ml RX07, in which the target structures remain in the microwells due to their bond to the bottoms, a solution of each 50 nM 3D_1_1, 3D_1_2, 3D_2_1, 3D_2_2, 3D_3_1 and 3D_3_2 in RX07 is injected and incubated for 60 minutes. During incubation, many of the 3D DNA nanostructures bind to a corresponding target structure.

Three rinsing steps with each 0.5 ml RX07 follow.

Subsequently, fluorescence recordings of the region close to the bottom of the microwells previously selected for analysis are made, including the wave lengths used in the 3D DNA nanostructures. Since in the present Example, only 3 genes are analyzed, the 3D DNA nanostructures could be selected such that two types of 3D DNA nanostructures, herein 3D_1_1 and 3D_2_1, have a 1^{st} color, preferably red, two 2^{nd} types of 3D DNA nanostructures, herein 3D_1_2 and 3D_3_1, have a 2^{nd} color, preferably green, and two third types of 3D DNA nanostructures, herein 3D_2_2 and 3D_3_2 have a 3^{rd} color, preferably blue.

Subsequently, the evaluation, i.e. the detection and/or the quantification of the target structures, is carried out in that spots of the respective color are searched in each microwell that is selected for analysis. A red-green spot represents GAPDH mRNA, red-blue spot represents TFRC mRNA and a green-blue spot represents ACTB mRNA.

The representation of the number of the detected colored spots per microwell in two scatter plots allows for further evaluation, if needed. In the 1^{st} scatter plot, the number of detected spots per microwell is plotted for colors red versus green, while in the 2^{nd} scatter plot, the number of detected spots per microwell is plotted for colors green versus blue (or alternatively red versus blue). For example, the gene expression levels for the three analyzed genes as well as different sub-populations of cells can be inferred from the scatter plots.

### Formulations for Example 6

Buffer A: See buffer A in Example 3

RX07 buffer: See RX07 buffer in Example 3

Lysis Buffer
20 mM Tris pH 7.5
150 mM NaCl
1 mM EDTA
1 mM EGTA
1% protease inhibitor cocktail, P-8340 for mammalian cells, Sigma-Aldrich

PBS Buffer (Phosphate Buffered Saline)
137 mM NaCl
2.7 mM KCl
10 mM Na₂HPO₄
1.8 mM KH₂PO₄

SEQ IDs
SEQ_ID_NO: 1270 T4_gapdh
TTGTGTCGTGACGAGAAACACCAAATTTCAACTTTTTTTTCCATTGATGA
CAAGCTTCCCGTTCTCAGCCTT
SEQ_ID_NO: 1271 T4_tfrc
TTGTGTCGTGACGAGAAACACCAAATTTCAACTTTTTCGAGTTTTGAGCG
CTGTCTTTGACCTGAATCTTAAC
SEQ_ID_NO: 1272 T4_actb
TTGTGTCGTGACGAGAAACACCAAATTTCAACTTTTTAATGATCTTGATC
TTCATTGTGCTGGGTGCCAG
SEQ_ID_NO: 1273 carrier adapter_gapdh
GCCATGGGTGGAATCATATTGGAACATGTAAACCTTCGGTTGTACTGTGA
CCGATTC
SEQ_ID_NO: 1274 carrier adapter_tfrc
CACGCCAGACTTTGCTGAGTTTAAATTCACGTTCGGTTGTACTGTGACCG
ATTC
SEQ_ID_NO: 1275 carrier adapter_actb
ATGATGGAGTTGAAGGTAGTTTCGTGGATGCCACATTCGGTTGTACTGTG
ACCGATTC
SEQ_ID_NO 1276 T5_gapdh
TTGTGTCGTGACGAGAAACACCAAATTTCAACTTTTctgggtggcagtga
tggcatggactgtggtcatgagtcct
SEQ_ID_NO 1277 T5_TFRC
TTGTGTCGTGACGAGAAACACCAAATTTCAACTTTTTacccatcttttaa
gaccatatctgagaacatctgggc
SEQ_ID_NO 1278 T5_ACTB
TTGTGTCGTGACGAGAAACACCAAATTTCAACTTTTTcagtgtacaggta
agccctggctgcctccaccc

1. A method for the detection of a target structure, comprising:
p1) introduction of a group of host bodies including a host body with a target structure into a microwell array such that exactly one host body is present in at least one microwell;
p2) introduction of at least two 3D DNA nanostructures into the at least one microwell wherein each of the 3D DNA nanostructures comprises one or more inwardly disposed fluorescence dye molecules;
a) forming an identification structure in the at least one microwell, comprising:
(i) the target structure, and
(ii) the at least two 3D DNA nanostructures, wherein each of the 3D DNA nanostructures is specifically bound to the target structure and wherein the 3D DNA nanostructures are bound to pairwise different regions of the target structure;
b) detection of the target structure by measuring at least one fluorescence signal,
wherein the 3D DNA nanostructures and the parameters of fluorescence measurement are selected such that the at least one measured fluorescence signal of the identification structure formed in a) is distinguishable from the fluorescence signal of each of the at least two isolated 3D DNA nanostructures, when these are not bound in the respective identification structure, wherein the identification structure is bound to a first surface, preferably the bottom of the at least one microwell.

2. The method of claim 1, wherein the host body is a cell, a virus, an exosome, a vesicle and/or a droplet.

3. The method of claim 1, wherein the target structure is present in a host body, wherein the method further comprises:
c) disruption of the host bodies, preferably by liquid exchange with a disruption buffer in order to release the target structure from the host body.

4. The method of claim 1, wherein the target structure comprises or is one element of the group consisting of: a polynucleotide, a partially single-stranded polynucleotide, a single-stranded polynucleotide or an mRNA.

5. A microwell array for fluorescence microscopy, which comprises multiple microwells, wherein each microwell comprises a bottom and a circumferential wall, wherein the bottoms are suited for fluorescence microscopy and wherein each of the microwells has a top side,
wherein the microwell array further comprises:
a first layer which forms the bottoms of the microwells, wherein the first layer is suited for fluorescence microscopy;
a second layer, which is applied to the first layer, wherein the second layer forms the walls of the microwells.

6. The microwell array of claim 5, wherein one or more carrier adapters is/are bound to the bottom of at least one of the microwells.

7. The microwell array of claim 5, wherein the second layer comprises or consists of one element of the group consisting of: SU-8, PEG-DA, PDMS, CYTOP or liquid glass.

8. The method of claim 1, wherein the microwell array comprises multiple microwells, wherein each microwell comprises a bottom and a circumferential wall, wherein the bottoms are suited for fluorescence microscopy and wherein each of the microwells has a top side,
wherein the microwell array further comprises:
a first layer which forms the bottoms of the microwells, wherein the first layer is suited for fluorescence microscopy;
a second layer, which is applied to the first layer, wherein the second layer forms the walls of the microwells and
wherein the first surface is the bottom of the at least one microwell, wherein the target structure is bound and/or is being bound to the bottom of the microwell by mediation of a carrier adapter which specifically binds to the target structure.

9. The method of claim 8, wherein each of the microwells has an open top side, the method further comprising:
f) closing the top side of the microwell array by applying an oil layer to the second layer.

10. The method of claim 1, wherein the method is further additionally suited for the detection of one or more further target structures that are different from each other, wherein the different target structures are pairwise different,
wherein for each of the target structures, the group of host bodies in step p1) comprises at least one host body having the relevant target structure;
and wherein the method further comprises:
c) for each of the one or more further target structures that are different from each other:
forming of a respectively assigned identification structure, wherein each of the further identification structures comprises:
(i) the respective assigned further target structure, and
(ii) at least two 3D DNA nanostructures, wherein each of the at least two 3D DNA nanostructures comprises one or more inwardly disposed fluorescence dye molecules and wherein each of the at least two 3D DNA nanostructures is specifically bound to the respective further target structure and wherein the at least two 3D DNA nanostructures are bound to pairwise different regions of the respective target structure;
and wherein step a) further comprises:
d) detection of the one or more further target structures by measuring the at least one fluorescence signal,
wherein all 3D DNA nanostructures and the parameter of the fluorescence measurement are selected such that the at least one measured fluorescence signal of the identification structures formed in a) and c) is distinguishable from the fluorescence signal of all isolated 3D DNA nanostructures, when these are not bound in one of the identification structures, and that the measured fluorescence signals of all formed identifications structures are pairwise distinguishable from one another, wherein each of the different target structures may be present multiple times and the method may comprise the multiple detection of one or more of the different target structures.

11. The method of claim 8, wherein in step p1), the host bodies are isolated by applying a volume of a liquid containing the host bodies onto the open side of the microwell arrays and by allowing time for the host bodies to sink to the bottom of the microwells due to the effect of gravity.

12. The method of claim 1, wherein the method further comprises:
aa) preparing a carrier matrix in the at least one microwell.

13. The method of claim 1, wherein the method further comprises:
h) removing the circumferential walls of the microwells from the bottoms between steps p1) and a) or between steps a) and b).

14. A kit, comprising:
a microwell array; and
at least two 3D DNA nanostructures, wherein each of the 3D DNA nanostructures comprises at least one inwardly disposed fluorescence dye molecule.

15. The kit of claim 14, wherein for each of the at least two 3D DNA nanostructures, a distance of the at least one inwardly disposed fluorescence dye molecule to the rim of the 3D DNA nanostructure is at least 2 nm.

16. The method of claim 9, wherein the method further comprises:
g) draining off the oil by pouring an aqueous solution onto the microwell array.

17. The method of claim 12, wherein the method further comprises:
bb) attaching carrier adapters to the carrier matrix.

18. The kit of claim 16, wherein the microwell array comprises multiple microwells, wherein each microwell comprises a bottom and a circumferential wall, wherein the bottoms are suited for fluorescence microscopy and wherein each of the microwells has a top side,
wherein the microwell array further comprises:
a first layer which forms the bottoms of the microwells, wherein the first layer is suited for fluorescence microscopy;
a second layer, which is applied to the first layer, wherein the second layer forms the walls of the microwells.

19. The kit of claim 16, wherein at least one of the 3D DNA nanostructures comprises at least 2 inwardly disposed fluorescence dye molecules and wherein the pairwise distance of the at least two inwardly disposed fluorescence dye molecules is at least 2 nm.


SEQUENCE LISTING (1278 sequences)

SEQ ID NO: 1 - DNA, 45 bp, Artificial Sequence
  Staple strand_0000 of the 3D DNA nanostructure.
agagtccactattttaatgaacgctttccagtcgggggtcgacgt

SEQ ID NO: 2 - DNA, 45 bp, Artificial Sequence
  Staple strand_0001 of the 3D DNA nanostructure.
tatctaaaatatcttttgaatacctgaaagcgtaagagcaataca

SEQ ID NO: 3 - DNA, 45 bp, Artificial Sequence
  Staple strand_0002 of the 3D DNA nanostructure.
cagtacataaatcgcacgtaaatggaagggttagaaagattaggt

SEQ ID NO: 4 - DNA, 45 bp, Artificial Sequence
  Staple strand_0003 of the 3D DNA nanostructure.
aacatgttcagctccgtgtgaatcttctgacctaaatgcttctaa

SEQ ID NO: 5 - DNA, 45 bp, Artificial Sequence
  Staple strand_0004 of the 3D DNA nanostructure.
gggtaattgagcgttaaatcatagcgaacctcccgacaacaatac

SEQ ID NO: 6 - DNA, 45 bp, Artificial Sequence
  Staple strand_0005 of the 3D DNA nanostructure.
cgtcagactgtagaatagaaaaaagacaccacggaacaagaatga

SEQ ID NO: 7 - DNA, 45 bp, Artificial Sequence
  Staple strand_0006 of the 3D DNA nanostructure.
accgtactcaggagcgtcatatggaaagcgcagtctgtcatagag

SEQ ID NO: 8 - DNA, 45 bp, Artificial Sequence
  Staple strand_0007 of the 3D DNA nanostructure.
aacggctacagagcgaataatcggagtgagaatagaaaccgcctc

SEQ ID NO: 9 - DNA, 45 bp, Artificial Sequence
  Staple strand_0008 of the 3D DNA nanostructure.
aataaaacgaactgtacagacgaactgaccaacttttcatgaggc

SEQ ID NO: 10 - DNA, 45 bp, Artificial Sequence
  Staple strand_0009 of the 3D DNA nanostructure.
tgttttaaatatgatgaccatccccctcaaatgctttagaaagtt

SEQ ID NO: 11 - DNA, 45 bp, Artificial Sequence
  Staple strand_0010 of the 3D DNA nanostructure.
gtaatcgtaaaacgggagaagtgtaccaaaaacattagtttcaca

SEQ ID NO: 12 - DNA, 45 bp, Artificial Sequence
  Staple strand_0011 of the 3D DNA nanostructure.
aaaacgacggccacatcgtaaccgtaatgggataggccggttgcg

SEQ ID NO: 13 - DNA, 45 bp, Artificial Sequence
  Staple strand_0012 of the 3D DNA nanostructure.
tcccttataaatccggtttgcaagcctggggtgcctcaagtccgg

SEQ ID NO: 14 - DNA, 45 bp, Artificial Sequence
  Staple strand_0013 of the 3D DNA nanostructure.
aaccctcaatcaaaactgatacagtcacacgaccagtcacttgaa

SEQ ID NO: 15 - DNA, 45 bp, Artificial Sequence
  Staple strand_0014 of the 3D DNA nanostructure.
caagaaaacaaaaagattttcatcagatgatggcaattgaggaca

SEQ ID NO: 16 - DNA, 45 bp, Artificial Sequence
  Staple strand_0015 of the 3D DNA nanostructure.
aagagaatataaataaacacctccaatcgcaagacaattttccat

SEQ ID NO: 17 - DNA, 45 bp, Artificial Sequence
  Staple strand_0016 of the 3D DNA nanostructure.
ataaaaacagggacagctacaatagcaagcaaatcaaataataat

SEQ ID NO: 18 - DNA, 45 bp, Artificial Sequence
  Staple strand_0017 of the 3D DNA nanostructure.
caatgaaaccatcaaagacaagtatgttagcaaacgagcaagaac

SEQ ID NO: 19 - DNA, 45 bp, Artificial Sequence
  Staple strand_0018 of the 3D DNA nanostructure.
accaggcggataagtgtactgcaggtcagacgattgtcttttcac

SEQ ID NO: 20 - DNA, 45 bp, Artificial Sequence
  Staple strand_0019 of the 3D DNA nanostructure.
ggccgcttttgcgccaaaaaaacgttagtaaatgaaagagccagt

SEQ ID NO: 21 - DNA, 45 bp, Artificial Sequence
  Staple strand_0020 of the 3D DNA nanostructure.
tgcgattttaagattcatcaagacctgctccatgttaaatacgaa

SEQ ID NO: 22 - DNA, 45 bp, Artificial Sequence
  Staple strand_0021 of the 3D DNA nanostructure.
gaggtcatttttgcctgactaaaatgtttagactggaataccata

SEQ ID NO: 23 - DNA, 45 bp, Artificial Sequence
  Staple strand_0022 of the 3D DNA nanostructure.
ctacaaaggctataaaattttaaggcaaagaattagcaattctaa

SEQ ID NO: 24 - DNA, 45 bp, Artificial Sequence
  Staple strand_0023 of the 3D DNA nanostructure.
ggatgtgctgcaaggacgacgttaaatgtgagcgagacaggaaat

SEQ ID NO: 25 - DNA, 45 bp, Artificial Sequence
  Staple strand_0024 of the 3D DNA nanostructure.
acgctggtttgccttcttttctgaaattgttatccggctagtagc

SEQ ID NO: 26 - DNA, 45 bp, Artificial Sequence
  Staple strand_0025 of the 3D DNA nanostructure.
gctgagagccagcaataccgaatacctacattttgatatcggccc

SEQ ID NO: 27 - DNA, 45 bp, Artificial Sequence
  Staple strand_0026 of the 3D DNA nanostructure.
gcgcagaggcgaaacagtaaccattttgcggaacaaaaacaatac

SEQ ID NO: 28 - DNA, 45 bp, Artificial Sequence
  Staple strand_0027 of the 3D DNA nanostructure.
atcgccatatttagcctgtttcctttttaacctccggcgatagtc

SEQ ID NO: 29 - DNA, 45 bp, Artificial Sequence
  Staple strand_0028 of the 3D DNA nanostructure.
agaaacgatttttcgctaacgctcatcgagaacaagtgtagaaga

SEQ ID NO: 30 - DNA, 45 bp, Artificial Sequence
  Staple strand_0029 of the 3D DNA nanostructure.
aattagagccagcagggagggacgcaataataacggatcttacta

SEQ ID NO: 31 - DNA, 45 bp, Artificial Sequence
  Staple strand_0030 of the 3D DNA nanostructure.
agtattaagaggcgtgccttgcagagccgccaccagagagccagg

SEQ ID NO: 32 - DNA, 45 bp, Artificial Sequence
  Staple strand_0031 of the 3D DNA nanostructure.
aacaaccatcgccattgtatcattccacagacagccgcaagccaa

SEQ ID NO: 33 - DNA, 45 bp, Artificial Sequence
  Staple strand_0032 of the 3D DNA nanostructure.
gagtagtaaattgatattcatcgaaacaaagtacaaaacctaaac

SEQ ID NO: 34 - DNA, 45 bp, Artificial Sequence
  Staple strand_0033 of the 3D DNA nanostructure.
ccggaagcaaactttgcatcaaaaaccaaaatagcgtaacgccac

SEQ ID NO: 35 - DNA, 45 bp, Artificial Sequence
  Staple strand_0034 of the 3D DNA nanostructure.
gatattcaaccgttgcaatgcaaggtggcatcaattgaccattga

SEQ ID NO: 36 - DNA, 45 bp, Artificial Sequence
  Staple strand_0035 of the 3D DNA nanostructure.
aactgttgggaagcgcactccaggaacgccatcaaaaattgtaat

SEQ ID NO: 37 - DNA, 45 bp, Artificial Sequence
  Staple strand_0036 of the 3D DNA nanostructure.
ctggccaacagaggattagtgatagggttgagtgtacgcgcggct

SEQ ID NO: 38 - DNA, 45 bp, Artificial Sequence
  Staple strand_0037 of the 3D DNA nanostructure.
tgattgtttggataatagatcaaatcaacagttgaagtctttatt

SEQ ID NO: 39 - DNA, 45 bp, Artificial Sequence
  Staple strand_0038 of the 3D DNA nanostructure.
tttttcaaatatagtcgctaaatttcatttgaattataaagaacc

SEQ ID NO: 40 - DNA, 45 bp, Artificial Sequence
  Staple strand_0039 of the 3D DNA nanostructure.
ccggtattctaagtcctgaaaaagtaattctgtccggcgttaaac

SEQ ID NO: 41 - DNA, 45 bp, Artificial Sequence
  Staple strand_0040 of the 3D DNA nanostructure.
aaaggtggcaacaagcccaaagaattaactgaacattgctattat

SEQ ID NO: 42 - DNA, 45 bp, Artificial Sequence
  Staple strand_0041 of the 3D DNA nanostructure.
acaaataaatcctattagcgatcagtagcgacagatggtttacat

SEQ ID NO: 43 - DNA, 45 bp, Artificial Sequence
  Staple strand_0042 of the 3D DNA nanostructure.
ttgctaaacaactgaaccgcttgatataagtatagtttgatgaaa

SEQ ID NO: 44 - DNA, 45 bp, Artificial Sequence
  Staple strand_0043 of the 3D DNA nanostructure.
gcgcagacggtcaccattaagcagcgaaagacagctcacgttgtt

SEQ ID NO: 45 - DNA, 45 bp, Artificial Sequence
  Staple strand_0044 of the 3D DNA nanostructure.
cggaatcgtcataagttgagcagtcaggacgttggataggctgag

SEQ ID NO: 46 - DNA, 45 bp, Artificial Sequence
  Staple strand_0045 of the 3D DNA nanostructure.
agcctcagagcattaacagtttaattgctgaatataaatcaggtg

SEQ ID NO: 47 - DNA, 45 bp, Artificial Sequence
  Staple strand_0046 of the 3D DNA nanostructure.
tctccgtgggaacgaaaagcgagtctggagcaaacttcaacgcaa

SEQ ID NO: 48 - DNA, 45 bp, Artificial Sequence
  Staple strand_0047 of the 3D DNA nanostructure.
cacattaattgcgctttgattaacgccagggtttttctgccagat

SEQ ID NO: 49 - DNA, 45 bp, Artificial Sequence
  Staple strand_0048 of the 3D DNA nanostructure.
atggattatttacagaagaacctgtttgatggtgggcgccaggca

SEQ ID NO: 50 - DNA, 45 bp, Artificial Sequence
  Staple strand_0049 of the 3D DNA nanostructure.
agcggaattatcagtattagtaaagcatcaccttgaacatcgcaa

SEQ ID NO: 51 - DNA, 45 bp, Artificial Sequence
  Staple strand_0050 of the 3D DNA nanostructure.
ataactatatgtatccttgaacctgagcaaaagaaacgtcagagg

SEQ ID NO: 52 - DNA, 45 bp, Artificial Sequence
  Staple strand_0051 of the 3D DNA nanostructure.
tcatcgtaggaatctaatttaatttaggcagaggctaattactat

SEQ ID NO: 53 - DNA, 45 bp, Artificial Sequence
  Staple strand_0052 of the 3D DNA nanostructure.
ggcatgattaagaaaatagcatgaaaatagcagcccctgaatctt

SEQ ID NO: 54 - DNA, 45 bp, Artificial Sequence
  Staple strand_0053 of the 3D DNA nanostructure.
gccgccagcattgaaatcacaccattaccattagcacattcaact

SEQ ID NO: 55 - DNA, 45 bp, Artificial Sequence
  Staple strand_0054 of the 3D DNA nanostructure.
cgatctaaagtttcattttcgaaggattaggattagttttaaccc

SEQ ID NO: 56 - DNA, 45 bp, Artificial Sequence
  Staple strand_0055 of the 3D DNA nanostructure.
cgcctgataaattactacgatattcggtcgctgagcaaaaggaaa

SEQ ID NO: 57 - DNA, 45 bp, Artificial Sequence
  Staple strand_0056 of the 3D DNA nanostructure.
gaagttttgccagctaatgcaatttcaactttaatcttgacaaat

SEQ ID NO: 58 - DNA, 45 bp, Artificial Sequence
  Staple strand_0057 of the 3D DNA nanostructure.
cattaacatccaaagtagattagagagtacctttacagaagcaaa

SEQ ID NO: 59 - DNA, 45 bp, Artificial Sequence
  Staple strand_0058 of the 3D DNA nanostructure.
ccttcctgtagcctaagcaaaatgccggagagggtctcatataag

SEQ ID NO: 60 - DNA, 45 bp, Artificial Sequence
  Staple strand_0059 of the 3D DNA nanostructure.
acatacgagccggaccgtatcctcttcgctattaccggcctcagg

SEQ ID NO: 61 - DNA, 45 bp, Artificial Sequence
  Staple strand_0060 of the 3D DNA nanostructure.
attgcaacaggaagtaatatcctggccctgagagaagacgggccg

SEQ ID NO: 62 - DNA, 45 bp, Artificial Sequence
  Staple strand_0061 of the 3D DNA nanostructure.
taattttaaaagtctcgtatgtcagtattaacaccaccagcagcc

SEQ ID NO: 63 - DNA, 45 bp, Artificial Sequence
  Staple strand_0062 of the 3D DNA nanostructure.
tatcaaaatcatataagacgcctgattgctttgaatttacatcat

SEQ ID NO: 64 - DNA, 45 bp, Artificial Sequence
  Staple strand_0063 of the 3D DNA nanostructure.
agaacgggtattaaataatcagccaacgctcaacatatgcgtttt

SEQ ID NO: 65 - DNA, 45 bp, Artificial Sequence
  Staple strand_0064 of the 3D DNA nanostructure.
caaagttaccagactttttaaaacagccatattatttccagagca

SEQ ID NO: 66 - DNA, 45 bp, Artificial Sequence
  Staple strand_0065 of the 3D DNA nanostructure.
aaccgccaccctcaaccgccattatcaccgtcaccatattgacaa

SEQ ID NO: 67 - DNA, 45 bp, Artificial Sequence
  Staple strand_0066 of the 3D DNA nanostructure.
accagtacaaactaacccatctatttcggaacctagtgcccgtag

SEQ ID NO: 68 - DNA, 45 bp, Artificial Sequence
  Staple strand_0067 of the 3D DNA nanostructure.
tttgacccccagcgaggcaaagcttgataccgatacagcttgctc

SEQ ID NO: 69 - DNA, 45 bp, Artificial Sequence
  Staple strand_0068 of the 3D DNA nanostructure.
ataaccctcgtttattacgaataaggcttgccctgatcaacgttc

SEQ ID NO: 70 - DNA, 45 bp, Artificial Sequence
  Staple strand_0069 of the 3D DNA nanostructure.
atattttcatttgtttcgcagcgttttaattcgagtaagaggatc

SEQ ID NO: 71 - DNA, 45 bp, Artificial Sequence
  Staple strand_0070 of the 3D DNA nanostructure.
gttaaatcagctcatattttaaggccggagacagtatgtgtagct

SEQ ID NO: 72 - DNA, 45 bp, Artificial Sequence
  Staple strand_0071 of the 3D DNA nanostructure.
taatcatggtcataaggatcaggcaaagcgccatttttccggctt

SEQ ID NO: 73 - DNA, 42 bp, Artificial Sequence
  Staple strand_0072 of the 3D DNA nanostructure.
acgtggacgggactgcgcgagggtaggaccagctgcaaaaga

SEQ ID NO: 74 - DNA, 40 bp, Artificial Sequence
  Staple strand_0073 of the 3D DNA nanostructure.
gtggcacagtgggcacactcgagaactcccgttgtaatac

SEQ ID NO: 75 - DNA, 42 bp, Artificial Sequence
  Staple strand_0074 of the 3D DNA nanostructure.
gagcactaacgggcccgaattgagggcaagacaatattttag

SEQ ID NO: 76 - DNA, 40 bp, Artificial Sequence
  Staple strand_0075 of the 3D DNA nanostructure.
catatcactgaggatatgagacgatgacaactaatcctac

SEQ ID NO: 77 - DNA, 42 bp, Artificial Sequence
  Staple strand_0076 of the 3D DNA nanostructure.
atgtgagtccagtcgccccccgcagttgaaattatttaatat

SEQ ID NO: 78 - DNA, 40 bp, Artificial Sequence
  Staple strand_0077 of the 3D DNA nanostructure.
tggtttgatctgggtagcagcggtaggaataaccttttaa

SEQ ID NO: 79 - DNA, 42 bp, Artificial Sequence
  Staple strand_0078 of the 3D DNA nanostructure.
agaacgcgttgcccacgagtgcggtgataaataccgaaatgc

SEQ ID NO: 80 - DNA, 40 bp, Artificial Sequence
  Staple strand_0079 of the 3D DNA nanostructure.
gggaggtatcccggaccacatatatccctgtttatcttgc

SEQ ID NO: 81 - DNA, 42 bp, Artificial Sequence
  Staple strand_0080 of the 3D DNA nanostructure.
atcagagactgtgggccgcgcaccgccctttgaagccctaat

SEQ ID NO: 82 - DNA, 40 bp, Artificial Sequence
  Staple strand_0081 of the 3D DNA nanostructure.
ttattttgaggcgacggctattgaccgataacccataagt

SEQ ID NO: 83 - DNA, 42 bp, Artificial Sequence
  Staple strand_0082 of the 3D DNA nanostructure.
tttcatcgctagcggttctaagtcgggtgtcacaatccgcgt

SEQ ID NO: 84 - DNA, 40 bp, Artificial Sequence
  Staple strand_0083 of the 3D DNA nanostructure.
tttaccgtgtaaatgcgcggccgagggcattttcgctgaa

SEQ ID NO: 85 - DNA, 42 bp, Artificial Sequence
  Staple strand_0084 of the 3D DNA nanostructure.
agtaccgctgctggaccgtggaccaaggttccagtaaggttt

SEQ ID NO: 86 - DNA, 40 bp, Artificial Sequence
  Staple strand_0085 of the 3D DNA nanostructure.
acaactatgagggtacgggaccatggcaccctcagaagga

SEQ ID NO: 87 - DNA, 42 bp, Artificial Sequence
  Staple strand_0086 of the 3D DNA nanostructure.
gaggactaggagttgtgccaatggcgagaaggaattggcttt

SEQ ID NO: 88 - DNA, 40 bp, Artificial Sequence
  Staple strand_0087 of the 3D DNA nanostructure.
aggacaggcactgggcgccttgttcaaagacttttgaaag

SEQ ID NO: 89 - DNA, 42 bp, Artificial Sequence
  Staple strand_0088 of the 3D DNA nanostructure.
aacaacatgctctagtcggacgtcgactatgaacggtaacgg

SEQ ID NO: 90 - DNA, 40 bp, Artificial Sequence
  Staple strand_0089 of the 3D DNA nanostructure.
agttcagcgcaacgggacgcacagcatattacaggtaaac

SEQ ID NO: 91 - DNA, 42 bp, Artificial Sequence
  Staple strand_0090 of the 3D DNA nanostructure.
aaagtacgcgagggtagggcctaactcaaaaacgagacaact

SEQ ID NO: 92 - DNA, 40 bp, Artificial Sequence
  Staple strand_0091 of the 3D DNA nanostructure.
cctgtaagcgtctagggactcggcacgtgtctggaatgac

SEQ ID NO: 93 - DNA, 42 bp, Artificial Sequence
  Staple strand_0092 of the 3D DNA nanostructure.
tgtcaatcgcggtggccgtactggcccatacttttgctagca

SEQ ID NO: 94 - DNA, 40 bp, Artificial Sequence
  Staple strand_0093 of the 3D DNA nanostructure.
ttggtgttaatctgtggataatccgaatatgtacctcacg

SEQ ID NO: 95 - DNA, 42 bp, Artificial Sequence
  Staple strand_0094 of the 3D DNA nanostructure.
aagcttgccggctcaaggcctcgcgccaagatgggcggtgcc

SEQ ID NO: 96 - DNA, 40 bp, Artificial Sequence
  Staple strand_0095 of the 3D DNA nanostructure.
tgtcgtgggccaagacgtagcgcatgatgcctgcaaaacc

SEQ ID NO: 97 - DNA, 42 bp, Artificial Sequence
  Staple strand_0096 of the 3D DNA nanostructure.
cagtttggccggagacccagggataagccctcggccatgttc

SEQ ID NO: 98 - DNA, 40 bp, Artificial Sequence
  Staple strand_0097 of the 3D DNA nanostructure.
acccttcgaggttgagggcgcagatgaacttctttataga

SEQ ID NO: 99 - DNA, 42 bp, Artificial Sequence
  Staple strand_0098 of the 3D DNA nanostructure.
attgaggagcggctgcgtacgttcgagctgggctattaagga

SEQ ID NO: 100 - DNA, 40 bp, Artificial Sequence
  Staple strand_0099 of the 3D DNA nanostructure.
ttctgaaatgccgcttctccagtgttagagccgtctatac

SEQ ID NO: 101 - DNA, 42 bp, Artificial Sequence
  Staple strand_0100 of the 3D DNA nanostructure.
ttttaatgtactacccgaggacggagcttaaacagaaacctt

SEQ ID NO: 102 - DNA, 40 bp, Artificial Sequence
  Staple strand_0101 of the 3D DNA nanostructure.
gttaattcgctgttccctgcgcgcttgagtaaatctttta

SEQ ID NO: 103 - DNA, 42 bp, Artificial Sequence
  Staple strand_0102 of the 3D DNA nanostructure.
acgacaatcacacacacggcgaattatgtctaaataaagacg

SEQ ID NO: 104 - DNA, 40 bp, Artificial Sequence
  Staple strand_0103 of the 3D DNA nanostructure.
gaggcgttgtggcgtcgtgcgatggcaaagataagaacgc

SEQ ID NO: 105 - DNA, 42 bp, Artificial Sequence
  Staple strand_0104 of the 3D DNA nanostructure.
aacaaagtgtttcgaggaggccgttcgtttagattagccctg

SEQ ID NO: 106 - DNA, 40 bp, Artificial Sequence
  Staple strand_0105 of the 3D DNA nanostructure.
aagaaacgttacatccccctcacaagcatgagttatataa

SEQ ID NO: 107 - DNA, 42 bp, Artificial Sequence
  Staple strand_0106 of the 3D DNA nanostructure.
gtttgcctcagtgcgtgcgccacgtggtgcattcataatcaa

SEQ ID NO: 108 - DNA, 40 bp, Artificial Sequence
  Staple strand_0107 of the 3D DNA nanostructure.
aagccagtacctcccaggctcctacattcccccttcatta

SEQ ID NO: 109 - DNA, 42 bp, Artificial Sequence
  Staple strand_0108 of the 3D DNA nanostructure.
aataggtggaggccggtcaccctagctgaacatggctcccgg

SEQ ID NO: 110 - DNA, 40 bp, Artificial Sequence
  Staple strand_0109 of the 3D DNA nanostructure.
cagtttctggcaagggctgagggcagtaaccctcattcaa

SEQ ID NO: 111 - DNA, 42 bp, Artificial Sequence
  Staple strand_0110 of the 3D DNA nanostructure.
aacgaggggaactcgctgtgcgggtctgagaatttttatcgg

SEQ ID NO: 112 - DNA, 40 bp, Artificial Sequence
  Staple strand_0111 of the 3D DNA nanostructure.
aagggaacacgttgcgcatgaagccatagaagtttatcat

SEQ ID NO: 113 - DNA, 42 bp, Artificial Sequence
  Staple strand_0112 of the 3D DNA nanostructure.
aaaatctaggtgaccgttggcgtaccgtcccaggcgcgaaga

SEQ ID NO: 114 - DNA, 40 bp, Artificial Sequence
  Staple strand_0113 of the 3D DNA nanostructure.
tcattgaaggttggccgctgacttctcgattcatcaatat

SEQ ID NO: 115 - DNA, 42 bp, Artificial Sequence
  Staple strand_0114 of the 3D DNA nanostructure.
tgtagctcattcgccgtggacgcctttgataaatcaaaatgc

SEQ ID NO: 116 - DNA, 40 bp, Artificial Sequence
  Staple strand_0115 of the 3D DNA nanostructure.
taaatcgcctatgcgcgtagacgcagaattccataaaagc

SEQ ID NO: 117 - DNA, 42 bp, Artificial Sequence
  Staple strand_0116 of the 3D DNA nanostructure.
aatcgatgcgcagcccgccagcacactcgtcctttataagag

SEQ ID NO: 118 - DNA, 40 bp, Artificial Sequence
  Staple strand_0117 of the 3D DNA nanostructure.
gcggattgcttcctgagattcttactaaataatcaaaacg

SEQ ID NO: 119 - DNA, 42 bp, Artificial Sequence
  Staple strand_0118 of the 3D DNA nanostructure.
tcacgacgcggccaaggatttactgctcgaccgtgcacccag

SEQ ID NO: 120 - DNA, 40 bp, Artificial Sequence
  Staple strand_0119 of the 3D DNA nanostructure.
ctcactgtgctctgctccgctgcggttttctagacttgcg

SEQ ID NO: 121 - DNA, 42 bp, Artificial Sequence
  Staple strand_0120 of the 3D DNA nanostructure.
aatagcccaaacgacgaggggcacgaaaaaggagaggaaaag

SEQ ID NO: 122 - DNA, 40 bp, Artificial Sequence
  Staple strand_0121 of the 3D DNA nanostructure.
aaagggaggcagcactgcaagaacgggaaataacataata

SEQ ID NO: 123 - DNA, 42 bp, Artificial Sequence
  Staple strand_0122 of the 3D DNA nanostructure.
ggtcagttatcagggagtcaccgcctctcaatgcgcgtatct

SEQ ID NO: 124 - DNA, 40 bp, Artificial Sequence
  Staple strand_0123 of the 3D DNA nanostructure.
caatatagcccgcctgaaagacgcctggaatacatttcat

SEQ ID NO: 125 - DNA, 42 bp, Artificial Sequence
  Staple strand_0124 of the 3D DNA nanostructure.
tacatttactggcgtccccctgagtcccatattgcgtttaat

SEQ ID NO: 126 - DNA, 40 bp, Artificial Sequence
  Staple strand_0125 of the 3D DNA nanostructure.
cgcgagatatcactcgtgagcccagtacttaattaaagaa

SEQ ID NO: 127 - DNA, 42 bp, Artificial Sequence
  Staple strand_0126 of the 3D DNA nanostructure.
gacaaaagctaagccgaagcggaaagccaaataagaagtacc

SEQ ID NO: 128 - DNA, 40 bp, Artificial Sequence
  Staple strand_0127 of the 3D DNA nanostructure.
agaaggcggcaccacacgagaaggacgtcaagaaagatat

SEQ ID NO: 129 - DNA, 42 bp, Artificial Sequence
  Staple strand_0128 of the 3D DNA nanostructure.
attagacgcgtggctcgcaagggcagcgttttgcaccagcgc

SEQ ID NO: 130 - DNA, 40 bp, Artificial Sequence
  Staple strand_0129 of the 3D DNA nanostructure.
aatacattgctgttggttacacgccgggtaataagtagaa

SEQ ID NO: 131 - DNA, 42 bp, Artificial Sequence
  Staple strand_0130 of the 3D DNA nanostructure.
cagcaccgtgctgccgcaagcgagaaaaaccagcgccgatag

SEQ ID NO: 132 - DNA, 40 bp, Artificial Sequence
  Staple strand_0131 of the 3D DNA nanostructure.
gatattccaagatggtaggtccttgatatttgccagcctt

SEQ ID NO: 133 - DNA, 42 bp, Artificial Sequence
  Staple strand_0132 of the 3D DNA nanostructure.
gtcgagagcaactggagtggcgaccggtactacaggagtgcc

SEQ ID NO: 134 - DNA, 40 bp, Artificial Sequence
  Staple strand_0133 of the 3D DNA nanostructure.
tgtatgggtgcgcgagaccgttctagggcaccctcttttc

SEQ ID NO: 135 - DNA, 42 bp, Artificial Sequence
  Staple strand_0134 of the 3D DNA nanostructure.
gtcacccttcaaagcacgcccaccgtgtgaaaaatctggatc

SEQ ID NO: 136 - DNA, 40 bp, Artificial Sequence
  Staple strand_0135 of the 3D DNA nanostructure.
gccggaaccacgcagtgtcagtcggacaacgggtaactta

SEQ ID NO: 137 - DNA, 42 bp, Artificial Sequence
  Staple strand_0136 of the 3D DNA nanostructure.
ctcattatcaacgggcacagcccatcggcggctgaccactgg

SEQ ID NO: 138 - DNA, 40 bp, Artificial Sequence
  Staple strand_0137 of the 3D DNA nanostructure.
gtccaatcgcaaggttctccgactcaacatttaggatagc

SEQ ID NO: 139 - DNA, 42 bp, Artificial Sequence
  Staple strand_0138 of the 3D DNA nanostructure.
ggcttagacgtccctgcgcaataggccgactctttaccggat

SEQ ID NO: 140 - DNA, 40 bp, Artificial Sequence
  Staple strand_0139 of the 3D DNA nanostructure.
ttaagcagagtgctgtgtttcgacgggctgattcccaaaa

SEQ ID NO: 141 - DNA, 42 bp, Artificial Sequence
  Staple strand_0140 of the 3D DNA nanostructure.
cattgcctgagagtcgtggtacgatgtgataaggatacaggt

SEQ ID NO: 142 - DNA, 40 bp, Artificial Sequence
  Staple strand_0141 of the 3D DNA nanostructure.
acccgtcagataggctggctccgtgtgacccaaaataaca

SEQ ID NO: 143 - DNA, 42 bp, Artificial Sequence
  Staple strand_0142 of the 3D DNA nanostructure.
ttaagttgcaaggctgttccgcaacgggggtttgaggggcga

SEQ ID NO: 144 - DNA, 40 bp, Artificial Sequence
  Staple strand_0143 of the 3D DNA nanostructure.
gtgagctggactgtgagcgaccggtcggagcgaggaatga

SEQ ID NO: 145 - DNA, 42 bp, Artificial Sequence
  Staple strand_0144 of the 3D DNA nanostructure.
aaatcggccagcattccctagcctcccgtagtattggttccg

SEQ ID NO: 146 - DNA, 40 bp, Artificial Sequence
  Staple strand_0145 of the 3D DNA nanostructure.
cagattcgggaacaccaccactgatgaacctgagtattgg

SEQ ID NO: 147 - DNA, 42 bp, Artificial Sequence
  Staple strand_0146 of the 3D DNA nanostructure.
cctcaaatggacttgcaaggcggcgaaaacgccctaactgaa

SEQ ID NO: 148 - DNA, 40 bp, Artificial Sequence
  Staple strand_0147 of the 3D DNA nanostructure.
ttcctgaggcttcttgcggcatgatgattttagaatcata

SEQ ID NO: 149 - DNA, 42 bp, Artificial Sequence
  Staple strand_0148 of the 3D DNA nanostructure.
tgaaacaaggtcccgacagcttcgcgtattaggtttagatga

SEQ ID NO: 150 - DNA, 40 bp, Artificial Sequence
  Staple strand_0149 of the 3D DNA nanostructure.
tgatgcaaagcacggagcattgccgtaccttagaaaatgc

SEQ ID NO: 151 - DNA, 42 bp, Artificial Sequence
  Staple strand_0150 of the 3D DNA nanostructure.
cgagccagtgccgcgactcgaaggtaacaaggaatcaatttt

SEQ ID NO: 152 - DNA, 40 bp, Artificial Sequence
  Staple strand_0151 of the 3D DNA nanostructure.
ccgcgcccgggagcagacgtgtggtgtatcccatccatta

SEQ ID NO: 153 - DNA, 42 bp, Artificial Sequence
  Staple strand_0152 of the 3D DNA nanostructure.
agagagaagaacgaacgagcgggattcacaattttattttac

SEQ ID NO: 154 - DNA, 40 bp, Artificial Sequence
  Staple strand_0153 of the 3D DNA nanostructure.
tattacgacattcccattagccgggataaacaatgctcct

SEQ ID NO: 155 - DNA, 42 bp, Artificial Sequence
  Staple strand_0154 of the 3D DNA nanostructure.
cggaaacgcgacgatttgcagcgcttctcaaagggcgaaggc

SEQ ID NO: 156 - DNA, 40 bp, Artificial Sequence
  Staple strand_0155 of the 3D DNA nanostructure.
aggttgaacgagcagaccggcacgggtcataatcaacagg

SEQ ID NO: 157 - DNA, 42 bp, Artificial Sequence
  Staple strand_0156 of the 3D DNA nanostructure.
gttttgctgtgcgtcgaggcgactaactgggtaataagcggg

SEQ ID NO: 158 - DNA, 40 bp, Artificial Sequence
  Staple strand_0157 of the 3D DNA nanostructure.
tctttcctagttgggccggtcccgtccaccacccttgtcg

SEQ ID NO: 159 - DNA, 42 bp, Artificial Sequence
  Staple strand_0158 of the 3D DNA nanostructure.
cagggagtcgggatgccttctgcttctcagaaggctcgcttg

SEQ ID NO: 160 - DNA, 40 bp, Artificial Sequence
  Staple strand_0159 of the 3D DNA nanostructure.
gaaatccggtgcgaactgtggccgactataatgccgtgtc

SEQ ID NO: 161 - DNA, 42 bp, Artificial Sequence
  Staple strand_0160 of the 3D DNA nanostructure.
tgaattacgggagtctgccgagaatggcgcgagtaatcattg

SEQ ID NO: 162 - DNA, 40 bp, Artificial Sequence
  Staple strand_0161 of the 3D DNA nanostructure.
gtaatagcgtgcttttccatgtcggtctcattcaaagggg

SEQ ID NO: 163 - DNA, 42 bp, Artificial Sequence
  Staple strand_0162 of the 3D DNA nanostructure.
tccttttgctacctgggccactgcacgctattatagtattgc

SEQ ID NO: 164 - DNA, 40 bp, Artificial Sequence
  Staple strand_0163 of the 3D DNA nanostructure.
catacagacgcattacctggaacgacatgcgaacgtaaat

SEQ ID NO: 165 - DNA, 42 bp, Artificial Sequence
  Staple strand_0164 of the 3D DNA nanostructure.
tttttgagctcacaccgcagtagcccgagctagaaccagcta

SEQ ID NO: 166 - DNA, 40 bp, Artificial Sequence
  Staple strand_0165 of the 3D DNA nanostructure.
tcatcaacctacgtagaccgcacgataggattgtaagctt

SEQ ID NO: 167 - DNA, 42 bp, Artificial Sequence
  Staple strand_0166 of the 3D DNA nanostructure.
ctggcgaagtagtgccgcgacgctgatgcaacagtatgccag

SEQ ID NO: 168 - DNA, 40 bp, Artificial Sequence
  Staple strand_0167 of the 3D DNA nanostructure.
taaagtgcgtcccttcggacccacgtaggctagcgaagca

SEQ ID NO: 169 - DNA, 42 bp, Artificial Sequence
  Staple strand_0168 of the 3D DNA nanostructure.
aggcgaaaacggtggtcaccggtgcgtgcagtggtttccagc

SEQ ID NO: 170 - DNA, 40 bp, Artificial Sequence
  Staple strand_0169 of the 3D DNA nanostructure.
aatcgtcccgactgccggatggttcgatctcaaaccgctc

SEQ ID NO: 171 - DNA, 42 bp, Artificial Sequence
  Staple strand_0170 of the 3D DNA nanostructure.
atgaaaaactttccttctagcttatggctgcattaaaagcaa

SEQ ID NO: 172 - DNA, 40 bp, Artificial Sequence
  Staple strand_0171 of the 3D DNA nanostructure.
ccaccagcctagggcgcccggtgagatcactttacagaaa

SEQ ID NO: 173 - DNA, 42 bp, Artificial Sequence
  Staple strand_0172 of the 3D DNA nanostructure.
catttcaagcattgaggccctcggaaggaatgaatatttatt

SEQ ID NO: 174 - DNA, 40 bp, Artificial Sequence
  Staple strand_0173 of the 3D DNA nanostructure.
ggttggggcatggacccaggtgaagattaaacatagctta

SEQ ID NO: 175 - DNA, 42 bp, Artificial Sequence
  Staple strand_0174 of the 3D DNA nanostructure.
gccaacatgtcagctgctctccgtccggttagaaaaaacaac

SEQ ID NO: 176 - DNA, 40 bp, Artificial Sequence
  Staple strand_0175 of the 3D DNA nanostructure.
cgtttttcgaggcacacgtctaggttgtacgagcacaagc

SEQ ID NO: 177 - DNA, 42 bp, Artificial Sequence
  Staple strand_0176 of the 3D DNA nanostructure.
aacgtcaacgctctgttcgcgcactaccatttaccaatgttt

SEQ ID NO: 178 - DNA, 40 bp, Artificial Sequence
  Staple strand_0177 of the 3D DNA nanostructure.
ccaaaagccgcgttcggcgaaacgggaaaatagctaatac

SEQ ID NO: 179 - DNA, 42 bp, Artificial Sequence
  Staple strand_0178 of the 3D DNA nanostructure.
caccagtaggccggaagttgtttatccaaaccgattgaaaat

SEQ ID NO: 180 - DNA, 40 bp, Artificial Sequence
  Staple strand_0179 of the 3D DNA nanostructure.
ccaccagtacaggcatgagtgcggcggccggaaccaacca

SEQ ID NO: 181 - DNA, 42 bp, Artificial Sequence
  Staple strand_0180 of the 3D DNA nanostructure.
ctcctcaagacgtaaatgactaaacccgagggggtcatgaga

SEQ ID NO: 182 - DNA, 40 bp, Artificial Sequence
  Staple strand_0181 of the 3D DNA nanostructure.
agttagcatggataccggcggctggcgaagggatactcat

SEQ ID NO: 183 - DNA, 42 bp, Artificial Sequence
  Staple strand_0182 of the 3D DNA nanostructure.
ataaccgatgccgctgctgtgctcgcaggtgcctttacacgc

SEQ ID NO: 184 - DNA, 40 bp, Artificial Sequence
  Staple strand_0183 of the 3D DNA nanostructure.
atttgtacctggcccaaaactggagataaggcacccggag

SEQ ID NO: 185 - DNA, 42 bp, Artificial Sequence
  Staple strand_0184 of the 3D DNA nanostructure.
gagatggttcgcatatgcggatcccaactcgaaccggggctt

SEQ ID NO: 186 - DNA, 40 bp, Artificial Sequence
  Staple strand_0185 of the 3D DNA nanostructure.
cttttgcttcgcgcgttggagtcgatttagatacaagagg

SEQ ID NO: 187 - DNA, 42 bp, Artificial Sequence
  Staple strand_0186 of the 3D DNA nanostructure.
aggtcaggggctagtgccagcccgtcctaaaagcggaccaac

SEQ ID NO: 188 - DNA, 40 bp, Artificial Sequence
  Staple strand_0187 of the 3D DNA nanostructure.
aatagtagcctacagtttgtggccgaatttagtttctact

SEQ ID NO: 189 - DNA, 42 bp, Artificial Sequence
  Staple strand_0188 of the 3D DNA nanostructure.
ctgataaagcgcgagcagtcgatagcgcgtttttaaatctag

SEQ ID NO: 190 - DNA, 40 bp, Artificial Sequence
  Staple strand_0189 of the 3D DNA nanostructure.
ttcgcgtactcgaggctaggttcaacttatatttaaataa

SEQ ID NO: 191 - DNA, 42 bp, Artificial Sequence
  Staple strand_0190 of the 3D DNA nanostructure.
tcggtgcggcggaattgacgtcaatgccctggaagatggcga

SEQ ID NO: 192 - DNA, 40 bp, Artificial Sequence
  Staple strand_0191 of the 3D DNA nanostructure.
aattccagttgttgttctctctccgcggacgcatgctcac

SEQ ID NO: 193 - DNA, 42 bp, Artificial Sequence
  Staple strand_0192 of the 3D DNA nanostructure.
agcaagcgcttcacgttaagcactgcgctgaccagtggttgc

SEQ ID NO: 194 - DNA, 40 bp, Artificial Sequence
  Staple strand_0193 of the 3D DNA nanostructure.
ctcatggaccctgtaggcctccctcggtcttgctgaaacg

SEQ ID NO: 195 - DNA, 42 bp, Artificial Sequence
  Staple strand_0194 of the 3D DNA nanostructure.
caacagtgcggacccggctgtggagctaaaacgaaccgcctg

SEQ ID NO: 196 - DNA, 40 bp, Artificial Sequence
  Staple strand_0195 of the 3D DNA nanostructure.
taacattggaggcctctgcatctgcacctcgacaattgag

SEQ ID NO: 197 - DNA, 42 bp, Artificial Sequence
  Staple strand_0196 of the 3D DNA nanostructure.
agttacaaagaccgcgtaggaccgctgaatagtaccttacca

SEQ ID NO: 198 - DNA, 40 bp, Artificial Sequence
  Staple strand_0197 of the 3D DNA nanostructure.
gagagacgatgagtacgcatcgtccaaacttagatggtct

SEQ ID NO: 199 - DNA, 42 bp, Artificial Sequence
  Staple strand_0198 of the 3D DNA nanostructure.
gcttaattgtaggccggcactggcacggtaagtatcagtagg

SEQ ID NO: 200 - DNA, 40 bp, Artificial Sequence
  Staple strand_0199 of the 3D DNA nanostructure.
agtaccgtgtcgcaggccacaggtgagaaccaatcaacca

SEQ ID NO: 201 - DNA, 42 bp, Artificial Sequence
  Staple strand_0200 of the 3D DNA nanostructure.
ccaatccacaaaccgttccagcccacatcaagcgtctttatc

SEQ ID NO: 202 - DNA, 40 bp, Artificial Sequence
  Staple strand_0201 of the 3D DNA nanostructure.
accgaggacatcgggtggtgccctagaacgaagccaggaa

SEQ ID NO: 203 - DNA, 42 bp, Artificial Sequence
  Staple strand_0202 of the 3D DNA nanostructure.
gagccattagggcgacgcacgtcataacaaaaggtaagactt

SEQ ID NO: 204 - DNA, 40 bp, Artificial Sequence
  Staple strand_0203 of the 3D DNA nanostructure.
caccacctctgtatgctaggtgctcgtgccaccggagagc

SEQ ID NO: 205 - DNA, 42 bp, Artificial Sequence
  Staple strand_0204 of the 3D DNA nanostructure.
ctgaaacagcggccctgccaagctgcccctagtaacattatt

SEQ ID NO: 206 - DNA, 40 bp, Artificial Sequence
  Staple strand_0205 of the 3D DNA nanostructure.
gcctgtaatcgggcgtccctaacttctgcaataggacaac

SEQ ID NO: 207 - DNA, 42 bp, Artificial Sequence
  Staple strand_0206 of the 3D DNA nanostructure.
gccgacaacggcatggcgaacaaggacagcggtttatgttgc

SEQ ID NO: 208 - DNA, 40 bp, Artificial Sequence
  Staple strand_0207 of the 3D DNA nanostructure.
taccaagtctcgccaccctcaccttgtgaacgaaagatta

SEQ ID NO: 209 - DNA, 42 bp, Artificial Sequence
  Staple strand_0208 of the 3D DNA nanostructure.
aaacaccacaccttctgcactgtcgtcacgtacccaaacgag

SEQ ID NO: 210 - DNA, 40 bp, Artificial Sequence
  Staple strand_0209 of the 3D DNA nanostructure.
acgacgagcttcacagaccctaccgcgaaaaaggaaccag

SEQ ID NO: 211 - DNA, 42 bp, Artificial Sequence
  Staple strand_0210 of the 3D DNA nanostructure.
aagcgaacaacacctccgtactccaccctaaaaagatcttca

SEQ ID NO: 212 - DNA, 40 bp, Artificial Sequence
  Staple strand_0211 of the 3D DNA nanostructure.
cgagctgttgcgcaaaaggcgcgaagcaagatacagggcg

SEQ ID NO: 213 - DNA, 42 bp, Artificial Sequence
  Staple strand_0212 of the 3D DNA nanostructure.
caccatcatgttgagtccgctgccatgaaactgagtacaaat

SEQ ID NO: 214 - DNA, 40 bp, Artificial Sequence
  Staple strand_0213 of the 3D DNA nanostructure.
ttaaccactccgaggtcgccggagggataacgttaatttt

SEQ ID NO: 215 - DNA, 42 bp, Artificial Sequence
  Staple strand_0214 of the 3D DNA nanostructure.
ttcaggctcacgcaacactcccgtctagatagccagccgcca

SEQ ID NO: 216 - DNA, 40 bp, Artificial Sequence
  Staple strand_0215 of the 3D DNA nanostructure.
tttcctgactcgaaccgcaccctgcagccccgtatagctg

SEQ ID NO: 217 - DNA, 42 bp, Artificial Sequence
  Staple strand_0216 of the 3D DNA nanostructure.
cccttcactgtgcaccgcccggatggtgttaacagctgattg

SEQ ID NO: 218 - DNA, 40 bp, Artificial Sequence
  Staple strand_0217 of the 3D DNA nanostructure.
taccgccaagacaacagcgccaggctcgccagaacaatat

SEQ ID NO: 219 - DNA, 42 bp, Artificial Sequence
  Staple strand_0218 of the 3D DNA nanostructure.
aggtgagggtatagcgccatcgggcataagaagataaaacag

SEQ ID NO: 220 - DNA, 40 bp, Artificial Sequence
  Staple strand_0219 of the 3D DNA nanostructure.
ccgaacgggacccagccgagagccgacgtaaatcctttgc

SEQ ID NO: 221 - DNA, 42 bp, Artificial Sequence
  Staple strand_0220 of the 3D DNA nanostructure.
aacggattaactgcgtgagtgaaggaacttgggagaaacaat

SEQ ID NO: 222 - DNA, 40 bp, Artificial Sequence
  Staple strand_0221 of the 3D DNA nanostructure.
aatagtgacaggtgtaggatcgggcgcgctgagaagagtc

SEQ ID NO: 223 - DNA, 42 bp, Artificial Sequence
  Staple strand_0222 of the 3D DNA nanostructure.
accagtatggcatcaactgtggtgacggaaatacaaattctt

SEQ ID NO: 224 - DNA, 40 bp, Artificial Sequence
  Staple strand_0223 of the 3D DNA nanostructure.
ttatcatagacctctgcttgtgccacaaggctgtctttcc

SEQ ID NO: 225 - DNA, 42 bp, Artificial Sequence
  Staple strand_0224 of the 3D DNA nanostructure.
gttacaaagcgccagcgactgggcgtcctccctaatttgcca

SEQ ID NO: 226 - DNA, 40 bp, Artificial Sequence
  Staple strand_0225 of the 3D DNA nanostructure.
agatagctctgctcaacactcaccgcatagaaaagtaagc

SEQ ID NO: 227 - DNA, 42 bp, Artificial Sequence
  Staple strand_0226 of the 3D DNA nanostructure.
ttaaaggtagtccgccctacagaagttccgggaaattattca

SEQ ID NO: 228 - DNA, 40 bp, Artificial Sequence
  Staple strand_0227 of the 3D DNA nanostructure.
gccaccccccggtcgaaagagcaaacgatccctcagagcc

SEQ ID NO: 229 - DNA, 42 bp, Artificial Sequence
  Staple strand_0228 of the 3D DNA nanostructure.
tgccccctagcagcccgtccgtgatgagtcataaacagttaa

SEQ ID NO: 230 - DNA, 40 bp, Artificial Sequence
  Staple strand_0229 of the 3D DNA nanostructure.
tgagtttaggccagagtcgagtgcatgcgtaccgtaacac

SEQ ID NO: 231 - DNA, 42 bp, Artificial Sequence
  Staple strand_0230 of the 3D DNA nanostructure.
tttcttaaatccgggcatttgggaagcccgtttcgaggtgaa

SEQ ID NO: 232 - DNA, 40 bp, Artificial Sequence
  Staple strand_0231 of the 3D DNA nanostructure.
aaacactggccggcgtcttagttacgacaagaatacacta

SEQ ID NO: 233 - DNA, 42 bp, Artificial Sequence
  Staple strand_0232 of the 3D DNA nanostructure.
cattcagtccgggccgcgcatacggacccaaacaaagctgct

SEQ ID NO: 234 - DNA, 40 bp, Artificial Sequence
  Staple strand_0233 of the 3D DNA nanostructure.
gcaacactgactgggcagtggttgctgaggcatagtaaga

SEQ ID NO: 235 - DNA, 42 bp, Artificial Sequence
  Staple strand_0234 of the 3D DNA nanostructure.
ttcaaatatcgtcgcgcgtgtagcatattaagcccgaaagac

SEQ ID NO: 236 - DNA, 40 bp, Artificial Sequence
  Staple strand_0235 of the 3D DNA nanostructure.
cctgtttgttcgttcgatgattgtcgtcaatggtcaataa

SEQ ID NO: 237 - DNA, 42 bp, Artificial Sequence
  Staple strand_0236 of the 3D DNA nanostructure.
aagggtgaaccctgtcgggccacatggtaggtaaagattcaa

SEQ ID NO: 238 - DNA, 40 bp, Artificial Sequence
  Staple strand_0237 of the 3D DNA nanostructure.
attaaatcttacgtgggcgtcgagaagagttaaaattcgc

SEQ ID NO: 239 - DNA, 42 bp, Artificial Sequence
  Staple strand_0238 of the 3D DNA nanostructure.
gccggaaacttgttgtgtcacctcggagttaccgcttctggt

SEQ ID NO: 240 - DNA, 40 bp, Artificial Sequence
  Staple strand_0239 of the 3D DNA nanostructure.
gctcgaagcccgaagaggacgtgtgccccccgggtaccga

SEQ ID NO: 241 - DNA, 39 bp, Artificial Sequence
  Staple strand_0240 of the 3D DNA nanostructure.
tcgggcccgtcatcgtctcatatcctcagcaactgcggg

SEQ ID NO: 242 - DNA, 39 bp, Artificial Sequence
  Staple strand_0241 of the 3D DNA nanostructure.
gggcgactggctaccgctgctacccagatatcaccgcac

SEQ ID NO: 243 - DNA, 39 bp, Artificial Sequence
  Staple strand_0242 of the 3D DNA nanostructure.
tcgtgggcaagatatatgtggtccgggatgggcggtgcg

SEQ ID NO: 244 - DNA, 39 bp, Artificial Sequence
  Staple strand_0243 of the 3D DNA nanostructure.
cggcccacagggtcaatagccgtcgcctcacccgactta

SEQ ID NO: 245 - DNA, 39 bp, Artificial Sequence
  Staple strand_0244 of the 3D DNA nanostructure.
gaaccgctagcctcggccgcgcatttacaccttggtcca

SEQ ID NO: 246 - DNA, 39 bp, Artificial Sequence
  Staple strand_0245 of the 3D DNA nanostructure.
cggtccagcaccatggtcccgtaccctcactcgccattg

SEQ ID NO: 247 - DNA, 39 bp, Artificial Sequence
  Staple strand_0246 of the 3D DNA nanostructure.
gcacaactcctgaacaaggcgcccagtgcagtcgacgtc

SEQ ID NO: 248 - DNA, 39 bp, Artificial Sequence
  Staple strand_0247 of the 3D DNA nanostructure.
cgactagagctgctgtgcgtcccgttgcgtgagttaggc

SEQ ID NO: 249 - DNA, 39 bp, Artificial Sequence
  Staple strand_0248 of the 3D DNA nanostructure.
cctaccctcggtgccgagtccctagacgctgggccagta

SEQ ID NO: 250 - DNA, 39 bp, Artificial Sequence
  Staple strand_0249 of the 3D DNA nanostructure.
cggccaccgctcggattatccacagattatggcgcgagg

SEQ ID NO: 251 - DNA, 39 bp, Artificial Sequence
  Staple strand_0250 of the 3D DNA nanostructure.
ccttgagccgcatgcgctacgtcttggcctcctaccctc

SEQ ID NO: 252 - DNA, 39 bp, Artificial Sequence
  Staple strand_0251 of the 3D DNA nanostructure.
gcgcagtcccgttctcgagtgtgcccacttgccctcaat

SEQ ID NO: 253 - DNA, 60 bp, Artificial Sequence
  Staple strand_0252 of the 3D DNA nanostructure.
actccctgataggcgtctttcaggcgggcgggactcaggttcctctaccacctacatcac

SEQ ID NO: 254 - DNA, 60 bp, Artificial Sequence
  Staple strand_0253 of the 3D DNA nanostructure.
gggacgccagactgggctcacgagtgataggctttccgcttcctctaccacctacatcac

SEQ ID NO: 255 - DNA, 60 bp, Artificial Sequence
  Staple strand_0254 of the 3D DNA nanostructure.
ttcggcttaggtccttctcgtgtggtgcccgctgcccttttcctctaccacctacatcac

SEQ ID NO: 256 - DNA, 60 bp, Artificial Sequence
  Staple strand_0255 of the 3D DNA nanostructure.
gcgagccacgcggcgtgtaaccaacagcattttctcgctttcctctaccacctacatcac

SEQ ID NO: 257 - DNA, 60 bp, Artificial Sequence
  Staple strand_0256 of the 3D DNA nanostructure.
tgcggcagcatcaaggacctaccatcttgaccggtcgccttcctctaccacctacatcac

SEQ ID NO: 258 - DNA, 60 bp, Artificial Sequence
  Staple strand_0257 of the 3D DNA nanostructure.
actccagttgctagaacggtctcgcgcacacacggtgggttcctctaccacctacatcac

SEQ ID NO: 259 - DNA, 60 bp, Artificial Sequence
  Staple strand_0258 of the 3D DNA nanostructure.
cgtgctttgatccgactgacactgcgtggccgatgggctttcctctaccacctacatcac

SEQ ID NO: 260 - DNA, 60 bp, Artificial Sequence
  Staple strand_0259 of the 3D DNA nanostructure.
gtgcccgttgtgagtcggagaaccttgcgcggcctattgttcctctaccacctacatcac

SEQ ID NO: 261 - DNA, 60 bp, Artificial Sequence
  Staple strand_0260 of the 3D DNA nanostructure.
cgcagggacgccgtcgaaacacagcactccacatcgtacttcctctaccacctacatcac

SEQ ID NO: 262 - DNA, 60 bp, Artificial Sequence
  Staple strand_0261 of the 3D DNA nanostructure.
cacgactctcacacggagccagcctatctcccgttgcggttcctctaccacctacatcac

SEQ ID NO: 263 - DNA, 60 bp, Artificial Sequence
  Staple strand_0262 of the 3D DNA nanostructure.
aacagccttggaccggtcgctcacagtcctttcgtgcccttcctctaccacctacatcac

SEQ ID NO: 264 - DNA, 60 bp, Artificial Sequence
  Staple strand_0263 of the 3D DNA nanostructure.
ctcgtcgtttccgttcttgcagtgctgccagaggcggtgttcctctaccacctacatcac

SEQ ID NO: 265 - DNA, 60 bp, Artificial Sequence
  Staple strand_0264 of the 3D DNA nanostructure.
agaaggaaagtctcaccgggcgccctaggccttccgaggttcctctaccacctacatcac

SEQ ID NO: 266 - DNA, 60 bp, Artificial Sequence
  Staple strand_0265 of the 3D DNA nanostructure.
gcctcaatgctcttcacctgggtccatgcccggacggagttcctctaccacctacatcac

SEQ ID NO: 267 - DNA, 60 bp, Artificial Sequence
  Staple strand_0266 of the 3D DNA nanostructure.
agcagctgacaacctagacgtgtgcctcgggtagtgcgcttcctctaccacctacatcac

SEQ ID NO: 268 - DNA, 60 bp, Artificial Sequence
  Staple strand_0267 of the 3D DNA nanostructure.
gaacagagcgcccgtttcgccgaacgcggtggataaacattcctctaccacctacatcac

SEQ ID NO: 269 - DNA, 60 bp, Artificial Sequence
  Staple strand_0268 of the 3D DNA nanostructure.
acttccggcccgccgcactcatgcctgtacgggtttagtttcctctaccacctacatcac

SEQ ID NO: 270 - DNA, 60 bp, Artificial Sequence
  Staple strand_0269 of the 3D DNA nanostructure.
catttacgtcgccagccgccggtatccatctgcgagcacttcctctaccacctacatcac

SEQ ID NO: 271 - DNA, 60 bp, Artificial Sequence
  Staple strand_0270 of the 3D DNA nanostructure.
agcagcggcatctccagttttgggccagggttgggatccttcctctaccacctacatcac

SEQ ID NO: 272 - DNA, 60 bp, Artificial Sequence
  Staple strand_0271 of the 3D DNA nanostructure.
gcatatgcgaatcgactccaacgcgcgaaaggacgggctttcctctaccacctacatcac

SEQ ID NO: 273 - DNA, 60 bp, Artificial Sequence
  Staple strand_0272 of the 3D DNA nanostructure.
ggcactagcctcggccacaaactgtaggcgcgctatcgattcctctaccacctacatcac

SEQ ID NO: 274 - DNA, 60 bp, Artificial Sequence
  Staple strand_0273 of the 3D DNA nanostructure.
ctgctcgcgcgttgaacctagcctcgagtggcattgacgttcctctaccacctacatcac

SEQ ID NO: 275 - DNA, 60 bp, Artificial Sequence
  Staple strand_0274 of the 3D DNA nanostructure.
tcaattccgcgcggagagagaacaacaaccacgcaccggttcctctaccacctacatcac

SEQ ID NO: 276 - DNA, 60 bp, Artificial Sequence
  Staple strand_0275 of the 3D DNA nanostructure.
tgaccaccgtcgaaccatccggcagtcgggccataagctttcctctaccacctacatcac

SEQ ID NO: 277 - DNA, 39 bp, Artificial Sequence
  Staple strand_0276 of the 3D DNA nanostructure.
ggcgctatactcggctctcggctgggtccgttccttcac

SEQ ID NO: 278 - DNA, 39 bp, Artificial Sequence
  Staple strand_0277 of the 3D DNA nanostructure.
tcacgcagttcgcccgatcctacacctgtccgtcaccac

SEQ ID NO: 279 - DNA, 39 bp, Artificial Sequence
  Staple strand_0278 of the 3D DNA nanostructure.
agttgatgccgtggcacaagcagaggtctggacgcccag

SEQ ID NO: 280 - DNA, 39 bp, Artificial Sequence
  Staple strand_0279 of the 3D DNA nanostructure.
tcgctggcgcgcggtgagtgttgagcagagaacttctgt

SEQ ID NO: 281 - DNA, 39 bp, Artificial Sequence
  Staple strand_0280 of the 3D DNA nanostructure.
agggcggactgtttgctctttcgaccgggctcatcacgg

SEQ ID NO: 282 - DNA, 39 bp, Artificial Sequence
  Staple strand_0281 of the 3D DNA nanostructure.
acgggctgctatgcactcgactctggcctggcttcccaa

SEQ ID NO: 283 - DNA, 39 bp, Artificial Sequence
  Staple strand_0282 of the 3D DNA nanostructure.
atgcccggatcgtaactaagacgccggccggtccgtatg

SEQ ID NO: 284 - DNA, 39 bp, Artificial Sequence
  Staple strand_0283 of the 3D DNA nanostructure.
cgcggcccggagcaaccactgcccagtcaatatgctaca

SEQ ID NO: 285 - DNA, 39 bp, Artificial Sequence
  Staple strand_0284 of the 3D DNA nanostructure.
cgcgcgacgacgacaatcatcgaacgaacaccatgtggc

SEQ ID NO: 286 - DNA, 39 bp, Artificial Sequence
  Staple strand_0285 of the 3D DNA nanostructure.
ccgacagggtttctcgacgcccacgtaagctccgaggtg

SEQ ID NO: 287 - DNA, 39 bp, Artificial Sequence
  Staple strand_0286 of the 3D DNA nanostructure.
acacaacaaggcacacgtcctcttcgggccaccatccgg

SEQ ID NO: 288 - DNA, 39 bp, Artificial Sequence
  Staple strand_0287 of the 3D DNA nanostructure.
gcggtgcacaagcctggcgctgttgtctttatgcccgat

SEQ ID NO: 289 - DNA, 60 bp, Artificial Sequence
  Staple strand_0288 of the 3D DNA nanostructure.
cctcaacctcgctcgaacgtacgcagccgcaacactggattcctctaccacctacatcac

SEQ ID NO: 290 - DNA, 60 bp, Artificial Sequence
  Staple strand_0289 of the 3D DNA nanostructure.
gaagcggcatagctccgtcctcgggtagtaaagcgcgcattcctctaccacctacatcac

SEQ ID NO: 291 - DNA, 60 bp, Artificial Sequence
  Staple strand_0290 of the 3D DNA nanostructure.
gggaacagcgcataattcgccgtgtgtgtggccatcgcattcctctaccacctacatcac

SEQ ID NO: 292 - DNA, 60 bp, Artificial Sequence
  Staple strand_0291 of the 3D DNA nanostructure.
cgacgccacaacgaacggcctcctcgaaaccttgtgaggttcctctaccacctacatcac

SEQ ID NO: 293 - DNA, 60 bp, Artificial Sequence
  Staple strand_0292 of the 3D DNA nanostructure.
gggatgtaacaccacgtggcgcacgcactgtgtaggagcttcctctaccacctacatcac

SEQ ID NO: 294 - DNA, 60 bp, Artificial Sequence
  Staple strand_0293 of the 3D DNA nanostructure.
ctgggaggtacagctagggtgaccggcctcctgccctcattcctctaccacctacatcac

SEQ ID NO: 295 - DNA, 60 bp, Artificial Sequence
  Staple strand_0294 of the 3D DNA nanostructure.
gcccttgccacagacccgcacagcgagttctggcttcatttcctctaccacctacatcac

SEQ ID NO: 296 - DNA, 60 bp, Artificial Sequence
  Staple strand_0295 of the 3D DNA nanostructure.
gcgcaacgtgacggtacgccaacggtcaccagaagtcagttcctctaccacctacatcac

SEQ ID NO: 297 - DNA, 60 bp, Artificial Sequence
  Staple strand_0296 of the 3D DNA nanostructure.
cggccaacctcaaaggcgtccacggcgaatctgcgtctattcctctaccacctacatcac

SEQ ID NO: 298 - DNA, 60 bp, Artificial Sequence
  Staple strand_0297 of the 3D DNA nanostructure.
cgcgcatagggagtgtgctggcgggctgcgagtaagaatttcctctaccacctacatcac

SEQ ID NO: 299 - DNA, 60 bp, Artificial Sequence
  Staple strand_0298 of the 3D DNA nanostructure.
ctcaggaagcgagcagtaaatccttggccgaccgcagcgttcctctaccacctacatcac

SEQ ID NO: 300 - DNA, 60 bp, Artificial Sequence
  Staple strand_0299 of the 3D DNA nanostructure.
gagcagagcagcttatccctgggtctccggcatctgcgcttcctctaccacctacatcac

SEQ ID NO: 301 - DNA, 60 bp, Artificial Sequence
  Staple strand_0300 of the 3D DNA nanostructure.
tggtgttccctttcgccgccttgcaagtcccatcatgccttcctctaccacctacatcac

SEQ ID NO: 302 - DNA, 60 bp, Artificial Sequence
  Staple strand_0301 of the 3D DNA nanostructure.
gcaagaagcctacgcgaagctgtcgggaccacggcaatgttcctctaccacctacatcac

SEQ ID NO: 303 - DNA, 60 bp, Artificial Sequence
  Staple strand_0302 of the 3D DNA nanostructure.
ctccgtgcttgttaccttcgagtcgcggcacaccacacgttcctctaccacctacatcac

SEQ ID NO: 304 - DNA, 60 bp, Artificial Sequence
  Staple strand_0303 of the 3D DNA nanostructure.
tctgctcccgtgaatcccgctcgttcgttctcccggctattcctctaccacctacatcac

SEQ ID NO: 305 - DNA, 60 bp, Artificial Sequence
  Staple strand_0304 of the 3D DNA nanostructure.
atgggaatgtagaagcgctgcaaatcgtcgcccgtgccgttcctctaccacctacatcac

SEQ ID NO: 306 - DNA, 60 bp, Artificial Sequence
  Staple strand_0305 of the 3D DNA nanostructure.
gtctgctcgtagttagtcgcctcgacgcacgacgggaccttcctctaccacctacatcac

SEQ ID NO: 307 - DNA, 60 bp, Artificial Sequence
  Staple strand_0306 of the 3D DNA nanostructure.
ggcccaactagagaagcagaaggcatcccggtcggccacttcctctaccacctacatcac

SEQ ID NO: 308 - DNA, 60 bp, Artificial Sequence
  Staple strand_0307 of the 3D DNA nanostructure.
agttcgcaccgccattctcggcagactcccaccgacatgttcctctaccacctacatcac

SEQ ID NO: 309 - DNA, 60 bp, Artificial Sequence
  Staple strand_0308 of the 3D DNA nanostructure.
gaaaagcacggcgtgcagtggcccaggtaggtcgttccattcctctaccacctacatcac

SEQ ID NO: 310 - DNA, 60 bp, Artificial Sequence
  Staple strand_0309 of the 3D DNA nanostructure.
ggtaatgcgttcgggctactgcggtgtgagatcgtgcggttcctctaccacctacatcac

SEQ ID NO: 311 - DNA, 60 bp, Artificial Sequence
  Staple strand_0310 of the 3D DNA nanostructure.
tctacgtaggcatcagcgtcgcggcactacacgtgggtcttcctctaccacctacatcac

SEQ ID NO: 312 - DNA, 60 bp, Artificial Sequence
  Staple strand_0311 of the 3D DNA nanostructure.
cgaagggacgcgggaggctagggaatgctgcatcagtggttcctctaccacctacatcac

SEQ ID NO: 313 - DNA, 39 bp, Artificial Sequence
  Staple strand_0312 of the 3D DNA nanostructure.
cctacagggttagctccacagccgggtccgtgcagatgc

SEQ ID NO: 314 - DNA, 39 bp, Artificial Sequence
  Staple strand_0313 of the 3D DNA nanostructure.
agaggcctcctcagcggtcctacgcggtcttggacgatg

SEQ ID NO: 315 - DNA, 39 bp, Artificial Sequence
  Staple strand_0314 of the 3D DNA nanostructure.
cgtactcatcccgtgccagtgccggcctactcacctgtg

SEQ ID NO: 316 - DNA, 39 bp, Artificial Sequence
  Staple strand_0315 of the 3D DNA nanostructure.
gcctgcgacaatgtgggctggaacggtttgctagggcac

SEQ ID NO: 317 - DNA, 39 bp, Artificial Sequence
  Staple strand_0316 of the 3D DNA nanostructure.
cacccgatgtgttatgacgtgcgtcgccctcgagcacct

SEQ ID NO: 318 - DNA, 39 bp, Artificial Sequence
  Staple strand_0317 of the 3D DNA nanostructure.
agcatacagagggcagcttggcagggccgcgaagttagg

SEQ ID NO: 319 - DNA, 39 bp, Artificial Sequence
  Staple strand_0318 of the 3D DNA nanostructure.
gacgcccgattgtccttgttcgccatgccgcaaggtgag

SEQ ID NO: 320 - DNA, 39 bp, Artificial Sequence
  Staple strand_0319 of the 3D DNA nanostructure.
ggtggcgagatgacgacagtgcagaaggtggcggtaggg

SEQ ID NO: 321 - DNA, 39 bp, Artificial Sequence
  Staple strand_0320 of the 3D DNA nanostructure.
tctgtgaagcgggtggagtacggaggtgttcttcgcgcc

SEQ ID NO: 322 - DNA, 39 bp, Artificial Sequence
  Staple strand_0321 of the 3D DNA nanostructure.
ttttgcgcaatcatggcagcggactcaacaccctccggc

SEQ ID NO: 323 - DNA, 39 bp, Artificial Sequence
  Staple strand_0322 of the 3D DNA nanostructure.
gacctcggagctagacgggagtgttgcgtgtgcagggtg

SEQ ID NO: 324 - DNA, 39 bp, Artificial Sequence
  Staple strand_0323 of the 3D DNA nanostructure.
cggttcgagtgcgcagtgcttaacgtgaagcgagggagg

SEQ ID NO: 325 - DNA, 60 bp, Artificial Sequence
  Staple strand_0324 of the 3D DNA nanostructure.
actccctgataggcgtctttcaggcgggcgggactcaggtaacattcctaacttctcata

SEQ ID NO: 326 - DNA, 60 bp, Artificial Sequence
  Staple strand_0325 of the 3D DNA nanostructure.
gggacgccagactgggctcacgagtgataggctttccgctaacattcctaacttctcata

SEQ ID NO: 327 - DNA, 60 bp, Artificial Sequence
  Staple strand_0326 of the 3D DNA nanostructure.
ttcggcttaggtccttctcgtgtggtgcccgctgccctttaacattcctaacttctcata

SEQ ID NO: 328 - DNA, 60 bp, Artificial Sequence
  Staple strand_0327 of the 3D DNA nanostructure.
gcgagccacgcggcgtgtaaccaacagcattttctcgcttaacattcctaacttctcata

SEQ ID NO: 329 - DNA, 60 bp, Artificial Sequence
  Staple strand_0328 of the 3D DNA nanostructure.
tgcggcagcatcaaggacctaccatcttgaccggtcgcctaacattcctaacttctcata

SEQ ID NO: 330 - DNA, 60 bp, Artificial Sequence
  Staple strand_0329 of the 3D DNA nanostructure.
actccagttgctagaacggtctcgcgcacacacggtgggtaacattcctaacttctcata

SEQ ID NO: 331 - DNA, 60 bp, Artificial Sequence
  Staple strand_0330 of the 3D DNA nanostructure.
cgtgctttgatccgactgacactgcgtggccgatgggcttaacattcctaacttctcata

SEQ ID NO: 332 - DNA, 60 bp, Artificial Sequence
  Staple strand_0331 of the 3D DNA nanostructure.
gtgcccgttgtgagtcggagaaccttgcgcggcctattgtaacattcctaacttctcata

SEQ ID NO: 333 - DNA, 60 bp, Artificial Sequence
  Staple strand_0332 of the 3D DNA nanostructure.
cgcagggacgccgtcgaaacacagcactccacatcgtactaacattcctaacttctcata

SEQ ID NO: 334 - DNA, 60 bp, Artificial Sequence
  Staple strand_0333 of the 3D DNA nanostructure.
cacgactctcacacggagccagcctatctcccgttgcggtaacattcctaacttctcata

SEQ ID NO: 335 - DNA, 60 bp, Artificial Sequence
  Staple strand_0334 of the 3D DNA nanostructure.
aacagccttggaccggtcgctcacagtcctttcgtgccctaacattcctaacttctcata

SEQ ID NO: 336 - DNA, 60 bp, Artificial Sequence
  Staple strand_0335 of the 3D DNA nanostructure.
ctcgtcgtttccgttcttgcagtgctgccagaggcggtgtaacattcctaacttctcata

SEQ ID NO: 337 - DNA, 60 bp, Artificial Sequence
  Staple strand_0336 of the 3D DNA nanostructure.
agaaggaaagtctcaccgggcgccctaggccttccgaggtaacattcctaacttctcata

SEQ ID NO: 338 - DNA, 60 bp, Artificial Sequence
  Staple strand_0337 of the 3D DNA nanostructure.
gcctcaatgctcttcacctgggtccatgcccggacggagtaacattcctaacttctcata

SEQ ID NO: 339 - DNA, 60 bp, Artificial Sequence
  Staple strand_0338 of the 3D DNA nanostructure.
agcagctgacaacctagacgtgtgcctcgggtagtgcgctaacattcctaacttctcata

SEQ ID NO: 340 - DNA, 60 bp, Artificial Sequence
  Staple strand_0339 of the 3D DNA nanostructure.
gaacagagcgcccgtttcgccgaacgcggtggataaacataacattcctaacttctcata

SEQ ID NO: 341 - DNA, 60 bp, Artificial Sequence
  Staple strand_0340 of the 3D DNA nanostructure.
acttccggcccgccgcactcatgcctgtacgggtttagttaacattcctaacttctcata

SEQ ID NO: 342 - DNA, 60 bp, Artificial Sequence
  Staple strand_0341 of the 3D DNA nanostructure.
catttacgtcgccagccgccggtatccatctgcgagcactaacattcctaacttctcata

SEQ ID NO: 343 - DNA, 60 bp, Artificial Sequence
  Staple strand_0342 of the 3D DNA nanostructure.
agcagcggcatctccagttttgggccagggttgggatcctaacattcctaacttctcata

SEQ ID NO: 344 - DNA, 60 bp, Artificial Sequence
  Staple strand_0343 of the 3D DNA nanostructure.
gcatatgcgaatcgactccaacgcgcgaaaggacgggcttaacattcctaacttctcata

SEQ ID NO: 345 - DNA, 60 bp, Artificial Sequence
  Staple strand_0344 of the 3D DNA nanostructure.
ggcactagcctcggccacaaactgtaggcgcgctatcgataacattcctaacttctcata

SEQ ID NO: 346 - DNA, 60 bp, Artificial Sequence
  Staple strand_0345 of the 3D DNA nanostructure.
ctgctcgcgcgttgaacctagcctcgagtggcattgacgtaacattcctaacttctcata

SEQ ID NO: 347 - DNA, 60 bp, Artificial Sequence
  Staple strand_0346 of the 3D DNA nanostructure.
tcaattccgcgcggagagagaacaacaaccacgcaccggtaacattcctaacttctcata

SEQ ID NO: 348 - DNA, 60 bp, Artificial Sequence
  Staple strand_0347 of the 3D DNA nanostructure.
tgaccaccgtcgaaccatccggcagtcgggccataagcttaacattcctaacttctcata

SEQ ID NO: 349 - DNA, 60 bp, Artificial Sequence
  Staple strand_0348 of the 3D DNA nanostructure.
cctcaacctcgctcgaacgtacgcagccgcaacactggataacattcctaacttctcata

SEQ ID NO: 350 - DNA, 60 bp, Artificial Sequence
  Staple strand_0349 of the 3D DNA nanostructure.
gaagcggcatagctccgtcctcgggtagtaaagcgcgcataacattcctaacttctcata

SEQ ID NO: 351 - DNA, 60 bp, Artificial Sequence
  Staple strand_0350 of the 3D DNA nanostructure.
gggaacagcgcataattcgccgtgtgtgtggccatcgcataacattcctaacttctcata

SEQ ID NO: 352 - DNA, 60 bp, Artificial Sequence
  Staple strand_0351 of the 3D DNA nanostructure.
cgacgccacaacgaacggcctcctcgaaaccttgtgaggtaacattcctaacttctcata

SEQ ID NO: 353 - DNA, 60 bp, Artificial Sequence
  Staple strand_0352 of the 3D DNA nanostructure.
gggatgtaacaccacgtggcgcacgcactgtgtaggagctaacattcctaacttctcata

SEQ ID NO: 354 - DNA, 60 bp, Artificial Sequence
  Staple strand_0353 of the 3D DNA nanostructure.
ctgggaggtacagctagggtgaccggcctcctgccctcataacattcctaacttctcata

SEQ ID NO: 355 - DNA, 60 bp, Artificial Sequence
  Staple strand_0354 of the 3D DNA nanostructure.
gcccttgccacagacccgcacagcgagttctggcttcattaacattcctaacttctcata

SEQ ID NO: 356 - DNA, 60 bp, Artificial Sequence
  Staple strand_0355 of the 3D DNA nanostructure.
gcgcaacgtgacggtacgccaacggtcaccagaagtcagtaacattcctaacttctcata

SEQ ID NO: 357 - DNA, 60 bp, Artificial Sequence
  Staple strand_0356 of the 3D DNA nanostructure.
cggccaacctcaaaggcgtccacggcgaatctgcgtctataacattcctaacttctcata

SEQ ID NO: 358 - DNA, 60 bp, Artificial Sequence
  Staple strand_0357 of the 3D DNA nanostructure.
cgcgcatagggagtgtgctggcgggctgcgagtaagaattaacattcctaacttctcata

SEQ ID NO: 359 - DNA, 60 bp, Artificial Sequence
  Staple strand_0358 of the 3D DNA nanostructure.
ctcaggaagcgagcagtaaatccttggccgaccgcagcgtaacattcctaacttctcata

SEQ ID NO: 360 - DNA, 60 bp, Artificial Sequence
  Staple strand_0359 of the 3D DNA nanostructure.
gagcagagcagcttatccctgggtctccggcatctgcgctaacattcctaacttctcata

SEQ ID NO: 361 - DNA, 60 bp, Artificial Sequence
  Staple strand_0360 of the 3D DNA nanostructure.
tggtgttccctttcgccgccttgcaagtcccatcatgcctaacattcctaacttctcata

SEQ ID NO: 362 - DNA, 60 bp, Artificial Sequence
  Staple strand_0361 of the 3D DNA nanostructure.
gcaagaagcctacgcgaagctgtcgggaccacggcaatgtaacattcctaacttctcata

SEQ ID NO: 363 - DNA, 60 bp, Artificial Sequence
  Staple strand_0362 of the 3D DNA nanostructure.
ctccgtgcttgttaccttcgagtcgcggcacaccacacgtaacattcctaacttctcata

SEQ ID NO: 364 - DNA, 60 bp, Artificial Sequence
  Staple strand_0363 of the 3D DNA nanostructure.
tctgctcccgtgaatcccgctcgttcgttctcccggctataacattcctaacttctcata

SEQ ID NO: 365 - DNA, 60 bp, Artificial Sequence
  Staple strand_0364 of the 3D DNA nanostructure.
atgggaatgtagaagcgctgcaaatcgtcgcccgtgccgtaacattcctaacttctcata

SEQ ID NO: 366 - DNA, 60 bp, Artificial Sequence
  Staple strand_0365 of the 3D DNA nanostructure.
gtctgctcgtagttagtcgcctcgacgcacgacgggacctaacattcctaacttctcata

SEQ ID NO: 367 - DNA, 60 bp, Artificial Sequence
  Staple strand_0366 of the 3D DNA nanostructure.
ggcccaactagagaagcagaaggcatcccggtcggccactaacattcctaacttctcata

SEQ ID NO: 368 - DNA, 60 bp, Artificial Sequence
  Staple strand_0367 of the 3D DNA nanostructure.
agttcgcaccgccattctcggcagactcccaccgacatgtaacattcctaacttctcata

SEQ ID NO: 369 - DNA, 60 bp, Artificial Sequence
  Staple strand_0368 of the 3D DNA nanostructure.
gaaaagcacggcgtgcagtggcccaggtaggtcgttccataacattcctaacttctcata

SEQ ID NO: 370 - DNA, 60 bp, Artificial Sequence
  Staple strand_0369 of the 3D DNA nanostructure.
ggtaatgcgttcgggctactgcggtgtgagatcgtgcggtaacattcctaacttctcata

SEQ ID NO: 371 - DNA, 60 bp, Artificial Sequence
  Staple strand_0370 of the 3D DNA nanostructure.
tctacgtaggcatcagcgtcgcggcactacacgtgggtctaacattcctaacttctcata

SEQ ID NO: 372 - DNA, 60 bp, Artificial Sequence
  Staple strand_0371 of the 3D DNA nanostructure.
cgaagggacgcgggaggctagggaatgctgcatcagtggtaacattcctaacttctcata

SEQ ID NO: 373 - DNA, 60 bp, Artificial Sequence
  Staple strand_0372 of the 3D DNA nanostructure.
actccctgataggcgtctttcaggcgggcgggactcaggttaccatctctcctaaactcg

SEQ ID NO: 374 - DNA, 60 bp, Artificial Sequence
  Staple strand_0373 of the 3D DNA nanostructure.
gggacgccagactgggctcacgagtgataggctttccgcttaccatctctcctaaactcg

SEQ ID NO: 375 - DNA, 60 bp, Artificial Sequence
  Staple strand_0374 of the 3D DNA nanostructure.
ttcggcttaggtccttctcgtgtggtgcccgctgcccttttaccatctctcctaaactcg

SEQ ID NO: 376 - DNA, 60 bp, Artificial Sequence
  Staple strand_0375 of the 3D DNA nanostructure.
gcgagccacgcggcgtgtaaccaacagcattttctcgctttaccatctctcctaaactcg

SEQ ID NO: 377 - DNA, 60 bp, Artificial Sequence
  Staple strand_0376 of the 3D DNA nanostructure.
tgcggcagcatcaaggacctaccatcttgaccggtcgccttaccatctctcctaaactcg

SEQ ID NO: 378 - DNA, 60 bp, Artificial Sequence
  Staple strand_0377 of the 3D DNA nanostructure.
actccagttgctagaacggtctcgcgcacacacggtgggttaccatctctcctaaactcg

SEQ ID NO: 379 - DNA, 60 bp, Artificial Sequence
  Staple strand_0378 of the 3D DNA nanostructure.
cgtgctttgatccgactgacactgcgtggccgatgggctttaccatctctcctaaactcg

SEQ ID NO: 380 - DNA, 60 bp, Artificial Sequence
  Staple strand_0379 of the 3D DNA nanostructure.
gtgcccgttgtgagtcggagaaccttgcgcggcctattgttaccatctctcctaaactcg

SEQ ID NO: 381 - DNA, 60 bp, Artificial Sequence
  Staple strand_0380 of the 3D DNA nanostructure.
cgcagggacgccgtcgaaacacagcactccacatcgtacttaccatctctcctaaactcg

SEQ ID NO: 382 - DNA, 60 bp, Artificial Sequence
  Staple strand_0381 of the 3D DNA nanostructure.
cacgactctcacacggagccagcctatctcccgttgcggttaccatctctcctaaactcg

SEQ ID NO: 383 - DNA, 60 bp, Artificial Sequence
  Staple strand_0382 of the 3D DNA nanostructure.
aacagccttggaccggtcgctcacagtcctttcgtgcccttaccatctctcctaaactcg

SEQ ID NO: 384 - DNA, 60 bp, Artificial Sequence
  Staple strand_0383 of the 3D DNA nanostructure.
ctcgtcgtttccgttcttgcagtgctgccagaggcggtgttaccatctctcctaaactcg

SEQ ID NO: 385 - DNA, 60 bp, Artificial Sequence
  Staple strand_0384 of the 3D DNA nanostructure.
agaaggaaagtctcaccgggcgccctaggccttccgaggttaccatctctcctaaactcg

SEQ ID NO: 386 - DNA, 60 bp, Artificial Sequence
  Staple strand_0385 of the 3D DNA nanostructure.
gcctcaatgctcttcacctgggtccatgcccggacggagttaccatctctcctaaactcg

SEQ ID NO: 387 - DNA, 60 bp, Artificial Sequence
  Staple strand_0386 of the 3D DNA nanostructure.
agcagctgacaacctagacgtgtgcctcgggtagtgcgcttaccatctctcctaaactcg

SEQ ID NO: 388 - DNA, 60 bp, Artificial Sequence
  Staple strand_0387 of the 3D DNA nanostructure.
gaacagagcgcccgtttcgccgaacgcggtggataaacattaccatctctcctaaactcg

SEQ ID NO: 389 - DNA, 60 bp, Artificial Sequence
  Staple strand_0388 of the 3D DNA nanostructure.
acttccggcccgccgcactcatgcctgtacgggtttagtttaccatctctcctaaactcg

SEQ ID NO: 390 - DNA, 60 bp, Artificial Sequence
  Staple strand_0389 of the 3D DNA nanostructure.
catttacgtcgccagccgccggtatccatctgcgagcacttaccatctctcctaaactcg

SEQ ID NO: 391 - DNA, 60 bp, Artificial Sequence
  Staple strand_0390 of the 3D DNA nanostructure.
agcagcggcatctccagttttgggccagggttgggatccttaccatctctcctaaactcg

SEQ ID NO: 392 - DNA, 60 bp, Artificial Sequence
  Staple strand_0391 of the 3D DNA nanostructure.
gcatatgcgaatcgactccaacgcgcgaaaggacgggctttaccatctctcctaaactcg

SEQ ID NO: 393 - DNA, 60 bp, Artificial Sequence
  Staple strand_0392 of the 3D DNA nanostructure.
ggcactagcctcggccacaaactgtaggcgcgctatcgattaccatctctcctaaactcg

SEQ ID NO: 394 - DNA, 60 bp, Artificial Sequence
  Staple strand_0393 of the 3D DNA nanostructure.
ctgctcgcgcgttgaacctagcctcgagtggcattgacgttaccatctctcctaaactcg

SEQ ID NO: 395 - DNA, 60 bp, Artificial Sequence
  Staple strand_0394 of the 3D DNA nanostructure.
tcaattccgcgcggagagagaacaacaaccacgcaccggttaccatctctcctaaactcg

SEQ ID NO: 396 - DNA, 60 bp, Artificial Sequence
  Staple strand_0395 of the 3D DNA nanostructure.
tgaccaccgtcgaaccatccggcagtcgggccataagctttaccatctctcctaaactcg

SEQ ID NO: 397 - DNA, 60 bp, Artificial Sequence
  Staple strand_0396 of the 3D DNA nanostructure.
cctcaacctcgctcgaacgtacgcagccgcaacactggattaccatctctcctaaactcg

SEQ ID NO: 398 - DNA, 60 bp, Artificial Sequence
  Staple strand_0397 of the 3D DNA nanostructure.
gaagcggcatagctccgtcctcgggtagtaaagcgcgcattaccatctctcctaaactcg

SEQ ID NO: 399 - DNA, 60 bp, Artificial Sequence
  Staple strand_0398 of the 3D DNA nanostructure.
gggaacagcgcataattcgccgtgtgtgtggccatcgcattaccatctctcctaaactcg

SEQ ID NO: 400 - DNA, 60 bp, Artificial Sequence
  Staple strand_0399 of the 3D DNA nanostructure.
cgacgccacaacgaacggcctcctcgaaaccttgtgaggttaccatctctcctaaactcg

SEQ ID NO: 401 - DNA, 60 bp, Artificial Sequence
  Staple strand_0400 of the 3D DNA nanostructure.
gggatgtaacaccacgtggcgcacgcactgtgtaggagcttaccatctctcctaaactcg

SEQ ID NO: 402 - DNA, 60 bp, Artificial Sequence
  Staple strand_0401 of the 3D DNA nanostructure.
ctgggaggtacagctagggtgaccggcctcctgccctcattaccatctctcctaaactcg

SEQ ID NO: 403 - DNA, 60 bp, Artificial Sequence
  Staple strand_0402 of the 3D DNA nanostructure.
gcccttgccacagacccgcacagcgagttctggcttcatttaccatctctcctaaactcg

SEQ ID NO: 404 - DNA, 60 bp, Artificial Sequence
  Staple strand_0403 of the 3D DNA nanostructure.
gcgcaacgtgacggtacgccaacggtcaccagaagtcagttaccatctctcctaaactcg

SEQ ID NO: 405 - DNA, 60 bp, Artificial Sequence
  Staple strand_0404 of the 3D DNA nanostructure.
cggccaacctcaaaggcgtccacggcgaatctgcgtctattaccatctctcctaaactcg

SEQ ID NO: 406 - DNA, 60 bp, Artificial Sequence
  Staple strand_0405 of the 3D DNA nanostructure.
cgcgcatagggagtgtgctggcgggctgcgagtaagaatttaccatctctcctaaactcg

SEQ ID NO: 407 - DNA, 60 bp, Artificial Sequence
  Staple strand_0406 of the 3D DNA nanostructure.
ctcaggaagcgagcagtaaatccttggccgaccgcagcgttaccatctctcctaaactcg

SEQ ID NO: 408 - DNA, 60 bp, Artificial Sequence
  Staple strand_0407 of the 3D DNA nanostructure.
gagcagagcagcttatccctgggtctccggcatctgcgcttaccatctctcctaaactcg

SEQ ID NO: 409 - DNA, 60 bp, Artificial Sequence
  Staple strand_0408 of the 3D DNA nanostructure.
tggtgttccctttcgccgccttgcaagtcccatcatgccttaccatctctcctaaactcg

SEQ ID NO: 410 - DNA, 60 bp, Artificial Sequence
  Staple strand_0409 of the 3D DNA nanostructure.
gcaagaagcctacgcgaagctgtcgggaccacggcaatgttaccatctctcctaaactcg

SEQ ID NO: 411 - DNA, 60 bp, Artificial Sequence
  Staple strand_0410 of the 3D DNA nanostructure.
ctccgtgcttgttaccttcgagtcgcggcacaccacacgttaccatctctcctaaactcg

SEQ ID NO: 412 - DNA, 60 bp, Artificial Sequence
  Staple strand_0411 of the 3D DNA nanostructure.
tctgctcccgtgaatcccgctcgttcgttctcccggctattaccatctctcctaaactcg

SEQ ID NO: 413 - DNA, 60 bp, Artificial Sequence
  Staple strand_0412 of the 3D DNA nanostructure.
atgggaatgtagaagcgctgcaaatcgtcgcccgtgccgttaccatctctcctaaactcg

SEQ ID NO: 414 - DNA, 60 bp, Artificial Sequence
  Staple strand_0413 of the 3D DNA nanostructure.
gtctgctcgtagttagtcgcctcgacgcacgacgggaccttaccatctctcctaaactcg

SEQ ID NO: 415 - DNA, 60 bp, Artificial Sequence
  Staple strand_0414 of the 3D DNA nanostructure.
ggcccaactagagaagcagaaggcatcccggtcggccacttaccatctctcctaaactcg

SEQ ID NO: 416 - DNA, 60 bp, Artificial Sequence
  Staple strand_0415 of the 3D DNA nanostructure.
agttcgcaccgccattctcggcagactcccaccgacatgttaccatctctcctaaactcg

SEQ ID NO: 417 - DNA, 60 bp, Artificial Sequence
  Staple strand_0416 of the 3D DNA nanostructure.
gaaaagcacggcgtgcagtggcccaggtaggtcgttccattaccatctctcctaaactcg

SEQ ID NO: 418 - DNA, 60 bp, Artificial Sequence
  Staple strand_0417 of the 3D DNA nanostructure.
ggtaatgcgttcgggctactgcggtgtgagatcgtgcggttaccatctctcctaaactcg

SEQ ID NO: 419 - DNA, 60 bp, Artificial Sequence
  Staple strand_0418 of the 3D DNA nanostructure.
tctacgtaggcatcagcgtcgcggcactacacgtgggtcttaccatctctcctaaactcg

SEQ ID NO: 420 - DNA, 60 bp, Artificial Sequence
  Staple strand_0419 of the 3D DNA nanostructure.
cgaagggacgcgggaggctagggaatgctgcatcagtggttaccatctctcctaaactcg

SEQ ID NO: 421 - DNA, 39 bp, Artificial Sequence
  Staple strand_0420 of the 3D DNA nanostructure.
actccctgataggcgtctttcaggcgggcgggactcagg

SEQ ID NO: 422 - DNA, 39 bp, Artificial Sequence
  Staple strand_0421 of the 3D DNA nanostructure.
gggacgccagactgggctcacgagtgataggctttccgc

SEQ ID NO: 423 - DNA, 39 bp, Artificial Sequence
  Staple strand_0422 of the 3D DNA nanostructure.
ttcggcttaggtccttctcgtgtggtgcccgctgccctt

SEQ ID NO: 424 - DNA, 39 bp, Artificial Sequence
  Staple strand_0423 of the 3D DNA nanostructure.
gcgagccacgcggcgtgtaaccaacagcattttctcgct

SEQ ID NO: 425 - DNA, 39 bp, Artificial Sequence
  Staple strand_0424 of the 3D DNA nanostructure.
tgcggcagcatcaaggacctaccatcttgaccggtcgcc

SEQ ID NO: 426 - DNA, 39 bp, Artificial Sequence
  Staple strand_0425 of the 3D DNA nanostructure.
actccagttgctagaacggtctcgcgcacacacggtggg

SEQ ID NO: 427 - DNA, 39 bp, Artificial Sequence
  Staple strand_0426 of the 3D DNA nanostructure.
cgtgctttgatccgactgacactgcgtggccgatgggct

SEQ ID NO: 428 - DNA, 39 bp, Artificial Sequence
  Staple strand_0427 of the 3D DNA nanostructure.
gtgcccgttgtgagtcggagaaccttgcgcggcctattg

SEQ ID NO: 429 - DNA, 39 bp, Artificial Sequence
  Staple strand_0428 of the 3D DNA nanostructure.
cgcagggacgccgtcgaaacacagcactccacatcgtac

SEQ ID NO: 430 - DNA, 39 bp, Artificial Sequence
  Staple strand_0429 of the 3D DNA nanostructure.
cacgactctcacacggagccagcctatctcccgttgcgg

SEQ ID NO: 431 - DNA, 39 bp, Artificial Sequence
  Staple strand_0430 of the 3D DNA nanostructure.
aacagccttggaccggtcgctcacagtcctttcgtgccc

SEQ ID NO: 432 - DNA, 39 bp, Artificial Sequence
  Staple strand_0431 of the 3D DNA nanostructure.
ctcgtcgtttccgttcttgcagtgctgccagaggcggtg

SEQ ID NO: 433 - DNA, 39 bp, Artificial Sequence
  Staple strand_0432 of the 3D DNA nanostructure.
agaaggaaagtctcaccgggcgccctaggccttccgagg

SEQ ID NO: 434 - DNA, 39 bp, Artificial Sequence
  Staple strand_0433 of the 3D DNA nanostructure.
gcctcaatgctcttcacctgggtccatgcccggacggag

SEQ ID NO: 435 - DNA, 39 bp, Artificial Sequence
  Staple strand_0434 of the 3D DNA nanostructure.
agcagctgacaacctagacgtgtgcctcgggtagtgcgc

SEQ ID NO: 436 - DNA, 39 bp, Artificial Sequence
  Staple strand_0435 of the 3D DNA nanostructure.
gaacagagcgcccgtttcgccgaacgcggtggataaaca

SEQ ID NO: 437 - DNA, 39 bp, Artificial Sequence
  Staple strand_0436 of the 3D DNA nanostructure.
acttccggcccgccgcactcatgcctgtacgggtttagt

SEQ ID NO: 438 - DNA, 39 bp, Artificial Sequence
  Staple strand_0437 of the 3D DNA nanostructure.
catttacgtcgccagccgccggtatccatctgcgagcac

SEQ ID NO: 439 - DNA, 39 bp, Artificial Sequence
  Staple strand_0438 of the 3D DNA nanostructure.
agcagcggcatctccagttttgggccagggttgggatcc

SEQ ID NO: 440 - DNA, 39 bp, Artificial Sequence
  Staple strand_0439 of the 3D DNA nanostructure.
gcatatgcgaatcgactccaacgcgcgaaaggacgggct

SEQ ID NO: 441 - DNA, 39 bp, Artificial Sequence
  Staple strand_0440 of the 3D DNA nanostructure.
ggcactagcctcggccacaaactgtaggcgcgctatcga

SEQ ID NO: 442 - DNA, 39 bp, Artificial Sequence
  Staple strand_0441 of the 3D DNA nanostructure.
ctgctcgcgcgttgaacctagcctcgagtggcattgacg

SEQ ID NO: 443 - DNA, 39 bp, Artificial Sequence
  Staple strand_0442 of the 3D DNA nanostructure.
tcaattccgcgcggagagagaacaacaaccacgcaccgg

SEQ ID NO: 444 - DNA, 39 bp, Artificial Sequence
  Staple strand_0443 of the 3D DNA nanostructure.
tgaccaccgtcgaaccatccggcagtcgggccataagct

SEQ ID NO: 445 - DNA, 39 bp, Artificial Sequence
  Staple strand_0444 of the 3D DNA nanostructure.
cctcaacctcgctcgaacgtacgcagccgcaacactgga

SEQ ID NO: 446 - DNA, 39 bp, Artificial Sequence
  Staple strand_0445 of the 3D DNA nanostructure.
gaagcggcatagctccgtcctcgggtagtaaagcgcgca

SEQ ID NO: 447 - DNA, 39 bp, Artificial Sequence
  Staple strand_0446 of the 3D DNA nanostructure.
gggaacagcgcataattcgccgtgtgtgtggccatcgca

SEQ ID NO: 448 - DNA, 39 bp, Artificial Sequence
  Staple strand_0447 of the 3D DNA nanostructure.
cgacgccacaacgaacggcctcctcgaaaccttgtgagg

SEQ ID NO: 449 - DNA, 39 bp, Artificial Sequence
  Staple strand_0448 of the 3D DNA nanostructure.
gggatgtaacaccacgtggcgcacgcactgtgtaggagc

SEQ ID NO: 450 - DNA, 39 bp, Artificial Sequence
  Staple strand_0449 of the 3D DNA nanostructure.
ctgggaggtacagctagggtgaccggcctcctgccctca

SEQ ID NO: 451 - DNA, 39 bp, Artificial Sequence
  Staple strand_0450 of the 3D DNA nanostructure.
gcccttgccacagacccgcacagcgagttctggcttcat

SEQ ID NO: 452 - DNA, 39 bp, Artificial Sequence
  Staple strand_0451 of the 3D DNA nanostructure.
gcgcaacgtgacggtacgccaacggtcaccagaagtcag

SEQ ID NO: 453 - DNA, 39 bp, Artificial Sequence
  Staple strand_0452 of the 3D DNA nanostructure.
cggccaacctcaaaggcgtccacggcgaatctgcgtcta

SEQ ID NO: 454 - DNA, 39 bp, Artificial Sequence
  Staple strand_0453 of the 3D DNA nanostructure.
cgcgcatagggagtgtgctggcgggctgcgagtaagaat

SEQ ID NO: 455 - DNA, 39 bp, Artificial Sequence
  Staple strand_0454 of the 3D DNA nanostructure.
ctcaggaagcgagcagtaaatccttggccgaccgcagcg

SEQ ID NO: 456 - DNA, 39 bp, Artificial Sequence
  Staple strand_0455 of the 3D DNA nanostructure.
gagcagagcagcttatccctgggtctccggcatctgcgc

SEQ ID NO: 457 - DNA, 39 bp, Artificial Sequence
  Staple strand_0456 of the 3D DNA nanostructure.
tggtgttccctttcgccgccttgcaagtcccatcatgcc

SEQ ID NO: 458 - DNA, 39 bp, Artificial Sequence
  Staple strand_0457 of the 3D DNA nanostructure.
gcaagaagcctacgcgaagctgtcgggaccacggcaatg

SEQ ID NO: 459 - DNA, 39 bp, Artificial Sequence
  Staple strand_0458 of the 3D DNA nanostructure.
ctccgtgcttgttaccttcgagtcgcggcacaccacacg

SEQ ID NO: 460 - DNA, 39 bp, Artificial Sequence
  Staple strand_0459 of the 3D DNA nanostructure.
tctgctcccgtgaatcccgctcgttcgttctcccggcta

SEQ ID NO: 461 - DNA, 39 bp, Artificial Sequence
  Staple strand_0460 of the 3D DNA nanostructure.
atgggaatgtagaagcgctgcaaatcgtcgcccgtgccg

SEQ ID NO: 462 - DNA, 39 bp, Artificial Sequence
  Staple strand_0461 of the 3D DNA nanostructure.
gtctgctcgtagttagtcgcctcgacgcacgacgggacc

SEQ ID NO: 463 - DNA, 39 bp, Artificial Sequence
  Staple strand_0462 of the 3D DNA nanostructure.
ggcccaactagagaagcagaaggcatcccggtcggccac

SEQ ID NO: 464 - DNA, 39 bp, Artificial Sequence
  Staple strand_0463 of the 3D DNA nanostructure.
agttcgcaccgccattctcggcagactcccaccgacatg

SEQ ID NO: 465 - DNA, 39 bp, Artificial Sequence
  Staple strand_0464 of the 3D DNA nanostructure.
gaaaagcacggcgtgcagtggcccaggtaggtcgttcca

SEQ ID NO: 466 - DNA, 39 bp, Artificial Sequence
  Staple strand_0465 of the 3D DNA nanostructure.
ggtaatgcgttcgggctactgcggtgtgagatcgtgcgg

SEQ ID NO: 467 - DNA, 39 bp, Artificial Sequence
  Staple strand_0466 of the 3D DNA nanostructure.
tctacgtaggcatcagcgtcgcggcactacacgtgggtc

SEQ ID NO: 468 - DNA, 39 bp, Artificial Sequence
  Staple strand_0467 of the 3D DNA nanostructure.
cgaagggacgcgggaggctagggaatgctgcatcagtgg

SEQ ID NO: 469 - DNA, 60 bp, Artificial Sequence
  Staple strand_0468 of the 3D DNA nanostructure.
cctacagggttagctccacagccgggtccgtgcagatgcttcctctaccacctacatcac

SEQ ID NO: 470 - DNA, 60 bp, Artificial Sequence
  Staple strand_0469 of the 3D DNA nanostructure.
agaggcctcctcagcggtcctacgcggtcttggacgatgttcctctaccacctacatcac

SEQ ID NO: 471 - DNA, 60 bp, Artificial Sequence
  Staple strand_0470 of the 3D DNA nanostructure.
cgtactcatcccgtgccagtgccggcctactcacctgtgttcctctaccacctacatcac

SEQ ID NO: 472 - DNA, 60 bp, Artificial Sequence
  Staple strand_0471 of the 3D DNA nanostructure.
gcctgcgacaatgtgggctggaacggtttgctagggcacttcctctaccacctacatcac

SEQ ID NO: 473 - DNA, 60 bp, Artificial Sequence
  Staple strand_0472 of the 3D DNA nanostructure.
cacccgatgtgttatgacgtgcgtcgccctcgagcacctttcctctaccacctacatcac

SEQ ID NO: 474 - DNA, 60 bp, Artificial Sequence
  Staple strand_0473 of the 3D DNA nanostructure.
agcatacagagggcagcttggcagggccgcgaagttaggttcctctaccacctacatcac

SEQ ID NO: 475 - DNA, 60 bp, Artificial Sequence
  Staple strand_0474 of the 3D DNA nanostructure.
gacgcccgattgtccttgttcgccatgccgcaaggtgagttcctctaccacctacatcac

SEQ ID NO: 476 - DNA, 60 bp, Artificial Sequence
  Staple strand_0475 of the 3D DNA nanostructure.
ggtggcgagatgacgacagtgcagaaggtggcggtagggttcctctaccacctacatcac

SEQ ID NO: 477 - DNA, 60 bp, Artificial Sequence
  Staple strand_0476 of the 3D DNA nanostructure.
tctgtgaagcgggtggagtacggaggtgttcttcgcgccttcctctaccacctacatcac

SEQ ID NO: 478 - DNA, 60 bp, Artificial Sequence
  Staple strand_0477 of the 3D DNA nanostructure.
ttttgcgcaatcatggcagcggactcaacaccctccggcttcctctaccacctacatcac

SEQ ID NO: 479 - DNA, 60 bp, Artificial Sequence
  Staple strand_0478 of the 3D DNA nanostructure.
gacctcggagctagacgggagtgttgcgtgtgcagggtgttcctctaccacctacatcac

SEQ ID NO: 480 - DNA, 60 bp, Artificial Sequence
  Staple strand_0479 of the 3D DNA nanostructure.
cggttcgagtgcgcagtgcttaacgtgaagcgagggaggttcctctaccacctacatcac

SEQ ID NO: 481 - DNA, 60 bp, Artificial Sequence
  Staple strand_0480 of the 3D DNA nanostructure.
cctacagggttagctccacagccgggtccgtgcagatgctaacattcctaacttctcata

SEQ ID NO: 482 - DNA, 60 bp, Artificial Sequence
  Staple strand_0481 of the 3D DNA nanostructure.
agaggcctcctcagcggtcctacgcggtcttggacgatgtaacattcctaacttctcata

SEQ ID NO: 483 - DNA, 60 bp, Artificial Sequence
  Staple strand_0482 of the 3D DNA nanostructure.
cgtactcatcccgtgccagtgccggcctactcacctgtgtaacattcctaacttctcata

SEQ ID NO: 484 - DNA, 60 bp, Artificial Sequence
  Staple strand_0483 of the 3D DNA nanostructure.
gcctgcgacaatgtgggctggaacggtttgctagggcactaacattcctaacttctcata

SEQ ID NO: 485 - DNA, 60 bp, Artificial Sequence
  Staple strand_0484 of the 3D DNA nanostructure.
cacccgatgtgttatgacgtgcgtcgccctcgagcaccttaacattcctaacttctcata

SEQ ID NO: 486 - DNA, 60 bp, Artificial Sequence
  Staple strand_0485 of the 3D DNA nanostructure.
agcatacagagggcagcttggcagggccgcgaagttaggtaacattcctaacttctcata

SEQ ID NO: 487 - DNA, 60 bp, Artificial Sequence
  Staple strand_0486 of the 3D DNA nanostructure.
gacgcccgattgtccttgttcgccatgccgcaaggtgagtaacattcctaacttctcata

SEQ ID NO: 488 - DNA, 60 bp, Artificial Sequence
  Staple strand_0487 of the 3D DNA nanostructure.
ggtggcgagatgacgacagtgcagaaggtggcggtagggtaacattcctaacttctcata

SEQ ID NO: 489 - DNA, 60 bp, Artificial Sequence
  Staple strand_0488 of the 3D DNA nanostructure.
tctgtgaagcgggtggagtacggaggtgttcttcgcgcctaacattcctaacttctcata

SEQ ID NO: 490 - DNA, 60 bp, Artificial Sequence
  Staple strand_0489 of the 3D DNA nanostructure.
ttttgcgcaatcatggcagcggactcaacaccctccggctaacattcctaacttctcata

SEQ ID NO: 491 - DNA, 60 bp, Artificial Sequence
  Staple strand_0490 of the 3D DNA nanostructure.
gacctcggagctagacgggagtgttgcgtgtgcagggtgtaacattcctaacttctcata

SEQ ID NO: 492 - DNA, 60 bp, Artificial Sequence
  Staple strand_0491 of the 3D DNA nanostructure.
cggttcgagtgcgcagtgcttaacgtgaagcgagggaggtaacattcctaacttctcata

SEQ ID NO: 493 - DNA, 60 bp, Artificial Sequence
  Staple strand_0492 of the 3D DNA nanostructure.
cctacagggttagctccacagccgggtccgtgcagatgcttaccatctctcctaaactcg

SEQ ID NO: 494 - DNA, 60 bp, Artificial Sequence
  Staple strand_0493 of the 3D DNA nanostructure.
agaggcctcctcagcggtcctacgcggtcttggacgatgttaccatctctcctaaactcg

SEQ ID NO: 495 - DNA, 60 bp, Artificial Sequence
  Staple strand_0494 of the 3D DNA nanostructure.
cgtactcatcccgtgccagtgccggcctactcacctgtgttaccatctctcctaaactcg

SEQ ID NO: 496 - DNA, 60 bp, Artificial Sequence
  Staple strand_0495 of the 3D DNA nanostructure.
gcctgcgacaatgtgggctggaacggtttgctagggcacttaccatctctcctaaactcg

SEQ ID NO: 497 - DNA, 60 bp, Artificial Sequence
  Staple strand_0496 of the 3D DNA nanostructure.
cacccgatgtgttatgacgtgcgtcgccctcgagcacctttaccatctctcctaaactcg

SEQ ID NO: 498 - DNA, 60 bp, Artificial Sequence
  Staple strand_0497 of the 3D DNA nanostructure.
agcatacagagggcagcttggcagggccgcgaagttaggttaccatctctcctaaactcg

SEQ ID NO: 499 - DNA, 60 bp, Artificial Sequence
  Staple strand_0498 of the 3D DNA nanostructure.
gacgcccgattgtccttgttcgccatgccgcaaggtgagttaccatctctcctaaactcg

SEQ ID NO: 500 - DNA, 60 bp, Artificial Sequence
  Staple strand_0499 of the 3D DNA nanostructure.
ggtggcgagatgacgacagtgcagaaggtggcggtagggttaccatctctcctaaactcg

SEQ ID NO: 501 - DNA, 60 bp, Artificial Sequence
  Staple strand_0500 of the 3D DNA nanostructure.
tctgtgaagcgggtggagtacggaggtgttcttcgcgccttaccatctctcctaaactcg

SEQ ID NO: 502 - DNA, 60 bp, Artificial Sequence
  Staple strand_0501 of the 3D DNA nanostructure.
ttttgcgcaatcatggcagcggactcaacaccctccggcttaccatctctcctaaactcg

SEQ ID NO: 503 - DNA, 60 bp, Artificial Sequence
  Staple strand_0502 of the 3D DNA nanostructure.
gacctcggagctagacgggagtgttgcgtgtgcagggtgttaccatctctcctaaactcg

SEQ ID NO: 504 - DNA, 60 bp, Artificial Sequence
  Staple strand_0503 of the 3D DNA nanostructure.
cggttcgagtgcgcagtgcttaacgtgaagcgagggaggttaccatctctcctaaactcg

SEQ ID NO: 505 - DNA, 31 bp, Artificial Sequence
  Staple strand_0504 of the 2D DNA nanostructure.
agaaaggaacaactaaaggaattcaaaaaaa

SEQ ID NO: 506 - DNA, 30 bp, Artificial Sequence
  Staple strand_0505 of the 2D DNA nanostructure.
aggctccagaggctttgaggacacgggtaa

SEQ ID NO: 507 - DNA, 32 bp, Artificial Sequence
  Staple strand_0506 of the 2D DNA nanostructure.
acggctacaaaaggagcctttaatgtgagaat

SEQ ID NO: 508 - DNA, 30 bp, Artificial Sequence
  Staple strand_0507 of the 2D DNA nanostructure.
aatacgtttgaaagaggacagactgacctt

SEQ ID NO: 509 - DNA, 32 bp, Artificial Sequence
  Staple strand_0508 of the 2D DNA nanostructure.
gaccaactaatgccactacgaagggggtagca

SEQ ID NO: 510 - DNA, 30 bp, Artificial Sequence
  Staple strand_0509 of the 2D DNA nanostructure.
catcaagtaaaacgaactaacgagttgaga

SEQ ID NO: 511 - DNA, 32 bp, Artificial Sequence
  Staple strand_0510 of the 2D DNA nanostructure.
tacgttaaagtaatcttgacaagaaccgaact

SEQ ID NO: 512 - DNA, 30 bp, Artificial Sequence
  Staple strand_0511 of the 2D DNA nanostructure.
tttaggacaaatgctttaaacaatcaggtc

SEQ ID NO: 513 - DNA, 32 bp, Artificial Sequence
  Staple strand_0512 of the 2D DNA nanostructure.
atccccctataccacattcaactagaaaaatc

SEQ ID NO: 514 - DNA, 30 bp, Artificial Sequence
  Staple strand_0513 of the 2D DNA nanostructure.
tttaccccaacatgttttaaatttccatat

SEQ ID NO: 515 - DNA, 32 bp, Artificial Sequence
  Staple strand_0514 of the 2D DNA nanostructure.
ctgtagcttgactattatagtcagttcattga

SEQ ID NO: 516 - DNA, 30 bp, Artificial Sequence
  Staple strand_0515 of the 2D DNA nanostructure.
aacagttttgtaccaaaaacattttatttc

SEQ ID NO: 517 - DNA, 32 bp, Artificial Sequence
  Staple strand_0516 of the 2D DNA nanostructure.
acaactttcaacagtttcagcggatgtatcgg

SEQ ID NO: 518 - DNA, 32 bp, Artificial Sequence
  Staple strand_0517 of the 2D DNA nanostructure.
tttatcaggacagcatcggaacgacaccaacc

SEQ ID NO: 519 - DNA, 30 bp, Artificial Sequence
  Staple strand_0518 of the 2D DNA nanostructure.
cagcgaaacttgctttcgaggtgttgctaa

SEQ ID NO: 520 - DNA, 32 bp, Artificial Sequence
  Staple strand_0519 of the 2D DNA nanostructure.
taaaacgaggtcaatcataagggaaccggata

SEQ ID NO: 521 - DNA, 30 bp, Artificial Sequence
  Staple strand_0520 of the 2D DNA nanostructure.
gcgcagacaagaggcaaaagaatccctcag

SEQ ID NO: 522 - DNA, 32 bp, Artificial Sequence
  Staple strand_0521 of the 2D DNA nanostructure.
ttcattacgtcaggacgttgggaaatgcagat

SEQ ID NO: 523 - DNA, 30 bp, Artificial Sequence
  Staple strand_0522 of the 2D DNA nanostructure.
ttataccaccaaatcaacgtaacgaacgag

SEQ ID NO: 524 - DNA, 32 bp, Artificial Sequence
  Staple strand_0523 of the 2D DNA nanostructure.
acataacgggaatcgtcataaataaagcaaag

SEQ ID NO: 525 - DNA, 30 bp, Artificial Sequence
  Staple strand_0524 of the 2D DNA nanostructure.
aatactgcccaaaaggaattacgtggctca

SEQ ID NO: 526 - DNA, 32 bp, Artificial Sequence
  Staple strand_0525 of the 2D DNA nanostructure.
cggattgcagagcttaattgctgaaacgagta

SEQ ID NO: 527 - DNA, 30 bp, Artificial Sequence
  Staple strand_0526 of the 2D DNA nanostructure.
gatggcttatcaaaaagattaagagcgtcc

SEQ ID NO: 528 - DNA, 32 bp, Artificial Sequence
  Staple strand_0527 of the 2D DNA nanostructure.
gatttagtcaataaagcctcagagaaccctca

SEQ ID NO: 529 - DNA, 32 bp, Artificial Sequence
  Staple strand_0528 of the 2D DNA nanostructure.
taaatgaattttctgtatgggattaatttctt

SEQ ID NO: 530 - DNA, 32 bp, Artificial Sequence
  Staple strand_0529 of the 2D DNA nanostructure.
aaacagctttttgcgggatcgtcaacactaaa

SEQ ID NO: 531 - DNA, 32 bp, Artificial Sequence
  Staple strand_0530 of the 2D DNA nanostructure.
aaggccgctgataccgatagttgcgacgttag

SEQ ID NO: 532 - DNA, 32 bp, Artificial Sequence
  Staple strand_0531 of the 2D DNA nanostructure.
acactcatccatgttacttagccgaaagctgc

SEQ ID NO: 533 - DNA, 32 bp, Artificial Sequence
  Staple strand_0532 of the 2D DNA nanostructure.
gacctgctctttgacccccagcgagggagtta

SEQ ID NO: 534 - DNA, 32 bp, Artificial Sequence
  Staple strand_0533 of the 2D DNA nanostructure.
tcattcagatgcgattttaagaacaggcatag

SEQ ID NO: 535 - DNA, 32 bp, Artificial Sequence
  Staple strand_0534 of the 2D DNA nanostructure.
attacctttgaataaggcttgcccaaatccgc

SEQ ID NO: 536 - DNA, 32 bp, Artificial Sequence
  Staple strand_0535 of the 2D DNA nanostructure.
taagagcaaatgtttagactggataggaagcc

SEQ ID NO: 537 - DNA, 32 bp, Artificial Sequence
  Staple strand_0536 of the 2D DNA nanostructure.
aatagtaaacactatcataaccctcattgtga

SEQ ID NO: 538 - DNA, 32 bp, Artificial Sequence
  Staple strand_0537 of the 2D DNA nanostructure.
cgaaagactttgataagaggtcatatttcgca

SEQ ID NO: 539 - DNA, 32 bp, Artificial Sequence
  Staple strand_0538 of the 2D DNA nanostructure.
ttgctcctttcaaatatcgcgtttgagggggt

SEQ ID NO: 540 - DNA, 32 bp, Artificial Sequence
  Staple strand_0539 of the 2D DNA nanostructure.
aatggtcaacaggcaaggcaaagagtaatgtg

SEQ ID NO: 541 - DNA, 31 bp, Artificial Sequence
  Staple strand_0540 of the 2D DNA nanostructure.
tctaaagttttgtcgtctttccagccgacaa

SEQ ID NO: 542 - DNA, 44 bp, Artificial Sequence
  Staple strand_0541 of the 2D DNA nanostructure.
tgacaactcgctgaggcttgcattataccaagcgcgatgataaa

SEQ ID NO: 543 - DNA, 32 bp, Artificial Sequence
  Staple strand_0542 of the 2D DNA nanostructure.
atattcggaaccatcgcccacgcagagaagga

SEQ ID NO: 544 - DNA, 32 bp, Artificial Sequence
  Staple strand_0543 of the 2D DNA nanostructure.
tcatcgccaacaaagtacaacggacgccagca

SEQ ID NO: 545 - DNA, 32 bp, Artificial Sequence
  Staple strand_0544 of the 2D DNA nanostructure.
gatggtttgaacgagtagtaaatttaccatta

SEQ ID NO: 546 - DNA, 38 bp, Artificial Sequence
  Staple strand_0545 of the 2D DNA nanostructure.
cgtttaccagacgacaaagaagttttgccataattcga

SEQ ID NO: 547 - DNA, 32 bp, Artificial Sequence
  Staple strand_0546 of the 2D DNA nanostructure.
cttttgcagataaaaaccaaaataaagactcc

SEQ ID NO: 548 - DNA, 30 bp, Artificial Sequence
  Staple strand_0547 of the 2D DNA nanostructure.
gcttcaatcaggattagagagttattttca

SEQ ID NO: 549 - DNA, 32 bp, Artificial Sequence
  Staple strand_0548 of the 2D DNA nanostructure.
ccaacaggagcgaaccagaccggagcctttac

SEQ ID NO: 550 - DNA, 30 bp, Artificial Sequence
  Staple strand_0549 of the 2D DNA nanostructure.
tttggggatagtagtagcattaaaaggccg

SEQ ID NO: 551 - DNA, 32 bp, Artificial Sequence
  Staple strand_0550 of the 2D DNA nanostructure.
tccacagacagccctcatagttagcgtaacga

SEQ ID NO: 552 - DNA, 32 bp, Artificial Sequence
  Staple strand_0551 of the 2D DNA nanostructure.
ttaggattggctgagactcctcaataaccgat

SEQ ID NO: 553 - DNA, 30 bp, Artificial Sequence
  Staple strand_0552 of the 2D DNA nanostructure.
tattaagaagcggggttttgctcgtagcat

SEQ ID NO: 554 - DNA, 32 bp, Artificial Sequence
  Staple strand_0553 of the 2D DNA nanostructure.
ttgacaggccaccaccagagccgcgatttgta

SEQ ID NO: 555 - DNA, 30 bp, Artificial Sequence
  Staple strand_0554 of the 2D DNA nanostructure.
caccagaaaggttgaggcaggtcatgaaag

SEQ ID NO: 556 - DNA, 32 bp, Artificial Sequence
  Staple strand_0555 of the 2D DNA nanostructure.
gcaaggcctcaccagtagcaccatgggcttga

SEQ ID NO: 557 - DNA, 30 bp, Artificial Sequence
  Staple strand_0556 of the 2D DNA nanostructure.
cagcaaaaggaaacgtcaccaatgagccgc

SEQ ID NO: 558 - DNA, 32 bp, Artificial Sequence
  Staple strand_0557 of the 2D DNA nanostructure.
ttattacgaagaactggcatgattgcgagagg

SEQ ID NO: 559 - DNA, 30 bp, Artificial Sequence
  Staple strand_0558 of the 2D DNA nanostructure.
atacccaacagtatgttagcaaattagagc

SEQ ID NO: 560 - DNA, 32 bp, Artificial Sequence
  Staple strand_0559 of the 2D DNA nanostructure.
agagagaaaaaaatgaaaatagcaagcaaact

SEQ ID NO: 561 - DNA, 30 bp, Artificial Sequence
  Staple strand_0560 of the 2D DNA nanostructure.
ttaacgtctaacataaaaacaggtaacgga

SEQ ID NO: 562 - DNA, 32 bp, Artificial Sequence
  Staple strand_0561 of the 2D DNA nanostructure.
ccaatagctcatcgtaggaatcatggcatcaa

SEQ ID NO: 563 - DNA, 32 bp, Artificial Sequence
  Staple strand_0562 of the 2D DNA nanostructure.
tcaccagtacaaactacaacgcctagtaccag

SEQ ID NO: 564 - DNA, 32 bp, Artificial Sequence
  Staple strand_0563 of the 2D DNA nanostructure.
gcggataacctattattctgaaacagacgatt

SEQ ID NO: 565 - DNA, 32 bp, Artificial Sequence
  Staple strand_0564 of the 2D DNA nanostructure.
tttcggaagtgccgtcgagagggtgagtttcg

SEQ ID NO: 566 - DNA, 32 bp, Artificial Sequence
  Staple strand_0565 of the 2D DNA nanostructure.
ggccttgaagagccaccaccctcagaaaccat

SEQ ID NO: 567 - DNA, 32 bp, Artificial Sequence
  Staple strand_0566 of the 2D DNA nanostructure.
ccaccctctattcacaaacaaatacctgccta

SEQ ID NO: 568 - DNA, 32 bp, Artificial Sequence
  Staple strand_0567 of the 2D DNA nanostructure.
cgatagcattgagccatttgggaacgtagaaa

SEQ ID NO: 569 - DNA, 32 bp, Artificial Sequence
  Staple strand_0568 of the 2D DNA nanostructure.
tcaccgacgcaccgtaatcagtagcagaaccg

SEQ ID NO: 570 - DNA, 32 bp, Artificial Sequence
  Staple strand_0569 of the 2D DNA nanostructure.
atacataccgaggaaacgcaataagaagcgca

SEQ ID NO: 571 - DNA, 32 bp, Artificial Sequence
  Staple strand_0570 of the 2D DNA nanostructure.
aaggaaacataaaggtggcaacattatcaccg

SEQ ID NO: 572 - DNA, 32 bp, Artificial Sequence
  Staple strand_0571 of the 2D DNA nanostructure.
ttagacggccaaataagaaacgatagaaggct

SEQ ID NO: 573 - DNA, 32 bp, Artificial Sequence
  Staple strand_0572 of the 2D DNA nanostructure.
atcccaatgagaattaactgaacagttaccag

SEQ ID NO: 574 - DNA, 32 bp, Artificial Sequence
  Staple strand_0573 of the 2D DNA nanostructure.
tatccggtctcatcgagaacaagcgacaaaag

SEQ ID NO: 575 - DNA, 31 bp, Artificial Sequence
  Staple strand_0574 of the 2D DNA nanostructure.
aggaacccatgtaccgtaacacttgatataa

SEQ ID NO: 576 - DNA, 30 bp, Artificial Sequence
  Staple strand_0575 of the 2D DNA nanostructure.
gtatagcaaacagttaatgcccaatcctca

SEQ ID NO: 577 - DNA, 32 bp, Artificial Sequence
  Staple strand_0576 of the 2D DNA nanostructure.
gcccgtatccggaataggtgtatcagcccaat

SEQ ID NO: 578 - DNA, 30 bp, Artificial Sequence
  Staple strand_0577 of the 2D DNA nanostructure.
ttaaagccagagccgccaccctcgacagaa

SEQ ID NO: 579 - DNA, 32 bp, Artificial Sequence
  Staple strand_0578 of the 2D DNA nanostructure.
gcctccctcagaatggaaagcgcagtaacagt

SEQ ID NO: 580 - DNA, 30 bp, Artificial Sequence
  Staple strand_0579 of the 2D DNA nanostructure.
tcaagtttcattaaaggtgaatataaaaga

SEQ ID NO: 581 - DNA, 32 bp, Artificial Sequence
  Staple strand_0580 of the 2D DNA nanostructure.
gaaattattgcctttagcgtcagaccggaacc

SEQ ID NO: 582 - DNA, 30 bp, Artificial Sequence
  Staple strand_0581 of the 2D DNA nanostructure.
aacgcaaagatagccgaacaaaccctgaac

SEQ ID NO: 583 - DNA, 32 bp, Artificial Sequence
  Staple strand_0582 of the 2D DNA nanostructure.
aagtaagcagacaccacggaataatattgacg

SEQ ID NO: 584 - DNA, 30 bp, Artificial Sequence
  Staple strand_0583 of the 2D DNA nanostructure.
aaagtcacaaaataaacagccagcgtttta

SEQ ID NO: 585 - DNA, 32 bp, Artificial Sequence
  Staple strand_0584 of the 2D DNA nanostructure.
gccagttagagggtaattgagcgctttaagaa

SEQ ID NO: 586 - DNA, 30 bp, Artificial Sequence
  Staple strand_0585 of the 2D DNA nanostructure.
gcgaacctccaagaacgggtatgacaataa

SEQ ID NO: 587 - DNA, 32 bp, Artificial Sequence
  Staple strand_0586 of the 2D DNA nanostructure.
ccaccctcattttcagggatagcaaccgtact

SEQ ID NO: 588 - DNA, 32 bp, Artificial Sequence
  Staple strand_0587 of the 2D DNA nanostructure.
caggaggtggggtcagtgccttgagtctctga

SEQ ID NO: 589 - DNA, 30 bp, Artificial Sequence
  Staple strand_0588 of the 2D DNA nanostructure.
gttttaacttagtaccgccacccagagcca

SEQ ID NO: 590 - DNA, 32 bp, Artificial Sequence
  Staple strand_0589 of the 2D DNA nanostructure.
atttaccgggaaccagagccaccactgtagcg

SEQ ID NO: 591 - DNA, 30 bp, Artificial Sequence
  Staple strand_0590 of the 2D DNA nanostructure.
aaatcaccttccagtaagcgtcagtaataa

SEQ ID NO: 592 - DNA, 32 bp, Artificial Sequence
  Staple strand_0591 of the 2D DNA nanostructure.
cgttttcaagggagggaaggtaaagtttattt

SEQ ID NO: 593 - DNA, 30 bp, Artificial Sequence
  Staple strand_0592 of the 2D DNA nanostructure.
accgattgtcggcattttcggtcataatca

SEQ ID NO: 594 - DNA, 32 bp, Artificial Sequence
  Staple strand_0593 of the 2D DNA nanostructure.
tgtcacaatcttaccgaagccctttaatatca

SEQ ID NO: 595 - DNA, 30 bp, Artificial Sequence
  Staple strand_0594 of the 2D DNA nanostructure.
aatagctatcaatagaaaattcaacattca

SEQ ID NO: 596 - DNA, 32 bp, Artificial Sequence
  Staple strand_0595 of the 2D DNA nanostructure.
gagagatagagcgtctttccagaggttttgaa

SEQ ID NO: 597 - DNA, 30 bp, Artificial Sequence
  Staple strand_0596 of the 2D DNA nanostructure.
acgctaacacccacaagaattgaaaatagc

SEQ ID NO: 598 - DNA, 32 bp, Artificial Sequence
  Staple strand_0597 of the 2D DNA nanostructure.
gccttaaaccaatcaataatcggcacgcgcct

SEQ ID NO: 599 - DNA, 32 bp, Artificial Sequence
  Staple strand_0598 of the 2D DNA nanostructure.
taaatcgggattcccaattctgcgatataatg

SEQ ID NO: 600 - DNA, 30 bp, Artificial Sequence
  Staple strand_0599 of the 2D DNA nanostructure.
aacgcaaaatcgatgaacggtaccggttga

SEQ ID NO: 601 - DNA, 32 bp, Artificial Sequence
  Staple strand_0600 of the 2D DNA nanostructure.
aacaagagggataaaaatttttagcataaagc

SEQ ID NO: 602 - DNA, 30 bp, Artificial Sequence
  Staple strand_0601 of the 2D DNA nanostructure.
taatcagcggattgaccgtaatcgtaaccg

SEQ ID NO: 603 - DNA, 32 bp, Artificial Sequence
  Staple strand_0602 of the 2D DNA nanostructure.
acaaacggaaaagccccaaaaacactggagca

SEQ ID NO: 604 - DNA, 30 bp, Artificial Sequence
  Staple strand_0603 of the 2D DNA nanostructure.
tgcatctttcccagtcacgacggcctgcag

SEQ ID NO: 605 - DNA, 32 bp, Artificial Sequence
  Staple strand_0604 of the 2D DNA nanostructure.
ccagggttgccagtttgaggggacccgtggga

SEQ ID NO: 606 - DNA, 30 bp, Artificial Sequence
  Staple strand_0605 of the 2D DNA nanostructure.
gtcgacttcggccaacgcgcggggtttttc

SEQ ID NO: 607 - DNA, 32 bp, Artificial Sequence
  Staple strand_0606 of the 2D DNA nanostructure.
ttaatgaactagaggatccccggggggtaacg

SEQ ID NO: 608 - DNA, 30 bp, Artificial Sequence
  Staple strand_0607 of the 2D DNA nanostructure.
ttttcactcaaagggcgaaaaaccatcacc

SEQ ID NO: 609 - DNA, 32 bp, Artificial Sequence
  Staple strand_0608 of the 2D DNA nanostructure.
ctccaacgcagtgagacgggcaaccagctgca

SEQ ID NO: 610 - DNA, 31 bp, Artificial Sequence
  Staple strand_0609 of the 2D DNA nanostructure.
caaatcaagttttttggggtcgaaacgtgga

SEQ ID NO: 611 - DNA, 30 bp, Artificial Sequence
  Staple strand_0610 of the 2D DNA nanostructure.
aaattaagttgaccattagatacttttgcg

SEQ ID NO: 612 - DNA, 32 bp, Artificial Sequence
  Staple strand_0611 of the 2D DNA nanostructure.
tatattttgtcattgcctgagagtggaagatt

SEQ ID NO: 613 - DNA, 30 bp, Artificial Sequence
  Staple strand_0612 of the 2D DNA nanostructure.
gctatcagaaatgcaatgcctgaattagca

SEQ ID NO: 614 - DNA, 32 bp, Artificial Sequence
  Staple strand_0613 of the 2D DNA nanostructure.
gtataagccaacccgtcggattctgacgacag

SEQ ID NO: 615 - DNA, 30 bp, Artificial Sequence
  Staple strand_0614 of the 2D DNA nanostructure.
gcgagtaaaaatatttaaattgttacaaag

SEQ ID NO: 616 - DNA, 32 bp, Artificial Sequence
  Staple strand_0615 of the 2D DNA nanostructure.
tatcggccgcaaggcgattaagtttaccgagc

SEQ ID NO: 617 - DNA, 30 bp, Artificial Sequence
  Staple strand_0616 of the 2D DNA nanostructure.
gatgtgcttcaggaagatcgcacaatgtga

SEQ ID NO: 618 - DNA, 32 bp, Artificial Sequence
  Staple strand_0617 of the 2D DNA nanostructure.
tcgaattcgggaaacctgtcgtgcagctgatt

SEQ ID NO: 619 - DNA, 30 bp, Artificial Sequence
  Staple strand_0618 of the 2D DNA nanostructure.
ttccagtcgtaatcatggtcataaaagggg

SEQ ID NO: 620 - DNA, 32 bp, Artificial Sequence
  Staple strand_0619 of the 2D DNA nanostructure.
gcccttcagagtccactattaaagggtgccgt

SEQ ID NO: 621 - DNA, 30 bp, Artificial Sequence
  Staple strand_0620 of the 2D DNA nanostructure.
tggaacaaccgcctggccctgaggcccgct

SEQ ID NO: 622 - DNA, 30 bp, Artificial Sequence
  Staple strand_0621 of the 2D DNA nanostructure.
aaagcactaaatcggaaccctaatccagtt

SEQ ID NO: 623 - DNA, 32 bp, Artificial Sequence
  Staple strand_0622 of the 2D DNA nanostructure.
taaatcatataacctgtttagctaacctttaa

SEQ ID NO: 624 - DNA, 32 bp, Artificial Sequence
  Staple strand_0623 of the 2D DNA nanostructure.
taggtaaactatttttgagagatcaaacgtta

SEQ ID NO: 625 - DNA, 32 bp, Artificial Sequence
  Staple strand_0624 of the 2D DNA nanostructure.
gagggtaggattcaaaagggtgagacatccaa

SEQ ID NO: 626 - DNA, 32 bp, Artificial Sequence
  Staple strand_0625 of the 2D DNA nanostructure.
atattttggctttcatcaacattatccagcca

SEQ ID NO: 627 - DNA, 32 bp, Artificial Sequence
  Staple strand_0626 of the 2D DNA nanostructure.
tgtagccattaaaattcgcattaaatgccgga

SEQ ID NO: 628 - DNA, 32 bp, Artificial Sequence
  Staple strand_0627 of the 2D DNA nanostructure.
gctttccgattacgccagctggcggctgtttc

SEQ ID NO: 629 - DNA, 32 bp, Artificial Sequence
  Staple strand_0628 of the 2D DNA nanostructure.
tcttcgctgcaccgcttctggtgcggccttcc

SEQ ID NO: 630 - DNA, 32 bp, Artificial Sequence
  Staple strand_0629 of the 2D DNA nanostructure.
ctgtgtgattgcgttgcgctcactagagttgc

SEQ ID NO: 631 - DNA, 32 bp, Artificial Sequence
  Staple strand_0630 of the 2D DNA nanostructure.
cacattaaaattgttatccgctcatgcgggcc

SEQ ID NO: 632 - DNA, 32 bp, Artificial Sequence
  Staple strand_0631 of the 2D DNA nanostructure.
agcaagcgtagggttgagtgttgtagggagcc

SEQ ID NO: 633 - DNA, 32 bp, Artificial Sequence
  Staple strand_0632 of the 2D DNA nanostructure.
gcccgagagtccacgctggtttgcagctaact

SEQ ID NO: 634 - DNA, 32 bp, Artificial Sequence
  Staple strand_0633 of the 2D DNA nanostructure.
cccgatttagagcttgacggggaaaaagaata

SEQ ID NO: 635 - DNA, 32 bp, Artificial Sequence
  Staple strand_0634 of the 2D DNA nanostructure.
ttctactacgcgagctgaaaaggttaccgcgc

SEQ ID NO: 636 - DNA, 30 bp, Artificial Sequence
  Staple strand_0635 of the 2D DNA nanostructure.
gagacagctagctgataaattaatttttgt

SEQ ID NO: 637 - DNA, 32 bp, Artificial Sequence
  Staple strand_0636 of the 2D DNA nanostructure.
caaccgtttcaaatcaccatcaattcgagcca

SEQ ID NO: 638 - DNA, 44 bp, Artificial Sequence
  Staple strand_0637 of the 2D DNA nanostructure.
taaatcaaaataattcgcgtctcggaaaccaggcaaagggaagg

SEQ ID NO: 639 - DNA, 32 bp, Artificial Sequence
  Staple strand_0638 of the 2D DNA nanostructure.
gccatcaagctcattttttaaccacaaatcca

SEQ ID NO: 640 - DNA, 32 bp, Artificial Sequence
  Staple strand_0639 of the 2D DNA nanostructure.
caactgttgcgccattcgccattcaaacatca

SEQ ID NO: 641 - DNA, 32 bp, Artificial Sequence
  Staple strand_0640 of the 2D DNA nanostructure.
aagcctggtacgagccggaagcatagatgatg

SEQ ID NO: 642 - DNA, 38 bp, Artificial Sequence
  Staple strand_0641 of the 2D DNA nanostructure.
cccagcaggcgaaaaatcccttataaatcaagccggcg

SEQ ID NO: 643 - DNA, 32 bp, Artificial Sequence
  Staple strand_0642 of the 2D DNA nanostructure.
tcggcaaatcctgtttgatggtggaccctcaa

SEQ ID NO: 644 - DNA, 31 bp, Artificial Sequence
  Staple strand_0643 of the 2D DNA nanostructure.
aacgtggcgagaaaggaagggaaaccagtaa

SEQ ID NO: 645 - DNA, 30 bp, Artificial Sequence
  Staple strand_0644 of the 2D DNA nanostructure.
ttttatttaagcaaatcagatattttttgt

SEQ ID NO: 646 - DNA, 32 bp, Artificial Sequence
  Staple strand_0645 of the 2D DNA nanostructure.
gtaataagttaggcagaggcatttatgatatt

SEQ ID NO: 647 - DNA, 30 bp, Artificial Sequence
  Staple strand_0646 of the 2D DNA nanostructure.
catgtaatagaatataaagtaccaagccgt

SEQ ID NO: 648 - DNA, 32 bp, Artificial Sequence
  Staple strand_0647 of the 2D DNA nanostructure.
atcgcaagtatgtaaatgctgatgataggaac

SEQ ID NO: 649 - DNA, 30 bp, Artificial Sequence
  Staple strand_0648 of the 2D DNA nanostructure.
tataactaacaaagaacgcgagaacgccaa

SEQ ID NO: 650 - DNA, 32 bp, Artificial Sequence
  Staple strand_0649 of the 2D DNA nanostructure.
agaaaacaaagaagatgatgaaacaggctgcg

SEQ ID NO: 651 - DNA, 30 bp, Artificial Sequence
  Staple strand_0650 of the 2D DNA nanostructure.
ctgagcaaaaattaattacattttgggtta

SEQ ID NO: 652 - DNA, 32 bp, Artificial Sequence
  Staple strand_0651 of the 2D DNA nanostructure.
gcaattcacatattcctgattatcaaagtgta

SEQ ID NO: 653 - DNA, 30 bp, Artificial Sequence
  Staple strand_0652 of the 2D DNA nanostructure.
attatcattcaatataatcctgacaattac

SEQ ID NO: 654 - DNA, 32 bp, Artificial Sequence
  Staple strand_0653 of the 2D DNA nanostructure.
tcaatatcgaacctcaaatatcaattccgaaa

SEQ ID NO: 655 - DNA, 30 bp, Artificial Sequence
  Staple strand_0654 of the 2D DNA nanostructure.
accttgcttggtcagttggcaaagagcgga

SEQ ID NO: 656 - DNA, 30 bp, Artificial Sequence
  Staple strand_0655 of the 2D DNA nanostructure.
taaaagggacattctggccaacaaagcatc

SEQ ID NO: 657 - DNA, 32 bp, Artificial Sequence
  Staple strand_0656 of the 2D DNA nanostructure.
gtaccgcaattctaagaacgcgagtattattt

SEQ ID NO: 658 - DNA, 32 bp, Artificial Sequence
  Staple strand_0657 of the 2D DNA nanostructure.
gtaaagtaatcgccatatttaacaaaactttt

SEQ ID NO: 659 - DNA, 32 bp, Artificial Sequence
  Staple strand_0658 of the 2D DNA nanostructure.
aattgagaattctgtccagacgactaaaccaa

SEQ ID NO: 660 - DNA, 32 bp, Artificial Sequence
  Staple strand_0659 of the 2D DNA nanostructure.
tcaaatataacctccggcttaggtaacaattt

SEQ ID NO: 661 - DNA, 32 bp, Artificial Sequence
  Staple strand_0660 of the 2D DNA nanostructure.
acctttttattttagttaatttcatagggctt

SEQ ID NO: 662 - DNA, 32 bp, Artificial Sequence
  Staple strand_0661 of the 2D DNA nanostructure.
catttgaaggcgaattattcatttttgtttgg

SEQ ID NO: 663 - DNA, 32 bp, Artificial Sequence
  Staple strand_0662 of the 2D DNA nanostructure.
cgcgcagattaccttttttaatgggagagact

SEQ ID NO: 664 - DNA, 32 bp, Artificial Sequence
  Staple strand_0663 of the 2D DNA nanostructure.
attatactaagaaaccaccagaagtcaacagt

SEQ ID NO: 665 - DNA, 32 bp, Artificial Sequence
  Staple strand_0664 of the 2D DNA nanostructure.
gcggaacatctgaataatggaaggtacaaaat

SEQ ID NO: 666 - DNA, 32 bp, Artificial Sequence
  Staple strand_0665 of the 2D DNA nanostructure.
tgaaaggagcaaatgaaaaatctagagataga

SEQ ID NO: 667 - DNA, 32 bp, Artificial Sequence
  Staple strand_0666 of the 2D DNA nanostructure.
agccagcaattgaggaaggttatcatcatttt

SEQ ID NO: 668 - DNA, 32 bp, Artificial Sequence
  Staple strand_0667 of the 2D DNA nanostructure.
acccttctgacctgaaagcgtaagacgctgag

SEQ ID NO: 669 - DNA, 32 bp, Artificial Sequence
  Staple strand_0668 of the 2D DNA nanostructure.
cttatcattcccgacttgcgggagcctaattt

SEQ ID NO: 670 - DNA, 30 bp, Artificial Sequence
  Staple strand_0669 of the 2D DNA nanostructure.
acaacatgccaacgctcaacagtcttctga

SEQ ID NO: 671 - DNA, 32 bp, Artificial Sequence
  Staple strand_0670 of the 2D DNA nanostructure.
agtataaagttcagctaatgcagatgtctttc

SEQ ID NO: 672 - DNA, 30 bp, Artificial Sequence
  Staple strand_0671 of the 2D DNA nanostructure.
cctaaatcaaaatcataggtctaaacagta

SEQ ID NO: 673 - DNA, 32 bp, Artificial Sequence
  Staple strand_0672 of the 2D DNA nanostructure.
gaatttatttaatggtttgaaatattcttacc

SEQ ID NO: 674 - DNA, 30 bp, Artificial Sequence
  Staple strand_0673 of the 2D DNA nanostructure.
cataaatctttgaataccaagtgttagaac

SEQ ID NO: 675 - DNA, 32 bp, Artificial Sequence
  Staple strand_0674 of the 2D DNA nanostructure.
cctgattgcaatatatgtgagtgatcaatagt

SEQ ID NO: 676 - DNA, 30 bp, Artificial Sequence
  Staple strand_0675 of the 2D DNA nanostructure.
ctaccatagtttgagtaacatttaaaatat

SEQ ID NO: 677 - DNA, 32 bp, Artificial Sequence
  Staple strand_0676 of the 2D DNA nanostructure.
attttaaaatcaaaattatttgcacggattcg

SEQ ID NO: 678 - DNA, 30 bp, Artificial Sequence
  Staple strand_0677 of the 2D DNA nanostructure.
ctttagggcctgcaacagtgccaatacgtg

SEQ ID NO: 679 - DNA, 32 bp, Artificial Sequence
  Staple strand_0678 of the 2D DNA nanostructure.
ttaacaccagcactaacaactaatcgttatta

SEQ ID NO: 680 - DNA, 31 bp, Artificial Sequence
  Staple strand_0679 of the 2D DNA nanostructure.
gcacagacaatatttttgaatggggtcagta

SEQ ID NO: 681 - DNA, 30 bp, Artificial Sequence
  Staple strand_0680 of the 2D DNA nanostructure.
tgtagaaatcaagattagttgctcttacca

SEQ ID NO: 682 - DNA, 32 bp, Artificial Sequence
  Staple strand_0681 of the 2D DNA nanostructure.
gtttatcaatatgcgttatacaaaccgaccgt

SEQ ID NO: 683 - DNA, 30 bp, Artificial Sequence
  Staple strand_0682 of the 2D DNA nanostructure.
ttagtatcacaatagataagtccacgagca

SEQ ID NO: 684 - DNA, 32 bp, Artificial Sequence
  Staple strand_0683 of the 2D DNA nanostructure.
gtgataaaaagacgctgagaagagataacctt

SEQ ID NO: 685 - DNA, 30 bp, Artificial Sequence
  Staple strand_0684 of the 2D DNA nanostructure.
cttagatttaaggcgttaaataaagcctgt

SEQ ID NO: 686 - DNA, 32 bp, Artificial Sequence
  Staple strand_0685 of the 2D DNA nanostructure.
gcttctgttcgggagaaacaataacgtaaaac

SEQ ID NO: 687 - DNA, 30 bp, Artificial Sequence
  Staple strand_0686 of the 2D DNA nanostructure.
cttttacaaaatcgtcgctattagcgatag

SEQ ID NO: 688 - DNA, 32 bp, Artificial Sequence
  Staple strand_0687 of the 2D DNA nanostructure.
agaaataaaaatcctttgcccgaaagattaga

SEQ ID NO: 689 - DNA, 30 bp, Artificial Sequence
  Staple strand_0688 of the 2D DNA nanostructure.
ctcgtattagaaattgcgtagatacagtac

SEQ ID NO: 690 - DNA, 32 bp, Artificial Sequence
  Staple strand_0689 of the 2D DNA nanostructure.
gccgtcaaaaaacagaggtgaggcctattagt

SEQ ID NO: 691 - DNA, 30 bp, Artificial Sequence
  Staple strand_0690 of the 2D DNA nanostructure.
cagaagattagataatacatttgtcgacaa

SEQ ID NO: 692 - DNA, 30 bp, Artificial Sequence
  Staple strand_0691 of the 2D DNA nanostructure.
ctttaatgcgcgaactgatagccccaccag

SEQ ID NO: 693 - DNA, 52 bp, Artificial Sequence
  Staple strand_0692 of the 2D DNA nanostructure.
agaaaggaacaactaaaggaattcaaaaaaattcctctaccacctacatcac

SEQ ID NO: 694 - DNA, 51 bp, Artificial Sequence
  Staple strand_0693 of the 2D DNA nanostructure.
aggctccagaggctttgaggacacgggtaattcctctaccacctacatcac

SEQ ID NO: 695 - DNA, 53 bp, Artificial Sequence
  Staple strand_0694 of the 2D DNA nanostructure.
acggctacaaaaggagcctttaatgtgagaatttcctctaccacctacatcac

SEQ ID NO: 696 - DNA, 51 bp, Artificial Sequence
  Staple strand_0695 of the 2D DNA nanostructure.
aatacgtttgaaagaggacagactgaccttttcctctaccacctacatcac

SEQ ID NO: 697 - DNA, 53 bp, Artificial Sequence
  Staple strand_0696 of the 2D DNA nanostructure.
gaccaactaatgccactacgaagggggtagcattcctctaccacctacatcac

SEQ ID NO: 698 - DNA, 51 bp, Artificial Sequence
  Staple strand_0697 of the 2D DNA nanostructure.
catcaagtaaaacgaactaacgagttgagattcctctaccacctacatcac

SEQ ID NO: 699 - DNA, 53 bp, Artificial Sequence
  Staple strand_0698 of the 2D DNA nanostructure.
tacgttaaagtaatcttgacaagaaccgaactttcctctaccacctacatcac

SEQ ID NO: 700 - DNA, 51 bp, Artificial Sequence
  Staple strand_0699 of the 2D DNA nanostructure.
tttaggacaaatgctttaaacaatcaggtcttcctctaccacctacatcac

SEQ ID NO: 701 - DNA, 53 bp, Artificial Sequence
  Staple strand_0700 of the 2D DNA nanostructure.
atccccctataccacattcaactagaaaaatcttcctctaccacctacatcac

SEQ ID NO: 702 - DNA, 51 bp, Artificial Sequence
  Staple strand_0701 of the 2D DNA nanostructure.
tttaccccaacatgttttaaatttccatatttcctctaccacctacatcac

SEQ ID NO: 703 - DNA, 53 bp, Artificial Sequence
  Staple strand_0702 of the 2D DNA nanostructure.
ctgtagcttgactattatagtcagttcattgattcctctaccacctacatcac

SEQ ID NO: 704 - DNA, 51 bp, Artificial Sequence
  Staple strand_0703 of the 2D DNA nanostructure.
aacagttttgtaccaaaaacattttatttcttcctctaccacctacatcac

SEQ ID NO: 705 - DNA, 53 bp, Artificial Sequence
  Staple strand_0704 of the 2D DNA nanostructure.
acaactttcaacagtttcagcggatgtatcggttcctctaccacctacatcac

SEQ ID NO: 706 - DNA, 53 bp, Artificial Sequence
  Staple strand_0705 of the 2D DNA nanostructure.
tttatcaggacagcatcggaacgacaccaaccttcctctaccacctacatcac

SEQ ID NO: 707 - DNA, 51 bp, Artificial Sequence
  Staple strand_0706 of the 2D DNA nanostructure.
cagcgaaacttgctttcgaggtgttgctaattcctctaccacctacatcac

SEQ ID NO: 708 - DNA, 53 bp, Artificial Sequence
  Staple strand_0707 of the 2D DNA nanostructure.
taaaacgaggtcaatcataagggaaccggatattcctctaccacctacatcac

SEQ ID NO: 709 - DNA, 51 bp, Artificial Sequence
  Staple strand_0708 of the 2D DNA nanostructure.
gcgcagacaagaggcaaaagaatccctcagttcctctaccacctacatcac

SEQ ID NO: 710 - DNA, 53 bp, Artificial Sequence
  Staple strand_0709 of the 2D DNA nanostructure.
ttcattacgtcaggacgttgggaaatgcagatttcctctaccacctacatcac

SEQ ID NO: 711 - DNA, 51 bp, Artificial Sequence
  Staple strand_0710 of the 2D DNA nanostructure.
ttataccaccaaatcaacgtaacgaacgagttcctctaccacctacatcac

SEQ ID NO: 712 - DNA, 53 bp, Artificial Sequence
  Staple strand_0711 of the 2D DNA nanostructure.
acataacgggaatcgtcataaataaagcaaagttcctctaccacctacatcac

SEQ ID NO: 713 - DNA, 51 bp, Artificial Sequence
  Staple strand_0712 of the 2D DNA nanostructure.
aatactgcccaaaaggaattacgtggctcattcctctaccacctacatcac

SEQ ID NO: 714 - DNA, 53 bp, Artificial Sequence
  Staple strand_0713 of the 2D DNA nanostructure.
cggattgcagagcttaattgctgaaacgagtattcctctaccacctacatcac

SEQ ID NO: 715 - DNA, 51 bp, Artificial Sequence
  Staple strand_0714 of the 2D DNA nanostructure.
gatggcttatcaaaaagattaagagcgtccttcctctaccacctacatcac

SEQ ID NO: 716 - DNA, 53 bp, Artificial Sequence
  Staple strand_0715 of the 2D DNA nanostructure.
gatttagtcaataaagcctcagagaaccctcattcctctaccacctacatcac

SEQ ID NO: 717 - DNA, 53 bp, Artificial Sequence
  Staple strand_0716 of the 2D DNA nanostructure.
taaatgaattttctgtatgggattaatttcttttcctctaccacctacatcac

SEQ ID NO: 718 - DNA, 53 bp, Artificial Sequence
  Staple strand_0717 of the 2D DNA nanostructure.
aaacagctttttgcgggatcgtcaacactaaattcctctaccacctacatcac

SEQ ID NO: 719 - DNA, 53 bp, Artificial Sequence
  Staple strand_0718 of the 2D DNA nanostructure.
aaggccgctgataccgatagttgcgacgttagttcctctaccacctacatcac

SEQ ID NO: 720 - DNA, 53 bp, Artificial Sequence
  Staple strand_0719 of the 2D DNA nanostructure.
acactcatccatgttacttagccgaaagctgcttcctctaccacctacatcac

SEQ ID NO: 721 - DNA, 53 bp, Artificial Sequence
  Staple strand_0720 of the 2D DNA nanostructure.
gacctgctctttgacccccagcgagggagttattcctctaccacctacatcac

SEQ ID NO: 722 - DNA, 53 bp, Artificial Sequence
  Staple strand_0721 of the 2D DNA nanostructure.
tcattcagatgcgattttaagaacaggcatagttcctctaccacctacatcac

SEQ ID NO: 723 - DNA, 53 bp, Artificial Sequence
  Staple strand_0722 of the 2D DNA nanostructure.
attacctttgaataaggcttgcccaaatccgcttcctctaccacctacatcac

SEQ ID NO: 724 - DNA, 53 bp, Artificial Sequence
  Staple strand_0723 of the 2D DNA nanostructure.
taagagcaaatgtttagactggataggaagccttcctctaccacctacatcac

SEQ ID NO: 725 - DNA, 53 bp, Artificial Sequence
  Staple strand_0724 of the 2D DNA nanostructure.
aatagtaaacactatcataaccctcattgtgattcctctaccacctacatcac

SEQ ID NO: 726 - DNA, 53 bp, Artificial Sequence
  Staple strand_0725 of the 2D DNA nanostructure.
cgaaagactttgataagaggtcatatttcgcattcctctaccacctacatcac

SEQ ID NO: 727 - DNA, 53 bp, Artificial Sequence
  Staple strand_0726 of the 2D DNA nanostructure.
ttgctcctttcaaatatcgcgtttgagggggtttcctctaccacctacatcac

SEQ ID NO: 728 - DNA, 53 bp, Artificial Sequence
  Staple strand_0727 of the 2D DNA nanostructure.
aatggtcaacaggcaaggcaaagagtaatgtgttcctctaccacctacatcac

SEQ ID NO: 729 - DNA, 52 bp, Artificial Sequence
  Staple strand_0728 of the 2D DNA nanostructure.
tctaaagttttgtcgtctttccagccgacaattcctctaccacctacatcac

SEQ ID NO: 730 - DNA, 65 bp, Artificial Sequence
  Staple strand_0729 of the 2D DNA nanostructure.
tgacaactcgctgaggcttgcattataccaagcgcgatgataaattcctctaccacctacatcac

SEQ ID NO: 731 - DNA, 53 bp, Artificial Sequence
  Staple strand_0730 of the 2D DNA nanostructure.
atattcggaaccatcgcccacgcagagaaggattcctctaccacctacatcac

SEQ ID NO: 732 - DNA, 53 bp, Artificial Sequence
  Staple strand_0731 of the 2D DNA nanostructure.
tcatcgccaacaaagtacaacggacgccagcattcctctaccacctacatcac

SEQ ID NO: 733 - DNA, 53 bp, Artificial Sequence
  Staple strand_0732 of the 2D DNA nanostructure.
gatggtttgaacgagtagtaaatttaccattattcctctaccacctacatcac

SEQ ID NO: 734 - DNA, 59 bp, Artificial Sequence
  Staple strand_0733 of the 2D DNA nanostructure.
cgtttaccagacgacaaagaagttttgccataattcgattcctctaccacctacatcac

SEQ ID NO: 735 - DNA, 53 bp, Artificial Sequence
  Staple strand_0734 of the 2D DNA nanostructure.
cttttgcagataaaaaccaaaataaagactccttcctctaccacctacatcac

SEQ ID NO: 736 - DNA, 51 bp, Artificial Sequence
  Staple strand_0735 of the 2D DNA nanostructure.
gcttcaatcaggattagagagttattttcattcctctaccacctacatcac

SEQ ID NO: 737 - DNA, 53 bp, Artificial Sequence
  Staple strand_0736 of the 2D DNA nanostructure.
ccaacaggagcgaaccagaccggagcctttacttcctctaccacctacatcac

SEQ ID NO: 738 - DNA, 51 bp, Artificial Sequence
  Staple strand_0737 of the 2D DNA nanostructure.
tttggggatagtagtagcattaaaaggccgttcctctaccacctacatcac

SEQ ID NO: 739 - DNA, 53 bp, Artificial Sequence
  Staple strand_0738 of the 2D DNA nanostructure.
tccacagacagccctcatagttagcgtaacgattcctctaccacctacatcac

SEQ ID NO: 740 - DNA, 53 bp, Artificial Sequence
  Staple strand_0739 of the 2D DNA nanostructure.
ttaggattggctgagactcctcaataaccgatttcctctaccacctacatcac

SEQ ID NO: 741 - DNA, 51 bp, Artificial Sequence
  Staple strand_0740 of the 2D DNA nanostructure.
tattaagaagcggggttttgctcgtagcatttcctctaccacctacatcac

SEQ ID NO: 742 - DNA, 53 bp, Artificial Sequence
  Staple strand_0741 of the 2D DNA nanostructure.
ttgacaggccaccaccagagccgcgatttgtattcctctaccacctacatcac

SEQ ID NO: 743 - DNA, 51 bp, Artificial Sequence
  Staple strand_0742 of the 2D DNA nanostructure.
caccagaaaggttgaggcaggtcatgaaagttcctctaccacctacatcac

SEQ ID NO: 744 - DNA, 53 bp, Artificial Sequence
  Staple strand_0743 of the 2D DNA nanostructure.
gcaaggcctcaccagtagcaccatgggcttgattcctctaccacctacatcac

SEQ ID NO: 745 - DNA, 51 bp, Artificial Sequence
  Staple strand_0744 of the 2D DNA nanostructure.
cagcaaaaggaaacgtcaccaatgagccgcttcctctaccacctacatcac

SEQ ID NO: 746 - DNA, 53 bp, Artificial Sequence
  Staple strand_0745 of the 2D DNA nanostructure.
ttattacgaagaactggcatgattgcgagaggttcctctaccacctacatcac

SEQ ID NO: 747 - DNA, 51 bp, Artificial Sequence
  Staple strand_0746 of the 2D DNA nanostructure.
atacccaacagtatgttagcaaattagagcttcctctaccacctacatcac

SEQ ID NO: 748 - DNA, 53 bp, Artificial Sequence
  Staple strand_0747 of the 2D DNA nanostructure.
agagagaaaaaaatgaaaatagcaagcaaactttcctctaccacctacatcac

SEQ ID NO: 749 - DNA, 51 bp, Artificial Sequence
  Staple strand_0748 of the 2D DNA nanostructure.
ttaacgtctaacataaaaacaggtaacggattcctctaccacctacatcac

SEQ ID NO: 750 - DNA, 53 bp, Artificial Sequence
  Staple strand_0749 of the 2D DNA nanostructure.
ccaatagctcatcgtaggaatcatggcatcaattcctctaccacctacatcac

SEQ ID NO: 751 - DNA, 53 bp, Artificial Sequence
  Staple strand_0750 of the 2D DNA nanostructure.
tcaccagtacaaactacaacgcctagtaccagttcctctaccacctacatcac

SEQ ID NO: 752 - DNA, 53 bp, Artificial Sequence
  Staple strand_0751 of the 2D DNA nanostructure.
gcggataacctattattctgaaacagacgattttcctctaccacctacatcac

SEQ ID NO: 753 - DNA, 53 bp, Artificial Sequence
  Staple strand_0752 of the 2D DNA nanostructure.
tttcggaagtgccgtcgagagggtgagtttcgttcctctaccacctacatcac

SEQ ID NO: 754 - DNA, 53 bp, Artificial Sequence
  Staple strand_0753 of the 2D DNA nanostructure.
ggccttgaagagccaccaccctcagaaaccatttcctctaccacctacatcac

SEQ ID NO: 755 - DNA, 53 bp, Artificial Sequence
  Staple strand_0754 of the 2D DNA nanostructure.
ccaccctctattcacaaacaaatacctgcctattcctctaccacctacatcac

SEQ ID NO: 756 - DNA, 53 bp, Artificial Sequence
  Staple strand_0755 of the 2D DNA nanostructure.
cgatagcattgagccatttgggaacgtagaaattcctctaccacctacatcac

SEQ ID NO: 757 - DNA, 53 bp, Artificial Sequence
  Staple strand_0756 of the 2D DNA nanostructure.
tcaccgacgcaccgtaatcagtagcagaaccgttcctctaccacctacatcac

SEQ ID NO: 758 - DNA, 53 bp, Artificial Sequence
  Staple strand_0757 of the 2D DNA nanostructure.
atacataccgaggaaacgcaataagaagcgcattcctctaccacctacatcac

SEQ ID NO: 759 - DNA, 53 bp, Artificial Sequence
  Staple strand_0758 of the 2D DNA nanostructure.
aaggaaacataaaggtggcaacattatcaccgttcctctaccacctacatcac

SEQ ID NO: 760 - DNA, 53 bp, Artificial Sequence
  Staple strand_0759 of the 2D DNA nanostructure.
ttagacggccaaataagaaacgatagaaggctttcctctaccacctacatcac

SEQ ID NO: 761 - DNA, 53 bp, Artificial Sequence
  Staple strand_0760 of the 2D DNA nanostructure.
atcccaatgagaattaactgaacagttaccagttcctctaccacctacatcac

SEQ ID NO: 762 - DNA, 53 bp, Artificial Sequence
  Staple strand_0761 of the 2D DNA nanostructure.
tatccggtctcatcgagaacaagcgacaaaagttcctctaccacctacatcac

SEQ ID NO: 763 - DNA, 52 bp, Artificial Sequence
  Staple strand_0762 of the 2D DNA nanostructure.
aggaacccatgtaccgtaacacttgatataattcctctaccacctacatcac

SEQ ID NO: 764 - DNA, 51 bp, Artificial Sequence
  Staple strand_0763 of the 2D DNA nanostructure.
gtatagcaaacagttaatgcccaatcctcattcctctaccacctacatcac

SEQ ID NO: 765 - DNA, 53 bp, Artificial Sequence
  Staple strand_0764 of the 2D DNA nanostructure.
gcccgtatccggaataggtgtatcagcccaatttcctctaccacctacatcac

SEQ ID NO: 766 - DNA, 51 bp, Artificial Sequence
  Staple strand_0765 of the 2D DNA nanostructure.
ttaaagccagagccgccaccctcgacagaattcctctaccacctacatcac

SEQ ID NO: 767 - DNA, 53 bp, Artificial Sequence
  Staple strand_0766 of the 2D DNA nanostructure.
gcctccctcagaatggaaagcgcagtaacagtttcctctaccacctacatcac

SEQ ID NO: 768 - DNA, 51 bp, Artificial Sequence
  Staple strand_0767 of the 2D DNA nanostructure.
tcaagtttcattaaaggtgaatataaaagattcctctaccacctacatcac

SEQ ID NO: 769 - DNA, 53 bp, Artificial Sequence
  Staple strand_0768 of the 2D DNA nanostructure.
gaaattattgcctttagcgtcagaccggaaccttcctctaccacctacatcac

SEQ ID NO: 770 - DNA, 51 bp, Artificial Sequence
  Staple strand_0769 of the 2D DNA nanostructure.
aacgcaaagatagccgaacaaaccctgaacttcctctaccacctacatcac

SEQ ID NO: 771 - DNA, 53 bp, Artificial Sequence
  Staple strand_0770 of the 2D DNA nanostructure.
aagtaagcagacaccacggaataatattgacgttcctctaccacctacatcac

SEQ ID NO: 772 - DNA, 51 bp, Artificial Sequence
  Staple strand_0771 of the 2D DNA nanostructure.
aaagtcacaaaataaacagccagcgttttattcctctaccacctacatcac

SEQ ID NO: 773 - DNA, 53 bp, Artificial Sequence
  Staple strand_0772 of the 2D DNA nanostructure.
gccagttagagggtaattgagcgctttaagaattcctctaccacctacatcac

SEQ ID NO: 774 - DNA, 51 bp, Artificial Sequence
  Staple strand_0773 of the 2D DNA nanostructure.
gcgaacctccaagaacgggtatgacaataattcctctaccacctacatcac

SEQ ID NO: 775 - DNA, 53 bp, Artificial Sequence
  Staple strand_0774 of the 2D DNA nanostructure.
ccaccctcattttcagggatagcaaccgtactttcctctaccacctacatcac

SEQ ID NO: 776 - DNA, 53 bp, Artificial Sequence
  Staple strand_0775 of the 2D DNA nanostructure.
caggaggtggggtcagtgccttgagtctctgattcctctaccacctacatcac

SEQ ID NO: 777 - DNA, 51 bp, Artificial Sequence
  Staple strand_0776 of the 2D DNA nanostructure.
gttttaacttagtaccgccacccagagccattcctctaccacctacatcac

SEQ ID NO: 778 - DNA, 53 bp, Artificial Sequence
  Staple strand_0777 of the 2D DNA nanostructure.
atttaccgggaaccagagccaccactgtagcgttcctctaccacctacatcac

SEQ ID NO: 779 - DNA, 51 bp, Artificial Sequence
  Staple strand_0778 of the 2D DNA nanostructure.
aaatcaccttccagtaagcgtcagtaataattcctctaccacctacatcac

SEQ ID NO: 780 - DNA, 53 bp, Artificial Sequence
  Staple strand_0779 of the 2D DNA nanostructure.
cgttttcaagggagggaaggtaaagtttatttttcctctaccacctacatcac

SEQ ID NO: 781 - DNA, 51 bp, Artificial Sequence
  Staple strand_0780 of the 2D DNA nanostructure.
accgattgtcggcattttcggtcataatcattcctctaccacctacatcac

SEQ ID NO: 782 - DNA, 53 bp, Artificial Sequence
  Staple strand_0781 of the 2D DNA nanostructure.
tgtcacaatcttaccgaagccctttaatatcattcctctaccacctacatcac

SEQ ID NO: 783 - DNA, 51 bp, Artificial Sequence
  Staple strand_0782 of the 2D DNA nanostructure.
aatagctatcaatagaaaattcaacattcattcctctaccacctacatcac

SEQ ID NO: 784 - DNA, 53 bp, Artificial Sequence
  Staple strand_0783 of the 2D DNA nanostructure.
gagagatagagcgtctttccagaggttttgaattcctctaccacctacatcac

SEQ ID NO: 785 - DNA, 51 bp, Artificial Sequence
  Staple strand_0784 of the 2D DNA nanostructure.
acgctaacacccacaagaattgaaaatagcttcctctaccacctacatcac

SEQ ID NO: 786 - DNA, 53 bp, Artificial Sequence
  Staple strand_0785 of the 2D DNA nanostructure.
gccttaaaccaatcaataatcggcacgcgcctttcctctaccacctacatcac

SEQ ID NO: 787 - DNA, 53 bp, Artificial Sequence
  Staple strand_0786 of the 2D DNA nanostructure.
taaatcgggattcccaattctgcgatataatgttcctctaccacctacatcac

SEQ ID NO: 788 - DNA, 51 bp, Artificial Sequence
  Staple strand_0787 of the 2D DNA nanostructure.
aacgcaaaatcgatgaacggtaccggttgattcctctaccacctacatcac

SEQ ID NO: 789 - DNA, 53 bp, Artificial Sequence
  Staple strand_0788 of the 2D DNA nanostructure.
aacaagagggataaaaatttttagcataaagcttcctctaccacctacatcac

SEQ ID NO: 790 - DNA, 51 bp, Artificial Sequence
  Staple strand_0789 of the 2D DNA nanostructure.
taatcagcggattgaccgtaatcgtaaccgttcctctaccacctacatcac

SEQ ID NO: 791 - DNA, 53 bp, Artificial Sequence
  Staple strand_0790 of the 2D DNA nanostructure.
acaaacggaaaagccccaaaaacactggagcattcctctaccacctacatcac

SEQ ID NO: 792 - DNA, 51 bp, Artificial Sequence
  Staple strand_0791 of the 2D DNA nanostructure.
tgcatctttcccagtcacgacggcctgcagttcctctaccacctacatcac

SEQ ID NO: 793 - DNA, 53 bp, Artificial Sequence
  Staple strand_0792 of the 2D DNA nanostructure.
ccagggttgccagtttgaggggacccgtgggattcctctaccacctacatcac

SEQ ID NO: 794 - DNA, 51 bp, Artificial Sequence
  Staple strand_0793 of the 2D DNA nanostructure.
gtcgacttcggccaacgcgcggggtttttcttcctctaccacctacatcac

SEQ ID NO: 795 - DNA, 53 bp, Artificial Sequence
  Staple strand_0794 of the 2D DNA nanostructure.
ttaatgaactagaggatccccggggggtaacgttcctctaccacctacatcac

SEQ ID NO: 796 - DNA, 51 bp, Artificial Sequence
  Staple strand_0795 of the 2D DNA nanostructure.
ttttcactcaaagggcgaaaaaccatcaccttcctctaccacctacatcac

SEQ ID NO: 797 - DNA, 53 bp, Artificial Sequence
  Staple strand_0796 of the 2D DNA nanostructure.
ctccaacgcagtgagacgggcaaccagctgcattcctctaccacctacatcac

SEQ ID NO: 798 - DNA, 52 bp, Artificial Sequence
  Staple strand_0797 of the 2D DNA nanostructure.
caaatcaagttttttggggtcgaaacgtggattcctctaccacctacatcac

SEQ ID NO: 799 - DNA, 51 bp, Artificial Sequence
  Staple strand_0798 of the 2D DNA nanostructure.
aaattaagttgaccattagatacttttgcgttcctctaccacctacatcac

SEQ ID NO: 800 - DNA, 53 bp, Artificial Sequence
  Staple strand_0799 of the 2D DNA nanostructure.
tatattttgtcattgcctgagagtggaagattttcctctaccacctacatcac

SEQ ID NO: 801 - DNA, 51 bp, Artificial Sequence
  Staple strand_0800 of the 2D DNA nanostructure.
gctatcagaaatgcaatgcctgaattagcattcctctaccacctacatcac

SEQ ID NO: 802 - DNA, 53 bp, Artificial Sequence
  Staple strand_0801 of the 2D DNA nanostructure.
gtataagccaacccgtcggattctgacgacagttcctctaccacctacatcac

SEQ ID NO: 803 - DNA, 51 bp, Artificial Sequence
  Staple strand_0802 of the 2D DNA nanostructure.
gcgagtaaaaatatttaaattgttacaaagttcctctaccacctacatcac

SEQ ID NO: 804 - DNA, 53 bp, Artificial Sequence
  Staple strand_0803 of the 2D DNA nanostructure.
tatcggccgcaaggcgattaagtttaccgagcttcctctaccacctacatcac

SEQ ID NO: 805 - DNA, 51 bp, Artificial Sequence
  Staple strand_0804 of the 2D DNA nanostructure.
gatgtgcttcaggaagatcgcacaatgtgattcctctaccacctacatcac

SEQ ID NO: 806 - DNA, 53 bp, Artificial Sequence
  Staple strand_0805 of the 2D DNA nanostructure.
tcgaattcgggaaacctgtcgtgcagctgattttcctctaccacctacatcac

SEQ ID NO: 807 - DNA, 51 bp, Artificial Sequence
  Staple strand_0806 of the 2D DNA nanostructure.
ttccagtcgtaatcatggtcataaaaggggttcctctaccacctacatcac

SEQ ID NO: 808 - DNA, 53 bp, Artificial Sequence
  Staple strand_0807 of the 2D DNA nanostructure.
gcccttcagagtccactattaaagggtgccgtttcctctaccacctacatcac

SEQ ID NO: 809 - DNA, 51 bp, Artificial Sequence
  Staple strand_0808 of the 2D DNA nanostructure.
tggaacaaccgcctggccctgaggcccgctttcctctaccacctacatcac

SEQ ID NO: 810 - DNA, 51 bp, Artificial Sequence
  Staple strand_0809 of the 2D DNA nanostructure.
aaagcactaaatcggaaccctaatccagttttcctctaccacctacatcac

SEQ ID NO: 811 - DNA, 53 bp, Artificial Sequence
  Staple strand_0810 of the 2D DNA nanostructure.
taaatcatataacctgtttagctaacctttaattcctctaccacctacatcac

SEQ ID NO: 812 - DNA, 53 bp, Artificial Sequence
  Staple strand_0811 of the 2D DNA nanostructure.
taggtaaactatttttgagagatcaaacgttattcctctaccacctacatcac

SEQ ID NO: 813 - DNA, 53 bp, Artificial Sequence
  Staple strand_0812 of the 2D DNA nanostructure.
gagggtaggattcaaaagggtgagacatccaattcctctaccacctacatcac

SEQ ID NO: 814 - DNA, 53 bp, Artificial Sequence
  Staple strand_0813 of the 2D DNA nanostructure.
atattttggctttcatcaacattatccagccattcctctaccacctacatcac

SEQ ID NO: 815 - DNA, 53 bp, Artificial Sequence
  Staple strand_0814 of the 2D DNA nanostructure.
tgtagccattaaaattcgcattaaatgccggattcctctaccacctacatcac

SEQ ID NO: 816 - DNA, 53 bp, Artificial Sequence
  Staple strand_0815 of the 2D DNA nanostructure.
gctttccgattacgccagctggcggctgtttcttcctctaccacctacatcac

SEQ ID NO: 817 - DNA, 53 bp, Artificial Sequence
  Staple strand_0816 of the 2D DNA nanostructure.
tcttcgctgcaccgcttctggtgcggccttccttcctctaccacctacatcac

SEQ ID NO: 818 - DNA, 53 bp, Artificial Sequence
  Staple strand_0817 of the 2D DNA nanostructure.
ctgtgtgattgcgttgcgctcactagagttgcttcctctaccacctacatcac

SEQ ID NO: 819 - DNA, 53 bp, Artificial Sequence
  Staple strand_0818 of the 2D DNA nanostructure.
cacattaaaattgttatccgctcatgcgggccttcctctaccacctacatcac

SEQ ID NO: 820 - DNA, 53 bp, Artificial Sequence
  Staple strand_0819 of the 2D DNA nanostructure.
agcaagcgtagggttgagtgttgtagggagccttcctctaccacctacatcac

SEQ ID NO: 821 - DNA, 53 bp, Artificial Sequence
  Staple strand_0820 of the 2D DNA nanostructure.
gcccgagagtccacgctggtttgcagctaactttcctctaccacctacatcac

SEQ ID NO: 822 - DNA, 53 bp, Artificial Sequence
  Staple strand_0821 of the 2D DNA nanostructure.
cccgatttagagcttgacggggaaaaagaatattcctctaccacctacatcac

SEQ ID NO: 823 - DNA, 53 bp, Artificial Sequence
  Staple strand_0822 of the 2D DNA nanostructure.
ttctactacgcgagctgaaaaggttaccgcgcttcctctaccacctacatcac

SEQ ID NO: 824 - DNA, 51 bp, Artificial Sequence
  Staple strand_0823 of the 2D DNA nanostructure.
gagacagctagctgataaattaatttttgtttcctctaccacctacatcac

SEQ ID NO: 825 - DNA, 53 bp, Artificial Sequence
  Staple strand_0824 of the 2D DNA nanostructure.
caaccgtttcaaatcaccatcaattcgagccattcctctaccacctacatcac

SEQ ID NO: 826 - DNA, 65 bp, Artificial Sequence
  Staple strand_0825 of the 2D DNA nanostructure.
taaatcaaaataattcgcgtctcggaaaccaggcaaagggaaggttcctctaccacctacatcac

SEQ ID NO: 827 - DNA, 53 bp, Artificial Sequence
  Staple strand_0826 of the 2D DNA nanostructure.
gccatcaagctcattttttaaccacaaatccattcctctaccacctacatcac

SEQ ID NO: 828 - DNA, 53 bp, Artificial Sequence
  Staple strand_0827 of the 2D DNA nanostructure.
caactgttgcgccattcgccattcaaacatcattcctctaccacctacatcac

SEQ ID NO: 829 - DNA, 53 bp, Artificial Sequence
  Staple strand_0828 of the 2D DNA nanostructure.
aagcctggtacgagccggaagcatagatgatgttcctctaccacctacatcac

SEQ ID NO: 830 - DNA, 59 bp, Artificial Sequence
  Staple strand_0829 of the 2D DNA nanostructure.
cccagcaggcgaaaaatcccttataaatcaagccggcgttcctctaccacctacatcac

SEQ ID NO: 831 - DNA, 53 bp, Artificial Sequence
  Staple strand_0830 of the 2D DNA nanostructure.
tcggcaaatcctgtttgatggtggaccctcaattcctctaccacctacatcac

SEQ ID NO: 832 - DNA, 52 bp, Artificial Sequence
  Staple strand_0831 of the 2D DNA nanostructure.
aacgtggcgagaaaggaagggaaaccagtaattcctctaccacctacatcac

SEQ ID NO: 833 - DNA, 51 bp, Artificial Sequence
  Staple strand_0832 of the 2D DNA nanostructure.
ttttatttaagcaaatcagatattttttgtttcctctaccacctacatcac

SEQ ID NO: 834 - DNA, 53 bp, Artificial Sequence
  Staple strand_0833 of the 2D DNA nanostructure.
gtaataagttaggcagaggcatttatgatattttcctctaccacctacatcac

SEQ ID NO: 835 - DNA, 51 bp, Artificial Sequence
  Staple strand_0834 of the 2D DNA nanostructure.
catgtaatagaatataaagtaccaagccgtttcctctaccacctacatcac

SEQ ID NO: 836 - DNA, 53 bp, Artificial Sequence
  Staple strand_0835 of the 2D DNA nanostructure.
atcgcaagtatgtaaatgctgatgataggaacttcctctaccacctacatcac

SEQ ID NO: 837 - DNA, 51 bp, Artificial Sequence
  Staple strand_0836 of the 2D DNA nanostructure.
tataactaacaaagaacgcgagaacgccaattcctctaccacctacatcac

SEQ ID NO: 838 - DNA, 53 bp, Artificial Sequence
  Staple strand_0837 of the 2D DNA nanostructure.
agaaaacaaagaagatgatgaaacaggctgcgttcctctaccacctacatcac

SEQ ID NO: 839 - DNA, 51 bp, Artificial Sequence
  Staple strand_0838 of the 2D DNA nanostructure.
ctgagcaaaaattaattacattttgggttattcctctaccacctacatcac

SEQ ID NO: 840 - DNA, 53 bp, Artificial Sequence
  Staple strand_0839 of the 2D DNA nanostructure.
gcaattcacatattcctgattatcaaagtgtattcctctaccacctacatcac

SEQ ID NO: 841 - DNA, 51 bp, Artificial Sequence
  Staple strand_0840 of the 2D DNA nanostructure.
attatcattcaatataatcctgacaattacttcctctaccacctacatcac

SEQ ID NO: 842 - DNA, 53 bp, Artificial Sequence
  Staple strand_0841 of the 2D DNA nanostructure.
tcaatatcgaacctcaaatatcaattccgaaattcctctaccacctacatcac

SEQ ID NO: 843 - DNA, 51 bp, Artificial Sequence
  Staple strand_0842 of the 2D DNA nanostructure.
accttgcttggtcagttggcaaagagcggattcctctaccacctacatcac

SEQ ID NO: 844 - DNA, 51 bp, Artificial Sequence
  Staple strand_0843 of the 2D DNA nanostructure.
taaaagggacattctggccaacaaagcatcttcctctaccacctacatcac

SEQ ID NO: 845 - DNA, 53 bp, Artificial Sequence
  Staple strand_0844 of the 2D DNA nanostructure.
gtaccgcaattctaagaacgcgagtattatttttcctctaccacctacatcac

SEQ ID NO: 846 - DNA, 53 bp, Artificial Sequence
  Staple strand_0845 of the 2D DNA nanostructure.
gtaaagtaatcgccatatttaacaaaacttttttcctctaccacctacatcac

SEQ ID NO: 847 - DNA, 53 bp, Artificial Sequence
  Staple strand_0846 of the 2D DNA nanostructure.
aattgagaattctgtccagacgactaaaccaattcctctaccacctacatcac

SEQ ID NO: 848 - DNA, 53 bp, Artificial Sequence
  Staple strand_0847 of the 2D DNA nanostructure.
tcaaatataacctccggcttaggtaacaatttttcctctaccacctacatcac

SEQ ID NO: 849 - DNA, 53 bp, Artificial Sequence
  Staple strand_0848 of the 2D DNA nanostructure.
acctttttattttagttaatttcatagggcttttcctctaccacctacatcac

SEQ ID NO: 850 - DNA, 53 bp, Artificial Sequence
  Staple strand_0849 of the 2D DNA nanostructure.
catttgaaggcgaattattcatttttgtttggttcctctaccacctacatcac

SEQ ID NO: 851 - DNA, 53 bp, Artificial Sequence
  Staple strand_0850 of the 2D DNA nanostructure.
cgcgcagattaccttttttaatgggagagactttcctctaccacctacatcac

SEQ ID NO: 852 - DNA, 53 bp, Artificial Sequence
  Staple strand_0851 of the 2D DNA nanostructure.
attatactaagaaaccaccagaagtcaacagtttcctctaccacctacatcac

SEQ ID NO: 853 - DNA, 53 bp, Artificial Sequence
  Staple strand_0852 of the 2D DNA nanostructure.
gcggaacatctgaataatggaaggtacaaaatttcctctaccacctacatcac

SEQ ID NO: 854 - DNA, 53 bp, Artificial Sequence
  Staple strand_0853 of the 2D DNA nanostructure.
tgaaaggagcaaatgaaaaatctagagatagattcctctaccacctacatcac

SEQ ID NO: 855 - DNA, 53 bp, Artificial Sequence
  Staple strand_0854 of the 2D DNA nanostructure.
agccagcaattgaggaaggttatcatcattttttcctctaccacctacatcac

SEQ ID NO: 856 - DNA, 53 bp, Artificial Sequence
  Staple strand_0855 of the 2D DNA nanostructure.
acccttctgacctgaaagcgtaagacgctgagttcctctaccacctacatcac

SEQ ID NO: 857 - DNA, 53 bp, Artificial Sequence
  Staple strand_0856 of the 2D DNA nanostructure.
cttatcattcccgacttgcgggagcctaatttttcctctaccacctacatcac

SEQ ID NO: 858 - DNA, 51 bp, Artificial Sequence
  Staple strand_0857 of the 2D DNA nanostructure.
acaacatgccaacgctcaacagtcttctgattcctctaccacctacatcac

SEQ ID NO: 859 - DNA, 53 bp, Artificial Sequence
  Staple strand_0858 of the 2D DNA nanostructure.
agtataaagttcagctaatgcagatgtctttcttcctctaccacctacatcac

SEQ ID NO: 860 - DNA, 51 bp, Artificial Sequence
  Staple strand_0859 of the 2D DNA nanostructure.
cctaaatcaaaatcataggtctaaacagtattcctctaccacctacatcac

SEQ ID NO: 861 - DNA, 53 bp, Artificial Sequence
  Staple strand_0860 of the 2D DNA nanostructure.
gaatttatttaatggtttgaaatattcttaccttcctctaccacctacatcac

SEQ ID NO: 862 - DNA, 51 bp, Artificial Sequence
  Staple strand_0861 of the 2D DNA nanostructure.
cataaatctttgaataccaagtgttagaacttcctctaccacctacatcac

SEQ ID NO: 863 - DNA, 53 bp, Artificial Sequence
  Staple strand_0862 of the 2D DNA nanostructure.
cctgattgcaatatatgtgagtgatcaatagtttcctctaccacctacatcac

SEQ ID NO: 864 - DNA, 51 bp, Artificial Sequence
  Staple strand_0863 of the 2D DNA nanostructure.
ctaccatagtttgagtaacatttaaaatatttcctctaccacctacatcac

SEQ ID NO: 865 - DNA, 53 bp, Artificial Sequence
  Staple strand_0864 of the 2D DNA nanostructure.
attttaaaatcaaaattatttgcacggattcgttcctctaccacctacatcac

SEQ ID NO: 866 - DNA, 51 bp, Artificial Sequence
  Staple strand_0865 of the 2D DNA nanostructure.
ctttagggcctgcaacagtgccaatacgtgttcctctaccacctacatcac

SEQ ID NO: 867 - DNA, 53 bp, Artificial Sequence
  Staple strand_0866 of the 2D DNA nanostructure.
ttaacaccagcactaacaactaatcgttattattcctctaccacctacatcac

SEQ ID NO: 868 - DNA, 52 bp, Artificial Sequence
  Staple strand_0867 of the 2D DNA nanostructure.
gcacagacaatatttttgaatggggtcagtattcctctaccacctacatcac

SEQ ID NO: 869 - DNA, 51 bp, Artificial Sequence
  Staple strand_0868 of the 2D DNA nanostructure.
tgtagaaatcaagattagttgctcttaccattcctctaccacctacatcac

SEQ ID NO: 870 - DNA, 53 bp, Artificial Sequence
  Staple strand_0869 of the 2D DNA nanostructure.
gtttatcaatatgcgttatacaaaccgaccgtttcctctaccacctacatcac

SEQ ID NO: 871 - DNA, 51 bp, Artificial Sequence
  Staple strand_0870 of the 2D DNA nanostructure.
ttagtatcacaatagataagtccacgagcattcctctaccacctacatcac

SEQ ID NO: 872 - DNA, 53 bp, Artificial Sequence
  Staple strand_0871 of the 2D DNA nanostructure.
gtgataaaaagacgctgagaagagataaccttttcctctaccacctacatcac

SEQ ID NO: 873 - DNA, 51 bp, Artificial Sequence
  Staple strand_0872 of the 2D DNA nanostructure.
cttagatttaaggcgttaaataaagcctgtttcctctaccacctacatcac

SEQ ID NO: 874 - DNA, 53 bp, Artificial Sequence
  Staple strand_0873 of the 2D DNA nanostructure.
gcttctgttcgggagaaacaataacgtaaaacttcctctaccacctacatcac

SEQ ID NO: 875 - DNA, 51 bp, Artificial Sequence
  Staple strand_0874 of the 2D DNA nanostructure.
cttttacaaaatcgtcgctattagcgatagttcctctaccacctacatcac

SEQ ID NO: 876 - DNA, 53 bp, Artificial Sequence
  Staple strand_0875 of the 2D DNA nanostructure.
agaaataaaaatcctttgcccgaaagattagattcctctaccacctacatcac

SEQ ID NO: 877 - DNA, 51 bp, Artificial Sequence
  Staple strand_0876 of the 2D DNA nanostructure.
ctcgtattagaaattgcgtagatacagtacttcctctaccacctacatcac

SEQ ID NO: 878 - DNA, 53 bp, Artificial Sequence
  Staple strand_0877 of the 2D DNA nanostructure.
gccgtcaaaaaacagaggtgaggcctattagtttcctctaccacctacatcac

SEQ ID NO: 879 - DNA, 51 bp, Artificial Sequence
  Staple strand_0878 of the 2D DNA nanostructure.
cagaagattagataatacatttgtcgacaattcctctaccacctacatcac

SEQ ID NO: 880 - DNA, 51 bp, Artificial Sequence
  Staple strand_0879 of the 2D DNA nanostructure.
ctttaatgcgcgaactgatagccccaccagttcctctaccacctacatcac

SEQ ID NO: 881 - DNA, 52 bp, Artificial Sequence
  Staple strand_0880 of the 2D DNA nanostructure.
agaaaggaacaactaaaggaattcaaaaaaataacattcctaacttctcata

SEQ ID NO: 882 - DNA, 51 bp, Artificial Sequence
  Staple strand_0881 of the 2D DNA nanostructure.
aggctccagaggctttgaggacacgggtaataacattcctaacttctcata

SEQ ID NO: 883 - DNA, 53 bp, Artificial Sequence
  Staple strand_0882 of the 2D DNA nanostructure.
acggctacaaaaggagcctttaatgtgagaattaacattcctaacttctcata

SEQ ID NO: 884 - DNA, 51 bp, Artificial Sequence
  Staple strand_0883 of the 2D DNA nanostructure.
aatacgtttgaaagaggacagactgacctttaacattcctaacttctcata

SEQ ID NO: 885 - DNA, 53 bp, Artificial Sequence
  Staple strand_0884 of the 2D DNA nanostructure.
gaccaactaatgccactacgaagggggtagcataacattcctaacttctcata

SEQ ID NO: 886 - DNA, 51 bp, Artificial Sequence
  Staple strand_0885 of the 2D DNA nanostructure.
catcaagtaaaacgaactaacgagttgagataacattcctaacttctcata

SEQ ID NO: 887 - DNA, 53 bp, Artificial Sequence
  Staple strand_0886 of the 2D DNA nanostructure.
tacgttaaagtaatcttgacaagaaccgaacttaacattcctaacttctcata

SEQ ID NO: 888 - DNA, 51 bp, Artificial Sequence
  Staple strand_0887 of the 2D DNA nanostructure.
tttaggacaaatgctttaaacaatcaggtctaacattcctaacttctcata

SEQ ID NO: 889 - DNA, 53 bp, Artificial Sequence
  Staple strand_0888 of the 2D DNA nanostructure.
atccccctataccacattcaactagaaaaatctaacattcctaacttctcata

SEQ ID NO: 890 - DNA, 51 bp, Artificial Sequence
  Staple strand_0889 of the 2D DNA nanostructure.
tttaccccaacatgttttaaatttccatattaacattcctaacttctcata

SEQ ID NO: 891 - DNA, 53 bp, Artificial Sequence
  Staple strand_0890 of the 2D DNA nanostructure.
ctgtagcttgactattatagtcagttcattgataacattcctaacttctcata

SEQ ID NO: 892 - DNA, 51 bp, Artificial Sequence
  Staple strand_0891 of the 2D DNA nanostructure.
aacagttttgtaccaaaaacattttatttctaacattcctaacttctcata

SEQ ID NO: 893 - DNA, 53 bp, Artificial Sequence
  Staple strand_0892 of the 2D DNA nanostructure.
acaactttcaacagtttcagcggatgtatcggtaacattcctaacttctcata

SEQ ID NO: 894 - DNA, 53 bp, Artificial Sequence
  Staple strand_0893 of the 2D DNA nanostructure.
tttatcaggacagcatcggaacgacaccaacctaacattcctaacttctcata

SEQ ID NO: 895 - DNA, 51 bp, Artificial Sequence
  Staple strand_0894 of the 2D DNA nanostructure.
cagcgaaacttgctttcgaggtgttgctaataacattcctaacttctcata

SEQ ID NO: 896 - DNA, 53 bp, Artificial Sequence
  Staple strand_0895 of the 2D DNA nanostructure.
taaaacgaggtcaatcataagggaaccggatataacattcctaacttctcata

SEQ ID NO: 897 - DNA, 51 bp, Artificial Sequence
  Staple strand_0896 of the 2D DNA nanostructure.
gcgcagacaagaggcaaaagaatccctcagtaacattcctaacttctcata

SEQ ID NO: 898 - DNA, 53 bp, Artificial Sequence
  Staple strand_0897 of the 2D DNA nanostructure.
ttcattacgtcaggacgttgggaaatgcagattaacattcctaacttctcata

SEQ ID NO: 899 - DNA, 51 bp, Artificial Sequence
  Staple strand_0898 of the 2D DNA nanostructure.
ttataccaccaaatcaacgtaacgaacgagtaacattcctaacttctcata

SEQ ID NO: 900 - DNA, 53 bp, Artificial Sequence
  Staple strand_0899 of the 2D DNA nanostructure.
acataacgggaatcgtcataaataaagcaaagtaacattcctaacttctcata

SEQ ID NO: 901 - DNA, 51 bp, Artificial Sequence
  Staple strand_0900 of the 2D DNA nanostructure.
aatactgcccaaaaggaattacgtggctcataacattcctaacttctcata

SEQ ID NO: 902 - DNA, 53 bp, Artificial Sequence
  Staple strand_0901 of the 2D DNA nanostructure.
cggattgcagagcttaattgctgaaacgagtataacattcctaacttctcata

SEQ ID NO: 903 - DNA, 51 bp, Artificial Sequence
  Staple strand_0902 of the 2D DNA nanostructure.
gatggcttatcaaaaagattaagagcgtcctaacattcctaacttctcata

SEQ ID NO: 904 - DNA, 53 bp, Artificial Sequence
  Staple strand_0903 of the 2D DNA nanostructure.
gatttagtcaataaagcctcagagaaccctcataacattcctaacttctcata

SEQ ID NO: 905 - DNA, 53 bp, Artificial Sequence
  Staple strand_0904 of the 2D DNA nanostructure.
taaatgaattttctgtatgggattaatttctttaacattcctaacttctcata

SEQ ID NO: 906 - DNA, 53 bp, Artificial Sequence
  Staple strand_0905 of the 2D DNA nanostructure.
aaacagctttttgcgggatcgtcaacactaaataacattcctaacttctcata

SEQ ID NO: 907 - DNA, 53 bp, Artificial Sequence
  Staple strand_0906 of the 2D DNA nanostructure.
aaggccgctgataccgatagttgcgacgttagtaacattcctaacttctcata

SEQ ID NO: 908 - DNA, 53 bp, Artificial Sequence
  Staple strand_0907 of the 2D DNA nanostructure.
acactcatccatgttacttagccgaaagctgctaacattcctaacttctcata

SEQ ID NO: 909 - DNA, 53 bp, Artificial Sequence
  Staple strand_0908 of the 2D DNA nanostructure.
gacctgctctttgacccccagcgagggagttataacattcctaacttctcata

SEQ ID NO: 910 - DNA, 53 bp, Artificial Sequence
  Staple strand_0909 of the 2D DNA nanostructure.
tcattcagatgcgattttaagaacaggcatagtaacattcctaacttctcata

SEQ ID NO: 911 - DNA, 53 bp, Artificial Sequence
  Staple strand_0910 of the 2D DNA nanostructure.
attacctttgaataaggcttgcccaaatccgctaacattcctaacttctcata

SEQ ID NO: 912 - DNA, 53 bp, Artificial Sequence
  Staple strand_0911 of the 2D DNA nanostructure.
taagagcaaatgtttagactggataggaagcctaacattcctaacttctcata

SEQ ID NO: 913 - DNA, 53 bp, Artificial Sequence
  Staple strand_0912 of the 2D DNA nanostructure.
aatagtaaacactatcataaccctcattgtgataacattcctaacttctcata

SEQ ID NO: 914 - DNA, 53 bp, Artificial Sequence
  Staple strand_0913 of the 2D DNA nanostructure.
cgaaagactttgataagaggtcatatttcgcataacattcctaacttctcata

SEQ ID NO: 915 - DNA, 53 bp, Artificial Sequence
  Staple strand_0914 of the 2D DNA nanostructure.
ttgctcctttcaaatatcgcgtttgagggggttaacattcctaacttctcata

SEQ ID NO: 916 - DNA, 53 bp, Artificial Sequence
  Staple strand_0915 of the 2D DNA nanostructure.
aatggtcaacaggcaaggcaaagagtaatgtgtaacattcctaacttctcata

SEQ ID NO: 917 - DNA, 52 bp, Artificial Sequence
  Staple strand_0916 of the 2D DNA nanostructure.
tctaaagttttgtcgtctttccagccgacaataacattcctaacttctcata

SEQ ID NO: 918 - DNA, 65 bp, Artificial Sequence
  Staple strand_0917 of the 2D DNA nanostructure.
tgacaactcgctgaggcttgcattataccaagcgcgatgataaataacattcctaacttctcata

SEQ ID NO: 919 - DNA, 53 bp, Artificial Sequence
  Staple strand_0918 of the 2D DNA nanostructure.
atattcggaaccatcgcccacgcagagaaggataacattcctaacttctcata

SEQ ID NO: 920 - DNA, 53 bp, Artificial Sequence
  Staple strand_0919 of the 2D DNA nanostructure.
tcatcgccaacaaagtacaacggacgccagcataacattcctaacttctcata

SEQ ID NO: 921 - DNA, 53 bp, Artificial Sequence
  Staple strand_0920 of the 2D DNA nanostructure.
gatggtttgaacgagtagtaaatttaccattataacattcctaacttctcata

SEQ ID NO: 922 - DNA, 59 bp, Artificial Sequence
  Staple strand_0921 of the 2D DNA nanostructure.
cgtttaccagacgacaaagaagttttgccataattcgataacattcctaacttctcata

SEQ ID NO: 923 - DNA, 53 bp, Artificial Sequence
  Staple strand_0922 of the 2D DNA nanostructure.
cttttgcagataaaaaccaaaataaagactcctaacattcctaacttctcata

SEQ ID NO: 924 - DNA, 51 bp, Artificial Sequence
  Staple strand_0923 of the 2D DNA nanostructure.
gcttcaatcaggattagagagttattttcataacattcctaacttctcata

SEQ ID NO: 925 - DNA, 53 bp, Artificial Sequence
  Staple strand_0924 of the 2D DNA nanostructure.
ccaacaggagcgaaccagaccggagcctttactaacattcctaacttctcata

SEQ ID NO: 926 - DNA, 51 bp, Artificial Sequence
  Staple strand_0925 of the 2D DNA nanostructure.
tttggggatagtagtagcattaaaaggccgtaacattcctaacttctcata

SEQ ID NO: 927 - DNA, 53 bp, Artificial Sequence
  Staple strand_0926 of the 2D DNA nanostructure.
tccacagacagccctcatagttagcgtaacgataacattcctaacttctcata

SEQ ID NO: 928 - DNA, 53 bp, Artificial Sequence
  Staple strand_0927 of the 2D DNA nanostructure.
ttaggattggctgagactcctcaataaccgattaacattcctaacttctcata

SEQ ID NO: 929 - DNA, 51 bp, Artificial Sequence
  Staple strand_0928 of the 2D DNA nanostructure.
tattaagaagcggggttttgctcgtagcattaacattcctaacttctcata

SEQ ID NO: 930 - DNA, 53 bp, Artificial Sequence
  Staple strand_0929 of the 2D DNA nanostructure.
ttgacaggccaccaccagagccgcgatttgtataacattcctaacttctcata

SEQ ID NO: 931 - DNA, 51 bp, Artificial Sequence
  Staple strand_0930 of the 2D DNA nanostructure.
caccagaaaggttgaggcaggtcatgaaagtaacattcctaacttctcata

SEQ ID NO: 932 - DNA, 53 bp, Artificial Sequence
  Staple strand_0931 of the 2D DNA nanostructure.
gcaaggcctcaccagtagcaccatgggcttgataacattcctaacttctcata

SEQ ID NO: 933 - DNA, 51 bp, Artificial Sequence
  Staple strand_0932 of the 2D DNA nanostructure.
cagcaaaaggaaacgtcaccaatgagccgctaacattcctaacttctcata

SEQ ID NO: 934 - DNA, 53 bp, Artificial Sequence
  Staple strand_0933 of the 2D DNA nanostructure.
ttattacgaagaactggcatgattgcgagaggtaacattcctaacttctcata

SEQ ID NO: 935 - DNA, 51 bp, Artificial Sequence
  Staple strand_0934 of the 2D DNA nanostructure.
atacccaacagtatgttagcaaattagagctaacattcctaacttctcata

SEQ ID NO: 936 - DNA, 53 bp, Artificial Sequence
  Staple strand_0935 of the 2D DNA nanostructure.
agagagaaaaaaatgaaaatagcaagcaaacttaacattcctaacttctcata

SEQ ID NO: 937 - DNA, 51 bp, Artificial Sequence
  Staple strand_0936 of the 2D DNA nanostructure.
ttaacgtctaacataaaaacaggtaacggataacattcctaacttctcata

SEQ ID NO: 938 - DNA, 53 bp, Artificial Sequence
  Staple strand_0937 of the 2D DNA nanostructure.
ccaatagctcatcgtaggaatcatggcatcaataacattcctaacttctcata

SEQ ID NO: 939 - DNA, 53 bp, Artificial Sequence
  Staple strand_0938 of the 2D DNA nanostructure.
tcaccagtacaaactacaacgcctagtaccagtaacattcctaacttctcata

SEQ ID NO: 940 - DNA, 53 bp, Artificial Sequence
  Staple strand_0939 of the 2D DNA nanostructure.
gcggataacctattattctgaaacagacgatttaacattcctaacttctcata

SEQ ID NO: 941 - DNA, 53 bp, Artificial Sequence
  Staple strand_0940 of the 2D DNA nanostructure.
tttcggaagtgccgtcgagagggtgagtttcgtaacattcctaacttctcata

SEQ ID NO: 942 - DNA, 53 bp, Artificial Sequence
  Staple strand_0941 of the 2D DNA nanostructure.
ggccttgaagagccaccaccctcagaaaccattaacattcctaacttctcata

SEQ ID NO: 943 - DNA, 53 bp, Artificial Sequence
  Staple strand_0942 of the 2D DNA nanostructure.
ccaccctctattcacaaacaaatacctgcctataacattcctaacttctcata

SEQ ID NO: 944 - DNA, 53 bp, Artificial Sequence
  Staple strand_0943 of the 2D DNA nanostructure.
cgatagcattgagccatttgggaacgtagaaataacattcctaacttctcata

SEQ ID NO: 945 - DNA, 53 bp, Artificial Sequence
  Staple strand_0944 of the 2D DNA nanostructure.
tcaccgacgcaccgtaatcagtagcagaaccgtaacattcctaacttctcata

SEQ ID NO: 946 - DNA, 53 bp, Artificial Sequence
  Staple strand_0945 of the 2D DNA nanostructure.
atacataccgaggaaacgcaataagaagcgcataacattcctaacttctcata

SEQ ID NO: 947 - DNA, 53 bp, Artificial Sequence
  Staple strand_0946 of the 2D DNA nanostructure.
aaggaaacataaaggtggcaacattatcaccgtaacattcctaacttctcata

SEQ ID NO: 948 - DNA, 53 bp, Artificial Sequence
  Staple strand_0947 of the 2D DNA nanostructure.
ttagacggccaaataagaaacgatagaaggcttaacattcctaacttctcata

SEQ ID NO: 949 - DNA, 53 bp, Artificial Sequence
  Staple strand_0948 of the 2D DNA nanostructure.
atcccaatgagaattaactgaacagttaccagtaacattcctaacttctcata

SEQ ID NO: 950 - DNA, 53 bp, Artificial Sequence
  Staple strand_0949 of the 2D DNA nanostructure.
tatccggtctcatcgagaacaagcgacaaaagtaacattcctaacttctcata

SEQ ID NO: 951 - DNA, 52 bp, Artificial Sequence
  Staple strand_0950 of the 2D DNA nanostructure.
aggaacccatgtaccgtaacacttgatataataacattcctaacttctcata

SEQ ID NO: 952 - DNA, 51 bp, Artificial Sequence
  Staple strand_0951 of the 2D DNA nanostructure.
gtatagcaaacagttaatgcccaatcctcataacattcctaacttctcata

SEQ ID NO: 953 - DNA, 53 bp, Artificial Sequence
  Staple strand_0952 of the 2D DNA nanostructure.
gcccgtatccggaataggtgtatcagcccaattaacattcctaacttctcata

SEQ ID NO: 954 - DNA, 51 bp, Artificial Sequence
  Staple strand_0953 of the 2D DNA nanostructure.
ttaaagccagagccgccaccctcgacagaataacattcctaacttctcata

SEQ ID NO: 955 - DNA, 53 bp, Artificial Sequence
  Staple strand_0954 of the 2D DNA nanostructure.
gcctccctcagaatggaaagcgcagtaacagttaacattcctaacttctcata

SEQ ID NO: 956 - DNA, 51 bp, Artificial Sequence
  Staple strand_0955 of the 2D DNA nanostructure.
tcaagtttcattaaaggtgaatataaaagataacattcctaacttctcata

SEQ ID NO: 957 - DNA, 53 bp, Artificial Sequence
  Staple strand_0956 of the 2D DNA nanostructure.
gaaattattgcctttagcgtcagaccggaacctaacattcctaacttctcata

SEQ ID NO: 958 - DNA, 51 bp, Artificial Sequence
  Staple strand_0957 of the 2D DNA nanostructure.
aacgcaaagatagccgaacaaaccctgaactaacattcctaacttctcata

SEQ ID NO: 959 - DNA, 53 bp, Artificial Sequence
  Staple strand_0958 of the 2D DNA nanostructure.
aagtaagcagacaccacggaataatattgacgtaacattcctaacttctcata

SEQ ID NO: 960 - DNA, 51 bp, Artificial Sequence
  Staple strand_0959 of the 2D DNA nanostructure.
aaagtcacaaaataaacagccagcgttttataacattcctaacttctcata

SEQ ID NO: 961 - DNA, 53 bp, Artificial Sequence
  Staple strand_0960 of the 2D DNA nanostructure.
gccagttagagggtaattgagcgctttaagaataacattcctaacttctcata

SEQ ID NO: 962 - DNA, 51 bp, Artificial Sequence
  Staple strand_0961 of the 2D DNA nanostructure.
gcgaacctccaagaacgggtatgacaataataacattcctaacttctcata

SEQ ID NO: 963 - DNA, 53 bp, Artificial Sequence
  Staple strand_0962 of the 2D DNA nanostructure.
ccaccctcattttcagggatagcaaccgtacttaacattcctaacttctcata

SEQ ID NO: 964 - DNA, 53 bp, Artificial Sequence
  Staple strand_0963 of the 2D DNA nanostructure.
caggaggtggggtcagtgccttgagtctctgataacattcctaacttctcata

SEQ ID NO: 965 - DNA, 51 bp, Artificial Sequence
  Staple strand_0964 of the 2D DNA nanostructure.
gttttaacttagtaccgccacccagagccataacattcctaacttctcata

SEQ ID NO: 966 - DNA, 53 bp, Artificial Sequence
  Staple strand_0965 of the 2D DNA nanostructure.
atttaccgggaaccagagccaccactgtagcgtaacattcctaacttctcata

SEQ ID NO: 967 - DNA, 51 bp, Artificial Sequence
  Staple strand_0966 of the 2D DNA nanostructure.
aaatcaccttccagtaagcgtcagtaataataacattcctaacttctcata

SEQ ID NO: 968 - DNA, 53 bp, Artificial Sequence
  Staple strand_0967 of the 2D DNA nanostructure.
cgttttcaagggagggaaggtaaagtttattttaacattcctaacttctcata

SEQ ID NO: 969 - DNA, 51 bp, Artificial Sequence
  Staple strand_0968 of the 2D DNA nanostructure.
accgattgtcggcattttcggtcataatcataacattcctaacttctcata

SEQ ID NO: 970 - DNA, 53 bp, Artificial Sequence
  Staple strand_0969 of the 2D DNA nanostructure.
tgtcacaatcttaccgaagccctttaatatcataacattcctaacttctcata

SEQ ID NO: 971 - DNA, 51 bp, Artificial Sequence
  Staple strand_0970 of the 2D DNA nanostructure.
aatagctatcaatagaaaattcaacattcataacattcctaacttctcata

SEQ ID NO: 972 - DNA, 53 bp, Artificial Sequence
  Staple strand_0971 of the 2D DNA nanostructure.
gagagatagagcgtctttccagaggttttgaataacattcctaacttctcata

SEQ ID NO: 973 - DNA, 51 bp, Artificial Sequence
  Staple strand_0972 of the 2D DNA nanostructure.
acgctaacacccacaagaattgaaaatagctaacattcctaacttctcata

SEQ ID NO: 974 - DNA, 53 bp, Artificial Sequence
  Staple strand_0973 of the 2D DNA nanostructure.
gccttaaaccaatcaataatcggcacgcgccttaacattcctaacttctcata

SEQ ID NO: 975 - DNA, 53 bp, Artificial Sequence
  Staple strand_0974 of the 2D DNA nanostructure.
taaatcgggattcccaattctgcgatataatgtaacattcctaacttctcata

SEQ ID NO: 976 - DNA, 51 bp, Artificial Sequence
  Staple strand_0975 of the 2D DNA nanostructure.
aacgcaaaatcgatgaacggtaccggttgataacattcctaacttctcata

SEQ ID NO: 977 - DNA, 53 bp, Artificial Sequence
  Staple strand_0976 of the 2D DNA nanostructure.
aacaagagggataaaaatttttagcataaagctaacattcctaacttctcata

SEQ ID NO: 978 - DNA, 51 bp, Artificial Sequence
  Staple strand_0977 of the 2D DNA nanostructure.
taatcagcggattgaccgtaatcgtaaccgtaacattcctaacttctcata

SEQ ID NO: 979 - DNA, 53 bp, Artificial Sequence
  Staple strand_0978 of the 2D DNA nanostructure.
acaaacggaaaagccccaaaaacactggagcataacattcctaacttctcata

SEQ ID NO: 980 - DNA, 51 bp, Artificial Sequence
  Staple strand_0979 of the 2D DNA nanostructure.
tgcatctttcccagtcacgacggcctgcagtaacattcctaacttctcata

SEQ ID NO: 981 - DNA, 53 bp, Artificial Sequence
  Staple strand_0980 of the 2D DNA nanostructure.
ccagggttgccagtttgaggggacccgtgggataacattcctaacttctcata

SEQ ID NO: 982 - DNA, 51 bp, Artificial Sequence
  Staple strand_0981 of the 2D DNA nanostructure.
gtcgacttcggccaacgcgcggggtttttctaacattcctaacttctcata

SEQ ID NO: 983 - DNA, 53 bp, Artificial Sequence
  Staple strand_0982 of the 2D DNA nanostructure.
ttaatgaactagaggatccccggggggtaacgtaacattcctaacttctcata

SEQ ID NO: 984 - DNA, 51 bp, Artificial Sequence
  Staple strand_0983 of the 2D DNA nanostructure.
ttttcactcaaagggcgaaaaaccatcacctaacattcctaacttctcata

SEQ ID NO: 985 - DNA, 53 bp, Artificial Sequence
  Staple strand_0984 of the 2D DNA nanostructure.
ctccaacgcagtgagacgggcaaccagctgcataacattcctaacttctcata

SEQ ID NO: 986 - DNA, 52 bp, Artificial Sequence
  Staple strand_0985 of the 2D DNA nanostructure.
caaatcaagttttttggggtcgaaacgtggataacattcctaacttctcata

SEQ ID NO: 987 - DNA, 51 bp, Artificial Sequence
  Staple strand_0986 of the 2D DNA nanostructure.
aaattaagttgaccattagatacttttgcgtaacattcctaacttctcata

SEQ ID NO: 988 - DNA, 53 bp, Artificial Sequence
  Staple strand_0987 of the 2D DNA nanostructure.
tatattttgtcattgcctgagagtggaagatttaacattcctaacttctcata

SEQ ID NO: 989 - DNA, 51 bp, Artificial Sequence
  Staple strand_0988 of the 2D DNA nanostructure.
gctatcagaaatgcaatgcctgaattagcataacattcctaacttctcata

SEQ ID NO: 990 - DNA, 53 bp, Artificial Sequence
  Staple strand_0989 of the 2D DNA nanostructure.
gtataagccaacccgtcggattctgacgacagtaacattcctaacttctcata

SEQ ID NO: 991 - DNA, 51 bp, Artificial Sequence
  Staple strand_0990 of the 2D DNA nanostructure.
gcgagtaaaaatatttaaattgttacaaagtaacattcctaacttctcata

SEQ ID NO: 992 - DNA, 53 bp, Artificial Sequence
  Staple strand_0991 of the 2D DNA nanostructure.
tatcggccgcaaggcgattaagtttaccgagctaacattcctaacttctcata

SEQ ID NO: 993 - DNA, 51 bp, Artificial Sequence
  Staple strand_0992 of the 2D DNA nanostructure.
gatgtgcttcaggaagatcgcacaatgtgataacattcctaacttctcata

SEQ ID NO: 994 - DNA, 53 bp, Artificial Sequence
  Staple strand_0993 of the 2D DNA nanostructure.
tcgaattcgggaaacctgtcgtgcagctgatttaacattcctaacttctcata

SEQ ID NO: 995 - DNA, 51 bp, Artificial Sequence
  Staple strand_0994 of the 2D DNA nanostructure.
ttccagtcgtaatcatggtcataaaaggggtaacattcctaacttctcata

SEQ ID NO: 996 - DNA, 53 bp, Artificial Sequence
  Staple strand_0995 of the 2D DNA nanostructure.
gcccttcagagtccactattaaagggtgccgttaacattcctaacttctcata

SEQ ID NO: 997 - DNA, 51 bp, Artificial Sequence
  Staple strand_0996 of the 2D DNA nanostructure.
tggaacaaccgcctggccctgaggcccgcttaacattcctaacttctcata

SEQ ID NO: 998 - DNA, 51 bp, Artificial Sequence
  Staple strand_0997 of the 2D DNA nanostructure.
aaagcactaaatcggaaccctaatccagtttaacattcctaacttctcata

SEQ ID NO: 999 - DNA, 53 bp, Artificial Sequence
  Staple strand_0998 of the 2D DNA nanostructure.
taaatcatataacctgtttagctaacctttaataacattcctaacttctcata

SEQ ID NO: 1000 - DNA, 53 bp, Artificial Sequence
  Staple strand_0999 of the 2D DNA nanostructure.
taggtaaactatttttgagagatcaaacgttataacattcctaacttctcata

SEQ ID NO: 1001 - DNA, 53 bp, Artificial Sequence
  Staple strand_1000 of the 2D DNA nanostructure.
gagggtaggattcaaaagggtgagacatccaataacattcctaacttctcata

SEQ ID NO: 1002 - DNA, 53 bp, Artificial Sequence
  Staple strand_1001 of the 2D DNA nanostructure.
atattttggctttcatcaacattatccagccataacattcctaacttctcata

SEQ ID NO: 1003 - DNA, 53 bp, Artificial Sequence
  Staple strand_1002 of the 2D DNA nanostructure.
tgtagccattaaaattcgcattaaatgccggataacattcctaacttctcata

SEQ ID NO: 1004 - DNA, 53 bp, Artificial Sequence
  Staple strand_1003 of the 2D DNA nanostructure.
gctttccgattacgccagctggcggctgtttctaacattcctaacttctcata

SEQ ID NO: 1005 - DNA, 53 bp, Artificial Sequence
  Staple strand_1004 of the 2D DNA nanostructure.
tcttcgctgcaccgcttctggtgcggccttcctaacattcctaacttctcata

SEQ ID NO: 1006 - DNA, 53 bp, Artificial Sequence
  Staple strand_1005 of the 2D DNA nanostructure.
ctgtgtgattgcgttgcgctcactagagttgctaacattcctaacttctcata

SEQ ID NO: 1007 - DNA, 53 bp, Artificial Sequence
  Staple strand_1006 of the 2D DNA nanostructure.
cacattaaaattgttatccgctcatgcgggcctaacattcctaacttctcata

SEQ ID NO: 1008 - DNA, 53 bp, Artificial Sequence
  Staple strand_1007 of the 2D DNA nanostructure.
agcaagcgtagggttgagtgttgtagggagcctaacattcctaacttctcata

SEQ ID NO: 1009 - DNA, 53 bp, Artificial Sequence
  Staple strand_1008 of the 2D DNA nanostructure.
gcccgagagtccacgctggtttgcagctaacttaacattcctaacttctcata

SEQ ID NO: 1010 - DNA, 53 bp, Artificial Sequence
  Staple strand_1009 of the 2D DNA nanostructure.
cccgatttagagcttgacggggaaaaagaatataacattcctaacttctcata

SEQ ID NO: 1011 - DNA, 53 bp, Artificial Sequence
  Staple strand_1010 of the 2D DNA nanostructure.
ttctactacgcgagctgaaaaggttaccgcgctaacattcctaacttctcata

SEQ ID NO: 1012 - DNA, 51 bp, Artificial Sequence
  Staple strand_1011 of the 2D DNA nanostructure.
gagacagctagctgataaattaatttttgttaacattcctaacttctcata

SEQ ID NO: 1013 - DNA, 53 bp, Artificial Sequence
  Staple strand_1012 of the 2D DNA nanostructure.
caaccgtttcaaatcaccatcaattcgagccataacattcctaacttctcata

SEQ ID NO: 1014 - DNA, 65 bp, Artificial Sequence
  Staple strand_1013 of the 2D DNA nanostructure.
taaatcaaaataattcgcgtctcggaaaccaggcaaagggaaggtaacattcctaacttctcata

SEQ ID NO: 1015 - DNA, 53 bp, Artificial Sequence
  Staple strand_1014 of the 2D DNA nanostructure.
gccatcaagctcattttttaaccacaaatccataacattcctaacttctcata

SEQ ID NO: 1016 - DNA, 53 bp, Artificial Sequence
  Staple strand_1015 of the 2D DNA nanostructure.
caactgttgcgccattcgccattcaaacatcataacattcctaacttctcata

SEQ ID NO: 1017 - DNA, 53 bp, Artificial Sequence
  Staple strand_1016 of the 2D DNA nanostructure.
aagcctggtacgagccggaagcatagatgatgtaacattcctaacttctcata

SEQ ID NO: 1018 - DNA, 59 bp, Artificial Sequence
  Staple strand_1017 of the 2D DNA nanostructure.
cccagcaggcgaaaaatcccttataaatcaagccggcgtaacattcctaacttctcata

SEQ ID NO: 1019 - DNA, 53 bp, Artificial Sequence
  Staple strand_1018 of the 2D DNA nanostructure.
tcggcaaatcctgtttgatggtggaccctcaataacattcctaacttctcata

SEQ ID NO: 1020 - DNA, 52 bp, Artificial Sequence
  Staple strand_1019 of the 2D DNA nanostructure.
aacgtggcgagaaaggaagggaaaccagtaataacattcctaacttctcata

SEQ ID NO: 1021 - DNA, 51 bp, Artificial Sequence
  Staple strand_1020 of the 2D DNA nanostructure.
ttttatttaagcaaatcagatattttttgttaacattcctaacttctcata

SEQ ID NO: 1022 - DNA, 53 bp, Artificial Sequence
  Staple strand_1021 of the 2D DNA nanostructure.
gtaataagttaggcagaggcatttatgatatttaacattcctaacttctcata

SEQ ID NO: 1023 - DNA, 51 bp, Artificial Sequence
  Staple strand_1022 of the 2D DNA nanostructure.
catgtaatagaatataaagtaccaagccgttaacattcctaacttctcata

SEQ ID NO: 1024 - DNA, 53 bp, Artificial Sequence
  Staple strand_1023 of the 2D DNA nanostructure.
atcgcaagtatgtaaatgctgatgataggaactaacattcctaacttctcata

SEQ ID NO: 1025 - DNA, 51 bp, Artificial Sequence
  Staple strand_1024 of the 2D DNA nanostructure.
tataactaacaaagaacgcgagaacgccaataacattcctaacttctcata

SEQ ID NO: 1026 - DNA, 53 bp, Artificial Sequence
  Staple strand_1025 of the 2D DNA nanostructure.
agaaaacaaagaagatgatgaaacaggctgcgtaacattcctaacttctcata

SEQ ID NO: 1027 - DNA, 51 bp, Artificial Sequence
  Staple strand_1026 of the 2D DNA nanostructure.
ctgagcaaaaattaattacattttgggttataacattcctaacttctcata

SEQ ID NO: 1028 - DNA, 53 bp, Artificial Sequence
  Staple strand_1027 of the 2D DNA nanostructure.
gcaattcacatattcctgattatcaaagtgtataacattcctaacttctcata

SEQ ID NO: 1029 - DNA, 51 bp, Artificial Sequence
  Staple strand_1028 of the 2D DNA nanostructure.
attatcattcaatataatcctgacaattactaacattcctaacttctcata

SEQ ID NO: 1030 - DNA, 53 bp, Artificial Sequence
  Staple strand_1029 of the 2D DNA nanostructure.
tcaatatcgaacctcaaatatcaattccgaaataacattcctaacttctcata

SEQ ID NO: 1031 - DNA, 51 bp, Artificial Sequence
  Staple strand_1030 of the 2D DNA nanostructure.
accttgcttggtcagttggcaaagagcggataacattcctaacttctcata

SEQ ID NO: 1032 - DNA, 51 bp, Artificial Sequence
  Staple strand_1031 of the 2D DNA nanostructure.
taaaagggacattctggccaacaaagcatctaacattcctaacttctcata

SEQ ID NO: 1033 - DNA, 53 bp, Artificial Sequence
  Staple strand_1032 of the 2D DNA nanostructure.
gtaccgcaattctaagaacgcgagtattattttaacattcctaacttctcata

SEQ ID NO: 1034 - DNA, 53 bp, Artificial Sequence
  Staple strand_1033 of the 2D DNA nanostructure.
gtaaagtaatcgccatatttaacaaaactttttaacattcctaacttctcata

SEQ ID NO: 1035 - DNA, 53 bp, Artificial Sequence
  Staple strand_1034 of the 2D DNA nanostructure.
aattgagaattctgtccagacgactaaaccaataacattcctaacttctcata

SEQ ID NO: 1036 - DNA, 53 bp, Artificial Sequence
  Staple strand_1035 of the 2D DNA nanostructure.
tcaaatataacctccggcttaggtaacaattttaacattcctaacttctcata

SEQ ID NO: 1037 - DNA, 53 bp, Artificial Sequence
  Staple strand_1036 of the 2D DNA nanostructure.
acctttttattttagttaatttcatagggctttaacattcctaacttctcata

SEQ ID NO: 1038 - DNA, 53 bp, Artificial Sequence
  Staple strand_1037 of the 2D DNA nanostructure.
catttgaaggcgaattattcatttttgtttggtaacattcctaacttctcata

SEQ ID NO: 1039 - DNA, 53 bp, Artificial Sequence
  Staple strand_1038 of the 2D DNA nanostructure.
cgcgcagattaccttttttaatgggagagacttaacattcctaacttctcata

SEQ ID NO: 1040 - DNA, 53 bp, Artificial Sequence
  Staple strand_1039 of the 2D DNA nanostructure.
attatactaagaaaccaccagaagtcaacagttaacattcctaacttctcata

SEQ ID NO: 1041 - DNA, 53 bp, Artificial Sequence
  Staple strand_1040 of the 2D DNA nanostructure.
gcggaacatctgaataatggaaggtacaaaattaacattcctaacttctcata

SEQ ID NO: 1042 - DNA, 53 bp, Artificial Sequence
  Staple strand_1041 of the 2D DNA nanostructure.
tgaaaggagcaaatgaaaaatctagagatagataacattcctaacttctcata

SEQ ID NO: 1043 - DNA, 53 bp, Artificial Sequence
  Staple strand_1042 of the 2D DNA nanostructure.
agccagcaattgaggaaggttatcatcatttttaacattcctaacttctcata

SEQ ID NO: 1044 - DNA, 53 bp, Artificial Sequence
  Staple strand_1043 of the 2D DNA nanostructure.
acccttctgacctgaaagcgtaagacgctgagtaacattcctaacttctcata

SEQ ID NO: 1045 - DNA, 53 bp, Artificial Sequence
  Staple strand_1044 of the 2D DNA nanostructure.
cttatcattcccgacttgcgggagcctaattttaacattcctaacttctcata

SEQ ID NO: 1046 - DNA, 51 bp, Artificial Sequence
  Staple strand_1045 of the 2D DNA nanostructure.
acaacatgccaacgctcaacagtcttctgataacattcctaacttctcata

SEQ ID NO: 1047 - DNA, 53 bp, Artificial Sequence
  Staple strand_1046 of the 2D DNA nanostructure.
agtataaagttcagctaatgcagatgtctttctaacattcctaacttctcata

SEQ ID NO: 1048 - DNA, 51 bp, Artificial Sequence
  Staple strand_1047 of the 2D DNA nanostructure.
cctaaatcaaaatcataggtctaaacagtataacattcctaacttctcata

SEQ ID NO: 1049 - DNA, 53 bp, Artificial Sequence
  Staple strand_1048 of the 2D DNA nanostructure.
gaatttatttaatggtttgaaatattcttacctaacattcctaacttctcata

SEQ ID NO: 1050 - DNA, 51 bp, Artificial Sequence
  Staple strand_1049 of the 2D DNA nanostructure.
cataaatctttgaataccaagtgttagaactaacattcctaacttctcata

SEQ ID NO: 1051 - DNA, 53 bp, Artificial Sequence
  Staple strand_1050 of the 2D DNA nanostructure.
cctgattgcaatatatgtgagtgatcaatagttaacattcctaacttctcata

SEQ ID NO: 1052 - DNA, 51 bp, Artificial Sequence
  Staple strand_1051 of the 2D DNA nanostructure.
ctaccatagtttgagtaacatttaaaatattaacattcctaacttctcata

SEQ ID NO: 1053 - DNA, 53 bp, Artificial Sequence
  Staple strand_1052 of the 2D DNA nanostructure.
attttaaaatcaaaattatttgcacggattcgtaacattcctaacttctcata

SEQ ID NO: 1054 - DNA, 51 bp, Artificial Sequence
  Staple strand_1053 of the 2D DNA nanostructure.
ctttagggcctgcaacagtgccaatacgtgtaacattcctaacttctcata

SEQ ID NO: 1055 - DNA, 53 bp, Artificial Sequence
  Staple strand_1054 of the 2D DNA nanostructure.
ttaacaccagcactaacaactaatcgttattataacattcctaacttctcata

SEQ ID NO: 1056 - DNA, 52 bp, Artificial Sequence
  Staple strand_1055 of the 2D DNA nanostructure.
gcacagacaatatttttgaatggggtcagtataacattcctaacttctcata

SEQ ID NO: 1057 - DNA, 51 bp, Artificial Sequence
  Staple strand_1056 of the 2D DNA nanostructure.
tgtagaaatcaagattagttgctcttaccataacattcctaacttctcata

SEQ ID NO: 1058 - DNA, 53 bp, Artificial Sequence
  Staple strand_1057 of the 2D DNA nanostructure.
gtttatcaatatgcgttatacaaaccgaccgttaacattcctaacttctcata

SEQ ID NO: 1059 - DNA, 51 bp, Artificial Sequence
  Staple strand_1058 of the 2D DNA nanostructure.
ttagtatcacaatagataagtccacgagcataacattcctaacttctcata

SEQ ID NO: 1060 - DNA, 53 bp, Artificial Sequence
  Staple strand_1059 of the 2D DNA nanostructure.
gtgataaaaagacgctgagaagagataacctttaacattcctaacttctcata

SEQ ID NO: 1061 - DNA, 51 bp, Artificial Sequence
  Staple strand_1060 of the 2D DNA nanostructure.
cttagatttaaggcgttaaataaagcctgttaacattcctaacttctcata

SEQ ID NO: 1062 - DNA, 53 bp, Artificial Sequence
  Staple strand_1061 of the 2D DNA nanostructure.
gcttctgttcgggagaaacaataacgtaaaactaacattcctaacttctcata

SEQ ID NO: 1063 - DNA, 51 bp, Artificial Sequence
  Staple strand_1062 of the 2D DNA nanostructure.
cttttacaaaatcgtcgctattagcgatagtaacattcctaacttctcata

SEQ ID NO: 1064 - DNA, 53 bp, Artificial Sequence
  Staple strand_1063 of the 2D DNA nanostructure.
agaaataaaaatcctttgcccgaaagattagataacattcctaacttctcata

SEQ ID NO: 1065 - DNA, 51 bp, Artificial Sequence
  Staple strand_1064 of the 2D DNA nanostructure.
ctcgtattagaaattgcgtagatacagtactaacattcctaacttctcata

SEQ ID NO: 1066 - DNA, 53 bp, Artificial Sequence
  Staple strand_1065 of the 2D DNA nanostructure.
gccgtcaaaaaacagaggtgaggcctattagttaacattcctaacttctcata

SEQ ID NO: 1067 - DNA, 51 bp, Artificial Sequence
  Staple strand_1066 of the 2D DNA nanostructure.
cagaagattagataatacatttgtcgacaataacattcctaacttctcata

SEQ ID NO: 1068 - DNA, 51 bp, Artificial Sequence
  Staple strand_1067 of the 2D DNA nanostructure.
ctttaatgcgcgaactgatagccccaccagtaacattcctaacttctcata

SEQ ID NO: 1069 - DNA, 52 bp, Artificial Sequence
  Staple strand_1068 of the 2D DNA nanostructure.
agaaaggaacaactaaaggaattcaaaaaaattaccatctctcctaaactcg

SEQ ID NO: 1070 - DNA, 51 bp, Artificial Sequence
  Staple strand_1069 of the 2D DNA nanostructure.
aggctccagaggctttgaggacacgggtaattaccatctctcctaaactcg

SEQ ID NO: 1071 - DNA, 53 bp, Artificial Sequence
  Staple strand_1070 of the 2D DNA nanostructure.
acggctacaaaaggagcctttaatgtgagaatttaccatctctcctaaactcg

SEQ ID NO: 1072 - DNA, 51 bp, Artificial Sequence
  Staple strand_1071 of the 2D DNA nanostructure.
aatacgtttgaaagaggacagactgaccttttaccatctctcctaaactcg

SEQ ID NO: 1073 - DNA, 53 bp, Artificial Sequence
  Staple strand_1072 of the 2D DNA nanostructure.
gaccaactaatgccactacgaagggggtagcattaccatctctcctaaactcg

SEQ ID NO: 1074 - DNA, 51 bp, Artificial Sequence
  Staple strand_1073 of the 2D DNA nanostructure.
catcaagtaaaacgaactaacgagttgagattaccatctctcctaaactcg

SEQ ID NO: 1075 - DNA, 53 bp, Artificial Sequence
  Staple strand_1074 of the 2D DNA nanostructure.
tacgttaaagtaatcttgacaagaaccgaactttaccatctctcctaaactcg

SEQ ID NO: 1076 - DNA, 51 bp, Artificial Sequence
  Staple strand_1075 of the 2D DNA nanostructure.
tttaggacaaatgctttaaacaatcaggtcttaccatctctcctaaactcg

SEQ ID NO: 1077 - DNA, 53 bp, Artificial Sequence
  Staple strand_1076 of the 2D DNA nanostructure.
atccccctataccacattcaactagaaaaatcttaccatctctcctaaactcg

SEQ ID NO: 1078 - DNA, 51 bp, Artificial Sequence
  Staple strand_1077 of the 2D DNA nanostructure.
tttaccccaacatgttttaaatttccatatttaccatctctcctaaactcg

SEQ ID NO: 1079 - DNA, 53 bp, Artificial Sequence
  Staple strand_1078 of the 2D DNA nanostructure.
ctgtagcttgactattatagtcagttcattgattaccatctctcctaaactcg

SEQ ID NO: 1080 - DNA, 51 bp, Artificial Sequence
  Staple strand_1079 of the 2D DNA nanostructure.
aacagttttgtaccaaaaacattttatttcttaccatctctcctaaactcg

SEQ ID NO: 1081 - DNA, 53 bp, Artificial Sequence
  Staple strand_1080 of the 2D DNA nanostructure.
acaactttcaacagtttcagcggatgtatcggttaccatctctcctaaactcg

SEQ ID NO: 1082 - DNA, 53 bp, Artificial Sequence
  Staple strand_1081 of the 2D DNA nanostructure.
tttatcaggacagcatcggaacgacaccaaccttaccatctctcctaaactcg

SEQ ID NO: 1083 - DNA, 51 bp, Artificial Sequence
  Staple strand_1082 of the 2D DNA nanostructure.
cagcgaaacttgctttcgaggtgttgctaattaccatctctcctaaactcg

SEQ ID NO: 1084 - DNA, 53 bp, Artificial Sequence
  Staple strand_1083 of the 2D DNA nanostructure.
taaaacgaggtcaatcataagggaaccggatattaccatctctcctaaactcg

SEQ ID NO: 1085 - DNA, 51 bp, Artificial Sequence
  Staple strand_1084 of the 2D DNA nanostructure.
gcgcagacaagaggcaaaagaatccctcagttaccatctctcctaaactcg

SEQ ID NO: 1086 - DNA, 53 bp, Artificial Sequence
  Staple strand_1085 of the 2D DNA nanostructure.
ttcattacgtcaggacgttgggaaatgcagatttaccatctctcctaaactcg

SEQ ID NO: 1087 - DNA, 51 bp, Artificial Sequence
  Staple strand_1086 of the 2D DNA nanostructure.
ttataccaccaaatcaacgtaacgaacgagttaccatctctcctaaactcg

SEQ ID NO: 1088 - DNA, 53 bp, Artificial Sequence
  Staple strand_1087 of the 2D DNA nanostructure.
acataacgggaatcgtcataaataaagcaaagttaccatctctcctaaactcg

SEQ ID NO: 1089 - DNA, 51 bp, Artificial Sequence
  Staple strand_1088 of the 2D DNA nanostructure.
aatactgcccaaaaggaattacgtggctcattaccatctctcctaaactcg

SEQ ID NO: 1090 - DNA, 53 bp, Artificial Sequence
  Staple strand_1089 of the 2D DNA nanostructure.
cggattgcagagcttaattgctgaaacgagtattaccatctctcctaaactcg

SEQ ID NO: 1091 - DNA, 51 bp, Artificial Sequence
  Staple strand_1090 of the 2D DNA nanostructure.
gatggcttatcaaaaagattaagagcgtccttaccatctctcctaaactcg

SEQ ID NO: 1092 - DNA, 53 bp, Artificial Sequence
  Staple strand_1091 of the 2D DNA nanostructure.
gatttagtcaataaagcctcagagaaccctcattaccatctctcctaaactcg

SEQ ID NO: 1093 - DNA, 53 bp, Artificial Sequence
  Staple strand_1092 of the 2D DNA nanostructure.
taaatgaattttctgtatgggattaatttcttttaccatctctcctaaactcg

SEQ ID NO: 1094 - DNA, 53 bp, Artificial Sequence
  Staple strand_1093 of the 2D DNA nanostructure.
aaacagctttttgcgggatcgtcaacactaaattaccatctctcctaaactcg

SEQ ID NO: 1095 - DNA, 53 bp, Artificial Sequence
  Staple strand_1094 of the 2D DNA nanostructure.
aaggccgctgataccgatagttgcgacgttagttaccatctctcctaaactcg

SEQ ID NO: 1096 - DNA, 53 bp, Artificial Sequence
  Staple strand_1095 of the 2D DNA nanostructure.
acactcatccatgttacttagccgaaagctgcttaccatctctcctaaactcg

SEQ ID NO: 1097 - DNA, 53 bp, Artificial Sequence
  Staple strand_1096 of the 2D DNA nanostructure.
gacctgctctttgacccccagcgagggagttattaccatctctcctaaactcg

SEQ ID NO: 1098 - DNA, 53 bp, Artificial Sequence
  Staple strand_1097 of the 2D DNA nanostructure.
tcattcagatgcgattttaagaacaggcatagttaccatctctcctaaactcg

SEQ ID NO: 1099 - DNA, 53 bp, Artificial Sequence
  Staple strand_1098 of the 2D DNA nanostructure.
attacctttgaataaggcttgcccaaatccgcttaccatctctcctaaactcg

SEQ ID NO: 1100 - DNA, 53 bp, Artificial Sequence
  Staple strand_1099 of the 2D DNA nanostructure.
taagagcaaatgtttagactggataggaagccttaccatctctcctaaactcg

SEQ ID NO: 1101 - DNA, 53 bp, Artificial Sequence
  Staple strand_1100 of the 2D DNA nanostructure.
aatagtaaacactatcataaccctcattgtgattaccatctctcctaaactcg

SEQ ID NO: 1102 - DNA, 53 bp, Artificial Sequence
  Staple strand_1101 of the 2D DNA nanostructure.
cgaaagactttgataagaggtcatatttcgcattaccatctctcctaaactcg

SEQ ID NO: 1103 - DNA, 53 bp, Artificial Sequence
  Staple strand_1102 of the 2D DNA nanostructure.
ttgctcctttcaaatatcgcgtttgagggggtttaccatctctcctaaactcg

SEQ ID NO: 1104 - DNA, 53 bp, Artificial Sequence
  Staple strand_1103 of the 2D DNA nanostructure.
aatggtcaacaggcaaggcaaagagtaatgtgttaccatctctcctaaactcg

SEQ ID NO: 1105 - DNA, 52 bp, Artificial Sequence
  Staple strand_1104 of the 2D DNA nanostructure.
tctaaagttttgtcgtctttccagccgacaattaccatctctcctaaactcg

SEQ ID NO: 1106 - DNA, 65 bp, Artificial Sequence
  Staple strand_1105 of the 2D DNA nanostructure.
tgacaactcgctgaggcttgcattataccaagcgcgatgataaattaccatctctcctaaactcg

SEQ ID NO: 1107 - DNA, 53 bp, Artificial Sequence
  Staple strand_1106 of the 2D DNA nanostructure.
atattcggaaccatcgcccacgcagagaaggattaccatctctcctaaactcg

SEQ ID NO: 1108 - DNA, 53 bp, Artificial Sequence
  Staple strand_1107 of the 2D DNA nanostructure.
tcatcgccaacaaagtacaacggacgccagcattaccatctctcctaaactcg

SEQ ID NO: 1109 - DNA, 53 bp, Artificial Sequence
  Staple strand_1108 of the 2D DNA nanostructure.
gatggtttgaacgagtagtaaatttaccattattaccatctctcctaaactcg

SEQ ID NO: 1110 - DNA, 59 bp, Artificial Sequence
  Staple strand_1109 of the 2D DNA nanostructure.
cgtttaccagacgacaaagaagttttgccataattcgattaccatctctcctaaactcg

SEQ ID NO: 1111 - DNA, 53 bp, Artificial Sequence
  Staple strand_1110 of the 2D DNA nanostructure.
cttttgcagataaaaaccaaaataaagactccttaccatctctcctaaactcg

SEQ ID NO: 1112 - DNA, 51 bp, Artificial Sequence
  Staple strand_1111 of the 2D DNA nanostructure.
gcttcaatcaggattagagagttattttcattaccatctctcctaaactcg

SEQ ID NO: 1113 - DNA, 53 bp, Artificial Sequence
  Staple strand_1112 of the 2D DNA nanostructure.
ccaacaggagcgaaccagaccggagcctttacttaccatctctcctaaactcg

SEQ ID NO: 1114 - DNA, 51 bp, Artificial Sequence
  Staple strand_1113 of the 2D DNA nanostructure.
tttggggatagtagtagcattaaaaggccgttaccatctctcctaaactcg

SEQ ID NO: 1115 - DNA, 53 bp, Artificial Sequence
  Staple strand_1114 of the 2D DNA nanostructure.
tccacagacagccctcatagttagcgtaacgattaccatctctcctaaactcg

SEQ ID NO: 1116 - DNA, 53 bp, Artificial Sequence
  Staple strand_1115 of the 2D DNA nanostructure.
ttaggattggctgagactcctcaataaccgatttaccatctctcctaaactcg

SEQ ID NO: 1117 - DNA, 51 bp, Artificial Sequence
  Staple strand_1116 of the 2D DNA nanostructure.
tattaagaagcggggttttgctcgtagcatttaccatctctcctaaactcg

SEQ ID NO: 1118 - DNA, 53 bp, Artificial Sequence
  Staple strand_1117 of the 2D DNA nanostructure.
ttgacaggccaccaccagagccgcgatttgtattaccatctctcctaaactcg

SEQ ID NO: 1119 - DNA, 51 bp, Artificial Sequence
  Staple strand_1118 of the 2D DNA nanostructure.
caccagaaaggttgaggcaggtcatgaaagttaccatctctcctaaactcg

SEQ ID NO: 1120 - DNA, 53 bp, Artificial Sequence
  Staple strand_1119 of the 2D DNA nanostructure.
gcaaggcctcaccagtagcaccatgggcttgattaccatctctcctaaactcg

SEQ ID NO: 1121 - DNA, 51 bp, Artificial Sequence
  Staple strand_1120 of the 2D DNA nanostructure.
cagcaaaaggaaacgtcaccaatgagccgcttaccatctctcctaaactcg

SEQ ID NO: 1122 - DNA, 53 bp, Artificial Sequence
  Staple strand_1121 of the 2D DNA nanostructure.
ttattacgaagaactggcatgattgcgagaggttaccatctctcctaaactcg

SEQ ID NO: 1123 - DNA, 51 bp, Artificial Sequence
  Staple strand_1122 of the 2D DNA nanostructure.
atacccaacagtatgttagcaaattagagcttaccatctctcctaaactcg

SEQ ID NO: 1124 - DNA, 53 bp, Artificial Sequence
  Staple strand_1123 of the 2D DNA nanostructure.
agagagaaaaaaatgaaaatagcaagcaaactttaccatctctcctaaactcg

SEQ ID NO: 1125 - DNA, 51 bp, Artificial Sequence
  Staple strand_1124 of the 2D DNA nanostructure.
ttaacgtctaacataaaaacaggtaacggattaccatctctcctaaactcg

SEQ ID NO: 1126 - DNA, 53 bp, Artificial Sequence
  Staple strand_1125 of the 2D DNA nanostructure.
ccaatagctcatcgtaggaatcatggcatcaattaccatctctcctaaactcg

SEQ ID NO: 1127 - DNA, 53 bp, Artificial Sequence
  Staple strand_1126 of the 2D DNA nanostructure.
tcaccagtacaaactacaacgcctagtaccagttaccatctctcctaaactcg

SEQ ID NO: 1128 - DNA, 53 bp, Artificial Sequence
  Staple strand_1127 of the 2D DNA nanostructure.
gcggataacctattattctgaaacagacgattttaccatctctcctaaactcg

SEQ ID NO: 1129 - DNA, 53 bp, Artificial Sequence
  Staple strand_1128 of the 2D DNA nanostructure.
tttcggaagtgccgtcgagagggtgagtttcgttaccatctctcctaaactcg

SEQ ID NO: 1130 - DNA, 53 bp, Artificial Sequence
  Staple strand_1129 of the 2D DNA nanostructure.
ggccttgaagagccaccaccctcagaaaccatttaccatctctcctaaactcg

SEQ ID NO: 1131 - DNA, 53 bp, Artificial Sequence
  Staple strand_1130 of the 2D DNA nanostructure.
ccaccctctattcacaaacaaatacctgcctattaccatctctcctaaactcg

SEQ ID NO: 1132 - DNA, 53 bp, Artificial Sequence
  Staple strand_1131 of the 2D DNA nanostructure.
cgatagcattgagccatttgggaacgtagaaattaccatctctcctaaactcg

SEQ ID NO: 1133 - DNA, 53 bp, Artificial Sequence
  Staple strand_1132 of the 2D DNA nanostructure.
tcaccgacgcaccgtaatcagtagcagaaccgttaccatctctcctaaactcg

SEQ ID NO: 1134 - DNA, 53 bp, Artificial Sequence
  Staple strand_1133 of the 2D DNA nanostructure.
atacataccgaggaaacgcaataagaagcgcattaccatctctcctaaactcg

SEQ ID NO: 1135 - DNA, 53 bp, Artificial Sequence
  Staple strand_1134 of the 2D DNA nanostructure.
aaggaaacataaaggtggcaacattatcaccgttaccatctctcctaaactcg

SEQ ID NO: 1136 - DNA, 53 bp, Artificial Sequence
  Staple strand_1135 of the 2D DNA nanostructure.
ttagacggccaaataagaaacgatagaaggctttaccatctctcctaaactcg

SEQ ID NO: 1137 - DNA, 53 bp, Artificial Sequence
  Staple strand_1136 of the 2D DNA nanostructure.
atcccaatgagaattaactgaacagttaccagttaccatctctcctaaactcg

SEQ ID NO: 1138 - DNA, 53 bp, Artificial Sequence
  Staple strand_1137 of the 2D DNA nanostructure.
tatccggtctcatcgagaacaagcgacaaaagttaccatctctcctaaactcg

SEQ ID NO: 1139 - DNA, 52 bp, Artificial Sequence
  Staple strand_1138 of the 2D DNA nanostructure.
aggaacccatgtaccgtaacacttgatataattaccatctctcctaaactcg

SEQ ID NO: 1140 - DNA, 51 bp, Artificial Sequence
  Staple strand_1139 of the 2D DNA nanostructure.
gtatagcaaacagttaatgcccaatcctcattaccatctctcctaaactcg

SEQ ID NO: 1141 - DNA, 53 bp, Artificial Sequence
  Staple strand_1140 of the 2D DNA nanostructure.
gcccgtatccggaataggtgtatcagcccaatttaccatctctcctaaactcg

SEQ ID NO: 1142 - DNA, 51 bp, Artificial Sequence
  Staple strand_1141 of the 2D DNA nanostructure.
ttaaagccagagccgccaccctcgacagaattaccatctctcctaaactcg

SEQ ID NO: 1143 - DNA, 53 bp, Artificial Sequence
  Staple strand_1142 of the 2D DNA nanostructure.
gcctccctcagaatggaaagcgcagtaacagtttaccatctctcctaaactcg

SEQ ID NO: 1144 - DNA, 51 bp, Artificial Sequence
  Staple strand_1143 of the 2D DNA nanostructure.
tcaagtttcattaaaggtgaatataaaagattaccatctctcctaaactcg

SEQ ID NO: 1145 - DNA, 53 bp, Artificial Sequence
  Staple strand_1144 of the 2D DNA nanostructure.
gaaattattgcctttagcgtcagaccggaaccttaccatctctcctaaactcg

SEQ ID NO: 1146 - DNA, 51 bp, Artificial Sequence
  Staple strand_1145 of the 2D DNA nanostructure.
aacgcaaagatagccgaacaaaccctgaacttaccatctctcctaaactcg

SEQ ID NO: 1147 - DNA, 53 bp, Artificial Sequence
  Staple strand_1146 of the 2D DNA nanostructure.
aagtaagcagacaccacggaataatattgacgttaccatctctcctaaactcg

SEQ ID NO: 1148 - DNA, 51 bp, Artificial Sequence
  Staple strand_1147 of the 2D DNA nanostructure.
aaagtcacaaaataaacagccagcgttttattaccatctctcctaaactcg

SEQ ID NO: 1149 - DNA, 53 bp, Artificial Sequence
  Staple strand_1148 of the 2D DNA nanostructure.
gccagttagagggtaattgagcgctttaagaattaccatctctcctaaactcg

SEQ ID NO: 1150 - DNA, 51 bp, Artificial Sequence
  Staple strand_1149 of the 2D DNA nanostructure.
gcgaacctccaagaacgggtatgacaataattaccatctctcctaaactcg

SEQ ID NO: 1151 - DNA, 53 bp, Artificial Sequence
  Staple strand_1150 of the 2D DNA nanostructure.
ccaccctcattttcagggatagcaaccgtactttaccatctctcctaaactcg

SEQ ID NO: 1152 - DNA, 53 bp, Artificial Sequence
  Staple strand_1151 of the 2D DNA nanostructure.
caggaggtggggtcagtgccttgagtctctgattaccatctctcctaaactcg

SEQ ID NO: 1153 - DNA, 51 bp, Artificial Sequence
  Staple strand_1152 of the 2D DNA nanostructure.
gttttaacttagtaccgccacccagagccattaccatctctcctaaactcg

SEQ ID NO: 1154 - DNA, 53 bp, Artificial Sequence
  Staple strand_1153 of the 2D DNA nanostructure.
atttaccgggaaccagagccaccactgtagcgttaccatctctcctaaactcg

SEQ ID NO: 1155 - DNA, 51 bp, Artificial Sequence
  Staple strand_1154 of the 2D DNA nanostructure.
aaatcaccttccagtaagcgtcagtaataattaccatctctcctaaactcg

SEQ ID NO: 1156 - DNA, 53 bp, Artificial Sequence
  Staple strand_1155 of the 2D DNA nanostructure.
cgttttcaagggagggaaggtaaagtttatttttaccatctctcctaaactcg

SEQ ID NO: 1157 - DNA, 51 bp, Artificial Sequence
  Staple strand_1156 of the 2D DNA nanostructure.
accgattgtcggcattttcggtcataatcattaccatctctcctaaactcg

SEQ ID NO: 1158 - DNA, 53 bp, Artificial Sequence
  Staple strand_1157 of the 2D DNA nanostructure.
tgtcacaatcttaccgaagccctttaatatcattaccatctctcctaaactcg

SEQ ID NO: 1159 - DNA, 51 bp, Artificial Sequence
  Staple strand_1158 of the 2D DNA nanostructure.
aatagctatcaatagaaaattcaacattcattaccatctctcctaaactcg

SEQ ID NO: 1160 - DNA, 53 bp, Artificial Sequence
  Staple strand_1159 of the 2D DNA nanostructure.
gagagatagagcgtctttccagaggttttgaattaccatctctcctaaactcg

SEQ ID NO: 1161 - DNA, 51 bp, Artificial Sequence
  Staple strand_1160 of the 2D DNA nanostructure.
acgctaacacccacaagaattgaaaatagcttaccatctctcctaaactcg

SEQ ID NO: 1162 - DNA, 53 bp, Artificial Sequence
  Staple strand_1161 of the 2D DNA nanostructure.
gccttaaaccaatcaataatcggcacgcgcctttaccatctctcctaaactcg

SEQ ID NO: 1163 - DNA, 53 bp, Artificial Sequence
  Staple strand_1162 of the 2D DNA nanostructure.
taaatcgggattcccaattctgcgatataatgttaccatctctcctaaactcg

SEQ ID NO: 1164 - DNA, 51 bp, Artificial Sequence
  Staple strand_1163 of the 2D DNA nanostructure.
aacgcaaaatcgatgaacggtaccggttgattaccatctctcctaaactcg

SEQ ID NO: 1165 - DNA, 53 bp, Artificial Sequence
  Staple strand_1164 of the 2D DNA nanostructure.
aacaagagggataaaaatttttagcataaagcttaccatctctcctaaactcg

SEQ ID NO: 1166 - DNA, 51 bp, Artificial Sequence
  Staple strand_1165 of the 2D DNA nanostructure.
taatcagcggattgaccgtaatcgtaaccgttaccatctctcctaaactcg

SEQ ID NO: 1167 - DNA, 53 bp, Artificial Sequence
  Staple strand_1166 of the 2D DNA nanostructure.
acaaacggaaaagccccaaaaacactggagcattaccatctctcctaaactcg

SEQ ID NO: 1168 - DNA, 51 bp, Artificial Sequence
  Staple strand_1167 of the 2D DNA nanostructure.
tgcatctttcccagtcacgacggcctgcagttaccatctctcctaaactcg

SEQ ID NO: 1169 - DNA, 53 bp, Artificial Sequence
  Staple strand_1168 of the 2D DNA nanostructure.
ccagggttgccagtttgaggggacccgtgggattaccatctctcctaaactcg

SEQ ID NO: 1170 - DNA, 51 bp, Artificial Sequence
  Staple strand_1169 of the 2D DNA nanostructure.
gtcgacttcggccaacgcgcggggtttttcttaccatctctcctaaactcg

SEQ ID NO: 1171 - DNA, 53 bp, Artificial Sequence
  Staple strand_1170 of the 2D DNA nanostructure.
ttaatgaactagaggatccccggggggtaacgttaccatctctcctaaactcg

SEQ ID NO: 1172 - DNA, 51 bp, Artificial Sequence
  Staple strand_1171 of the 2D DNA nanostructure.
ttttcactcaaagggcgaaaaaccatcaccttaccatctctcctaaactcg

SEQ ID NO: 1173 - DNA, 53 bp, Artificial Sequence
  Staple strand_1172 of the 2D DNA nanostructure.
ctccaacgcagtgagacgggcaaccagctgcattaccatctctcctaaactcg

SEQ ID NO: 1174 - DNA, 52 bp, Artificial Sequence
  Staple strand_1173 of the 2D DNA nanostructure.
caaatcaagttttttggggtcgaaacgtggattaccatctctcctaaactcg

SEQ ID NO: 1175 - DNA, 51 bp, Artificial Sequence
  Staple strand_1174 of the 2D DNA nanostructure.
aaattaagttgaccattagatacttttgcgttaccatctctcctaaactcg

SEQ ID NO: 1176 - DNA, 53 bp, Artificial Sequence
  Staple strand_1175 of the 2D DNA nanostructure.
tatattttgtcattgcctgagagtggaagattttaccatctctcctaaactcg

SEQ ID NO: 1177 - DNA, 51 bp, Artificial Sequence
  Staple strand_1176 of the 2D DNA nanostructure.
gctatcagaaatgcaatgcctgaattagcattaccatctctcctaaactcg

SEQ ID NO: 1178 - DNA, 53 bp, Artificial Sequence
  Staple strand_1177 of the 2D DNA nanostructure.
gtataagccaacccgtcggattctgacgacagttaccatctctcctaaactcg

SEQ ID NO: 1179 - DNA, 51 bp, Artificial Sequence
  Staple strand_1178 of the 2D DNA nanostructure.
gcgagtaaaaatatttaaattgttacaaagttaccatctctcctaaactcg

SEQ ID NO: 1180 - DNA, 53 bp, Artificial Sequence
  Staple strand_1179 of the 2D DNA nanostructure.
tatcggccgcaaggcgattaagtttaccgagcttaccatctctcctaaactcg

SEQ ID NO: 1181 - DNA, 51 bp, Artificial Sequence
  Staple strand_1180 of the 2D DNA nanostructure.
gatgtgcttcaggaagatcgcacaatgtgattaccatctctcctaaactcg

SEQ ID NO: 1182 - DNA, 53 bp, Artificial Sequence
  Staple strand_1181 of the 2D DNA nanostructure.
tcgaattcgggaaacctgtcgtgcagctgattttaccatctctcctaaactcg

SEQ ID NO: 1183 - DNA, 51 bp, Artificial Sequence
  Staple strand_1182 of the 2D DNA nanostructure.
ttccagtcgtaatcatggtcataaaaggggttaccatctctcctaaactcg

SEQ ID NO: 1184 - DNA, 53 bp, Artificial Sequence
  Staple strand_1183 of the 2D DNA nanostructure.
gcccttcagagtccactattaaagggtgccgtttaccatctctcctaaactcg

SEQ ID NO: 1185 - DNA, 51 bp, Artificial Sequence
  Staple strand_1184 of the 2D DNA nanostructure.
tggaacaaccgcctggccctgaggcccgctttaccatctctcctaaactcg

SEQ ID NO: 1186 - DNA, 51 bp, Artificial Sequence
  Staple strand_1185 of the 2D DNA nanostructure.
aaagcactaaatcggaaccctaatccagttttaccatctctcctaaactcg

SEQ ID NO: 1187 - DNA, 53 bp, Artificial Sequence
  Staple strand_1186 of the 2D DNA nanostructure.
taaatcatataacctgtttagctaacctttaattaccatctctcctaaactcg

SEQ ID NO: 1188 - DNA, 53 bp, Artificial Sequence
  Staple strand_1187 of the 2D DNA nanostructure.
taggtaaactatttttgagagatcaaacgttattaccatctctcctaaactcg

SEQ ID NO: 1189 - DNA, 53 bp, Artificial Sequence
  Staple strand_1188 of the 2D DNA nanostructure.
gagggtaggattcaaaagggtgagacatccaattaccatctctcctaaactcg

SEQ ID NO: 1190 - DNA, 53 bp, Artificial Sequence
  Staple strand_1189 of the 2D DNA nanostructure.
atattttggctttcatcaacattatccagccattaccatctctcctaaactcg

SEQ ID NO: 1191 - DNA, 53 bp, Artificial Sequence
  Staple strand_1190 of the 2D DNA nanostructure.
tgtagccattaaaattcgcattaaatgccggattaccatctctcctaaactcg

SEQ ID NO: 1192 - DNA, 53 bp, Artificial Sequence
  Staple strand_1191 of the 2D DNA nanostructure.
gctttccgattacgccagctggcggctgtttcttaccatctctcctaaactcg

SEQ ID NO: 1193 - DNA, 53 bp, Artificial Sequence
  Staple strand_1192 of the 2D DNA nanostructure.
tcttcgctgcaccgcttctggtgcggccttccttaccatctctcctaaactcg

SEQ ID NO: 1194 - DNA, 53 bp, Artificial Sequence
  Staple strand_1193 of the 2D DNA nanostructure.
ctgtgtgattgcgttgcgctcactagagttgcttaccatctctcctaaactcg

SEQ ID NO: 1195 - DNA, 53 bp, Artificial Sequence
  Staple strand_1194 of the 2D DNA nanostructure.
cacattaaaattgttatccgctcatgcgggccttaccatctctcctaaactcg

SEQ ID NO: 1196 - DNA, 53 bp, Artificial Sequence
  Staple strand_1195 of the 2D DNA nanostructure.
agcaagcgtagggttgagtgttgtagggagccttaccatctctcctaaactcg

SEQ ID NO: 1197 - DNA, 53 bp, Artificial Sequence
  Staple strand_1196 of the 2D DNA nanostructure.
gcccgagagtccacgctggtttgcagctaactttaccatctctcctaaactcg

SEQ ID NO: 1198 - DNA, 53 bp, Artificial Sequence
  Staple strand_1197 of the 2D DNA nanostructure.
cccgatttagagcttgacggggaaaaagaatattaccatctctcctaaactcg

SEQ ID NO: 1199 - DNA, 53 bp, Artificial Sequence
  Staple strand_1198 of the 2D DNA nanostructure.
ttctactacgcgagctgaaaaggttaccgcgcttaccatctctcctaaactcg

SEQ ID NO: 1200 - DNA, 51 bp, Artificial Sequence
  Staple strand_1199 of the 2D DNA nanostructure.
gagacagctagctgataaattaatttttgtttaccatctctcctaaactcg

SEQ ID NO: 1201 - DNA, 53 bp, Artificial Sequence
  Staple strand_1200 of the 2D DNA nanostructure.
caaccgtttcaaatcaccatcaattcgagccattaccatctctcctaaactcg

SEQ ID NO: 1202 - DNA, 65 bp, Artificial Sequence
  Staple strand_1201 of the 2D DNA nanostructure.
taaatcaaaataattcgcgtctcggaaaccaggcaaagggaaggttaccatctctcctaaactcg

SEQ ID NO: 1203 - DNA, 53 bp, Artificial Sequence
  Staple strand_1202 of the 2D DNA nanostructure.
gccatcaagctcattttttaaccacaaatccattaccatctctcctaaactcg

SEQ ID NO: 1204 - DNA, 53 bp, Artificial Sequence
  Staple strand_1203 of the 2D DNA nanostructure.
caactgttgcgccattcgccattcaaacatcattaccatctctcctaaactcg

SEQ ID NO: 1205 - DNA, 53 bp, Artificial Sequence
  Staple strand_1204 of the 2D DNA nanostructure.
aagcctggtacgagccggaagcatagatgatgttaccatctctcctaaactcg

SEQ ID NO: 1206 - DNA, 59 bp, Artificial Sequence
  Staple strand_1205 of the 2D DNA nanostructure.
cccagcaggcgaaaaatcccttataaatcaagccggcgttaccatctctcctaaactcg

SEQ ID NO: 1207 - DNA, 53 bp, Artificial Sequence
  Staple strand_1206 of the 2D DNA nanostructure.
tcggcaaatcctgtttgatggtggaccctcaattaccatctctcctaaactcg

SEQ ID NO: 1208 - DNA, 52 bp, Artificial Sequence
  Staple strand_1207 of the 2D DNA nanostructure.
aacgtggcgagaaaggaagggaaaccagtaattaccatctctcctaaactcg

SEQ ID NO: 1209 - DNA, 51 bp, Artificial Sequence
  Staple strand_1208 of the 2D DNA nanostructure.
ttttatttaagcaaatcagatattttttgtttaccatctctcctaaactcg

SEQ ID NO: 1210 - DNA, 53 bp, Artificial Sequence
  Staple strand_1209 of the 2D DNA nanostructure.
gtaataagttaggcagaggcatttatgatattttaccatctctcctaaactcg

SEQ ID NO: 1211 - DNA, 51 bp, Artificial Sequence
  Staple strand_1210 of the 2D DNA nanostructure.
catgtaatagaatataaagtaccaagccgtttaccatctctcctaaactcg

SEQ ID NO: 1212 - DNA, 53 bp, Artificial Sequence
  Staple strand_1211 of the 2D DNA nanostructure.
atcgcaagtatgtaaatgctgatgataggaacttaccatctctcctaaactcg

SEQ ID NO: 1213 - DNA, 51 bp, Artificial Sequence
  Staple strand_1212 of the 2D DNA nanostructure.
tataactaacaaagaacgcgagaacgccaattaccatctctcctaaactcg

SEQ ID NO: 1214 - DNA, 53 bp, Artificial Sequence
  Staple strand_1213 of the 2D DNA nanostructure.
agaaaacaaagaagatgatgaaacaggctgcgttaccatctctcctaaactcg

SEQ ID NO: 1215 - DNA, 51 bp, Artificial Sequence
  Staple strand_1214 of the 2D DNA nanostructure.
ctgagcaaaaattaattacattttgggttattaccatctctcctaaactcg

SEQ ID NO: 1216 - DNA, 53 bp, Artificial Sequence
  Staple strand_1215 of the 2D DNA nanostructure.
gcaattcacatattcctgattatcaaagtgtattaccatctctcctaaactcg

SEQ ID NO: 1217 - DNA, 51 bp, Artificial Sequence
  Staple strand_1216 of the 2D DNA nanostructure.
attatcattcaatataatcctgacaattacttaccatctctcctaaactcg

SEQ ID NO: 1218 - DNA, 53 bp, Artificial Sequence
  Staple strand_1217 of the 2D DNA nanostructure.
tcaatatcgaacctcaaatatcaattccgaaattaccatctctcctaaactcg

SEQ ID NO: 1219 - DNA, 51 bp, Artificial Sequence
  Staple strand_1218 of the 2D DNA nanostructure.
accttgcttggtcagttggcaaagagcggattaccatctctcctaaactcg

SEQ ID NO: 1220 - DNA, 51 bp, Artificial Sequence
  Staple strand_1219 of the 2D DNA nanostructure.
taaaagggacattctggccaacaaagcatcttaccatctctcctaaactcg

SEQ ID NO: 1221 - DNA, 53 bp, Artificial Sequence
  Staple strand_1220 of the 2D DNA nanostructure.
gtaccgcaattctaagaacgcgagtattatttttaccatctctcctaaactcg

SEQ ID NO: 1222 - DNA, 53 bp, Artificial Sequence
  Staple strand_1221 of the 2D DNA nanostructure.
gtaaagtaatcgccatatttaacaaaacttttttaccatctctcctaaactcg

SEQ ID NO: 1223 - DNA, 53 bp, Artificial Sequence
  Staple strand_1222 of the 2D DNA nanostructure.
aattgagaattctgtccagacgactaaaccaattaccatctctcctaaactcg

SEQ ID NO: 1224 - DNA, 53 bp, Artificial Sequence
  Staple strand_1223 of the 2D DNA nanostructure.
tcaaatataacctccggcttaggtaacaatttttaccatctctcctaaactcg

SEQ ID NO: 1225 - DNA, 53 bp, Artificial Sequence
  Staple strand_1224 of the 2D DNA nanostructure.
acctttttattttagttaatttcatagggcttttaccatctctcctaaactcg

SEQ ID NO: 1226 - DNA, 53 bp, Artificial Sequence
  Staple strand_1225 of the 2D DNA nanostructure.
catttgaaggcgaattattcatttttgtttggttaccatctctcctaaactcg

SEQ ID NO: 1227 - DNA, 53 bp, Artificial Sequence
  Staple strand_1226 of the 2D DNA nanostructure.
cgcgcagattaccttttttaatgggagagactttaccatctctcctaaactcg

SEQ ID NO: 1228 - DNA, 53 bp, Artificial Sequence
  Staple strand_1227 of the 2D DNA nanostructure.
attatactaagaaaccaccagaagtcaacagtttaccatctctcctaaactcg

SEQ ID NO: 1229 - DNA, 53 bp, Artificial Sequence
  Staple strand_1228 of the 2D DNA nanostructure.
gcggaacatctgaataatggaaggtacaaaatttaccatctctcctaaactcg

SEQ ID NO: 1230 - DNA, 53 bp, Artificial Sequence
  Staple strand_1229 of the 2D DNA nanostructure.
tgaaaggagcaaatgaaaaatctagagatagattaccatctctcctaaactcg

SEQ ID NO: 1231 - DNA, 53 bp, Artificial Sequence
  Staple strand_1230 of the 2D DNA nanostructure.
agccagcaattgaggaaggttatcatcattttttaccatctctcctaaactcg

SEQ ID NO: 1232 - DNA, 53 bp, Artificial Sequence
  Staple strand_1231 of the 2D DNA nanostructure.
acccttctgacctgaaagcgtaagacgctgagttaccatctctcctaaactcg

SEQ ID NO: 1233 - DNA, 53 bp, Artificial Sequence
  Staple strand_1232 of the 2D DNA nanostructure.
cttatcattcccgacttgcgggagcctaatttttaccatctctcctaaactcg

SEQ ID NO: 1234 - DNA, 51 bp, Artificial Sequence
  Staple strand_1233 of the 2D DNA nanostructure.
acaacatgccaacgctcaacagtcttctgattaccatctctcctaaactcg

SEQ ID NO: 1235 - DNA, 53 bp, Artificial Sequence
  Staple strand_1234 of the 2D DNA nanostructure.
agtataaagttcagctaatgcagatgtctttcttaccatctctcctaaactcg

SEQ ID NO: 1236 - DNA, 51 bp, Artificial Sequence
  Staple strand_1235 of the 2D DNA nanostructure.
cctaaatcaaaatcataggtctaaacagtattaccatctctcctaaactcg

SEQ ID NO: 1237 - DNA, 53 bp, Artificial Sequence
  Staple strand_1236 of the 2D DNA nanostructure.
gaatttatttaatggtttgaaatattcttaccttaccatctctcctaaactcg

SEQ ID NO: 1238 - DNA, 51 bp, Artificial Sequence
  Staple strand_1237 of the 2D DNA nanostructure.
cataaatctttgaataccaagtgttagaacttaccatctctcctaaactcg

SEQ ID NO: 1239 - DNA, 53 bp, Artificial Sequence
  Staple strand_1238 of the 2D DNA nanostructure.
cctgattgcaatatatgtgagtgatcaatagtttaccatctctcctaaactcg

SEQ ID NO: 1240 - DNA, 51 bp, Artificial Sequence
  Staple strand_1239 of the 2D DNA nanostructure.
ctaccatagtttgagtaacatttaaaatatttaccatctctcctaaactcg

SEQ ID NO: 1241 - DNA, 53 bp, Artificial Sequence
  Staple strand_1240 of the 2D DNA nanostructure.
attttaaaatcaaaattatttgcacggattcgttaccatctctcctaaactcg

SEQ ID NO: 1242 - DNA, 51 bp, Artificial Sequence
  Staple strand_1241 of the 2D DNA nanostructure.
ctttagggcctgcaacagtgccaatacgtgttaccatctctcctaaactcg

SEQ ID NO: 1243 - DNA, 53 bp, Artificial Sequence
  Staple strand_1242 of the 2D DNA nanostructure.
ttaacaccagcactaacaactaatcgttattattaccatctctcctaaactcg

SEQ ID NO: 1244 - DNA, 52 bp, Artificial Sequence
  Staple strand_1243 of the 2D DNA nanostructure.
gcacagacaatatttttgaatggggtcagtattaccatctctcctaaactcg

SEQ ID NO: 1245 - DNA, 51 bp, Artificial Sequence
  Staple strand_1244 of the 2D DNA nanostructure.
tgtagaaatcaagattagttgctcttaccattaccatctctcctaaactcg

SEQ ID NO: 1246 - DNA, 53 bp, Artificial Sequence
  Staple strand_1245 of the 2D DNA nanostructure.
gtttatcaatatgcgttatacaaaccgaccgtttaccatctctcctaaactcg

SEQ ID NO: 1247 - DNA, 51 bp, Artificial Sequence
  Staple strand_1246 of the 2D DNA nanostructure.
ttagtatcacaatagataagtccacgagcattaccatctctcctaaactcg

SEQ ID NO: 1248 - DNA, 53 bp, Artificial Sequence
  Staple strand_1247 of the 2D DNA nanostructure.
gtgataaaaagacgctgagaagagataaccttttaccatctctcctaaactcg

SEQ ID NO: 1249 - DNA, 51 bp, Artificial Sequence
  Staple strand_1248 of the 2D DNA nanostructure.
cttagatttaaggcgttaaataaagcctgtttaccatctctcctaaactcg

SEQ ID NO: 1250 - DNA, 53 bp, Artificial Sequence
  Staple strand_1249 of the 2D DNA nanostructure.
gcttctgttcgggagaaacaataacgtaaaacttaccatctctcctaaactcg

SEQ ID NO: 1251 - DNA, 51 bp, Artificial Sequence
  Staple strand_1250 of the 2D DNA nanostructure.
cttttacaaaatcgtcgctattagcgatagttaccatctctcctaaactcg

SEQ ID NO: 1252 - DNA, 53 bp, Artificial Sequence
  Staple strand_1251 of the 2D DNA nanostructure.
agaaataaaaatcctttgcccgaaagattagattaccatctctcctaaactcg

SEQ ID NO: 1253 - DNA, 51 bp, Artificial Sequence
  Staple strand_1252 of the 2D DNA nanostructure.
ctcgtattagaaattgcgtagatacagtacttaccatctctcctaaactcg

SEQ ID NO: 1254 - DNA, 53 bp, Artificial Sequence
  Staple strand_1253 of the 2D DNA nanostructure.
gccgtcaaaaaacagaggtgaggcctattagtttaccatctctcctaaactcg

SEQ ID NO: 1255 - DNA, 51 bp, Artificial Sequence
  Staple strand_1254 of the 2D DNA nanostructure.
cagaagattagataatacatttgtcgacaattaccatctctcctaaactcg

SEQ ID NO: 1256 - DNA, 51 bp, Artificial Sequence
  Staple strand_1255 of the 2D DNA nanostructure.
ctttaatgcgcgaactgatagccccaccagttaccatctctcctaaactcg

SEQ ID NO: 1257 - DNA, 7249 bp, Phage M13mp18
  scaffold DNA p7249_tillibit.txt
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 1258 - DNA, 7308 bp, Phage M13mp18
  scaffold DNA p7308_NatChem2012.txt
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatccttatacgggtactagccatgcgtatacggtcgctagcggacttgcctcgctatcaaaggtctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 1259 - DNA, 21 bp, Artificial Sequence
  DNA dye adapter red, 3′ modification Atto 647N
gtgatgtaggtggtagaggaa

SEQ ID NO: 1260 - DNA, 21 bp, Artificial Sequence
  DNA dye adapter green, 3′ modification Atto 565
tatgagaagttaggaatgtta

SEQ ID NO: 1261 - DNA, 21 bp, Artificial Sequence
  DNA dye adapter blue, 3′ modification Atto 488
cgagtttaggagagatggtaa

SEQ ID NO: 1262 - DNA, 21 bp, Artificial Sequence
  DNA carrier adapter and/or intermediate carrier adapter, 5′ modification biotin
gaatcggtcacagtacaaccg

SEQ ID NO: 1263 - DNA, 68 bp, Artificial Sequence
  DNA T1 target adapter 3D
taattttaaaagtctcgtatgtcagtattaacaccaccagcagccttgagacgatgttgaccttaacc

SEQ ID NO: 1264 - DNA, 68 bp, Artificial Sequence
  DNA T2 target adapter 3D
taattttaaaagtctcgtatgtcagtattaacaccaccagcagccttcatgtcaggagattttcagcc

SEQ ID NO: 1265 - DNA, 68 bp, Artificial Sequence
  DNA T3 target adapter 3D
taattttaaaagtctcgtatgtcagtattaacaccaccagcagcctttaccctatctgagtgagtagc

SEQ ID NO: 1266 - DNA, 61 bp, Artificial Sequence
  DNA T1 target adapter 2D
ttgtgtcgtgacgagaaacaccaaatttcaactttaatttgagacgatgttgaccttaacc

SEQ ID NO: 1267 - DNA, 61 bp, Artificial Sequence
  DNA T2 target adapter 2D
ttgtgtcgtgacgagaaacaccaaatttcaactttaatttcatgtcaggagattttcagcc

SEQ ID NO: 1268 - DNA, 61 bp, Artificial Sequence
  DNA T3 target adapter 2D
ttgtgtcgtgacgagaaacaccaaatttcaactttaattttaccctatctgagtgagtagc

SEQ ID NO: 1269 - DNA, 87 bp, Artificial Sequence
  DNA target structure
gctactcactcagatagggtatggctgaaaatctcctgacatgtggttaaggtcaacatcgtctctcggttgtactgtgaccgattc

SEQ ID NO: 1270 - DNA, 72 bp, Artificial Sequence
  DNA T4_gapdh target adapter 3D
ttgtgtcgtgacgagaaacaccaaatttcaacttttttttccattgatgacaagcttcccgttctcagcctt

SEQ ID NO: 1271 - DNA, 73 bp, Artificial Sequence
  DNA T4_tfrc target adapter 3D
ttgtgtcgtgacgagaaacaccaaatttcaactttttcgagttttgagcgctgtctttgacctgaatcttaac

SEQ ID NO: 1272 - DNA, 70 bp, Artificial Sequence
  DNA T4_actb target adapter 3D
ttgtgtcgtgacgagaaacaccaaatttcaactttttaatgatcttgatcttcattgtgctgggtgccag

SEQ ID NO: 1273 - DNA, 57 bp, Artificial Sequence
  DNA carrier adapter gapdh
gccatgggtggaatcatattggaacatgtaaaccttcggttgtactgtgaccgattc

SEQ ID NO: 1274 - DNA, 54 bp, Artificial Sequence
  DNA carrier adapter tfrc
cacgccagactttgctgagtttaaattcacgttcggttgtactgtgaccgattc

SEQ ID NO: 1275 - DNA, 58 bp, Artificial Sequence
  DNA carrier adapter actb
atgatggagttgaaggtagtttcgtggatgccacattcggttgtactgtgaccgattc

SEQ ID NO: 1276 - DNA, 76 bp, Artificial Sequence
  DNA T5_gapdh target adapter 3D
ttgtgtcgtgacgagaaacaccaaatttcaacttttctgggtggcagtgatggcatggactgtggtcatgagtcct

SEQ ID NO: 1277 - DNA, 74 bp, Artificial Sequence
  DNA T5_tfrc target Adapter 3D
ttgtgtcgtgacgagaaacaccaaatttcaactttttacccatcttttaagaccatatctgagaacatctgggc

SEQ ID NO: 1278 - DNA, 70 bp, Artificial Sequence
  DNA T5_actb target adapter 3D
ttgtgtcgtgacgagaaacaccaaatttcaactttttcagtgtacaggtaagccctggctgcctccaccc